(19) United States
(12) Patent Application Publication
(10) Pub. No.: US 2026/0117223 A1
(43) Pub. Date: Apr. 30, 2026

(54) NUCLEIC ACIDS AND USES THEREOF

(71) Applicant: PETER MACCALLUM CANCER INSTITUTE, Melbourne (AU)

(72) Inventors: Mohamed FAREH, Maribyrnong (AU); Wenxin HU, North Melbourne (AU); Paul Gerald EKERT, Elsternwick (AU); Carolyn Elizabeth SHEMBREY, North Melbourne (AU); Joseph Albert TRAPANI, Camberwell (AU)

(21) Appl. No.: 18/860,400

(22) PCT Filed: Apr. 26, 2023
(86) PCT No.: PCT/AU2023/050339

§ 371 Date: Oct. 25, 2024

(30) Foreign Application Priority Data
Apr. 26, 2022 (AU) 2022901093

(51) Int. Cl. C12N 15/11; C12N 9/22; C12N 15/10; C12N 15/52

(52) U.S. Cl. CPC C12N 15/111; C12N 9/226; C12N 15/1089; C12N 15/52; C12N 2310/20

(57) ABSTRACT

The present disclosure relates generally to (CRISPR) RNA (crRNA) for the precision silencing of transcripts. In some embodiments, the crRNA are enriched for guanosine (G) nucleotides at key spacer positions, which is useful in enhancing the silencing efficacy of otherwise inefficient crRNA, thereby expanding the targeting spectrum of Cas13 endonucleases, e.g., Cas13b and Cas13d. In other embodiments, the crRNA comprise a spacer sequence having at least one nucleotide mismatch relative to the target RNA sequence, wherein the target RNA sequence is a wild-type transcript and/or a variant transcript (e.g., a transcript comprising a single nucleotide variant (SNV)). The present disclosure also provides RNA editing systems comprising the crRNA described herein in complex a Cas13 effector protein and a target RNA sequence, methods for the selective targeting of transcripts encoding proteins that are difficult to target, or are not amenable to pharmacological targeting, e.g., oncogenic fusion transcripts or oncogenic transcripts comprising single nucleotide variant(s), and methods for the design and selection of potent crRNA.

### RELATED APPLICATIONS

This application claims priority from Australian Provisional Patent Application No. 2022901093 filed on 26 Apr. 2022, the entire content of which is hereby incorporated by reference.

### STATEMENT REGARDING SEQUENCE LISTING

This application contains a Sequence Listing, which has been submitted electronically in xml format and is hereby incorporated by reference in its entirety. Said xml copy, created on Jan. 7, 2026, is named FINAL-PCT-Sequence-Listing.xml and is 574,215 bytes in size.

### FIELD

The present disclosure relates generally to (CRISPR) RNA (crRNA) for the precision silencing of transcripts. In some embodiments, the crRNA are enriched for guanosine (G) nucleotides at key spacer positions, which is useful in enhancing the silencing efficacy of otherwise inefficient crRNA, thereby expanding the targeting spectrum of Cas13 endonucleases, e.g., Cas13b and Cas13d. In other embodiments, the crRNA comprise a spacer sequence having at least one nucleotide mismatch relative to the target RNA sequence, wherein the target RNA sequence is a wild-type transcript and/or a variant transcript (e.g., a transcript comprising a single nucleotide variant (SNV)). The present disclosure also provides RNA editing systems comprising the crRNA described herein in complex a Cas13 effector protein and a target RNA sequence, methods for the selective targeting of transcripts encoding proteins that are difficult to target, or are not amenable to pharmacological targeting, e.g., oncogenic fusion transcripts or oncogenic transcripts comprising single nucleotide variant(s), and methods for the design and selection of potent crRNA.

### BACKGROUND

CRISPR (clustered regularly interspaced short palindromic repeats) systems endow bacteria with adaptive immunity against invading pathogens through sequence-specific recognition and cleavage of foreign nucleic acids. The type VI CRISPR effectors termed CRISPR-Cas13 (Cas13) are programmable RNA-guided targeting enzymes that exclusively degrade single-stranded RNAs (ssRNAs) with high efficacy and specificity. Cas13 systems have been deployed in a variety of applications including RNA knockdown (Abudayyeh et al., 2017, Nature, 550: 280-284), nucleic-acid detection (Gootenberg et al., 2017, Science, 356: 438-442), precise RNA base editing (Cox et al., 2017, Science, 358), live-cell RNA imaging (Yang et al., 2019, Molecular Cell, 76: 981-997), and viral suppression (Blanchard et al., 2021, Nature Biotechnology, 39: 717-726). The target recognition process of Cas13 is guided by a single CRISPR RNA (crRNA) consisting of a direct repeat (DR) and a programmable spacer sequence. The DR sequence forms a highly ordered stem-loop structure that facilitates crRNA loading into Cas13 protein, whereas the spacer sequence mediates RNA target recognition through RNA-RNA base pairing. The efficiency and reversibility of RNA targeting with Cas13 represents a promising modality to specifically edit coding and non-coding transcriptomes without risking permanent alteration of the genome, which is an inherent limitation of DNA-editing CRISPR enzymes. Compared to classical eukaryotic RNA interference (RNAi), RNA knockdown with Cas13 in mammalian cells consistently demonstrates superior specificity, attributable to its extended spacer sequence. Therefore, Cas13 is highly attractive for targeting aberrant transcripts that drive various human genetic diseases, e.g., cancer.

Recent advances in next-generation sequencing enable rapid identification of targetable oncogenic transcripts in individual cancer patients within actionable periods. However, the majority of such driver mutations are not capable of pharmacological targeting, due to the lack of specific inhibitory molecules (Dang et al., 2017, Nature Reviews Cancer, 17: 502-508). For example, numerous fusion genes generated by chromosomal translocations demonstrate cogent oncogenic activity, however, personalized targeting of these fusion structural variants at the protein level is extremely challenging. Accordingly, there is a need to develop compositions and methods for targeting aberrant transcripts with Cas13, and to develop methods for the design of crRNA with high silencing efficiency and limited collateral activity, which would be suitable for targeted gene silencing in human cells.

### SUMMARY

In one aspect, the present disclosure provides a crRNA comprising from 5′ to 3′:
a. a spacer sequence that is capable of hybridizing to a target RNA sequence; and
b. a direct repeat sequence,
   wherein the nucleotide content of the spacer sequence is enriched for G nucleotides.

In another aspect disclosed herein, there is provided a crRNA comprising a spacer sequence that is capable of hybridizing to a target RNA sequence, wherein the target RNA sequence is a variant transcript, wherein the spacer sequence comprises at least one nucleotide mismatch relative to a corresponding nucleotide of the target RNA sequence, and wherein the spacer sequence selectively targets the variant transcript relative to a corresponding wild-type transcript from the same gene locus.

In another aspect disclosed herein, there is provided an RNA editing system comprising:
a. a polynucleotide encoding a Cas13 effector protein; and
b. the crRNA described herein, or a polynucleotide encoding the crRNA described herein.

In another embodiment disclosed herein, there is provided an RNA editing system comprising:
a. a Cas13 effector protein; and
b. the crRNA described herein.

In another embodiment disclosed herein, there is provided a cell or cell extract comprising the RNA editing system described herein.

In another embodiment disclosed herein, there is provided a method of altering a target RNA sequence in a cell, the method comprising providing to the cell the RNA editing system described herein, wherein the Cas13 effector protein when in conjunction with the crRNA, specifically hybridizes to the target RNA sequence, and wherein the Cas13 effector protein alters the hybridized target RNA sequence.

In another embodiment disclosed herein, there is provided a method for selecting a potent crRNA, the method comprising:
a. generating a plurality of crRNA in silico, wherein each of the plurality of crRNA comprises from 5′ to 3′: (i) a spacer sequence that is capable of hybridizing to the target RNA sequence, and (ii) a direct repeat sequence;
b. determining the spacer nucleotide content for each of the plurality of crRNA; and
c. selecting the crRNA described herein from the plurality of crRNA, wherein the selected crRNA comprise a spacer sequence that is enriched for G nucleotides.

In another embodiment disclosed herein, there is provided a method for selecting crRNA having a spacer sequence that hybridizes to a target RNA sequence, wherein the target RNA sequence is within a variant transcript comprising at least one SNV relative to a corresponding wild-type transcript from the same gene locus, the method comprising:
a. generating a plurality of crRNA in silico, wherein each of the plurality of crRNA comprises a spacer sequence that is capable of hybridizing to the variant transcript;
b. determining the spacer nucleotide content for each of the plurality of crRNA; and
c. selecting a crRNA from the plurality of crRNA, wherein the selected crRNA comprises a spacer sequence comprising at least one nucleotide mismatch relative to a corresponding nucleotide of the target RNA sequence, and wherein the spacer sequence selectively targets the variant transcript relative to a corresponding wild-type transcript from the same gene locus.

### BRIEF DESCRIPTION OF THE DRAWINGS

Embodiments of the disclosure are described herein, by way of non-limiting example only, with reference to the accompanying drawings.

FIG. 1 shows that the silencing efficiency of PspCas13b crRNAs are highly variable. (A) A schematic representation of the PspCas13b silencing assay used to track the recognition and degradation of mCherry RNA. (B) A series of photographic representations of fluorescence microscopy images show the silencing of mCherry transcripts with a targeting crRNA versus a non-targeting (NT) control crRNA in HEK 293T cells. (C) A schematic representation of 16 PspCas13b crRNAs targeting mCherry RNA. (D) A series of graphical representations of crRNA dose-dependent (ng/100 μL; x-axis) silencing of mCherry transcripts (relative expression (arbitrary units, A.U.); y-axis) with either NT or targeting crRNAs. Errors are SD and p-values of one-way ANOVA are indicated (95% confidence interval).

FIG. 2 shows the dose-dependent silencing of mCherry transcript with non-targeting crRNA and targeting crRNA. (A) A graphical representation of relative expression of mCherry transcript (A.U.; y-axis) and dose of targeting or non-targeting crRNA (Log 10[crRNA] (fM); x-axis). (B) A tabulated summary of IC50 values for 16 crRNA targeting mCherry transcripts.

FIG. 3 shows that a silencing assay by tiled crRNAs reveals that RNA sequence, position and/or landscape influence PspCas13b silencing efficiency. (A) A schematic representation of mCherry RNA covered by 10 tiled crRNAs targeting mCherry regions surrounding crRNA 12 and crRNA16 with 3-nucleotide increments. (B-C) A series of photographic representations of fluorescence microscopy images show the silencing of mCherry transcripts with tiled crRNAs targeting regions surrounding crRNA12 (B, left panel) and crRNA16 (C, left panel) in HEK 293T cells. NT is a non-targeting control crRNA. Quantification of silencing efficiency with tiled crRNAs targeting the mCherry region surrounding crRNA12 (B, right panel) and crRNA16 (C, right panel) in HEK 293T cells. The data are represented in arbitrary units (A.U.). Errors are SD with 95% confidence interval. (D) A schematic representation of the sequence of mCherry RNA covered by 61 single-nucleotide resolved tiled crRNAs around crRNA12. (E) A graphical representation of silencing efficiency (relative expression (A.U.); y-axis) obtained with 61 tiled crRNAs (x-axis) in HEK293T cells. Data points in the graph are normalized mean fluorescence from 4 representative fields of view imaged in N=2. The data are represented in arbitrary units (A.U.). Errors are SD with 95% confidence interval. N is the number of independent biological experiments.

FIG. 4 shows a Pearson correlation analysis between crRNAs silencing efficiency and spacer folding. A series of graphical representations of silencing efficiency (y-axis) and (A) spacer folding; (B) entire crRNA folding (spacer and direct repeat); (C) target sequence folding; (D) spacer-target hybridization energy; and (E) spacer-target interaction. Data points in the graph are values of the silencing efficiency of individual crRNAs and their predicted folding (MFE) or hybridization/interaction energy. r (correlation coefficient) and p-value (95% confidence interval) are indicated in each graph.

FIG. 5 shows a Pearson correlation analysis between spacer silencing efficiency and the nucleotide content of spacer. A series of graphical representations of silencing efficiency (y-axis) and (A) A; (B) U; (C) C; (D) G and (E) CG nucleotide content. Data points in the graph show the silencing efficiency and base content of individual spacer sequences. r (correlation coefficient) and p-value (95% confidence interval) are indicated in each graph.

FIG. 6 shows that in silico analysis of silencing profiles from 201 PspCas13b crRNAs revealed key design principles. (A) A schematic representation of the bioinformatics pipeline used to investigate various parameters that affect PspCas13b silencing. PFS positions (4 nucleotides surrounding the 5′ and 3′ end of the targeted region that base pair with the spacer) are indicated. (B) A graphical representation of 201 crRNAs ranked (x-axis) based on their silencing efficiency (%, y-axis). The highly potent crRNAs that achieved >90% silencing efficiency and the ineffective crRNAs that achieved <50% silencing efficiency are analysed for PFS and spacer nucleotide positions. The PFS of the 4 most potent and least effective crRNAs are indicated. (C-D) A graphical representation of Position Weight Matrices (PWMs) depicting the positional nucleotide probabilities of upstream or downstream PFS in either the (C) highly potent or (D) ineffective crRNAs. (E) A graphical representation of Position Weight Matrices (PWMs) depicting the positional nucleotide probabilities of the highly potent crRNA spacer sequences. (F) A graphical representation of delta nucleotide probabilities (y-axis) of the highly potent crRNA spacer sequences that compare filtered spacer nucleotide positions (x-axis) to the baseline nucleotide distribution. (G) A graphical representation of PWMs depicting the positional nucleotide probabilities of ineffective crRNA spacer sequences. (H) A graphical representation of delta nucleotide probabilities (y-axis) of the ineffective crRNA spacer sequences that compare filtered spacer nucleotide positions (x-axis) to the baseline nucleotide distribution.

FIG. 7 shows the functional validation of PspCas13b crRNA prediction and design. (A) Design of predicted potent crRNAs harbouring a ‘GG’ motif at 5′ end of spacers targeting EGFP transcript and validation of predicted potent crRNAs (x-axis) by EGFP expression (relative expression of EGFP (A.U.); y-axis) in HEK293T cells. (B) Design of predicted ineffective crRNAs lacking 5′ GG motif and harbouring ‘C’ bases at the central region of spacers targeting EGFP transcript and validation of predicted ineffective crRNAs (x-axis) by EGFP expression (relative expression of EGFP (A.U.); y-axis) in HEK293T cells. Data points in the graph are mean fluorescence from 4 representative field of views per condition imaged; N=3 or 4. The data are represented in arbitrary units (A.U.). Errors are SD and p-values of one-way ANOVA are indicated (95% confidence interval). (C) A graphical representation of average silencing efficiency of EGFP (A.U.; y-axis) of predicted potent and ineffective crRNAs (x-axis). Data points in the graph represent independent biological replicates. N=3 or 4; Data are normalized means and errors are SE. Results are analysed by unpaired two-tailed Student's t-test (95% confidence interval). (D) Design of predicted potent crRNAs harbouring a ‘GG’ motif at 5′ end of spacers targeting TagBFP transcript and validation of predicted potent crRNAs (x-axis) by TagBFP expression (relative expression of TagBFP (A.U.); y-axis) in HEK293T cells. (E) Design of predicted ineffective crRNAs lacking 5′ GG motif and harbouring ‘C’ bases at the central region of spacers targeting TagBFP transcript and validation of predicted ineffective crRNAs (x-axis) by TagBFP expression (relative expression of TagBFP (A.U.); y-axis) in HEK293T cells. Data points in the graph are mean fluorescence from 4 representative fields of view per condition imaged; N=3 or 4. The data are represented in arbitrary units (A.U.). Errors are SD and p-values of one-way ANOVA test are indicated (95% confidence interval). (F) A graphical representation of average silencing efficiency of TagBFP (A.U.; y-axis) of predicted potent and ineffective crRNAs (x-axis). Data points in the graph represent independent biological replicates. N=3 or 4; Data are normalized means and errors are SE. Results are analysed by unpaired two-tailed Student's t-test (95% confidence interval). (G) A schematic representation of RfxCas13d silencing assay to target mCherry transcript in HEK293T cells using potent crRNAs predicted by the RfxCas13d guide prediction platform published by Wessels et al. (2020, Nature Biotechnology, 38: 722-727). (H) Design of top 10 potent RfxCas13d crRNAs targeting mCherry transcripts predicted with Wessels et al. method and validation of predicted potent crRNAs (x-axis) by mCherry expression (relative expression of mCherry (A.U.); y-axis) in HEK293T cells. Data points are mean fluorescence from 4 representative field of views per condition imaged; N=3. The data are represented in arbitrary units (A.U.). Errors are SD and p-values of one-way ANOVA test are indicated (95% confidence interval). A graphical representation of average silencing efficiency (A.U.; y-axis) of predicted potent RfxCas13d crRNAs targeting mCherry transcripts (x-axis) is shown at the right-side graph. Data points in the graph represent independent biological replicates. N=3; Data are normalized means and errors are SE (95% confidence interval). (I-O) A series of graphical representations of relative expression of mCherry (A.U.: y-axis) following incorporation of a G-rich motif at the 5′ end (x-axis) of ineffective spacer sequences targeting mCherry through G-nucleotide insertion or substitution greatly enhanced their silencing efficiency. Data points in the graph are mean fluorescence from 4 representative field of views per condition imaged; N=3 or 4. The data are represented in arbitrary units (A.U.). Errors are SD and p-values of one-way ANOVA test are indicated (95% confidence interval). Nis the number of independent biological replicates.

FIG. 8 shows the frequency of A, C, G, and U nucleotides in crRNA spacer sequences. (A) A graphical representation of base content in unfiltered crRNAs by reference to nucleotide frequency (y-axis) and nucleotide (A, C, G, U; x-axis). (B) A graphical representation of base content in potent crRNAs by reference to nucleotide frequency (y-axis) and nucleotide (A, C, G, U; x-axis). (C) A graphical representation of the delta base content in potent crRNAs by reference to delta frequency (y-axis) and nucleotide (A, C, G, U; x-axis). (D) A graphical representation of the delta base content in ineffective crRNAs by reference to delta frequency (y-axis) and nucleotide (A, C, G, U; x-axis).

FIG. 9 shows that enrichment of C nucleotides at the 5′ end of the spacer sequence compromises silencing efficiency in a dose-dependent manner. (A) A series of graphical representations of relative expression (A.U.; y-axis) of mCherry transcript following transfection with non-targeting crRNA (NT), wild-type crRNA1-11 (WT) harbouring 1-3 mismatched nucleotides at the 5′end of the spacer sequences to introduce a 5′end ‘CC’ sequence instead of a ‘GG’ sequence. Data points in the graph are normalized mean fluorescence from 4 different field of views imaged in 112. The data are represented in arbitrary units (A.U.). Errors are SD with 95% confidence interval. (B) Design of crRNA to examine the impact of C to G substitutions on crRNA silencing efficiency (top panel), and a graphical representation of relative expression (A.U.; y-axis) for each of the mutagenized crRNA (x-axis) (bottom panel). Data points in the graph are mean fluorescence from 4 representative field of views per condition imaged; N=3. The data are represented in arbitrary units (A.U.). (C) A series of photographic representations of fluorescence microscopy images (right panel) show the silencing efficiency of the mCherry transcripts with NT, WT and mutant crRNAs in HEK293T cells. NT is a non-targeting control crRNA. Scale bar=400 μm. Similar results were obtained in 3 independent experiments in HEK 293T cells.

FIG. 10 shows that comprehensive mutagenesis of PspCas13b spacer-target interaction revealed specificity and the interface between mismatch tolerance and loss of activity. (A-B, top panel) Design of crRNAs harbouring mismatched nucleotides at various positions of crRNA spacer sequence. Perturbation of spacer-target interaction through spacer mutagenesis to introduce 3, 6, 9, 12, 15, 18, 21, 24, 27, and 30-nucleotide consecutive mismatches at the (A) 3′ end, and (B) 5′ end of the spacer. (A-B, bottom panel) A graphical representation of expression (relative expression (A.U.; y-axis) and mismatch position (x-axis). Data points in the graph are mean fluorescence from 4 representative field of views per condition imaged, N=3 or 4. The data are represented in arbitrary units (A.U.). Errors are SD and p-values of one-way ANOVA test are indicated (95% confidence interval). N is the number of independent biological replicates. (C-F, top panel) Design of crRNAs harbouring consecutive mismatched nucleotides at various positions of crRNA spacer sequence. Perturbation of spacer-target interaction through spacer mutagenesis to introduce (C) 6. (D) 5, (E) 4 and (F) 3 consecutive mismatched nucleotides at various positions the spacer. (C-F, bottom panel) A graphical representation of expression (relative expression (A.U., y-axis) and mismatch position (x-axis). Data points in the graph are mean fluorescence from 4 representative field of views per condition imaged; N=3 or 4. The data are represented in arbitrary units (A.U.). Errors are SD and p-values of one-way ANOVA test are indicated (95% confidence interval). N is the number of independent biological replicates. (H) Design of crRNAs harbouring non-consecutive mismatched nucleotides at various positions of crRNA spacer sequence (top panel), and a graphical representation of expression (relative expression (A.U.; y-axis) and the number/position of mismatch (x-axis). Data points in the graph are mean fluorescence from 4 representative field of views per condition imaged; N=3 or 4. The data are represented in arbitrary units (A.U.). Errors are SD and p-values of one-way ANOVA test are indicated (95% confidence interval). N is the number of independent biological replicates.

FIG. 11 shows that incorporation of G-rich motif at the 5′end of the spacer increases crRNA expression or stability. (A-D, top panel) Design of crRNAs enriched for G nucleotides, i.e., starting with an extra G, crRNAs with the first nucleotide substituted to a G, and crRNAs with the first and second nucleotides substituted to GG. (A-D, bottom panel) A graphical representation of relative expression (y-axis) of crRNAs enriched for G nucleotides (x-axis). (E) A graphical representation of averaged relative expression (y-axis) of wild-type crRNA or crRNA enriched for G nucleotides (x-axis). crRNA expression was measured 48 h post-transfection in HEK293T cells, N=3. Data are normalized means and errors are SEM; Results analysed with one-way ANOVA with p-value indicated (95% confidence interval).

FIG. 12 shows that incorporation of target-mismatched ‘G’ nucleotides at the 5′end and/or central regions of spacer sequence greatly enhance PspCas13b crRNA efficiency. (A-F, top panel) Design of crRNAs targeting the breakpoint of gene fusion transcripts enriched for Cl nucleotides, i.e., with or without incorporation of mismatched G-bases at the 5′end and/or central regions of the spacer. (A-F, bottom panel) A graphical representation of relative expression (A.U.; y-axis) for wild-type crRNA or crRNA enriched for G nucleotides (x-axis). Data points in the graphs are mean fluorescence from 4 representative field of views per condition imaged. The data are represented in arbitrary units (A. U.). Errors are SD and p-values of unpaired two-tailed Student's t-test are indicated (95% confidence interval). (G-L, top panel) Design of crRNAs targeting the breakpoint of gene fusion transcripts enriched for Cl nucleotides, i.e., with or without incorporation of mismatched G-bases at the 5′end and/or central regions of the spacer. (G-L, bottom panel) A graphical representation of relative expression (A.U.; y-axis) for wild-type crRNA or crRNA enriched for G nucleotides (x-axis). Data points in the graphs are mean fluorescence from 4 representative field of views per condition imaged. The data are represented in arbitrary units (A.U.). Errors are SD and p-values of unpaired two-tailed Student's t-test are indicated (95% confidence interval).

FIG. 13 shows that reprogrammed PspCas13b suppresses fusion gene transcripts with high efficiency. (A-C, top panel) Tiled PspCas13b crRNAs with 3-nucleotide resolution targeting the breakpoint region of gene fusion transcripts (A) BCR-ABL1, (B) SNX2-ABL1 and (C) SFPQ-ABL1. (A-C, bottom panel) A graphical representation of expression (relative expression (A.U.); y-axis) and tiled crRNAs targeting the fusion breakpoint (x-axis) Data points in the graphs are mean fluorescence from 4 representative fields of view per condition imaged; N=3. The data are represented in arbitrary units (A.U.). Errors are SD and p-values of one-way ANOVA are indicated (95% confidence interval). (D-F) A series of graphical representations of silencing efficiency (relative expression (RT-PCR), y-axis) of tiled PspCas13b crRNAs (x-axis) targeting the breakpoint regions of fusion transcripts (D) BCR-ABL1, (E) SNX2-ABL1 and (F) SFPQ-ABL1. Data are normalized means and errors are SD; Results are analysed by one-way ANOVA with p-values indicated (95% confidence interval). (G) A photographic representation of expression of BCR-ABL1 protein in HEK293T cells expressing tiled crRNAs with 3-nucleotide increment targeting the breakpoint region of BCR-ABL1 transcripts 24 h post-transfection. (H) A schematic representation of BCR-ABL1 dependent phosphorylation of ERK and Stat proteins, and inhibition of BCR-ABL1 oncogenic activity with imatinib. (I) A photographic representation of BCR-ABL1 expression and subsequent inhibition of STAT5 and ERK phosphorylation in HEK293T cells expressing BCR-ABL1, PspCas13b and either NT or crRNA targeting the BCR-ABL1 at 24 h post-transfection. HEK293T cells expressing BCR-ABL1 and PspCas13b treated with 1 μM imatinib for 4 hours were used as a positive control. Parental cells are HEK293T cells transfected with PspCas13b, NT and a random control plasmid. This condition shows the baseline expression of pSTAT5 and pERK in BCR-ABL1 independent manner. (J) A graphical representation of 41 single-nucleotide tiled crRNAs targeting mRNA region surrounding the breakpoint of BCR-ABL1 (x-axis) and silencing efficiency (relative expression (A.U.); y-axis). The schematic shows the sequence of BCR-ABL1 RNA covered by 41 tiled crRNAs and RNA-RNA duplex formed by spacer-target interaction. The dashed box highlights two adjacent crRNAs (14 & 15) with markedly contrasted silencing efficiency. Data points in the graph are normalized mean fluorescence from 4 representative fields of view imaged in N=2. The data are represented in arbitrary units (A.U.). Errors are SD with 95% confidence interval. (K) A photographic representation of silencing efficiency of single-base resolved crRNAs 14 & 15 that target BCR-ABL1 mRNA. crRNA potency is examined through the silencing of BCR-ABL1 protein and phosphorylation of STAT5 and ERK proteins. Cells expressing BCR-ABL1, PspCas13b and either NT or crRNA targeting the BCR-ABL1 were harvested for WB analysis 24 h post-transfection. 1 μM imatinib treatment for 4 hours was used as a positive control to inhibit BCR-ABL1 kinase activity. Parental cells are HEK293T cells transfected with PspCas13b, NT and a control plasmid to examine the baseline expression of pSTAT5 and pERK in a BCR-ABL1 independent manner.

FIG. 14 shows that the targeting of the breakpoint of gene fusions can efficiently discriminate between translocated tumor RNAs and wild type variants despite extensive sequence homology. (A-B, top panel) Design of crRNAs targeting the breakpoint region of BCR-ABL1 transcript to examine spacer-target interaction, specificity and mismatch tolerance of PspCas13b. (A-B, bottom panel) A graphical representation of expression (relative expression (A.U.; y-axis) and (A) the number of mismatched nucleotides per spacer, or (B) mismatch position (x-axis). Data points in the graph are mean fluorescence from 4 representative field of views per condition imaged; N=4. The data are represented in arbitrary units (A.U.). Errors are SD and p-values of one-way ANOVA are indicated (95% confidence interval). (C) Design of crRNAs targeting the breakpoint region of BCR-ABL1 transcript (top panel) and a photographic representation of the expression level of BCR-ABL1 protein and phosphorylation status of STAT5 and ERK in HEK293T cells expressing crRNAs with various mismatches 24 h post-transfection (bottom panel). (D-F, top panel) A schematic representation and a photographic representation of 3 colour fluorescence-based reporter assays to assess the on-target specificity of crRNA targeting the breakpoint region of (D) BCR-ABL1 (BCR-ABL1-mCherry mRNA) and potential off-targeting of wild-type (E) ABL1 (ABL1-eGFP mRNA) and (F) BCR (BCR-TagBFP mRNA) transcripts and their interaction with crBCR, crBCR-ABL1 and crABL1crRNAs through full, partial, or no spacer-target base pairing in HEK293T cells 48 h post-transfection. Scale bar=100 μm. (D-F, bottom panel) A graphical representation of gene silencing (relative expression (A.U.); y-axis) of (D) BCR-ABL1-mCherry, (E) ABL1-eGFP and (F) BCR-TagBFP. Data points are normalized mean fluorescence from 4 representative fields of view per condition imaged. The data are represented in arbitrary units (A.U.). Errors are SD and p-values of one-way ANOVA test are indicated (95% confidence interval). (G) A schematic representation of imatinib-sensitivity or imatinib-resistance of wild-type and T315I variants, respectively (left panel); a photographic representation of protein expression to examine the suppression of imatinib-resistant T315I BCR-ABL1 with PspCas13b in HEK293T cells expressing wild-type or T315I BCR-ABL1 variants (right panel), PspCas13b and either NT or crRNAs targeting the BCR-ABL1 breakpoint 24 h post-transfection. HEK293T cells expressing BCR-ABL1 variants and PspCas13b were treated with 1 μM imatinib for 4 hours as a positive control. Parental cells are HEK293T cells transfected with PspCas13b, NT and a control plasmid, which shows the baseline expression of pSTAT5 and pERK in BCR-ABL1 independent manner.

FIG. 15 shows that parental crRNAs achieve equipotent silencing of wild type and single nucleotide variant tumor transcripts. (A) A schematic representation of the PspCas13b fluorescence reporter assay used to assess the silencing efficiency of wild type and single nucleotide variant tumor transcripts. (B) A graphical representation of silencing efficiency (normalized mean fluorescence intensity (MFI); y-axis) of four crRNAs (x-axis) in HEK293T cells at 48 h post-knock in of wild-type BRAF (left panel) and single nucleotide variant, BRAF-V600E (right panel) constructs, normalized against a non-targeting control crRNA (gNT). Data is shown as mean±SD, where individual data points are normalised MFI averaged from four representative fields of view (n=3 independent experiments). (C) A series of photographic representations of fluorescence micrographs of the data presented in (B). Scale bar=300 μm.

FIG. 16 shows that single nucleotide mutagenesis of parental crRNAs allows for single nucleotide variant-specific transcriptional repression. (A) A graphical representation of silencing efficiency (normalized MFI; y-axis) of crRNA-1 and its mutagenesis products (x-axis) in HEK293T cells at 48 h post-knock-in of a wild-type construct, normalized against a non-targeting control crRNA (crNT). crRNAs indicated with arrows are those that show the greatest loss of silencing efficiency upon perturbation of the crRNA-1 sequence. (B) A graphical representation of silencing efficacy of the top performing crRNA in (A) in HEK293T cells at 48 h post-knock-in of a single nucleotide variant construct (filled bar), normalised against a non-targeting control crRNA (crNT). (C) A graphical representation of a parallel comparison of crMut13 and crMut14 silencing efficiency in wild type and single nucleotide variant transcripts. (D) A graphical representation of dose (Log gRNA; x-axis) and response (normalized MFI; y-axis) derived from the titration of crNT, (E) parental crBRAF-1 (F) crMut-13 and (G) crMut-14 in HEK293T cells transfected with wild-type or single nucleotide variant constructs. (H) A graphical representation of delta silencing efficiencies from highest dose in (D) and (G) (fold change; y-axis) between wild type and single nucleotide variant constructs (x-axis). All data is shown as mean±SD from n=3 independent experiments.

FIG. 17 shows that V600E-specific silencing efficiency of full-length BRAF is achievable with PspCas13b but not SpCas9. (A) A photographic representation of silencing efficiency assessed by western blot in HEK293T cells transfected with PspCas13b and full-length BRAF wild type or V600E constructs. (B) A graphical representation of gene expression (2\hat{ }(ΔΔCt); y-axis) in cancer cell lines with endogenous BRAF expression transfected with PspCas13b and full-length BRAF wild type or V600E constructs (x-axis). (C) A schematic representation of divergent crRNA design requirements for SpCas9 and PspCas13b, respectively. (D) A photographic representation of silencing efficiency assessed by western blot in HEK293T cells transfected with PspCas13b or SpCas9 and full-length BRAF wild type or V600E constructs.

FIG. 18 shows that the single-nucleotide mismatch tiling screen is effective for identifying Ruminococcus flavefaciens Cas13d (RfxCas13d) crRNA for potent and specific targeting of BRAF V600E RNA. (A) A graphical representation of silencing efficiency (normalized MFI; y-axis) of crBRAF-1 and mutagenesis of BRAF WT (grey bars) vs BRAF-V600E (dark grey bars), normalized against a non-targeting control (crNT) at 48 hours post-transfection. (B) A photographic representation of fluorescence micrographs showing equipotent silencing of BRAF WT and BRAF V600E variants with the non-selective crBRAF-1 and BRAF V600E-selective crMM2. (C) A graphical representation of delta silencing efficiency (fold change; y-axis) of crMM2 against BRAF WT and BRAF V600E constructs (x-axis). (D) Graphical representations of dose response (normalised MFI; y-axis) from titration (log gRNA (ng); x-axis) of crBRAF-1 (left panel) or crMM2 (right panel) against BRAF WT or BRAF V600E constructs. For all graphs error bars represent mean±SD from three independent experiments. Statistical significance was determined using unpaired t-tests, where * p<0.05, ** p<0.01, *** p<0.001, *** p<0.0001.

FIG. 19 is a schematic representation of the G12 mutation hotspot in exon 2 of the KRAS gene (codon 12, nucleotides 34-36). For wild-type KRAS, the consensus coding sequence of “GGT” at codon 12 encodes a glycine (i.e., G12). Missense mutations that affect the “G” nucleotide at position 34, collectively referred to as “c.34 variants”, change the amino acid sequence such that arginine (G12R, from c.34G>C substitution), serine (G12S, c.34G>A) or cysteine (G12C, c.34C>T) are encoded instead of glycine. “c.35 variants” arise from missense substitutions at nucleotide 35, causing glycine to be replaced by alanine (G12A, c.35G>C), aspartate (G12D, c.35G>A) or valine (G12V, c.35G>T).

FIG. 20 shows that bi-specific crRNA can selectively silence KRAS G12C and G12D variants. (A) A schematic representation of bi-specific G12-targeting crRNAs. (B) A graphical representation of silencing efficiency (normalized MFI; y-axis) of crC/D and its mutagenesis derivatives (x-axis) against KRAS WT (grey bars), KRAS G12C (dark grey bars) and KRAS G12D (light grey bars) constructs, normalized against a non-targeting control (crNT), at 48 hours post-transfection. Error bars represent mean±SD from three independent experiments.

FIG. 21 shows that crC/D-9 and crC/D-12 exhibit dose-dependent silencing of KRAS G12 mutants. A graphical representation of dose response (normalized MFI; y-axis) from a titration (concentration [pM]; x-axis) of crC/D (left panel) and its mutagenesis derivatives crC/D-9 (middle panel) and crC/D-12 (right panel) against KRAS WT, KRAS G12C and KRAS G12D constructs. Error bars represent mean±SD from three independent experiments.

FIG. 22 shows that mutagenesis of crC/D-9 and crC/D-12 generates novel crRNAs that selectively silence all KRAS G12 variants. (A) A graphical representation of silencing efficiency (normalised MFI; y-axis) of crC/D-9 (left panel) and crC/D-12 (right panel) against KRAS WT and KRAS c.34 and c.35 variants (G12R, G12S, G12A, and G12V; x-axis), normalized against crNT, at 48 hours post-transfection. (B) A schematic representation of the mutagenesis strategy to “switch” the silencing selectivity of G12C- or G12D-selective crRNAs to other G12 variants. (C) A series of graphical representations showing the silencing efficiency (normalised MFI; y-axis) or various crC/D-9 and crC/D-12 mutagenesis derivatives against six KRAS G12 variant constructs. Error bars represent mean±SD from three independent experiments. Statistical significance was determined using unpaired t-tests, where * p<0.05, **p<0.01, *** p<0.001, *** p<0.0001.

FIG. 23 shows that SNV-selectivity is enhanced in crRNAs containing two mismatches relative to the KRAS G12 target sequence. A series of graphical representations showing the SNV-selectivity profile of all G12-targeting crRNAs. Each point represents the mean silencing efficiency of a crRNA against KRAS WT (wild type expression/off-target silencing; y-axis) and G12-mutant targets (SNV expression/on-target silencing; x-axis). crRNAs that fall within the upper left quadrant (indicating <50% expression of the G12 variant whilst maintaining >50% expression of the WT) are considered SNV-selective crRNAs.

FIG. 24 shows potent and selective silencing of five KRAS G12 variants using re-programed RfxCas13d. (A) A series of graphical representation of dose response (normalized MFI; y-axis) from a titration (concentration, [pM]; x-axis) of crG12 guides against KRAS WT and KRAS G12 variant (G12A, G12C, G12D, G12R and G12S) constructs. (B) A series of photographic representations showing silencing efficiency by western blotting of the crRNA of (A) assessed in HEK293T cells transfected with KRAS WT or KRAS G12-mutated constructs. (C) A graphical representation of the quantification of silencing efficiency (normalized KRAS expression; y-axis) of (B) against KRAS WT and KRAS G12 variant constructs (x-axis) showing SNV-specificity for all crRNAs at the protein level. Error bars represent mean±SD from three independent experiments.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

The articles “a”, “an” and “the” include plural aspects unless the context clearly dictates otherwise. Thus, for example, reference to “a polynucleotide” includes a single polynucleotide, as well as two or more polynucleotides; reference to “an effector protein” includes a single effector protein, as well as two or more effector proteins; and so forth.

In the context of this specification, the term “about” is understood to refer to a range of numbers that a person of skill in the art would consider equivalent to the recited value in the context of achieving the same function or result. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements.

The term “optionally” is used herein to mean that the subsequent described feature may or may not be present or that the subsequently described event or circumstance may or may not occur. Hence the specification will be understood to include and encompass embodiments in which the feature is present and embodiments in which the feature is not present, and embodiment in which the event or circumstance occurs as well as embodiments in which it does not.

As used herein, the term “derived from” shall be taken to indicate that a particular integer or group of integers has originated from the species specified, but has not necessarily been obtained directly from the specified source.

Amino acids may be referred to herein by either the commonly known three letter symbols or by the single letter symbols recommended by the IUPAC-IUB Biochemical Nomenclature Commission. Similarly, nucleotides may be referred to by their commonly accepted single letter codes.

All sequence database identifiers (e.g., GenBank ID, EMBL-Bank ID, DNA Data Bank of Japan (DDBJ) ID, etc.), Addgene identifiers, Protein Data Base (PDB) identifiers provided herein were current at the filing date.

Unless otherwise defined, all technical and scientific terms used herein have the same meaning as commonly understood by one of ordinary skill in the art to which this invention belongs.

The present disclosure is predicated, in part, on the surprising finding that crRNAs harbouring spacer sequences that are enriched for guanosine (G) nucleotides greatly enhances the silencing efficiency of otherwise inefficient crRNAs, expanding the targeting spectrum of Cas13. In other embodiments, the crRNAs disclosed herein are optimized for mismatch tolerance and spacer-target interaction. These findings have been reduced to practice in the design, selection and generation of crRNAs, and the use of such crRNAs in RNA editing systems that can potently and selectively target transcripts (e.g., gene fusion transcripts, variant transcripts comprising at least one single nucleotide variant (SNV)), without the off-targeting of highly homologous transcripts (e.g., non-translocated variants, homologous wild-type transcripts). Accordingly, the present inventors have surprisingly shown that Cas13 can be efficiently reprogrammed to specifically silence various transcripts, including variant transcripts comprising oncogenic driver mutations in a personalized manner.

crRNA

In an aspect disclosed herein there is provided a crRNA comprising from 5′ to 3′:
a. a spacer sequence that is capable of hybridizing to a target RNA sequence; and
b. a direct repeat sequence,
   wherein the nucleotide content of the spacer sequence is enriched for G nucleotides.

In another aspect disclosed herein there is provided a crRNA comprising a spacer sequence that is capable of hybridizing to a target RNA sequence, wherein the target RNA sequence is a variant transcript, wherein the spacer sequence comprises at least one nucleotide mismatch relative to a corresponding nucleotide of the target RNA sequence.

The term “CRISPR RNA” or “crRNA” as used herein refers is a 60 to 70 nucleotide sequence comprising, consisting or consisting essentially of: (a) a spacer sequence that is capable of hybridizing to a target RNA sequence; and (b) a direct repeat sequence that forms a short hairpin structure, which is recognized by the Cas13 protein to form the CRISPR-Cas13 complex.

In an embodiment, the crRNA is a non-naturally occurring nucleotide sequence.

The terms “non-naturally occurring” or “engineered” may be interchangeably used herein to refer to nucleotides or nucleic acid molecules that are distinguished from their naturally occurring counterparts. For example, the crRNA of the present disclosure may be recombinant, synthetic, or comprise mixtures of naturally and non-naturally occurring nucleotides. Non-naturally occurring nucleotides or nucleotide analogs may be modified at the ribose, phosphate and/or base moiety.

In an embodiment, the crRNA comprises ribonucleotides and non-ribonucleotides. In one such embodiment, the crRNA comprises one or more ribonucleotides and one or more deoxyribonucleotides.

In an embodiment, the crRNA comprises one or more non-naturally occurring nucleotide or nucleotide analog such as a nucleotide with phosphorothioate linkage, boranophosphate linkage, a locked nucleic acid (LNA) nucleotides comprising a methylene bridge between the 2′ and 4′ carbons of the ribose ring, or bridged nucleic acids (BNA). Other examples of modified nucleotides include 2′-0-methyl analogs, 2′-deoxy analogs, 2-thiouridine analogs, N6-methyladenosine analogs, or 2′-fluoro analogs. Further examples of modified bases include, but are not limited to, 2-aminopurine, 5-bromo-uridine, pseudouridine (Ψ), N¹-methylpseudouridine (me¹Ψ), S-methoxyuridine (SmoU), inosine, 7-methylguanosine.

In an embodiment, the crRNA is a synthetic crRNA.

The crRNAs of the present disclosure may be produced using any method in the art, including synthetically or by recombinant techniques such as expression of polynucleotide constructs encoding the components. For example, a protein may be synthesized using the Fmoc-polyamide mode of solid-phase peptide synthesis. Other synthesis methods include solid phase t-Boc synthesis and liquid phase synthesis. Purification can be performed by any one of, or a combination of, techniques such as re-crystallization, size exclusion chromatography, ion-exchange chromatography, hydrophobic interaction chromatography and reverse-phase high performance liquid chromatography using, for example, acetonitrile/water gradient separation.

The crRNA of the present disclosure is arranged from 5′ to 3′. It would be known to persons skilled in the art that this orientation refers to the spacer sequence of the crRNA being located 5′ (i.e., “upstream”) with respect to the direct repeat sequence, or the direct spacer sequence being located 3′ (i.e., “downstream”) with respect to the spacer sequence.

The term “direct repeat sequence” refers to the sequence of the crRNA, which comprises a stem loop, an optimized stem loop structure or an optimized secondary structure.

Persons skilled in the art will appreciate that the direct repeat sequence comprises a self-complementary sequence that forms the stem loop, optimized stem loop structure or optimized secondary structure.

In an embodiment, the direct repeat sequence comprises at least one stem loop.

The term “spacer sequence” as used herein refers to the sequence of the crRNA that specifies the target site, i.e., which is capable of hybridizing to a target RNA sequence.

The term “target RNA sequence” as used herein refers to a RNA sequence within an RNA molecule to which a crRNA is designed to have complementarity, where hybridization between the target RNA sequence and the crRNA promotes the formation of a complex comprising the Cas13 effector protein, the crRNA and the target RNA sequence (i.e., an RNA editing complex).

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tₘ) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

By “capable of hybridizing” it is meant that the spacer sequence is complementary to, or substantially complementary to, the target RNA sequence.

By “complementary” or “substantially complementary” it is meant that a nucleic acid (e.g., RNA, DNA) comprises a sequence of nucleotides that enables it to non-covalently bind, i.e., form Watson-Crick base pairs and/or G/U base pairs, “anneal”, or “hybridize” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base pairing includes adenine/adenosine (A) pairing with thymidine/thymidine (T), A pairing with uracil/uridine (U), and guanine/guanosine (G) pairing with cytosine/cytidine (C). In addition, for hybridization between two RNA molecules (e.g., ssRNA), and for hybridization of a DNA molecule with an RNA molecule G can also base pair with U. For example, G/U base pairing is partially responsible for the degeneracy (i.e., redundancy) of the genetic code in the context of tRNA anti-codon base pairing with codons in mRNA. Thus, in the context of this disclosure, a G is considered complementary to both a U and to C. For example, when a G/U base-pair can be made at a given nucleotide position of a protein binding segment of a crRNA molecule, the position is not considered to be non-complementary, but is instead considered to be complementary.

In an embodiment, the degree of complementarity between the spacer sequence and the target RNA sequence is greater than about 60% (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%).

Accordingly, in an embodiment, the degree of complementarity between the spacer sequence and the target RNA sequence is preferably about 60%, preferably about 61%, preferably about 62%, preferably about 63%, preferably about 64%, preferably about 65%, preferably about 66%, preferably about 67%, preferably about 68%, preferably about 69%, preferably about 70%, preferably about 71%, preferably about 72%, preferably about 73%, preferably about 74%, preferably about 75%, preferably about 76%, preferably about 77%, preferably about 78%, preferably about 79%, preferably about 80%, preferably about 81%, preferably about 82%, preferably about 83%, preferably about 84%, preferably about 85%, preferably about 86%, preferably about 87%, preferably about 88%, preferably about 89%, preferably about 90%, preferably about 91%, preferably about 92%, preferably about 93%, preferably about 94%, preferably about 95%, preferably about 96%, preferably about 97%, preferably about 98%, preferably about 99%, or more preferably about 100%.

In an embodiment, the degree of complementarity between the spacer sequence and the target RNA sequence is greater than about 80%. In another embodiment, the degree of complementarity between the spacer sequence and the target RNA sequence is greater than about 90%.

In an embodiment, the spacer sequence comprises at least about 20 nucleotides (e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides).

Accordingly, in an embodiment, the spacer sequence comprises at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, or at least about 50 nucleotides, and so on and so forth.

In an embodiment, the spacer sequence comprises from about 20 nucleotides to about 40 nucleotides. In another embodiment, the spacer sequence comprises about 30 nucleotides.

The term “nucleotide” as used herein refers to the nucleotides adenosine, guanosine, cytidine, thymidine and uridine, each of which comprise a nucleotide base attached to a ribose ring. A person skilled in the art will appreciate that the terms “adenine/adenosine”, “uracil/uridine”, “guanine/guanosine”, “cytosine/cytidine” and “thymidine/thymine” (C) may be used interchangeably herein with the single letters A, U, G, T and T, respectively, which refer the nucleotide base comprised by the nucleotides.

The term “nucleotide content” as used herein refers to the composition and ratio of the constituent monomer units (e.g., A, U, G, C). As the number of nucleotides in each type of nucleic acid is equal to that of the corresponding bases, determination of the quantitative ratio of the basis can establish the nucleotide content of a given nucleic acid molecule (e.g., a crRNA).

In an embodiment, the nucleotide content of the spacer sequence disclosed herein is enriched for G nucleotides.

The term “enriched” is used herein to refer to a selectively higher level of G nucleotides in the spacer sequence. For example, a nucleotide content enriched for G nucleotides refers to a spacer sequence in which the number of G nucleotides is increased relative to the number of A, C or U nucleotides in the spacer sequence.

The nucleotide content of the spacer sequence is determined by reference to the corresponding (i.e., complementary) target RNA sequence. As such, the term “enriched” as used herein does not necessarily mean that the number of G nucleotides in the spacer sequence is greater than the number of A, C or U nucleotides in the spacer sequence. Rather, the spacer sequence may be “enriched” for G nucleotides by, e.g., selecting a target RNA sequence that has a greater number of C nucleotides, modifying the spacer sequence to add one or more G nucleotides, or substituting one or more A, C or U nucleotides for a G nucleotide. As disclosed elsewhere herein, the modification to the spacer sequence may be made despite the introduction of mismatched nucleotides relative to the target RNA sequence without reducing the efficiency or selectivity of the crRNA.

In an embodiment, the nucleotide content of the 5′ end of the spacer sequence has been enriched for G nucleotides.

In an embodiment, the spacer sequence comprises a G nucleotide at a position selected from 1, 2, 11, 12, 15, 16, 17 and combinations of the foregoing.

In an embodiment, the spacer sequence comprises a G nucleotide at a position 1 and 2.

In an embodiment, the spacer sequence comprises the nucleotide sequence of DDNNNNNNNNDDNNDDDNNNNNNNNNNNNN (SEQ ID NO: 1), wherein N is a G, U, A or C nucleotide and D is a G, U or A nucleotide.

In an embodiment, the spacer sequence comprises the nucleotide sequence of GDNNNNNNNNDDNNDDDNNNNNNNNNNNNN (SEQ ID NO:2), wherein N is a G, U, A or C nucleotide and D is a G, U or A nucleotide.

In an embodiment, the spacer sequence comprises the nucleotide sequence of GGNNNNNNNNDDNNDDDNNNNNNNNNNNNN (SEQ ID NO:3), wherein N is a G, U, A or C nucleotide and D is a G, U or A nucleotide.

In an embodiment, D is a G nucleotide.

In an embodiment, the crRNA comprises a functional fragment of SEQ ID NO: 1, 2, or 3, wherein the functional fragment retains the ability to hybridize to the target RNA sequence. A functional fragment may include, from 5′ to 3′, 15, 16, 17, 18, 19, 20, 21, 22, 23, 24, 25, 26, 27, 28, or 29 consecutive nucleotides of SEQ ID NO: 1, 2, or 3.

In an embodiment, the crRNA requires a minimum level of complementarity with the target RNA in order to hybridize and achieve RNA cleavage. In certain embodiments, the sequence comprising the minimum level of complementarity is referred to as the “seed sequence”.

In an embodiment, the spacer sequence comprises from about 20 to about 30 nucleotides that are capable of hybridizing to the target RNA sequence (e.g., 20, 21, 22, 23, 24, 25, 26, 27, 28, 29, 30 nucleotides).

Accordingly, in an embodiment, the spacer sequence comprises from about 20 to about 30 nucleotides, preferably about 20, preferably about 21, preferably about 22, preferably about 23, preferably about 24, preferably about 25, preferably about 26, preferably about 27, preferably about 28, preferably about 29, or more preferably about 30 nucleotides that are capable of hybridizing to the target RNA sequence.

In an embodiment, the spacer sequence comprises about 24 nucleotides that are capable of hybridizing to the target RNA sequence.

In an embodiment, the spacer sequence comprises about 23 nucleotides that are capable of hybridizing to the target RNA sequence.

In an embodiment, the target RNA sequence is a variant transcript or a wild-type transcript.

In an embodiment, the variant transcript comprises at least one single nucleotide variant (SNV) relative to a corresponding wild-type transcript from the same gene locus.

As described elsewhere herein, hybridization requires that the two nucleic acids contain complementary sequences, although mismatches between the bases of the crRNA and the target RNA sequence are possible (i.e., tolerated).

In an embodiment, the spacer sequence comprises at least one mismatched nucleotide relative to the target RNA sequence (e.g., 1, 2, 3, 4, 5, 6, 7, 8, 9, 10, 11, 12 mismatched nucleotides).

Accordingly, in an embodiment, the spacer sequence comprises at least one, preferably 1, preferably at least 2, preferably at least 3, preferably at least 4, preferably at least 5, preferably at least 6, preferably at least 7, preferably at least 8, preferably at least 9, preferably at least 10, preferably at least 11, or more preferably at least 12 mismatched nucleotides relative to the target RNA sequence.

Mismatched nucleotides can be introduced into the spacer sequence at the 5′ end (e.g., positions 1 to 6), the 3′ end (e.g., positions 25 to 30), or in the central region (e.g., positions 13 to 18) of the spacer sequence. The cleavage efficiency of the RNA editing system can be modulated by the positioning and extent of the mismatched nucleotides. In an embodiment, where the spacer sequence comprises from about 1 to about 3 mismatched nucleotides relative to the target RNA sequence, the mismatched nucleotides may be positioned in the central region, or in the 3′ region, but not in the 5′ region.

In an embodiment, the mismatched nucleotides are consecutive mismatched nucleotides.

By “consecutive” it is meant that two or more mismatched nucleotides are located successively or adjacent to each other in the spacer sequence, e.g., positions 3 and 4.

In an embodiment, the spacer sequence comprises not more than 3 consecutively mismatched nucleotides, wherein the mismatched nucleotides are located in the 5′ end, the 3′ end and/or the central region of the spacer sequence.

In an embodiment, the spacer sequence comprises not more than 3 consecutively mismatched nucleotides, wherein the mismatched nucleotides are located in the central region of the spacer sequence.

In an embodiment, the spacer sequence comprises not more than 3 consecutively mismatched nucleotides, wherein the mismatched nucleotides are located in the 3′ end of the spacer sequence.

In an embodiment, the mismatched nucleotides are non-consecutive mismatched nucleotides.

By “non-consecutive” it is meant that two or more mismatched nucleotides are located at different positions throughout the spacer sequence, e.g., positions 2 and 30.

In an embodiment, the spacer sequence comprises not more than 4 non consecutive mismatched nucleotides.

In an embodiment, the spacer sequence comprises not more than 4 non-consecutively mismatched nucleotides, wherein the mismatched nucleotides are located in the 5′ end, the 3′ end and/or the central region of the spacer sequence.

In an embodiment, the spacer sequence comprises not more than 4 non-consecutively mismatched nucleotides, wherein the mismatched nucleotides are located in the 3′ end of the spacer sequence.

In an embodiment, the spacer sequence comprises not more than 4 non-consecutively mismatched nucleotides, wherein the mismatched nucleotides are located in the central region of the spacer sequence.

In an embodiment, the mismatched nucleotide(s) are mismatched relative to a corresponding nucleotide of the target RNA sequence, wherein the target RNA sequence is a wild-type transcript.

In an embodiment, the mismatched nucleotide(s) are mismatched relative to a corresponding nucleotide of the target RNA sequence, wherein the target RNA sequence is a variant transcript, e.g., a variant transcript comprising at least one SNV.

In an embodiment, the target RNA sequence is a variant transcript, wherein the variant transcript comprises at least one SNV relative to a corresponding wild-type transcript from the same gene locus, and wherein the spacer sequence further comprises at least one mismatched nucleotide(s) relative to a corresponding nucleotide of a wild-type transcript from the same gene locus.

In an embodiment, the spacer sequence comprises:
a. at least one mismatched nucleotide relative to a corresponding nucleotide of the target RNA sequence; and
b. at least one mismatched nucleotide relative to a corresponding nucleotide of a wild-type transcript from the same gene locus.

In an embodiment, the spacer sequence comprises:
a. one or two mismatched nucleotides relative to a corresponding nucleotide of the target RNA sequence; and
b. from about one to about 3 mismatched nucleotides relative to a corresponding nucleotide of a wild-type transcript from the same gene locus.

In an embodiment, the selected crRNA selectively targets the variant transcript relative to a corresponding wild-type transcript from the same gene locus.

By “selectively targets” it is meant that the crRNA is capable of targeting the variant transcript at a higher frequency relative to a corresponding wild-type transcript from the same gene locus. Persons skilled in the art will appreciate that the selective targeting of a variant transcript can be determined with reference to any one or more, or all of RNA silencing, cleavage, degradation, hybridization, and the like.

In an embodiment, the crRNA is selected or modified to reduce the degree of secondary structure (e.g., stem-loop structure) formation within the crRNA. In accordance with this embodiment, no more than about 75% (e.g., 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74% or 75%) of the nucleotides in the crRNA are capable of self-complementary base pairing when optimally folded.

In an embodiment, the target RNA sequence is selected to reduce the degree of secondary structure formation within the target RNA sequence. In accordance with this embodiment, no more than about 75% (e.g., 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74% or 75%) of the nucleotides in the target RNA sequence are capable of self-complementary base pairing when optimally folded.

Methods for the determination of optimal folding of the crRNA or the target RNA sequence will be known to persons skilled in the art, illustrative examples of which include the calculation of minimum free energy (MFE) using, e.g., RNAfold (see, e.g., Gruber et al., 2008, Cell 106(1): 23-24).

In an embodiment, the crRNA comprises any one of the sequences in Table 1.

In an embodiment, the crRNA comprises any one of the sequences set forth in SEQ ID NOs: 419-423, 435-437, 439, 441 and 465-560, and those having at least about 90%, 90%, 91%, 92%, 93%, 94%, 95%, 96%, 98%, or 99% sequence identity to the to the spacer sequences set forth in SEQ ID NOs: 419-423, 435-437, 439, 441 and 465-560. Methods for selecting a potent crRNA

The crRNA of the present disclosure may be referred to as “potent crRNA”. By “potent crRNA” it is meant that the crRNA with the characteristics described herein provide higher silencing penetrance and selectively relative to other crRNA (e.g., ineffective crRNA). In some embodiments, the potency of the crRNA is attributed to, at least in part, to increased crRNA abundance, increased affinity between the Cas13 effector protein and the crRNA to thereby allow for preferential loading of the crRNAs to the Cas13 effector protein, and the enhancement of the catalytic activity and processivity of the Cas13 effector protein downstream of the loading process.

Accordingly, in another aspect, the present disclosure provides a method for selecting a potent crRNA, the method comprising:
a. generating a plurality of crRNA in silico, wherein each of the plurality of crRNA comprises from 5′ to 3′: (i) a spacer sequence that is capable of hybridizing to a target RNA sequence, and (ii) a direct repeat sequence;
b. determining the spacer nucleotide content for each of the plurality of crRNA; and
c. selecting potent crRNA from the plurality of crRNA, wherein potent crRNA comprise a spacer sequence that is enriched for G nucleotides.

In another aspect, there is provided a method for selecting a crRNA having a spacer sequence that hybridizes to a target RNA sequence within a variant transcript comprising at least one SNV relative to a corresponding wild-type transcript from the same gene locus, the method comprising:
a. generating a plurality of crRNA in silico, wherein each of the plurality of crRNA comprises a spacer sequence that is capable of hybridizing to the target RNA sequence within the variant transcript;
b. determining the spacer nucleotide content for each of the plurality of crRNA; and
c. selecting a crRNA from the plurality of crRNA, wherein the selected crRNA comprises a spacer sequence comprising at least one nucleotide mismatch relative to a corresponding nucleotide of the target RNA sequence, and wherein the selected crRNA selectively targets the variant transcript relative to a corresponding wild-type transcript from the same locus.

The term “potent crRNA” as used herein refers to a crRNA that is capable of achieving ≥80% silencing efficiency (e.g., 80%, 81%, 82%, 83%, 84%, 85%, 86%, 87%, 88%, 89%, 90%, 91%, 92%, 93%, 94%, 95%, 96%, 97%, 98%, 99% or 100% silencing efficiency).

The term “highly potent crRNA” as used herein refers to a crRNA that is capable of achieving ≥90% silencing efficiency (e.g., 90%, 91%, 92%, 93%, 94%, 95%, 96%, 97%, 98%, 99% or 100% silencing efficiency).

In an embodiment, the potent crRNA comprises a spacer sequence, wherein the nucleotide content of the 5′ end of the spacer sequence has been enriched for G nucleotides.

In an embodiment, the potent crRNA comprises a spacer sequence comprising a G nucleotide at a position selected from 1, 2, 11, 12, 15, 16, 17 and combinations of the foregoing.

In an embodiment, the potent crRNA comprises a spacer sequence comprising a G nucleotide at positions 1 and 2.

In an embodiment, the potent crRNA comprises a spacer sequence comprising the nucleotide sequence of DDNNNNNNNNDDNNDDDNNNNNNNNNNNNN (SEQ ID NO: 1), wherein N is a G, U, A or C nucleotide and D is a G, U or A nucleotide.

In an embodiment, the potent crRNA comprises a spacer sequence comprising the nucleotide sequence of GDNNNNNNNNDDNNDDDNNNNNNNNNNNNN (SEQ ID NO:2), wherein N is a G, U, A or C nucleotide and D is a G, U or A nucleotide.

In an embodiment, the potent crRNA comprises a spacer sequence comprising the nucleotide sequence of GGNNNNNNNNDDNNDDDNNNNNNNNNNNNN (SEQ ID NO:3), wherein N is a G, U, A or C nucleotide and D is a G, U or A nucleotide.

In an embodiment, D is a G nucleotide.

In an embodiment, the potent crRNA comprises a spacer sequence comprising from about 20 to about 30 nucleotides that are capable of hybridizing to the target RNA sequence.

In an embodiment, the potent crRNA comprises a spacer sequence comprising about 24 nucleotides that are capable of hybridizing to a corresponding nucleotide of the target RNA sequence.

In an embodiment, the potent crRNA comprises a spacer sequence comprising at least one mismatched nucleotide, wherein each of the mismatched nucleotides are mismatched relative to a corresponding nucleotide of the target RNA sequence.

In an embodiment, the potent crRNA comprises a spacer sequence comprising from about one to about 10 mismatched nucleotides relative to the target RNA sequence.

In an embodiment, the mismatched nucleotides are consecutive mismatched nucleotides. In another embodiment, the mismatched nucleotides are non-consecutive mismatched nucleotides.

The term “ineffective crRNA” as used herein refers to a crRNA that is capable of achieving ≤50% silencing efficiency (e.g., 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50% silencing efficiency).

In an embodiment, the ineffective crRNA comprise a spacer sequence that is enriched for C nucleotides.

In an embodiment, the ineffective crRNA comprise a spacer sequence comprising a C nucleotide at a position selected from 1, 2, 3, 4, 11, 12, 15, 16, 17, and combinations of the foregoing.

In an embodiment, the ineffective crRNA comprise a spacer sequence comprising the nucleotide sequence of CCCCNNNNNNCCNNCCCHNNNNNNNNNNNN (SEQ ID NO:4), wherein N is a G, U, A or C nucleotide and H is a C, U, or A nucleotide.

In an embodiment, H is a C nucleotide.

In an embodiment, the potent crRNA comprise no more than about 75% (e.g., 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74% or 75%) of nucleotides that are capable of self-complementary base pairing when optimally folded.

In an embodiment, the method further comprises selecting ineffective crRNA for modification to improve potency.

In an embodiment, the modification is one or both of:
a. the addition of at least one G nucleotide; and
b. the substitution of at least one A, U or C nucleotide to a G nucleotide.

The modifications contemplated herein can “rescue” an ineffective crRNA and generate potent crRNAs or highly potent crRNAs for any target RNA sequence.

In an embodiment, the selected crRNA preferentially hybridizes to the variant transcript relative to a corresponding wild-type transcript from the same gene locus.

In an embodiment, the method further comprises modifying the crRNA to alter specificity to an SNV in the target RNA sequence, wherein the target RNA sequence is a variant transcript.

In an embodiment, the modification is a substitution of a nucleotide at a position that is complementary to the position of an SNV in the target RNA sequence.

In an embodiment, the method further comprises modifying the spacer sequence of the selected crRNA, wherein the modification inhibits the hybridization of the spacer sequence to an SNV of the corresponding wild-type transcript from the same gene locus.

### RNA Editing Systems

In an aspect disclosed herein there is provided an RNA editing system comprising:
a. a Cas13 effector protein, or a polynucleotide encoding a Cas13 effector protein; and
b. the crRNA disclosed herein, or a polynucleotide encoding the crRNA disclosed herein.

In another aspect disclosed herein, there is provided an RNA editing system comprising:
a. a Cas13 effector protein; and
b. the crRNA as disclosed herein.

As used herein the terms “polynucleotide”, “nucleic acid” or “nucleic acid molecule” mean a single- or double-stranded polymer of deoxyribonucleotide, ribonucleotide bases or known analogues or natural nucleotides, or mixtures thereof, and can include molecules comprising coding and non-coding sequences of a gene, sense and antisense sequences and complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments.

As used herein, the terms “encode”, “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

The terms “protein”, “peptide” and “polypeptide” are used interchangeably herein to refer to a polymer of amino acid residues linked together by peptide (amide) bonds. The terms refer to a protein, peptide, or polypeptide of any size, structure or function.

The term “RNA editing” as used herein refers to the site-specific alteration of an RNA sequence that could have been copied from the template, excluding changes due to processes such as RNA splicing and polyadenylation. Any suitable RNA-guided effector proteins can be introduced into a cell to induce editing of a target RNA sequence, e.g., CRISPR-associated (Cas) endonucleases. Naturally occurring and synthetic Cas endonucleases are contemplated herein.

The “clustered regularly interspaced short palindromic repeat” (CRISPR)/“CRISPR-associated protein” (Cas) system (CRISPR/Cas system) evolved in bacteria and archaea as an adaptive immune system to defend against viral attack. The mechanisms of CRISPR-mediated gene editing would be known to persons skilled in the art and have been described, for example, by Doudna et al., (2014, Methods in Enzymology, 546).

Cas13 is an effector protein that has been identified in Type VI CRISPR systems for RNA-guided RNA-interfering activity (Abudayyeh et al., 2016, Science, 353: aaf5573). Cas13 comprise two enzymatically active higher eukaryotes and prokaryotes nucleotide-binding (HEPN) RNAse domains, which induce cis- and trans-RNA cleavage via crRNA-guided effector complex (crRNA-Cas13).

In an embodiment, the Cas13 effector protein is selected from the group consisting of Cas13a, Cas13b, Cas13c and Cas13d.

In an embodiment, the Cas13 effector protein is Cas13b.

Persons skilled in the art will appreciate that any of the systems and methods disclosed herein may be performed using Cas13 effector proteins from orthologs. The term “ortholog” as used herein refers to proteins of a different species that perform the same or a similar function.

In an embodiment, the Cas13b is an ortholog selected from the group consisting of Prevotella buccae Cas13b (pbuCas13b), Prevotella sp. P5-125 Cas13b (PspCas13b), Bergeyella zoohelcum Cas13b (bzCas13b), and Porphyromonas gulae (pguCas13b).

In an embodiment, the Cas13b is PspCas13b.

In an embodiment, the Cas13 effector protein is PspCas13b comprising the amino acid sequence of SEQ ID NO:451, or an amino acid sequence which is at least 80% identical to the amino acid sequence of SEQ ID NO:451. Accordingly, the sequence may be at least 80%, at least 81%, at least 82%, at least 83%, at least 84%, at least 85%, at least 86%, at least 87%, at least 89%, at least 90%, at least 91%, at least 92%, at least 93%, at least 94%, at least 95%, at least 96%, at least 97%, at least 98%, at least 99%, or 100% identical to the amino acid sequence of SEQ ID NO:451. Methods for the determination of amino acid sequence identity would be known to persons skilled in the art, illustrative examples of which include computer programs that employ algorithms such as protein BLAST (Altschul et al., 1997, Nucleic Acids Research, 25: 3389-3402).

In an embodiment, the Cas13 effector protein is PspCas13b encoded by the nucleic acid sequence of SEQ ID NO:452, or a nucleic acid sequence which is at least 80% identical to the nucleic acid sequence of SEQ ID NO: 452. Accordingly, the sequence may be at least 80%, at least 81%, at least 82%, at least 83%, at least 84%, at least 85%, at least 86%, at least 87%, at least 89%, at least 90%, at least 91%, at least 92%, at least 93%, at least 94%, at least 95%, at least 96%, at least 97%, at least 98%, at least 99%, or 100% identical to the nucleic acid sequence of SEQ ID NO:452. Methods for the determination of nucleic acid sequence identity would be known to persons skilled in the art, illustrative examples of which include computer programs that employ algorithms such as BLAST (Altschul et al., 1990, Journal of Molecular Biology, 215(3): 403-410).

In an embodiment, the Cas13 effector protein is Cas13d.

In an embodiment, the Cas13d is an ortholog selected from the group consisting of Eubacterium siraeum (EsCas13d), Ruminococcus sp. (RspCas13d), and Ruminococcus flavefaciens (RfxCas13d).

In an embodiment, the Cas13d is RfxCas13d.

In an embodiment, the Cas13 effector protein is RfxCas13d encoded by the nucleic acid sequence of SEQ ID NO: 561, or a nucleic acid sequence which is at least 80% identical to the nucleic acid sequence of SEQ ID NO: 561. Accordingly, the sequence may be at least 80%, at least 81%, at least 82%, at least 83%, at least 84%, at least 85%, at least 86%, at least 87%, at least 89%, at least 90%, at least 91%, at least 92%, at least 93%, at least 94%, at least 95%, at least 96%, at least 97%, at least 98%, at least 99%, or 100% identical to the nucleic acid sequence of SEQ ID NO: 561. Methods for the determination of nucleic acid sequence identity would be known to persons skilled in the art, illustrative examples of which include computer programs that employ algorithms such as BLAST (Altschul et al., 1990, Journal of Molecular Biology, 215(3): 403-410).

In an embodiment, the Cas13 effector protein is encoded by a codon optimized nucleic acid sequence for expression in particular cells, e.g., eukaryotic cells. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (i.e., differences in codon usage between organisms) often correlates with the efficiency of translation of mRNA, which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, e.g., the “Codon Usage Database” available at www.kazusa.orjp/codon/. Computer algorithms for codon optimizing a particular sequence for expression in a particular host cell are also available, such as Gene Forge (Aptagen; Jacobus, PA), are also available.

The RNA editing system of the present disclosure may comprise more than one crRNA or one or more polynucleotides encoding more than one crRNA, such as 2, 3, 4, 5 or more crRNAs. The multiple crRNAs have sequences that are complementary to different target RNA sequences, such that the crRNAs target or bind to different regions in a nucleic acid molecule. In some examples, the different target RNA sequences may encode the same gene or different genes, or may be in a non-coding region. Thus, in one embodiment, the RNA editing system further comprises a second crRNA or a polynucleotide encoding a second crRNA, wherein the second crRNA comprises a crRNA sequence that is capable of hybridizing to a second target RNA sequence.

The present disclosure also provides vectors comprising a polynucleotide sequence(s) encoding the components of the RNA editing system as described herein.

In an embodiment, the RNA editing system comprises:
a. a polynucleotide encoding a Cas13 effector protein; and
b. the crRNA disclosed herein.

In an embodiment, the polynucleotides of (a) and/or (b) are within one or more vectors.

The vectors can be episomal vectors (i.e., that do not integrate into the genome of a host cell), or can be vectors that integrate into a host cell genome. Vectors may be replication competent or replication-deficient. Exemplary vectors include, but are not limited to, plasmids, cosmids, and viral vectors, such as adeno-associated virus (AAV) vectors, lentiviral, retroviral, adenoviral, herpesviral, parvoviral and hepatitis viral vectors. The choice and design of an appropriate vector is within the ability and discretion of one of ordinary skill in the art. Preferably, however, the vector is suitable for use in biotechnology.

Vectors suitable for use in biotechnology would be known to persons skilled in the art, illustrative examples of which include viral vectors derived from adenovirus, adeno-associated virus (AAV), herpes simplex virus (HSV), retrovirus, lentivirus, self-amplifying single-strand RNA (ssRNA) viruses such as alphavirus (e.g., Semliki Forest virus, Sindbis virus, Venezuelan equine encephalitis, M1), and flavivirus (e.g., Kunjin virus, West Nile virus, Dengue virus), rhabdovirus (e.g., rabies, vesicular stomatitis virus), measles virus, Newcastle Disease virus (NDV) and poxivirus as described by, for example, Lundstrom (2019, Diseases, 6: 42).

In an embodiment, the vector is an adeno-associated virus (AAV) vector. Exemplary AAV vectors include, without limitation, those derived from serotypes AAV1, AAV2, AAV3, AAV4, AAV5, AAV6, AAV7, AAV8, AAV9, AAV10, AAV11, AAV12 or AAV13, or using synthetic or modified AAV capsid proteins such as those optimized for efficient in vivo transduction. A recombinant AAV vector describes replication-defective virus that includes an AAV capsid shell encapsidating an AAV genome. Typically, one or more of the wild-type AAV genes have been deleted from the genome in whole or part, preferably the rep and/or cap genes.

In another embodiment, the polynucleotides of (a) and (b) are within the same vector.

When multiple polynucleotides are combined within the same vector, the expression of each polynucleotide may be controlled by the same promoter or different promoters according to the optimal stoichiometry of the different components of the genome editing system disclosed herein. Thus, in some examples, the polynucleotide encoding the Cas13 effector protein will be operably linked to a first promoter and the polynucleotide encoding the gRNA operably linked to a second promoter.

The term “promoter” as used herein refers to an array of nucleic acid control sequences that direct the transcription of the polynucleotide. Suitable promoters would be known to persons skilled in the art, illustrative examples of which include retroviral LTR elements, constitutive promoters such as CMV, HSV1-TK, SV40, EF-1α, or β-actin, inducible promoters, such as those containing Tet-operator elements, and/or tissue specific promoters.

The polynucleotides may comprise other additional regulatory elements or sequences. Suitable regulatory sequences would be known to persons skilled in the art, illustrative examples of which include leader or signal sequences, ribosomal binding sites, transcriptional start and termination sequences, and enhancer or activator sequences. It is also contemplated herein that the polypeptides comprises elements and sequences associated with protein localization and interactions. For example, the polynucleotides encoding the polypeptide tag may comprise sequences encoding a nucleus localization sequence (NLS).

The present disclosure also provides non-viral delivery vehicles of the RNA editing systems as described herein. Suitable non-viral delivery vehicles will be known to persons skilled in the art, illustrative examples of which include using lipids, lipid-like materials or polymeric materials, as described, for example, by Rui et al. (2019, Trends in Biotechnology, 37(3): 281-293), and nanoparticles/nanocarriers, as described by, for example, Nguyen et al. (2020, Nature Biotechnology, 38: 44-49), Duan et al. (2021, Frontiers in Genetics, 12: 673286), and Rahimi et al. (2020, Nanotoday, 34: 100895).

In an embodiment, the Cas13 effector protein of (a) and the crRNA of (b) are combined to form a pre-assembled ribonucleoprotein. In accordance with this embodiment, the pre-assembled ribonucleoprotein can be delivered to cells by non-viral delivery methods, such as lipofection or electroporation.

In an embodiment, the polynucleotide encoding a Cas13 effector protein or Cas13 effector protein of (a) and/or the polynucleotide encoding the crRNA or the crRNA of (b) are within a nanoparticle.

As described elsewhere herein, multiple polynucleotides may be combined within the same vector. It is contemplated herein that any polynucleotides that are not comprised within the same vector may be provided to the cell using non-viral delivery vehicles. Accordingly, in an embodiment, the polynucleotide of (a) may be comprised in a vector and the polynucleotide of (b) in a non-viral delivery vehicles.

In another aspect, the present disclosure provides a cell or a cell extract comprising the RNA editing system as described herein.

Cells according to the present disclosure include any cell into which the polynucleotides, vectors and polypeptides described herein may be introduced and expressed. It is not intended that use of the RNA editing systems disclosed herein be limited by cell type. Accordingly, the cells of the present disclosure include eukaryotic cells, prokaryotic cells, animal cells, plant cells, fungal cells, archaeal cells, eubacterial cells and the like.

The cell or cell extract contemplated herein may be derived from any species, particularly a vertebrate, and even more particularly a mammal. Suitable vertebrates that fall within the scope of the disclosure include, but are not restricted to, any member of the subphylum Chordata including primates (e.g., humans, monkeys and apes, and includes species of monkeys such from the genus Macaca (e.g., cynomolgous monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. In a preferred embodiment, the cell or cell extract are derived from a human.

The cell or cell extract may be provided with the RNA editing systems described herein using any suitable method known in the art. Such methods include transfection, transduction, viral transduction, microinjection, lipofection, nucleofection, nanoparticle bombardment, transformation, conjugation and the like. The skilled person would readily understand and adapt any such method taking consideration of whether the components of genome editing system are provided as polynucleotides, vectors or polypeptides.

### Methods for Altering a Target RNA Sequence

In another aspect, the present disclosure provides a method of altering a target RNA sequence in a cell, the method comprising providing to the cell the RNA editing system as described herein, wherein the Cas13 effector protein when in conjunction with the crRNA, specifically hybridizes to the target RNA sequence, and wherein the Cas13 effector protein alters the hybridized target RNA sequence.

The term “altering” as used herein refers to any change to the target RNA sequence, which modifies the synthesis of a gene product, such as a protein, by cleavage, editing, splicing, etc.

By “gene” it is meant a unit of inheritance that, when present in its endogenous state, occupies a specific locus on a genome and comprises transcriptional and/or translational regulatory sequences and/or a coding region and/or non-translated sequences (e.g., introns, 5′ and 3′ untranslated sequences).

In an embodiment, the alterations contemplated herein can be applied to enhance translation, repress translation, exon skipping, exon inclusion, altering RNA localization, RNA degradation, and inhibition of non-coding RNA function.

In an embodiment, alteration of the target RNA sequence results in RNA knockdown, RNA base-editing, RNA binding, RNA pulldown, RNA imaging or RNA modification.

In an embodiment, the alteration to the target RNA sequence occurs via cleavage of the target RNA sequence, resulting in RNA knockdown (also referred to as “RNA interference” or “RNA degradation”).

In an embodiment, the alteration of the target RNA sequence results in the cell comprising altered expression of at least one gene product; and wherein:
a. the cell comprising altered expression of at least one gene product, wherein the expression of the one gene product is increased; or
b. the cell comprising altered expression of at least one gene product, wherein the expression of the one gene product is decreased.

The term “increased expression” as used herein means a level of expression that is lower than observed in cells that have not been contacted with the RNA editing system. It is to be understood that the term “increased” as used herein, does not necessarily imply that expression of a gene product encoded by the target RNA sequence has been increased. In some embodiments, the level of expression of at least one gene product associated with the target RNA sequence or a gene product encoded by the target RNA sequence may be increased by at least about 50% (e.g., at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100%).

The term “decreased expression” as used herein means a level of expression that is lower than observed in cells that have not been contacted with the RNA editing system. It is to be understood that the term “decreased” as used herein, does not necessarily imply that expression of a gene product encoded by the target RNA sequence has been eliminated or is reduced to an undetectable level. In some embodiments, the level of expression of at least one gene product associated with the target RNA sequence or a gene product encoded by the target RNA sequence may be reduced by at least about 50% (e.g., at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or effectively abolished to an undetectable level, i.e., 100%).

In an embodiment, the expression of the target RNA sequence is reduced to an undetectable level. Persons skilled in the art will appreciate that a reduction is expression to an undetectable level is intended to encompass embodiments whereby the expression of the target RNA sequence is effectively abolished.

The crRNA described herein have been demonstrated to exhibit minimal off-target effects, even when targeting transcripts with high levels of homology with one or more non-target transcripts. Such homologous transcripts would be known to persons skilled in the art, illustrative examples of which include gene fusion transcripts, RNA isoforms and variant transcripts comprising at least one SNV.

Accordingly, in an embodiment, the target RNA sequence shares homology with one or more non-target RNA sequences.

In an embodiment, the target RNA sequence is selected from an RNA isoform, a variant transcript and a gene fusion transcript.

In an embodiment, the target RNA sequence is a gene fusion transcript.

The term “gene fusion transcript” as used herein refers to aberrant RNA structures resulting from chromosomal translocations. Illustrative examples of gene fusion transcripts would be known to persons skilled in the art and include gene fusion transcripts that are frequently detected in cancer types.

In an embodiment, the spacer sequence is capable of hybridizing to a target RNA sequence comprising the fusion breakpoint of the gene fusion transcript.

In an embodiment, the gene fusion is selected from the group consisting of BCR-ABL1, SFPQ-ABL1 and SXN2-ABL1.

In an embodiment, the spacer sequence comprises any one of the nucleic acid sequences set forth in SEQ ID NOs: 103-161 and 457 to 462, and those having at least about 90%, 90%, 91%, 92%, 93%, 94%, 95%, 96%, 98%, or 99% sequence identity to the to the spacer sequences set forth in SEQ ID NOs: 103-161 and 457 to 462.

In an embodiment, the spacer sequence comprises any one of the nucleic acid sequences set forth in SEQ ID NOs: 123, 153, 161 and 457 to 462, and those having at least about 90%, 90%, 91%, 92%, 93%, 94%, 95%, 96%, 98%, or 99% sequence identity to the to the spacer sequences set forth in SEQ ID NOs: 123, 153, 161 and 457 to 462.

In an embodiment, the spacer sequence is capable of hybridizing to the gene fusion transcript and the gene fusion transcript comprising one or more secondary mutations.

The term “secondary mutations” as used herein refers to a second genetic change in a gene (e.g., an oncogenic driver) that confers acquired resistance to a targeted therapeutic agent. Such secondary mutations would be known to persons skilled in the art, illustrative examples of which include the BCR-ABL T315I mutation that confers resistance to ABL1 inhibitors, e.g., imatinib.

In an embodiment, the target RNA sequence is a variant transcript comprising at least one SNV.

“Single nucleotide variants” or “SNVs” are a target RNA sequence encoding a gene product comprising a somatic point mutation in which one nucleotide of a given gene sequence is substituted for another. The resulting amino acid change frequently results in the generation of an aberrant protein with a structure and/or function that differs from its wild-type homolog.

In an embodiment, the SNV is a pathogenic mutation.

By “pathogenic mutation” it is meant that the encoded gene product is increases susceptibility or predisposition to a disease or disorder. For example, pathogenic mutations are enriched in archetypical proto-oncogenes such as BRAF, KRAS and PIK3CA. Cancer cells which harbour such mutations in these tumour drivers are capable of sustained proliferative signaling in the absence of stimulatory input and are insensitive to the negative regulatory mechanisms designed to prevent over-activation of these pathways.

Pathogenic mutations would be known to persons skilled in the art, illustrative examples of which include BRAF V600E, KRAS G12C, KRAS G12R, KRAS G12S, KRAS G12A, KRAS G12V, KRAS G12D, and the SNVs reported in the Pan Cancer Analysis of Whole Genomes (PCAWG) by Campbell et al. (2020, Nature, 578: 82-93).

In an embodiment, the pathogenic mutation is BRAF^{V600E}.

The BRAF^{V600E} mutation, in which a single T>A nucleotide substitution results in the replacement of valine by glutamate at amino acid position 600, is the most common BRAF aberration and is found in approximately 7% of all human cancers and up to 60% of melanomas. Whilst wild type BRAF signals as a homo- or heterodimer with other RAF family members in response to phosphorylation of its kinase domain by RAS, BRAF^{V600E} functions as a constitutively active monomer in the absence of RAS stimulation and consequently drives cells into a hyperproliferative state.

In an embodiment, the crRNA comprises any one of the sequences set forth in SEQ ID NOs: 419-423, 435-437, 439, 441 and 465-560, and those having at least about 90%, 90%, 91%, 92%, 93%, 94%, 95%, 96%, 98%, or 99% sequence identity to the to the spacer sequences set forth in SEQ ID NOs: 419-422, 435-437, 439, 441, and 465-560.

In an embodiment, the pathogenic mutation is a KRAS mutation selected from the group consisting of KRAS G12C, KRAS G12R, KRAS G12S, KRAS G12A, KRAS G12D, KRAS G12V, KRAS G13D, KRAS G13C, KRAS Q61L, and combinations of the foregoing.

In an embodiment, the pathogenic mutation is a KRAS mutation selected from the group consisting of KRAS G12C, KRAS G12R, KRAS G12S, KRAS G12A, KRAS G12D, KRAS G12V, and combinations of the foregoing.

In an embodiment, the crRNA comprises any one of the sequences set forth in SEQ ID NOs: 489-560, and those having at least about 90%, 90%, 91%, 92%, 93%, 94%, 95%, 96%, 98%, or 99% sequence identity to the to the spacer sequences set forth in SEQ ID NOs: 489-560.

### Pharmaceutical Compositions

The present disclosure also provides for compositions, including pharmaceutical compositions, comprising the RNA systems described herein (e.g., vectors and/or non-viral delivery vehicles) as disclosed herein. In some embodiments, pharmaceutical compositions comprise an effective amount of the RNA systems as described herein and a pharmaceutically acceptable carrier. For instance, in certain embodiments, the pharmaceutical composition comprises an effective amount of one or more vectors and a pharmaceutically acceptable carrier. An effective amount can be readily determined by those skilled in the art based on factors such as body size, body weight, age, health, sex of the subject, ethnicity, and viral titres.

The phrases “pharmaceutically acceptable” or “pharmacologically acceptable” refer to molecular entities and compositions that do not produce adverse, allergic, or other untoward reactions when administered to an animal or a human. For example, an expression vector may be formulated with a pharmaceutically acceptable carrier. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. Methods for the formulation of compounds with pharmaceutical carriers are known in the art and are described in, for example, in Remington's Pharmaceutical Science, (17th ed. Mack Publishing Company, Easton, Pa. 1985); and Goodman & Gillman's: The Pharmacological Basis of Therapeutics (11th Edition, McGraw-Hill Professional, 2005); the disclosures of each of which are hereby incorporated herein by reference in their entirety.

Pharmaceutically acceptable carriers suitable for inclusion within any pharmaceutical composition include water, buffered water, saline solutions such as, for example, normal saline or balanced saline solutions such as Hank's or Earle's balanced solutions), glycine, hyaluronic acid etc. The pharmaceutical composition may be formulated for parenteral administration, such as intravenous, intramuscular or subcutaneous administration. Pharmaceutical compositions for parenteral administration may comprise pharmaceutically acceptable sterile aqueous or non-aqueous solutions, dispersions, suspensions or emulsions as well as sterile powders for reconstitution into sterile injectable solutions or dispersions. Examples of suitable aqueous and non-aqueous carriers, solvents, diluents or vehicles include water, ethanol, polyols (e.g., glycerol, propylene glycol, polyethylene glycol, etc.), carboxymethylcellulose and mixtures thereof, vegetable oils (e.g., olive oil), injectable organic esters (e.g., ethyl oleate).

### Methods of Gene Therapy

It is further contemplated that the RNA editing systems and methods described herein may be adapted for the treatment of diseases and disorders that are characterized by gene fusion transcripts, RNA isoforms or single-nucleotide variants. For example, it has been exemplified herein that the RNA editing systems comprising potent crRNA efficiently and selectively target RNA sequences encoding oncogenic gene fusions, which are associated with both hematologic malignancies and solid tumors. On this basis, it is reasonable to expect that the RNA editing systems and methods described herein may also be useful in the treatment of cancer

Accordingly, in an aspect disclosed herein there is provided a method for the treatment of cancer comprising the administration of a therapeutically effective amount of the RNA editing system, the cell or the cell extracts described herein to a subject in need thereof.

In an embodiment, the cancer is a gene fusion transcript-dependent cancer.

Gene fusion transcript-dependent cancers would be known to persons skilled in the art, illustrative examples of which include acute lymphoblastic leukaemia (e.g., SFPQ-ABL1 and SXN2-ABL1), chronic myeloid leukaemia (e.g., BCR-ABL1), adenoid cystic carcinoma (e.g., MYB-NFIB, NFIB-HMGA2), muceoepidermoid carcinoma (e.g., MECT-MAML2), follicular thyroid carcinoma (e.g., PAX8-PPARG), breast carcinoma (e.g., ETV6-NTRK3, FGFR3-AFF3, FGFR2-CASP7, FGFR2-CCDC6, ERLIN2-FGFR1), Ewing sarcoma (e.g., EWSR1-FLI1), small round cell tumours of bone (e.g., BCOR-CCNB3), synovial sarcoma (e.g., SS18-SSX1, SS18-SSX2), glioblastoma multiforme (e.g., FGFR3-TACC3, FGFR1-TACC1), pilocytic astrocytoma (e.g., KIAA1967-BRAF), lung cancer (e.g., EML4-ALK, FGFR3-TACC3, FGFR3-KIAA 1967, BAG4-FGFR1), clear cell renal cell carcinoma (e.g., SFPQ-TFE3, TFG-GPR128), bladder cancer (e.g., FGFR3-TACC3, FGFR3-BAIAP2L1), prostate cancer (e.g., TMPRSS2-ERG/ETV1/ETV4, SLC45A3-FGFR2), ovarian cancer (e.g., ESRRA-C11orf20) and colorectal cancer (e.g., PTPRK-RSPO3, EIF3E-RSPO2).

In an embodiment, the gene fusion transcript-dependent cancer is selected from acute lymphoblastic leukaemia (e.g., SFPQ-ABL1 and SXN2-ABL1) and chronic myeloid leukaemia (e.g., BCR-ABL1).

In an embodiment, the cancer is a SNV-dependent cancer.

SNV-dependent cancers would be known to persons skilled in the art, illustrative examples of which include melanoma, colorectal cancer, rectal cancer, thyroid cancer, ovarian cancer, brain tumors, lung cancer and pancreatic cancer.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

It will be appreciated by persons skilled in the art that numerous variations and/or modifications may be made to the present disclosure without departing from the spirit or scope of the disclosure as broadly described. The present embodiments are, therefore, to be considered in all respects as illustrative and not restrictive.

The present disclosure will now be further described in greater detail by reference to the following specific examples, which should not be construed as in any way limiting the scope of the disclosure.

### EXAMPLES

### General Methods

Design and Cloning of crRNAs for PspCas13b

The design and cloning of PspCas13b crRNAs were designed and cloned according to method described by Fareh et al. (2021, Nature Communications, 12: 4270). Briefly, individual guide RNAs were cloned into the pC0043-PspCas13b crRNA backbone (Addgene #103854, a gift from Feng Zhang lab, “crRNA backbone”, SEQ ID NO:453), which contains PspCas13b gRNA direct repeat (DR) sequence and two BbsI restriction sites for the cloning of spacer sequence. A total of 20 μg crRNA backbone was digested by BbsI restriction enzymes (NEB, R3539) following the manufacturer's instructions for 2 hours at 37° C. Backbone linearization was checked by 1% agarose gel. The digested backbone was purified with NucleoSpin Gel and PCR Clean-up Kit (Macherey-Nagel, 740609.50), aliquoted, and stored in −20° C.

For crRNA cloning, a forward and reverse single-stranded DNA oligonucleotides containing CACC and CAAC overhangs respectively, were obtained from Sigma or IDT (100 μM). A total of 1.5 μL of 100 μM the forward and reverse DNA oligonucleotides were annealed in 47 μL annealing buffer (5 μl NEB buffer 3.1 and 42 μL H₂O) by 5 min incubation at 95° C. and slow cool down in the heating block overnight. 1 μL of the annealed oligonucleotides were ligated with 0.04 ng digested PspCas13b crRNA backbone in 10 μL of T4 ligation buffer (3 h, RT) (Promega, M1801). All PspCas13b crRNA spacer sequences used in this study are listed in Table 1. All crRNAs and PspCas13b clones that are generated in this study were verified by Sanger sequencing. The primers used for PCR and Sanger sequencing are listed in Table 2.

### Cloning of BCR-ABL1, ABL1, BCR, BRAF-WT and BRAF^{V600E} Fragments

The partial sequence of BCR-ABL1, ABL1 and BCR was designed according to the full length BCR-ABL1 P190 (SEQ ID NO: 402). The IDT DNA synthesis platform provided the three sequences that were subsequently cloned into MSCV-IRES-mCherry, MSCV-IRES-eGFP and MSCV-IRES-tagBFP vectors respectively in frame with 3×HA tag using EcoRI/BamHI digestion (Promega, R6011/Promega, R6021), gel purification, and ligation with T4 DNA ligase. Similarly, the partial sequences of wild type BRAF (BRAF-WT) or BRAF^{V600E} were designed according to full length BRAF and these were cloned into MSCV-IRES-eGFP of MSCV-IRES-mCherry, respectively, as described above. The ligated product was transformed into chemically competent bacteria (TOP10 or Stbl3) and positive clones were screened by PCR and Sanger sequencing (AGRF, AUSTRALIA). The BCR-ABL1-3×HA-IRES-mcherry, BCR-3×HA-IRES-tagBFP and ABL1-3×HA-IRES-EGFP, BRAF-WT and BRAFV600E constructs are shown in SEQ ID NOs:400-406, 463 and 464. The primers used for PCR and Sanger sequencing are listed in Table 2.

### Plasmid Amplification and Purification

Plasmid amplification and purification were performed as described by Fareh et al. (2021, supra). Briefly, TOP10 or Stbl3 bacteria were used for transformation. A total of 5-10 μL ligated plasmids were transformed into 30 μL of chemically competent bacteria by heat shock at 42° C. for 45 s, followed by 2 min on ice. The transformed bacteria were incubated in 500 μL LB broth media containing 75 μg/mL ampicillin (Sigma-Aldrich, A9393) for 1 h at 37° C. in a shaking incubator (200 rpm). The bacteria were pelleted by centrifugation at 6,000 rpm for 1 min at room temperature (RT), re-suspended in 100 μL of LB broth, and plated onto a pre-warmed 10 cm LB agar plate containing 75 μg/mL ampicillin, and incubated at 37° C. overnight. The next day, single colonies were picked and transferred into bacterial starter cultures and incubated for ˜6 h for mini-prep (Macherey-Nagel, NucleoSpin Plasmid Mini kit for plasmid DNA, 740588.50) or maxi-prep (Macherey-Nagel, NucleoBond Xtra Maxi Plus, 740416.50) DNA purification according to the standard manufacturer's protocol.

### Cell Culture

The HEK 293 T (ATCC CRL-3216) and A375 (ATCC CCL-1619) cell lines were cultured in DMEM high glucose media (Thermo Fisher, 11965092) containing 10% heat-inactivated fetal bovine serum (Thermo Fisher, 10100147), 100 mg/ml Penicillin/-Streptomycin (Thermo Fisher, 151401220), and 2 mM GlutaMAX (Thermo Fisher, A1286001). The HCT116 (ATCC CCL-247) cell line was cultured in Advanced RPMI 1640 media (Thermo Fisher, 12633012) containing 10% heat-inactivated fetal bovine serum (Thermo Fisher, 10100147), 100 mg/ml Penicillin/-Streptomycin (Thermo Fisher, 151401220), and 2 mM GlutaMAX (Thermo Fisher, A1286001). All cells were routinely tested and were Mycoplasma negative.

### Nucleic Acid Silencing Assays by Transient Transfection

All transfection experiments were performed using an optimized Lipofectamine 3000 transfection protocol (Thermo Fisher, L3000015). For RNA silencing in HEK 293 T, cells were plated at approximately 30,000 cells/100 μL/96-well in tissue culture treated flat-bottom 96-well plates (Corning) 18 h prior to transfection. For each well, a total of 100 ng DNA plasmids (22 ng of Ef1a-PspCas13b-NES-3×FLAG-T2A-BFP (Addgene #173029; SEQ ID NO:454) or pC0046-EF1a-PspCas13b-NES-HIV (Addgene #103862; SEQ ID NO:455) or FUCas9-mCherry (Addgene #70182; SEQ ID NO:456), 22 ng crRNA plasmid, and 56 ng of the target gene) were mixed with 0.2 μL P3000 reagent in Opti-MEM Serum-free Medium (Thermo Fisher, 31985070) to a total of 5 μL (“Mix1”). Separately, 4.7 μL of Opti-MEM was mixed with 0.3 μL Lipofectamine 3000 (“Mix2”). Mix1 and Mix2 were added together and incubated for 20 min at room temperature, then 10 μL of transfection mixture was added to each well. Table 3 summarizes the transfection conditions used in 96, 24, and 12-well plates. After transfection, cells were incubated at 37° C., 10% CO₂, and the transfection efficacy was monitored 24-72 hours post-transfection by fluorescent microscopy.

### Fluorescent Microscopy Analysis

For RNA silencing experiments, the fluorescence intensity was monitored using EVOS M5000 FL Cell Imaging System (Thermo Fisher). Pictures were taken 48 h post-transfection, and the fluorescence intensity of each image was quantified using a lab-written macro in ImageJ software. Briefly, all images obtained from a single experiment are simultaneously processed using a batch mode macro. First, images were converted to 8-bit, threshold adjusted, converted to black and white using Convert to Mask function, and fluorescence intensity per pixel measured using Analyze Particles function. Each single mean fluorescence intensity was obtained from four different field of views for each crRNA, and subsequently normalized to the non-targeting (NT) control crRNA. Two-fold or higher reduction in fluorescence intensity is considered as biologically relevant.

### Western Blot

Cells were washed three times with ice-cold PBS± and lysed on ice in RIPA lysis buffer [50 mM Tris (Sigma-Aldrich, T1530), pH 8.0, 150 mM NaCl, 1% NP-40 (Sigma-Aldrich, I18896), 0.1% SDS, 0.5% sodium deoxycholate (Sigma-Aldrich, D6750)] containing protease inhibitor cocktail (Roche, 04693159001) and phosphatase inhibitor cocktail (Roche, 4906845001). Samples were incubated for 30 min at 4° C. with rotation (25 rpm), and centrifuged at 16,000 g for 10 min, 4° C. Supernatant was transferred to a new tube. Protein concentrations were quantified using the Pierce BCA Protein Assay Kit (Thermo Fisher, 23225) according to the manufacturer's instructions. A total of 10 μg proteins diluted in 1× Bolt LDS sample buffer (Thermo Fisher, B007) and 1× Bolt sample reducing agent (Thermo Fisher, B009) were denatured at 95° C. for 5 min. Samples were resolved by Bolt Bis-Tris Plus 4-12% gels (Thermo Fisher, NW04120BOX) in 1×MES SDS running buffer (Thermo Fisher, B0002) and transferred to 0.45 μM PVDF membranes (Thermo Fisher, 88518) by a Trans-Blot Semi-Dry electrophoretic transfer cell (Bio-Rad) at 20 Volt for 30 min. Alternatively, samples were resolved by 4-15% Criterion TGX Precast Midi Protein gels (Bio-Rad, 5671084) in 1× Tris/glycine/SDS running buffer (Bio-Rad, 1610732) and transferred to 0.20 μM nitrocellulose membranes (Bio-Rad, 1704159) by a Trans-Blot Turbo Transfer System (Bio-Rad) with a HIGH MW protocol. Membranes were incubated in blocking buffer 5% (w/v) BSA (Sigma-Aldrich, A3059) in TBST with 0.15% Tween 20 (Sigma-Aldrich, P1379) for 1 h at RT and probed overnight with primary antibodies at 4° C. Blots were washed three times in TBST with 0.15% Tween20, followed by incubation with fluorophore-conjugated or HRP-conjugated secondary antibodies for 1 h at RT. Membranes were washed in TBST (0.15% Tween20) three times and fluorescence or chemiluminescence was detected using the Odyssey CLx Imager 9140 (Li-cor), iBright CL 1500 Imaging System (Thermo Fisher), or ChemiDoc Imaging System (Bio-Rad). The antibodies used for western blots are listed in Table 4

RNA Extraction, cDNA Synthesis and RT-PCR

Total RNA was isolated from around 5×10⁵ to 1×10⁶ cells using the NucleoSpin RNA Plus (MACHEREY-NAGEL, 740984.50) or Quick-RNA Miniprep Kit (Zymo Research, R1055) following the manufacturer's instructions. 1 μg total RNA was converted to cDNA using the high-capacity cDNA reverse transcription kit (Thermo Fisher, 4368814) following the manufacturer's instructions. Quantitative RT-PCR reaction was performed in duplication in a StepOne Real-Time PCR system (Thermo Fisher) using PowerUp™ SYBR™ Green Master Mix (Thermo Fisher, A25742). Total reaction mixture contains 0.2 μl cDNA, 0.6 μM forward primer and 0.6 Mm reverse primer. Primers for RT-PCR are detailed in Table 2.

### Prediction of RNA Secondary Structure, RNA-MFE and RNA-RNA Hybridization Energy

RNAfold was used to predict the MFE of crRNA spacer, crRNA (DR and spacer), and the 70 nt target region in the target RNA (20 nt up/downstream from the 30 nt-spacer-binding region). RNAfold was also used to explore the secondary structure of crRNAs and the target regions in the target RNAs. RNAplex and intaRNA were used to predict the hybridization energy and interaction energy between crRNA spacer and target RNA, respectively.

### Data Analysis

Data analyses and visualizations (graphs) were performed in GraphPad Prism software version 9, unless stated otherwise. Specific statistical tests, numbers of independent biological replicates are mentioned in respective figure legends. The silencing efficiency of various crRNAs was analyzed using one-way ANOVA followed by Dunnett's multiple comparison test where we compare every mean to a control mean as indicated in the Figures (95% confidence interval). The P values (P) are indicated in the Figures. P<0.05 is considered as statistically significant. Pearson correlation coefficient was used to analyze correlation between the crRNA silencing efficiency and potential parameters including crRNA MFE, target MFE, crRNA spacer MFE, crRNA-target RNA hybridization/interaction energy, crRNA spacer GC content, and A/U/G/C content. The R package ‘ggseqlogo’ was used to assess nucleotide preference in crRNA spacer and PFS sequences (Wagih, 2017, Bioinformatics, 33(22):3645-3647). Delta probability graphs of spacer nucleotides were generated with Matplotlib.

### Example 1

### PspCas13b Silencing Efficiency is Highly Variable Among Various crRNAs

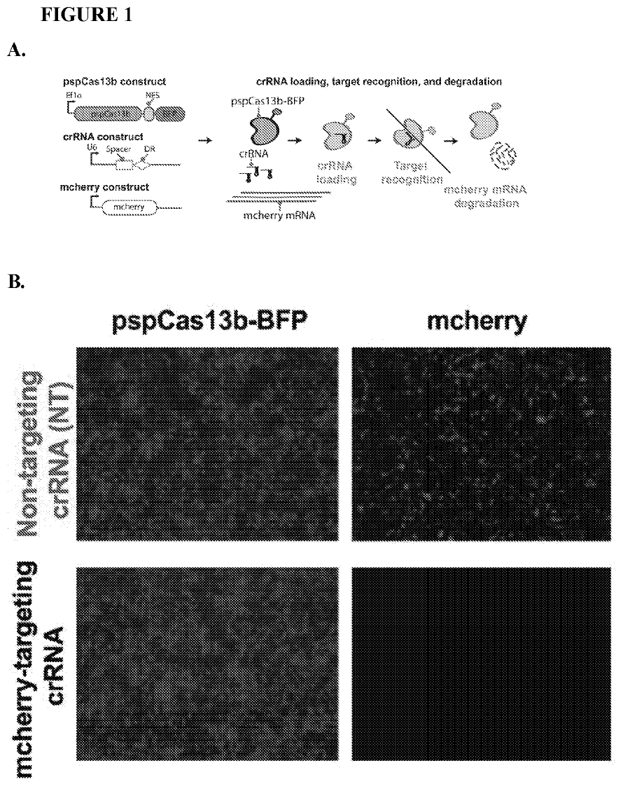

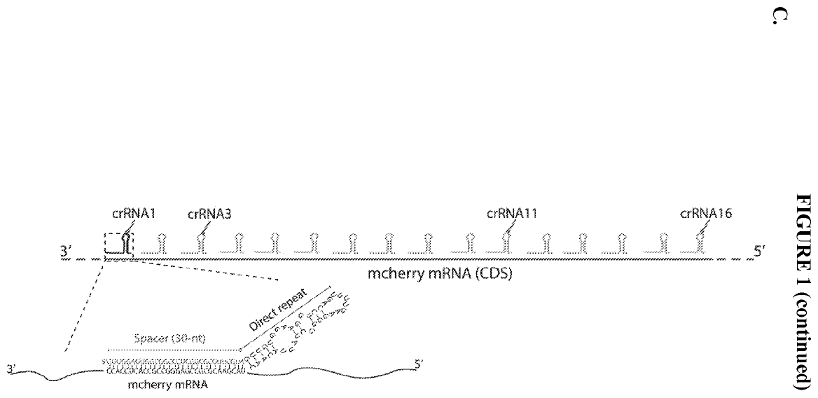

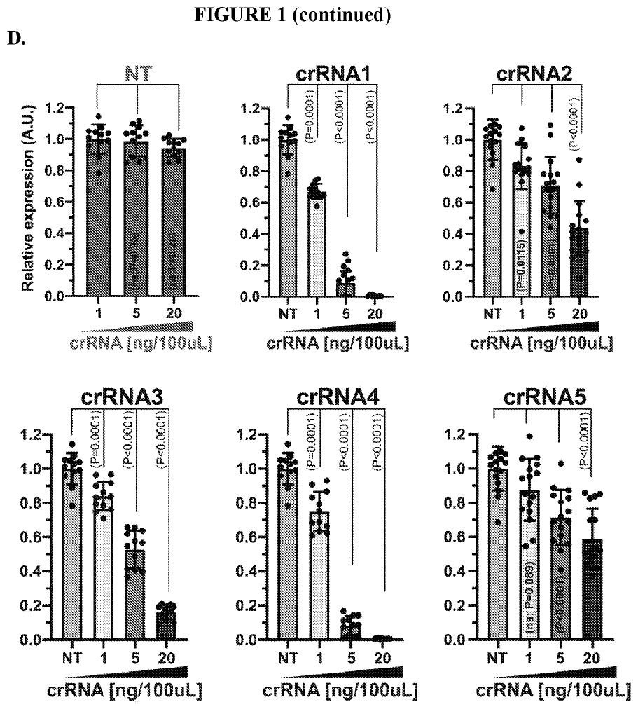

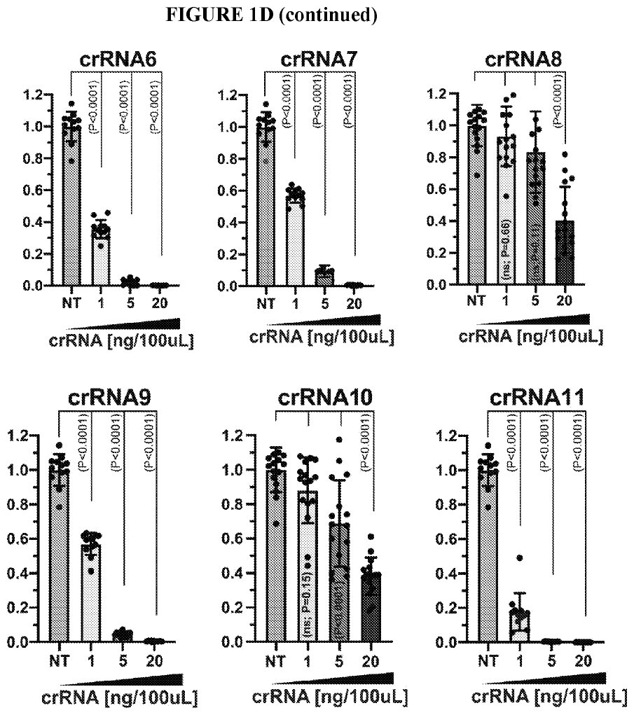

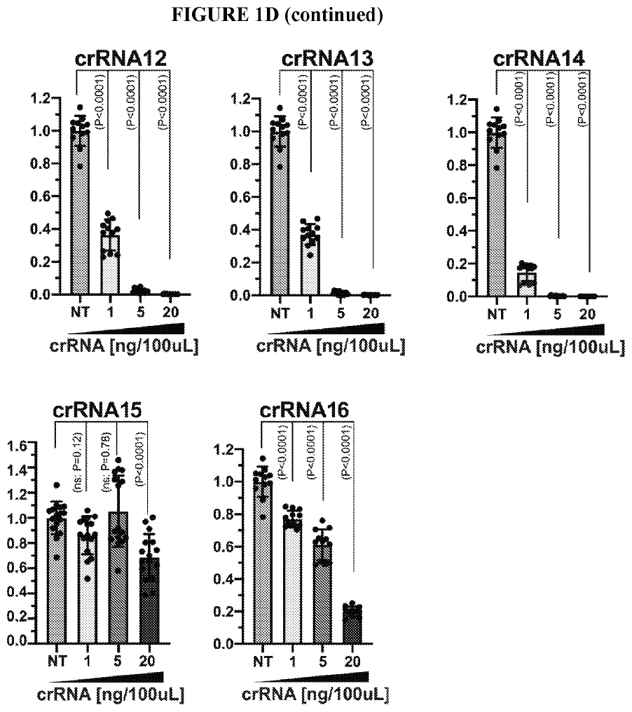

To elucidate PspCas13b crRNA design principles, we developed a quantitative fluorescence-based silencing assay in which PspCas13b crRNA was reprogrammed to target the transcript of the mCherry reporter gene in the cytoplasm of mammalian cells. We co-transfected HEK 293T cells with an mCherry plasmid together with PspCas13b tagged with blue fluorescent protein (BFP), and either a non-targeting (NT) or mCherry-targeting crRNAs (FIG. 1A). The intracellular expression of crRNAs and PspCas13b was anticipated to initiate crRNA loading into PspCas13b, prompting target recognition, Cas13b activation and mCherry mRNA degradation (FIG. 1A). Fluorescence microscopy analysis of cells transfected with on-target mCherry crRNAs showed pronounced silencing activity, contrasted with no appreciable silencing in cells receiving NT crRNAs (FIG. 1B). These data demonstrate the feasibility and tractability of PspCas13b reprogramming for high efficiency gene silencing in mammalian cells.

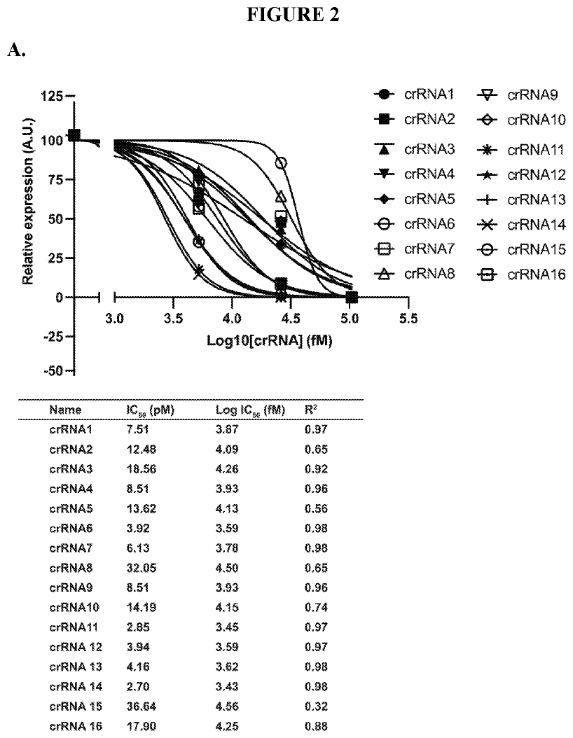

Next, we hypothesized that parameters such as efficiency of crRNA transcription, crRNA loading, spacer nucleotide composition, target accessibility, and the presence of a potential protospacer-flanking sequence (PFS) may influence the efficiency of PspCas13b and could lead to variability in the silencing profiles of various crRNAs. To address this question, we empirically designed 16 crRNAs with spacer sequences that fully basepair with the coding sequence of the mCherry mRNA at various positions (FIG. 1C). To accurately determine the silencing efficacy of each crRNA in this cohort, we performed crRNA dose-dependent silencing assays in which cells were transfected with 0, 1, 5, and 20 ng of each of 16 mCherry-targeting crRNAs and quantitated silencing efficiency. Irrespective of dose, NT crRNA did not exhibit any silencing, while mCherry targeting crRNAs typically demonstrated dose-dependent silencing. However, we noticed marked differences in the silencing efficacy of the various crRNAs. For example, crRNA6 (SEQ ID NO: 11), crRNA11 (SEQ ID NO: 16), crRNA12 (SEQ ID NO: 17), crRNA13 (SEQ ID NO: 18), and crRNA14 (SEQ ID NO: 20) were extremely potent and degraded the majority of mCherry mRNA at a very low dose of 1 ng plasmid (5.2 pM). Conversely, crRNA2 (SEQ ID NO: 7), crRNA5 (SEQ ID NO: 10), crRNA8 (SEQ ID NO: 13), crRNA10 (SEQ ID NO: 15), and crRNA15 (SEQ ID NO: 21) were inefficient and failed to completely degrade mCherry mRNA even at higher doses of 5 and 20 ng (26 and 104 pM) (FIG. 1D). crRNA potency was determined via calculation of the IC₅₀ value, a dose that achieved 50% degradation of the target RNA, which confirmed the high variability in the silencing efficiency of various crRNAs (FIGS. 2A and 2B). Surprisingly, although crRNA14 (SEQ ID NO: 20) and crRNA15 (SEQ ID NO: 21) target neighboring sequence regions, separated by just 8 nucleotides, their silencing efficiencies were markedly disparate. For example, 5 ng of crRNA14 silenced >99% of mCherry expression (P<0.0001), while the same amount of crRNA15 did not show significant silencing of mCherry (P=0.78) (FIG. 1D). As these two crRNAs target spatially adjacent sequences, this finding suggested there are determinants of PspCas13b efficacy beyond target accessibility. Identifying such determinants is crucial in optimizing crRNA design.

### Example 2

### Single-Nucleotide Resolution Screen Revealed the Interplay Between PspCas13b Silencing and RNA Landscape

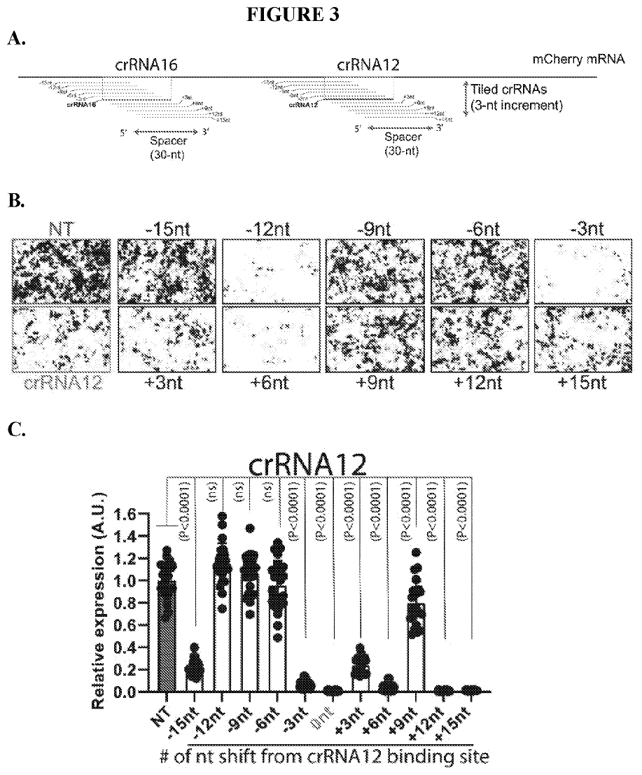

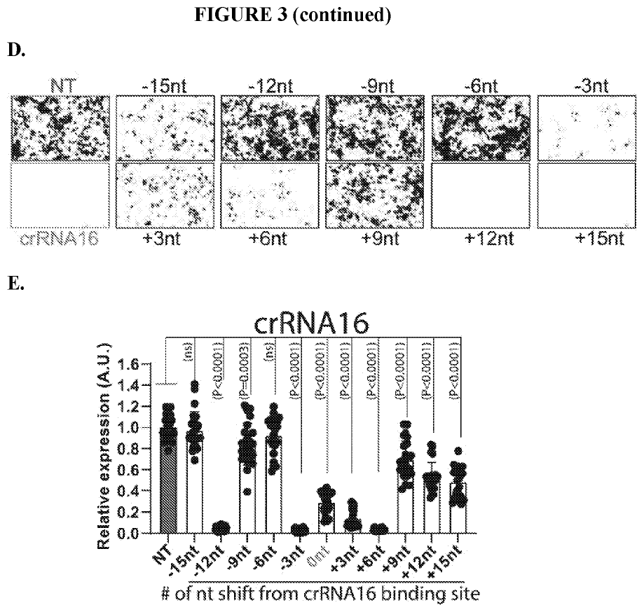

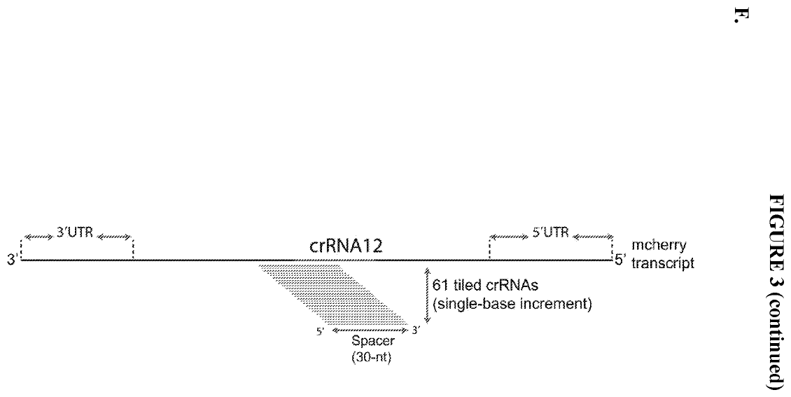

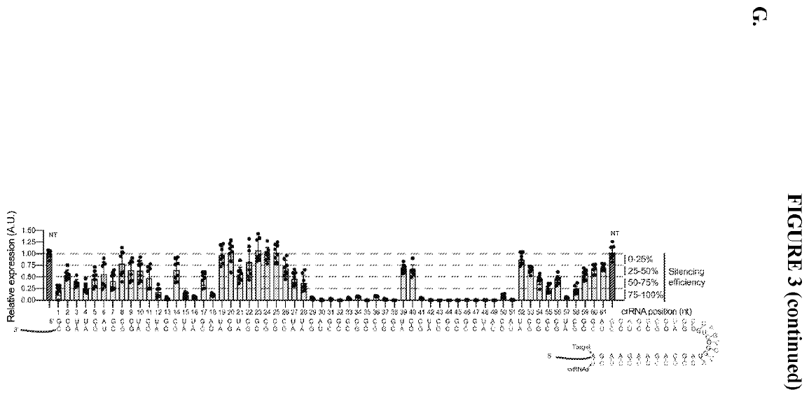

To further understand the spectrum of crRNA silencing activity, we investigated PspCas13b activity variation across a spatially defined targeted region, reasoning that silencing efficiency is likely intrinsically related to the spatial characteristics of the crRNA binding site. We focused our study on crRNA12 (SEQ ID NO: 17) and crRNA16 (SEQ ID NO: 21) that previously achieved high and moderate silencing, respectively. We designed 3-nucleotide resolution tiled crRNAs spanning a 30-nucleotide target region surrounding crRNA12 and crRNA16 (FIG. 3A). In this tiled design, each adjacent crRNAs are spaced by 3 nucleotides, thus silencing profiles should reveal the relationship between efficacy, the sequence of the spacer-target, and target accessibility. We again observed considerable heterogeneity in the potency of these tiled crRNAs despite their physical proximity, with some adjacent crRNAs demonstrating antipodal silencing efficacy (FIG. 3B-3C). These data indicated that physical barriers such as RNA binding proteins or structured RNA motifs are unlikely to explain the fluctuation in silencing between neighboring crRNAs. Rather, the variability in PspCas13b potency is possibly attributable to changes in the sequence of a potential PSF, spacer nucleotide composition, or nucleotide position within the spacer.

To further enhance our understanding, we maximized the spatial resolution of this approach by designing 61 tiled crRNAs with single nucleotide incremental targeting of the region surrounding crRNA12 (FIG. 3D; SEQ ID NOs: 42-102). Consistent with previous data, we again observed markedly diverse silencing profiles of neighboring crRNAs (FIG. 3E). For instance, crRNA13 (SEQ ID NO: 54) achieved silencing exceeding 95% efficiency, but shifting the targeted region by only 1 nucleotide (crRNA14; SEQ ID NO: 55) dramatically reduced efficiency to ˜30%. Similarly, crRNA51 (SEQ ID NO: 92) yielded ˜99% silencing efficiency while its adjacent crRNA52 did not show any appreciable silencing activity (FIG. 3E).

These data strengthen our contention that silencing efficacy cannot be solely dependent on the target accessibility, and that other factors including specific nucleotide positions within the spacer or target, a possible PFS, and changes in target accessibility, may all influence key steps of target silencing such as crRNA transcription, loading, and target recognition.

### Example 3

### In Silico Analysis of Silencing Profiles from 201 crRNAs Revealed Key Design Principles

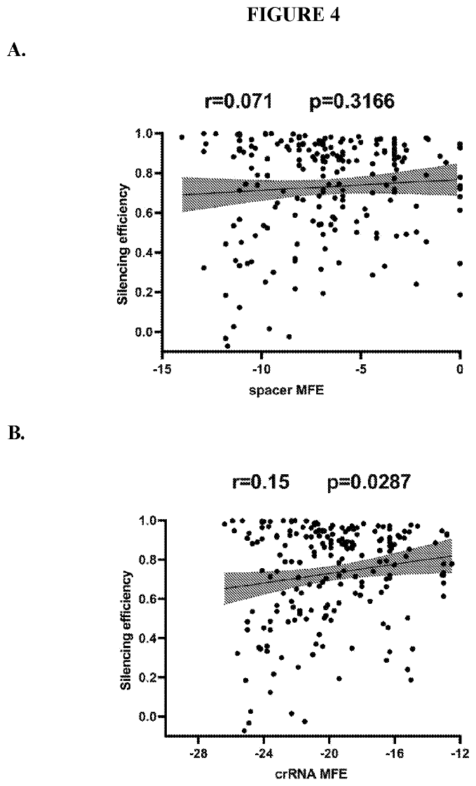

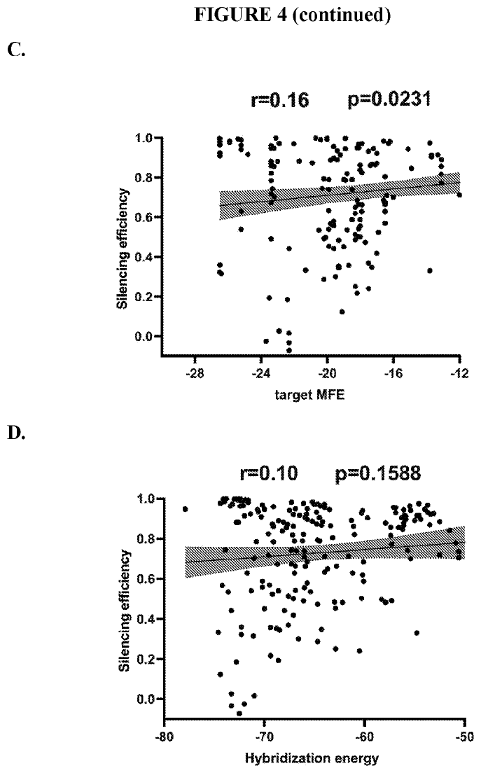

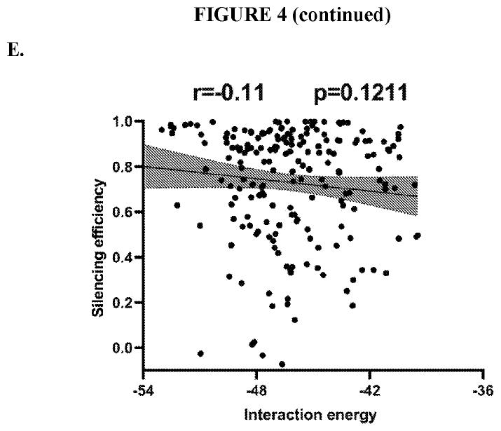

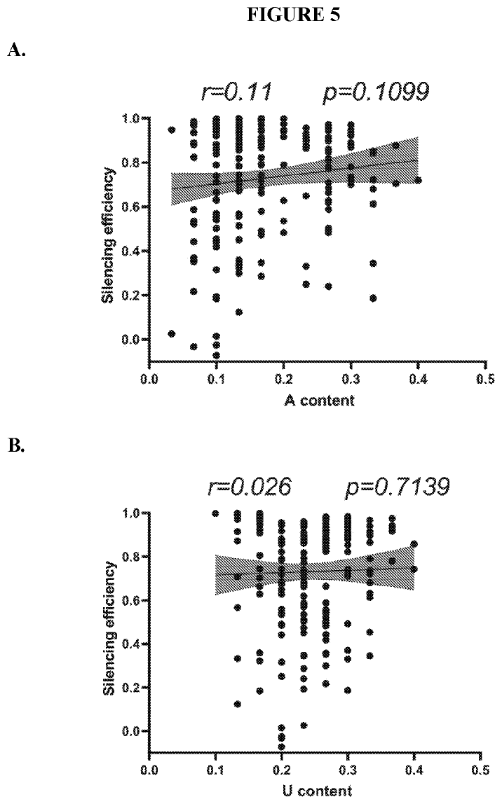

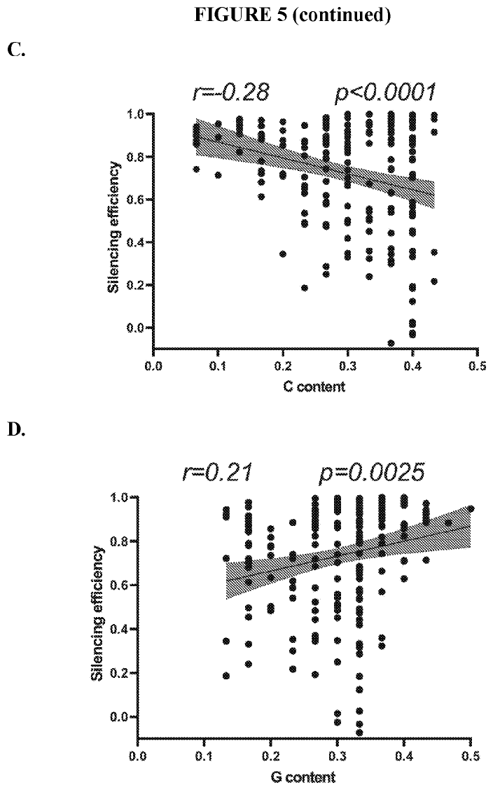

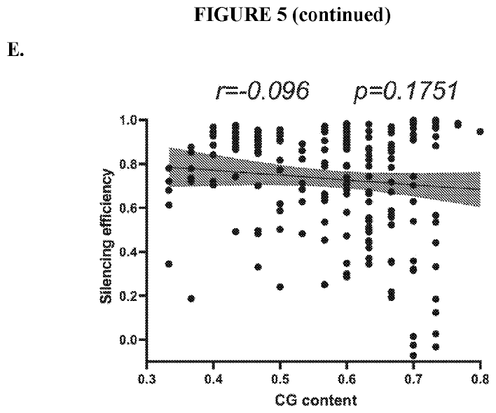

In an effort to uncover universal parameters that dictate crRNA efficiency, we expanded our dataset by analyzing the silencing profiles of 201 individual crRNAs targeting various transcripts. We analyzed a number of characteristics that may influence PspCas13b silencing efficiency in unfiltered crRNAs population including the predicted crRNA and target folding, target-spacer hybridization and interaction energy (FIGS. 4A-4E), and spacer nucleotide content (A, U, C, G, and CG) (FIGS. 5A-5E). We questioned whether the folding of the crRNA (the spacer and direct repeat together or just the spacer sequence) and the target into a complex secondary structure could impair crRNA loading into PspCas13b or alter target accessibility, respectively. We generated projected secondary structures of all 201 spacers and crRNAs in the library and calculated the minimum free energy (MFE) that predicts the probability of forming stem-loop secondary structures (FIGS. 4A-4C). We used Pearson correlation to probe the existence of any relationship between the predicted folding and PspCas13b silencing efficiency. The data revealed a moderate positive correlation between the minimum free energy (MFE) of the crRNA and PspCas13b silencing efficiency (r=0.15; p=0.0287), suggesting crRNA stem-loop structure formation can only moderately influence silencing efficacy (FIG. 4B). In contrast, the folding of the spacer without its direct repeat sequence was not correlated with silencing (r=0.071; p=0.3166) (FIG. 4A). We employed a similar approach to predict the folding of a 70 nt RNA sequence surrounding the targeted region. The data showed a moderate positive correlation between target unfolding and the silencing efficiency of crRNA (r=0.16; p=0.0231) (FIG. 4C). Together, these data suggest that the folding of the crRNA and the targeted sequence into complex secondary structures can moderately impair PspCas13b silencing efficiency, possibly perturbing crRNA loading or target accessibility.

The stability of the interaction between the spacer and the target RNA can define PspCas13b binding and dissociation kinetics, and therefore may dictate its affinity toward a given target. We predicted the hybridization and interaction energy of various spacer sequences in the library with their cognate targets. No significant correlation between the hybridization or interaction energy and crRNA silencing efficiency was demonstrable, suggesting that target affinity and PspCas13b potency is not determined by spacer-target duplex RNA stability (FIGS. 4D-4E).

Next, we used a similar approach to analyze the effect of differential ribonucleotide abundance within the spacer on crRNA activity. The analysis of spacer content in A, U and CG did not show any correlation with the silencing, whereas C and G nucleotide content were negatively and positively correlated with PspCas13b silencing respectively (FIGS. 5A-5E), indicating spacer nucleotide content is likely a vital determinant of PspCas13b silencing.

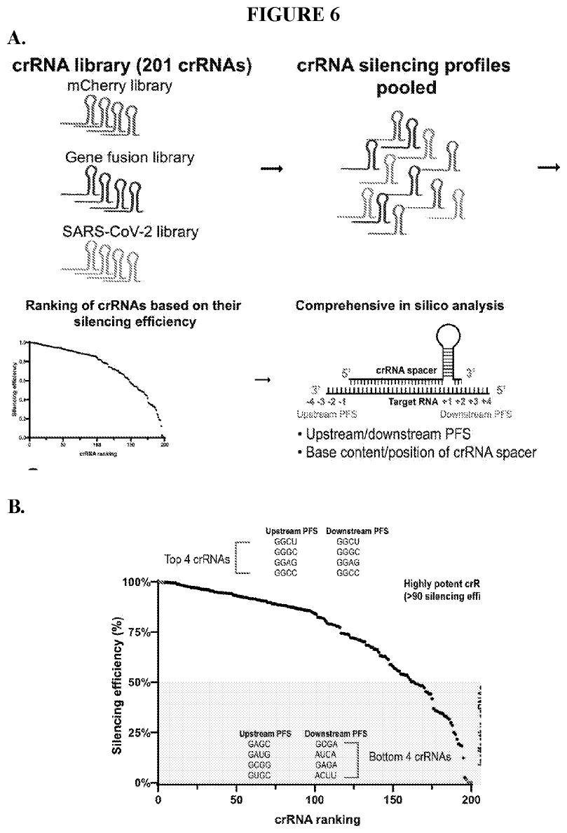

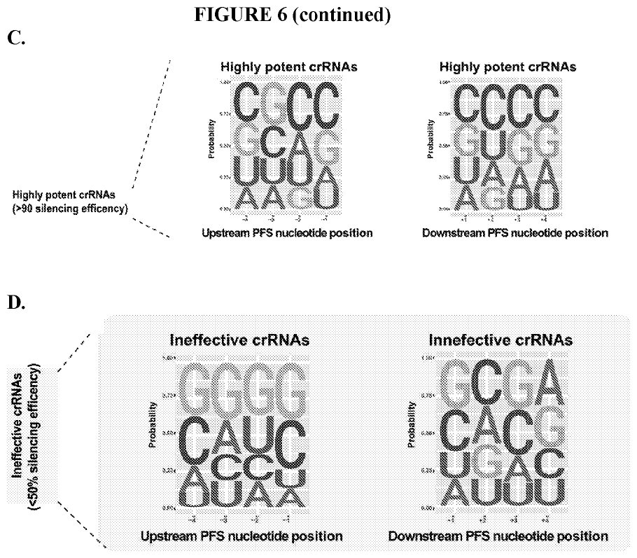

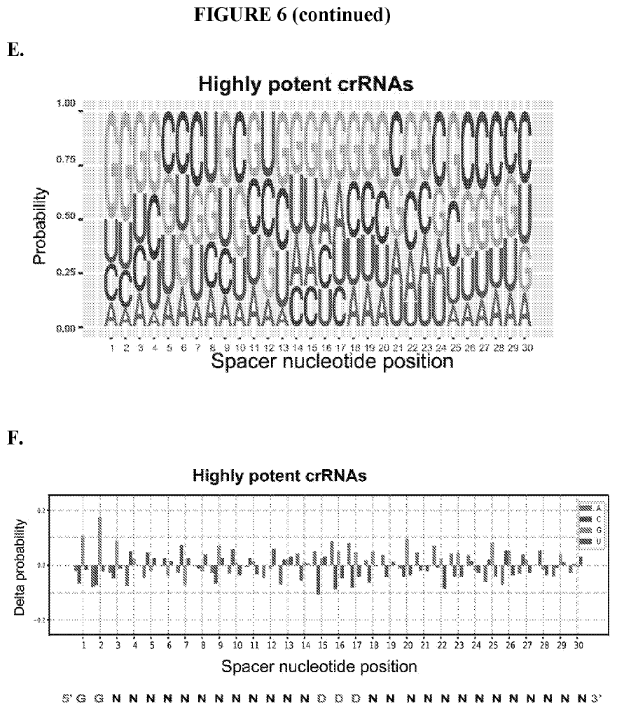

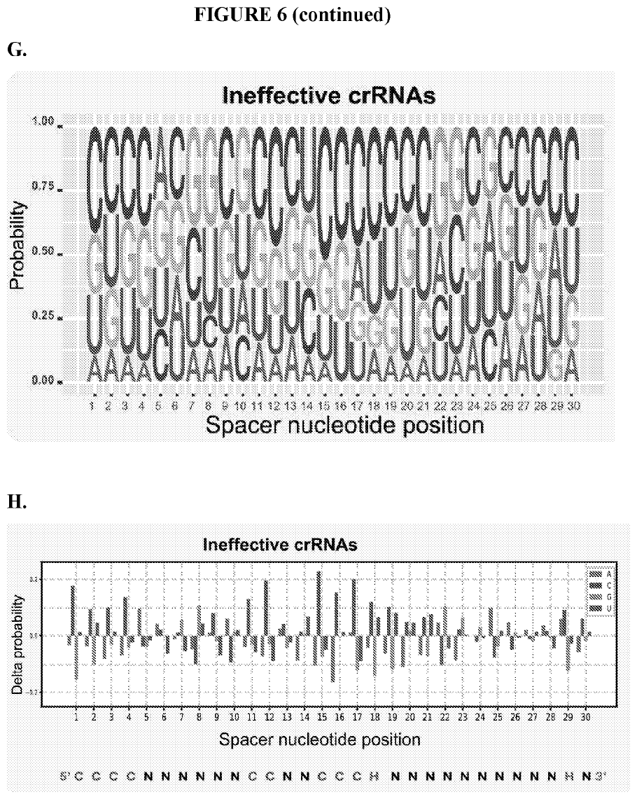

Subsequently, we pooled these 201 crRNAs and ranked them by silencing efficiency. crRNA that achieved >90% silencing efficiency were designated as potent crRNAs and those with less than 50% efficiency were considered ineffective crRNAs. crRNAs with ambiguous silencing profiles (efficiencies ranging from 50 to 90%) were excluded from the analysis. We sought to identify molecular features capable of differentiating potent and ineffective crRNA cohorts (FIG. 6D).

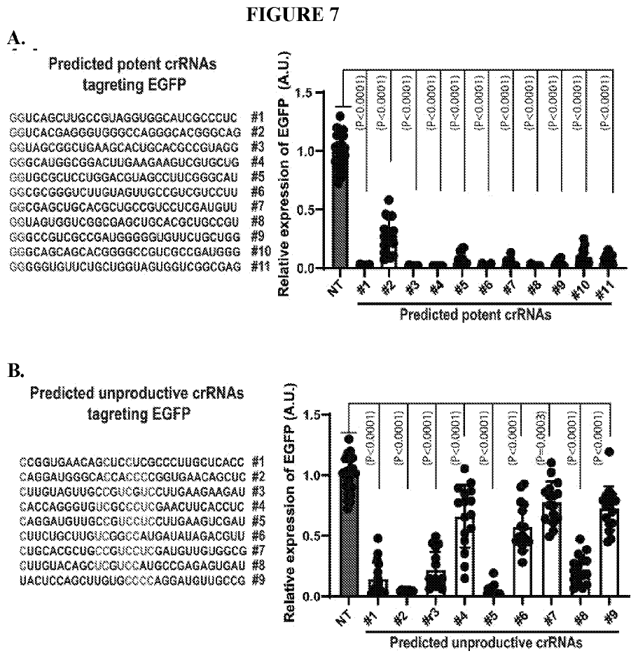

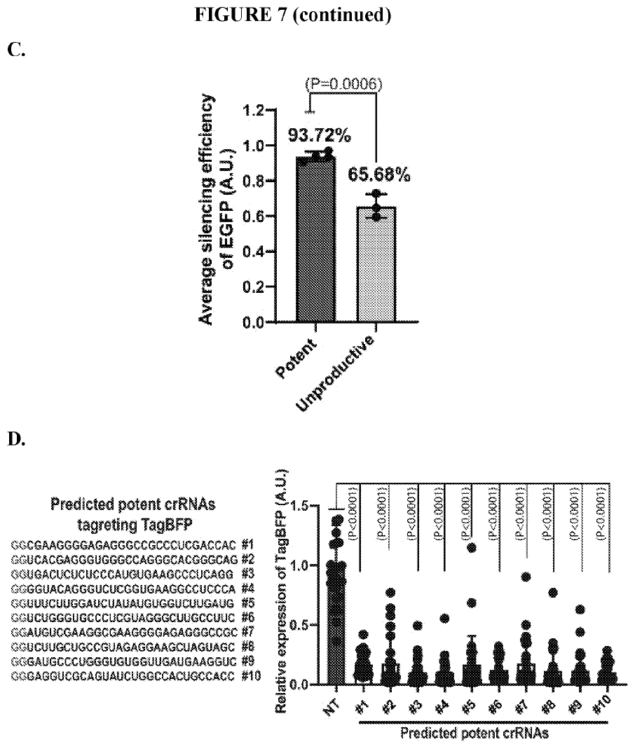

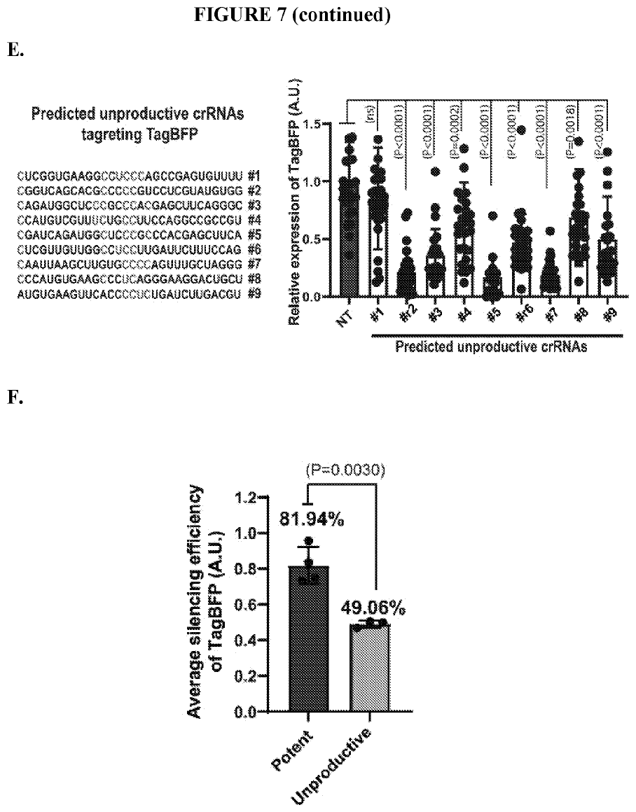

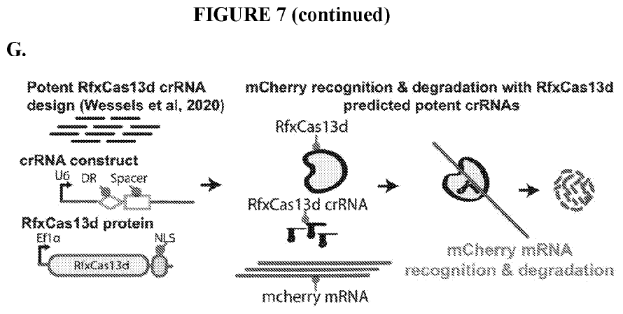

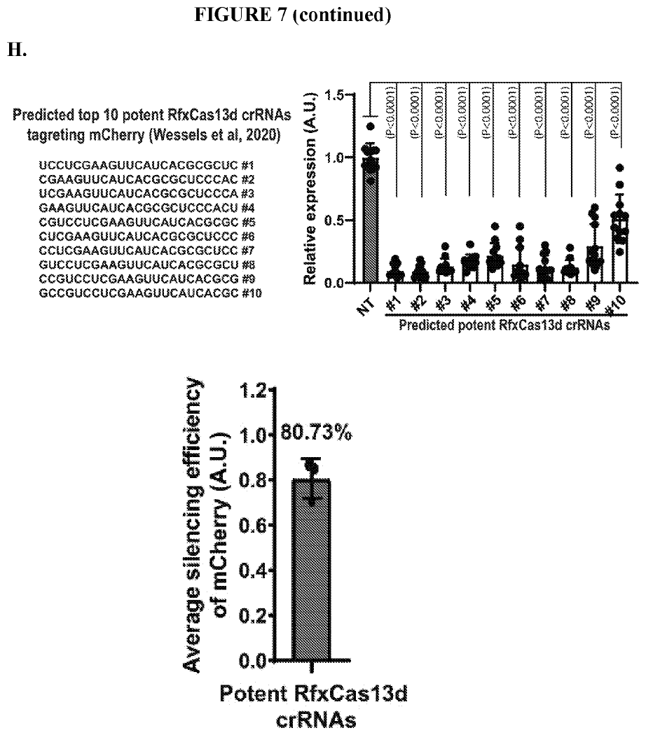

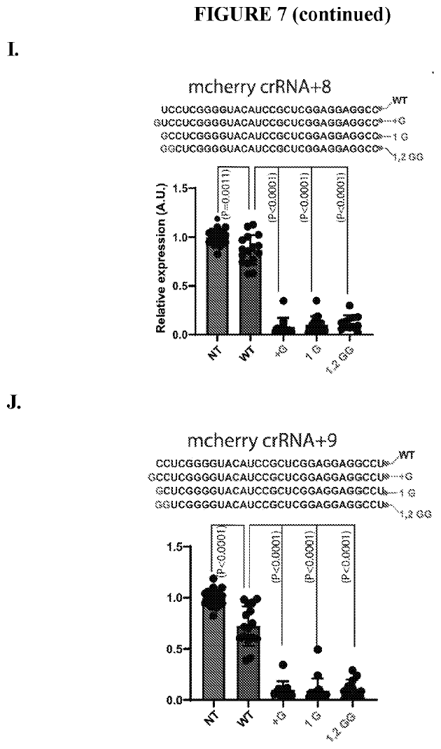

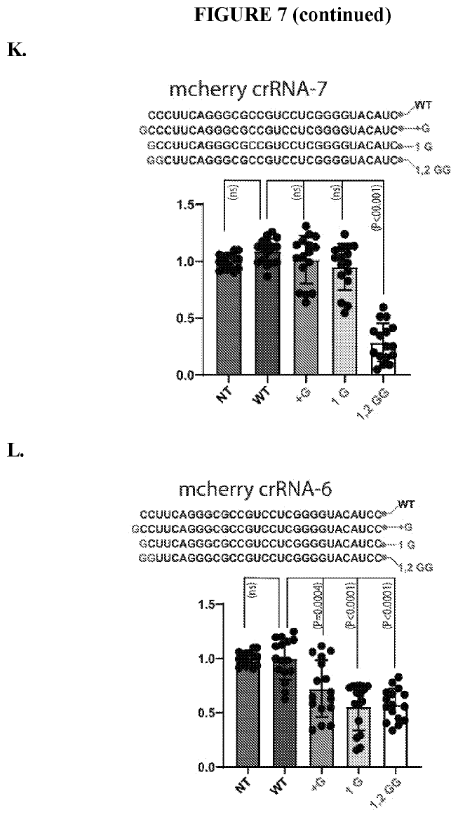

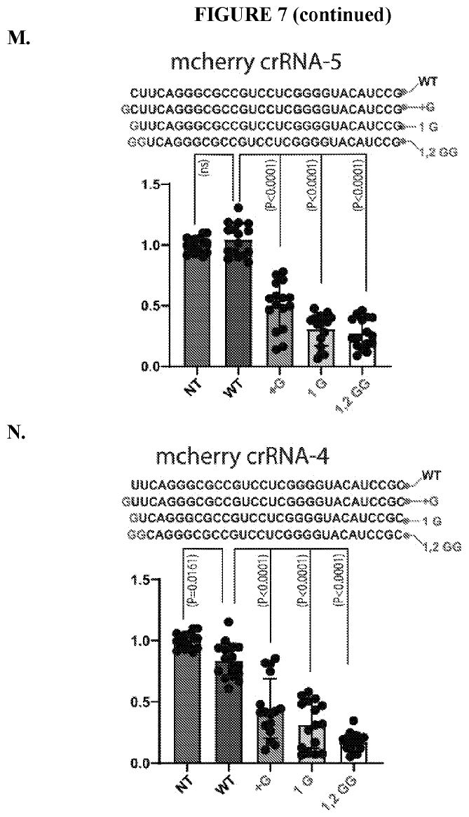

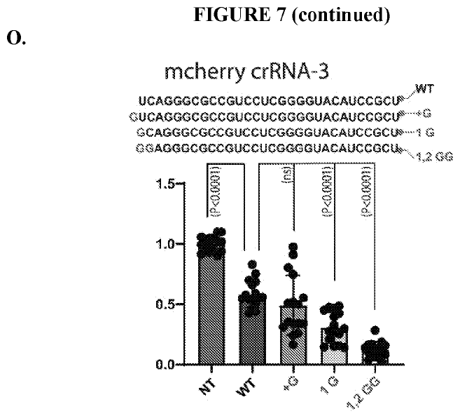

Many CRISPR variants possess an upstream or downstream protospacer flanking sequence (PFS) that restricts targeting activity and prevents degradation of their own nucleic acids. For instance, SpCas9 has an NGG PSF sequence known as protospacer adjacent motif (PAM) that enables this protein to discriminate between its own and foreign DNA. Previous PspCas13b screens in bacteria suggested the presence of a GG sequence that may act as a PSF (Cox et al., 2017, Science, 358), although this observation remains unverified in other organisms, including mammalian cells. To investigate the existence a flanking PFS that could constrain PspCas13b silencing, we generated weight matrix plots that analyze nucleotide composition at each position of four bases upstream and downstream of the targeted sequence in the highly potent and ineffective cohorts of crRNAs. There was no detectable bias in nucleotide composition at various target flanking sites, suggesting that PspCas13b activity is not subject to PFS motifs in mammalian cells (FIGS. 7B-7D).

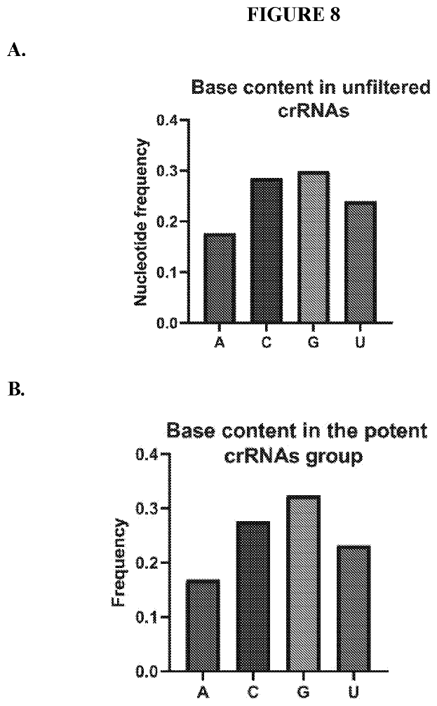

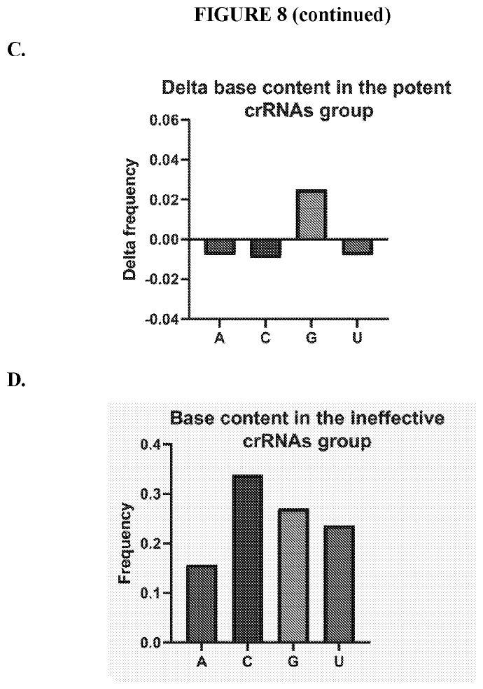

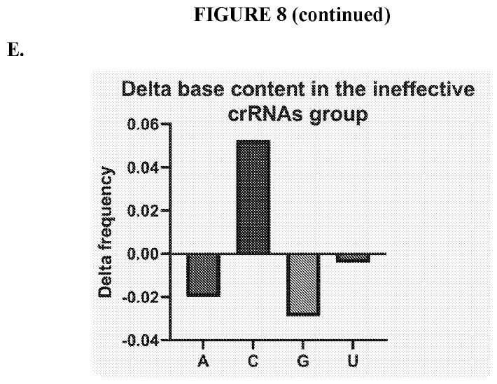

Finally, we questioned whether the nucleotide composition of the spacer could influence PspCas13b silencing efficiency. Concordant with the correlation data in unfiltered crRNAs (FIGS. 5C-5D), nucleotide content analysis of the filtered crRNA cohorts confirmed an enrichment of G bases in the potent group, and enrichment of C bases in the ineffective crRNA cohort (FIGS. 8A-8E). These data confirmed that a G-enriched spacer is associated with higher crRNA potency, whereas C-enriched spacers are associated with low potency. However, these data do not reveal the relevance of G and C bases at specific positions within the spacer sequence.

To answer this question, we conducted unbiased analyses of nucleotide composition at all 30 positions of the spacer in highly potent and ineffective crRNA cohorts. We used weight matrix plots and Delta probability analysis to compare spacer nucleotide composition at all positions between filtered and unfiltered samples (FIGS. 7E-7H), and revealed marked differences in nucleotide positions between crRNA cohorts. We noticed that G bases at the 5′end, particularly a GG sequence at the first and second positions was strongly associated with highly potent crRNAs (FIGS. 7E-7F). Conversely, G nucleotides were depleted and C bases were enriched at the 5′end of spacers in the ineffective crRNA cohort (FIGS. 7G-7H). In addition to this C-rich motif at the 5′end of ineffective crRNAs, we also noticed a significant enrichment of C bases at positions 11, 12, 15, 16, and 17 (FIG. 7G-7H). These data revealed key nucleotide positions that determine the potency of crRNAs.

### Example 4

### Functional Validation of PspCas13b crRNA Prediction and Design

The above in silico analysis enabled us to generate a formula to predict potent and ineffective crRNAs. Potent crRNAs should include GG sequence at the first and second position of the spacer and should lack C bases in position 11, 12, 15, 16, and 17 (GGNNNNNNNNDDNNDDDNNNNNNNNNNNNN; D is a G, U, or A nucleotide, SEQ ID NO:3). crRNAs containing C in spacer positions 1, 2, 3, 4, 11, 12, 15, 16, 17, and an H ribonucleotide (C, U, or A) at position 18 are predicted to yield poor silencing efficiency (CCCCNNNNNNCCNNCCCHNNNNNNNNNNNN, SEQ ID NO:4).

We tested the predictive accuracy of these spacer-based formulas through prospective unbiased design of crRNAs targeting EGFP and TagBFP, two mRNA targets we had not investigated previously. Notably, out of 21 predicted potent crRNAs, 20 achieved very high silencing efficiency of either EGFP or TagBFP mRNA (FIGS. 6A and 6D). Conversely, the majority of predicted ineffective crRNAs failed to efficiently silence EGFP and TagBFP transcripts (FIGS. 6B and 6E). The average silencing efficiency of potent crRNAs targeting EGFP and TagBFP was ˜94% and ˜85% respectively, whereas the average silencing efficiency of predicted ineffective crRNAs was 65% and 49%, respectively (FIGS. 6C and 6F). By formulating our prediction from a pre-existing dataset, and validating its accuracy in heretofore untargeted transcripts, these data demonstrate our formula to be both accurate and generalizable, and demonstrate its utility in crRNA design for silencing any transcript of interest.

Next, we compared the efficiency of our design to the gold standard crRNA design tool that is available for RfxCas13d (FIG. 6G). We selected 10 top predicted potent crRNAs for RfxCas13d targeting mCherry and probed their silencing efficiency, which achieved an average silencing of 80.7% (FIG. 6H). Our PspCas13b design of potent crRNAs showed ˜90.5% average silencing efficiency (EGFP and TagBFP together, FIGS. 6C and 6F) and outperformed RfxCas13d design, further validating the accuracy of our prediction tool (FIGS. 6C and 6F).

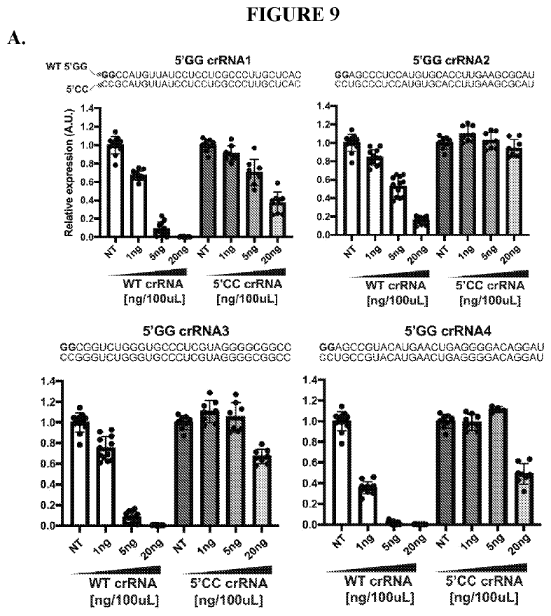

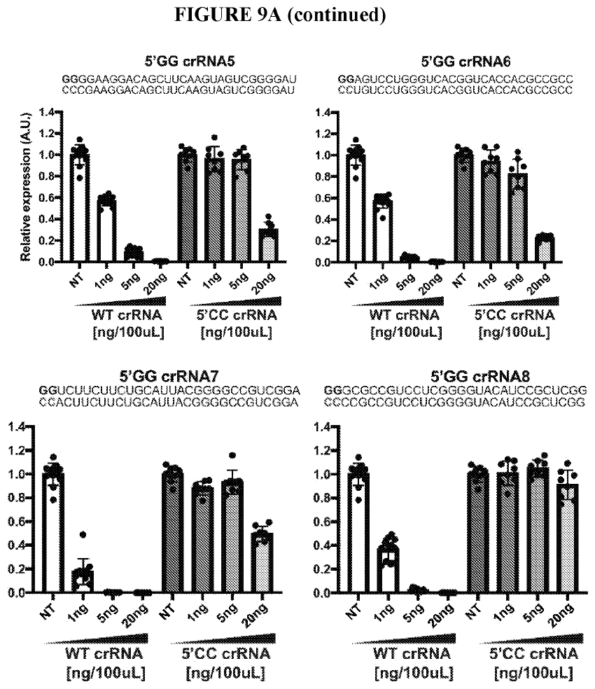

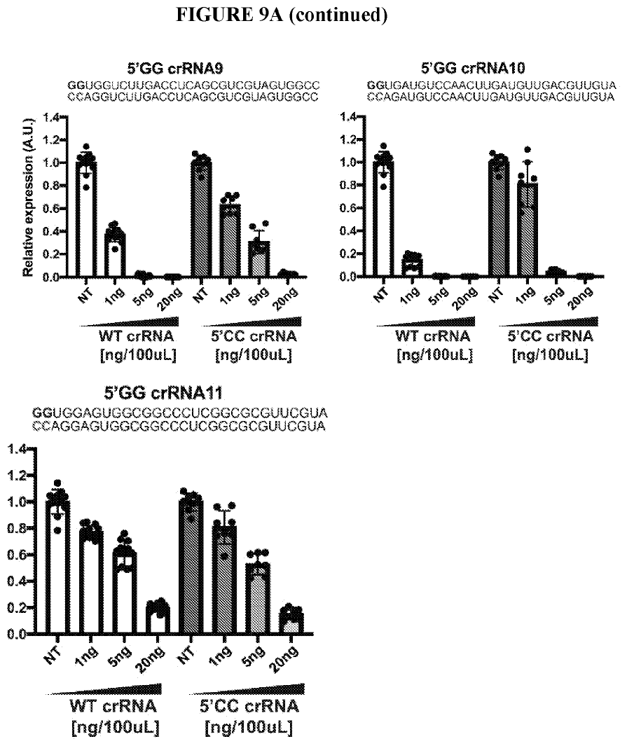

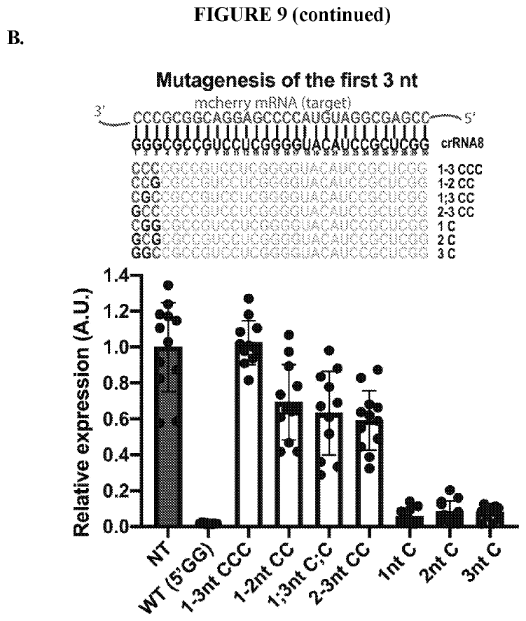

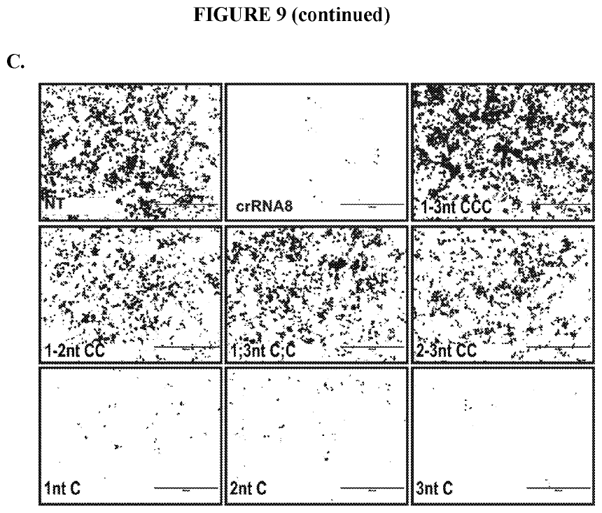

To further investigate the enrichment of a G-rich motif at the 5′end of potent crRNAs and C bases at the 5′end of ineffective crRNAs, we hypothesized that altering these sequences in a bona fide spacer sequence may either worsen or improve their silencing efficiency. First, we selected 11 crRNAs that possess a GG sequence at 1^{st} and 2^{nd} positions of the spacer which we altered to CC by spacer mutagenesis. As anticipated, the data showed substantial compromise in the silencing efficiency of the majority of these crRNAs (FIG. 9A). We also mutated 3, 2, or 1 G base(s) at the 5′end of the spacer to a C residue(s) and found that the substitution of 3 or 2 C bases at the 5′end of the spacer reduces silencing by >99% to ˜70% respectively, while a single C base at spacer position 1, 2, or 3 has a minor effect on the potency of the crRNA (FIGS. 9B-9C).

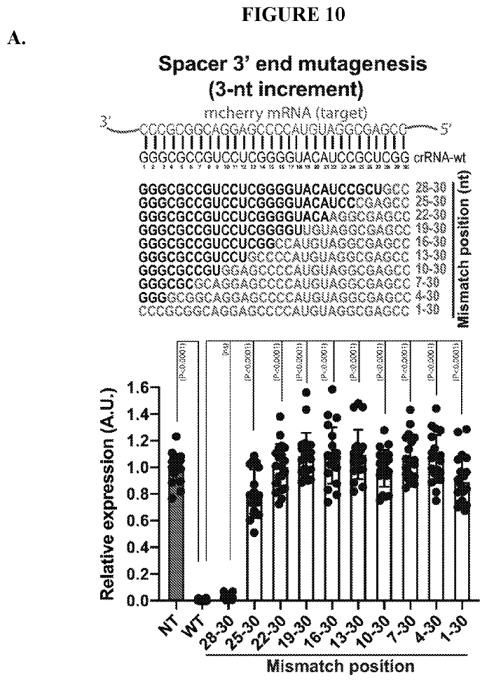

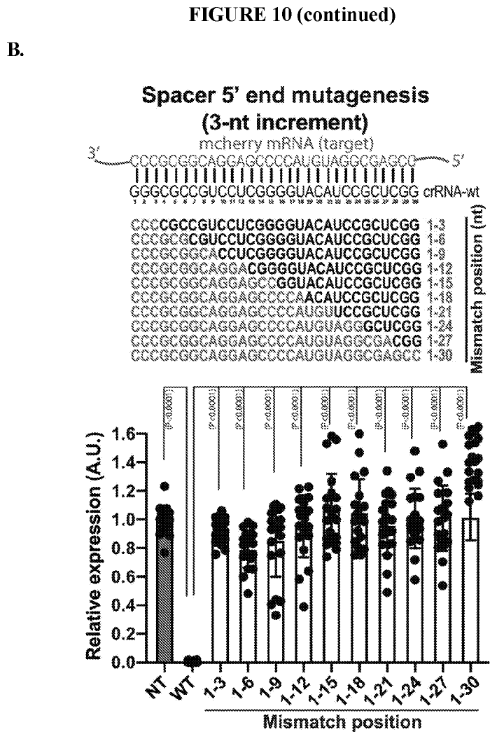

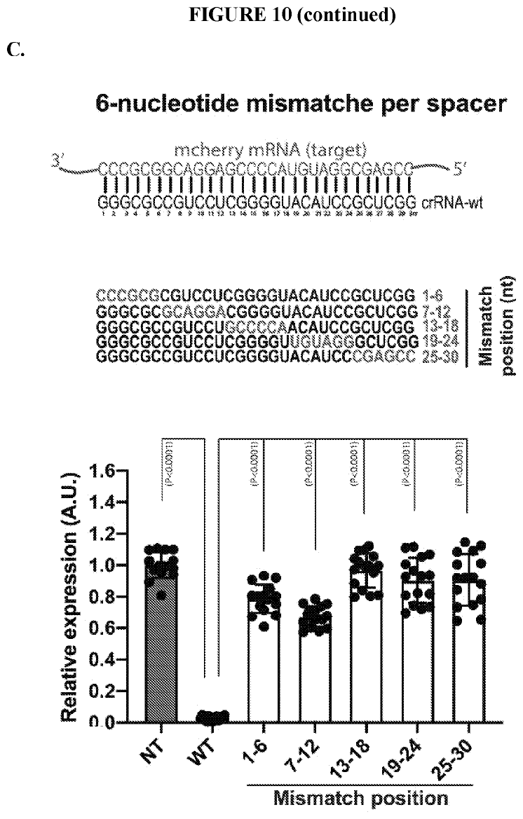

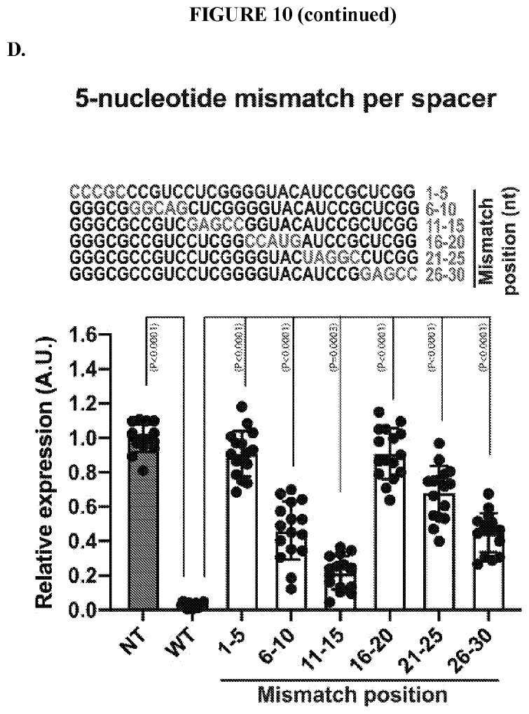

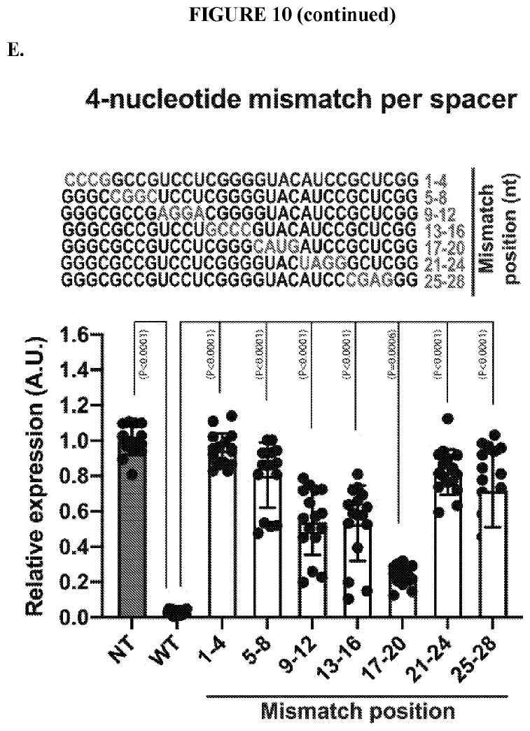

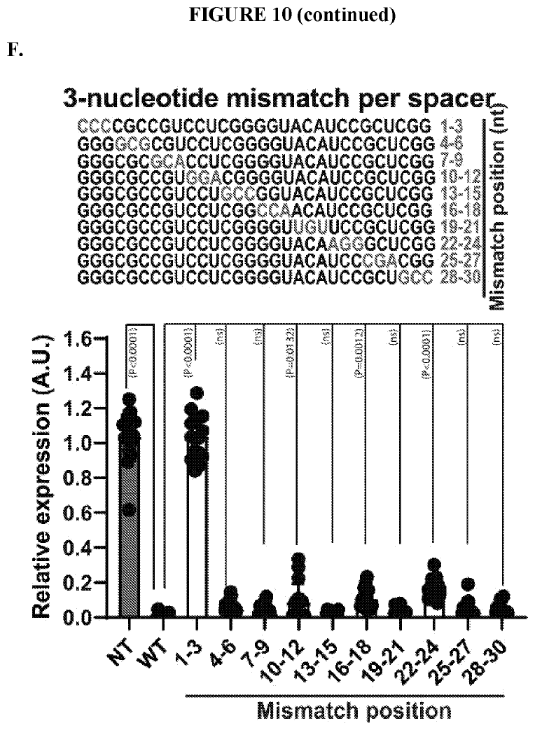

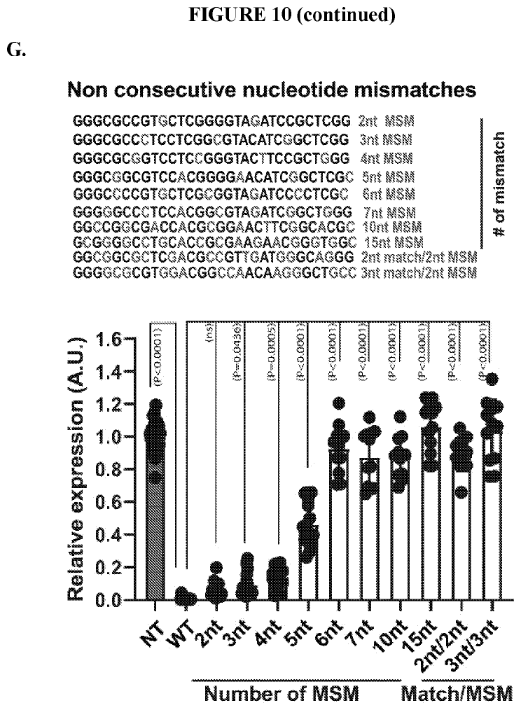

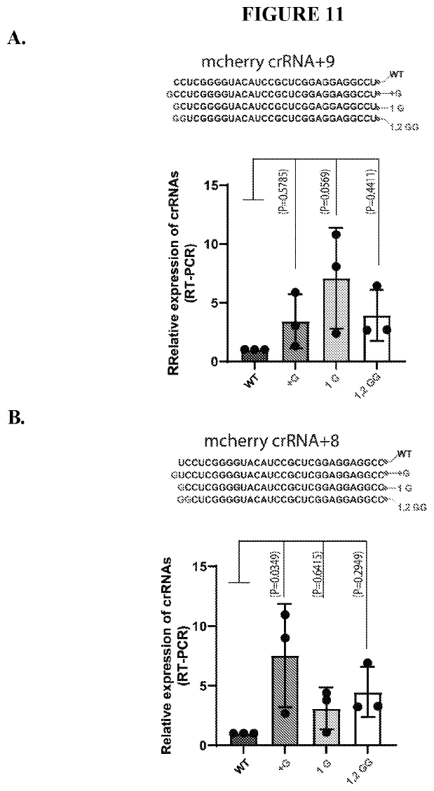

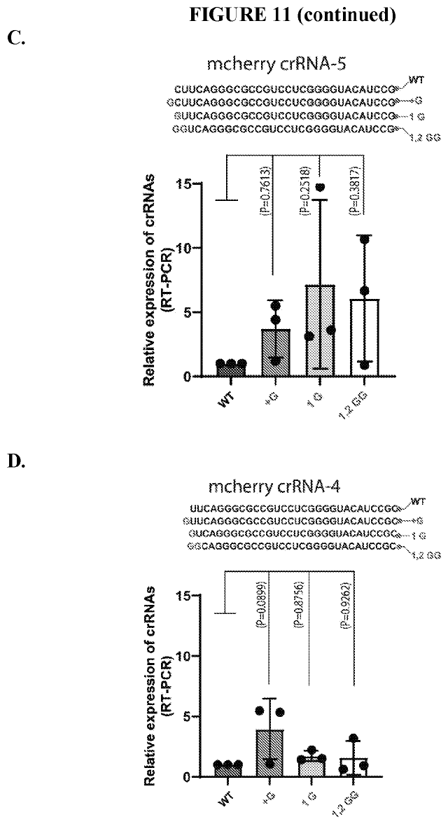

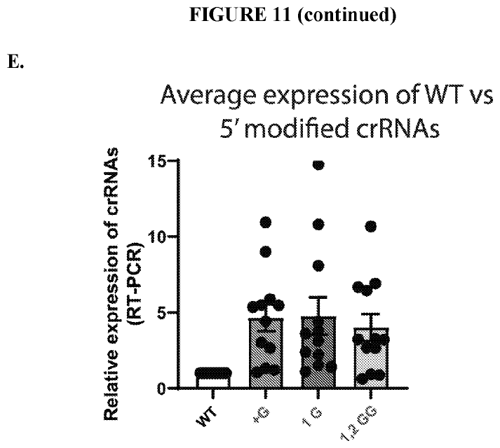

Next, we selected ineffective crRNAs lacking a GG sequence at their 5′end, and then modified them either by inserting an additional G at the first position, substituting the 1^{st} nucleotide to a G, or substituting the 1^{st} and 2^{nd} nucleotides to a GG (FIGS. 10I-10P). Importantly, the data demonstrated that G sequences at the 5′end of the spacer greatly increase the potency of crRNA despite the introduction of spacer-target mismatch (FIGS. 10I-10P). We questioned whether the improvement in silencing efficiency of crRNAs harboring a G-rich motif at their 5′end could be secondary to changes in crRNA abundance. We quantified the expression levels of original crRNA or mutated crRNAs harboring 5′end G motifs using quantitative real-time PCR (RT-PCR). Although not statistically significant, we observed an increase in crRNA abundance when a G-rich motif is present at the 5′end (FIG. 11).

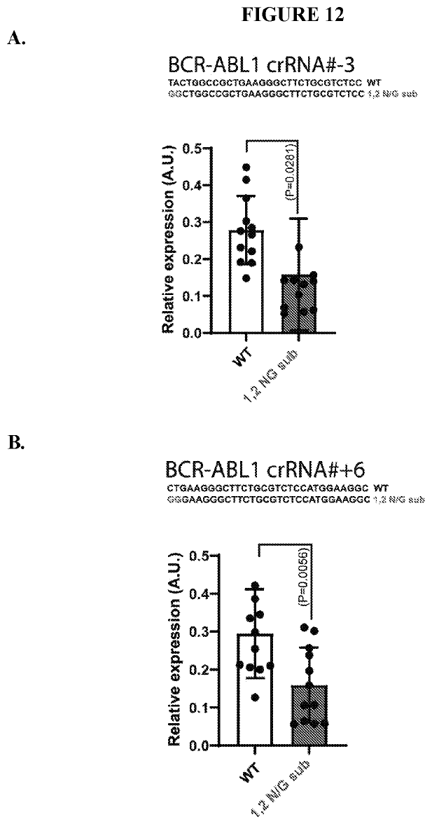

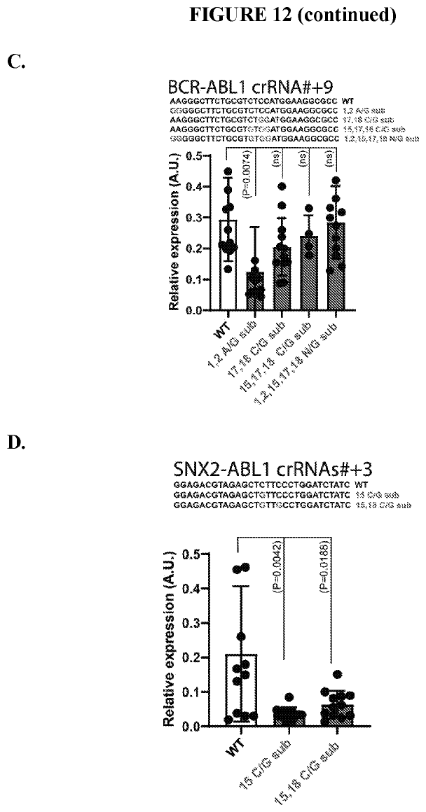

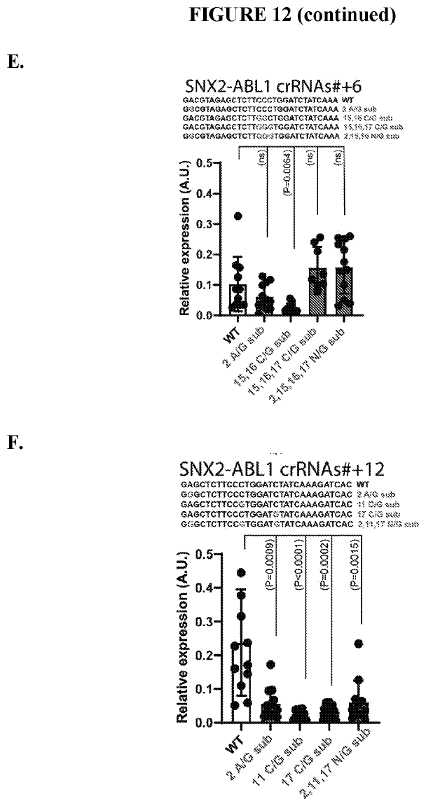

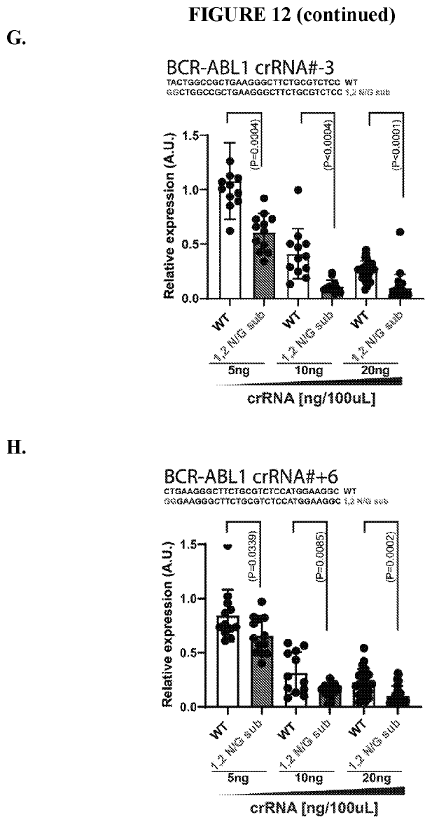

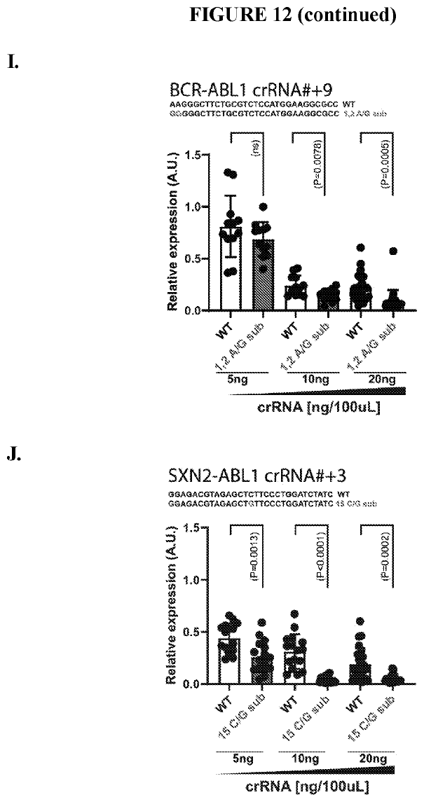

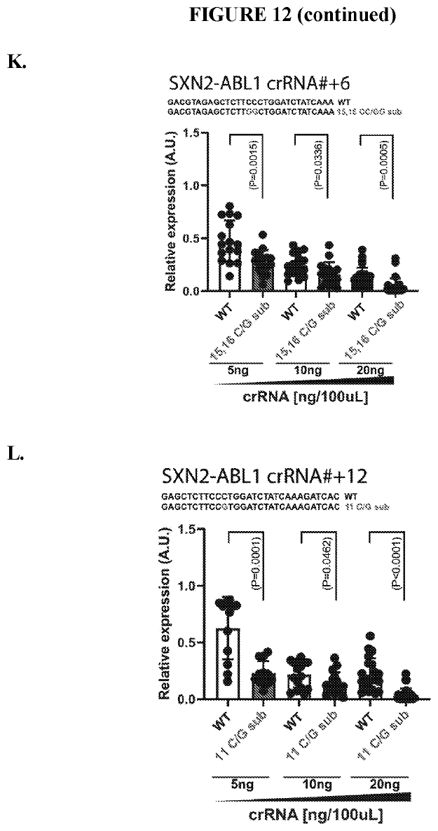

In addition to mCherry, we also show that nucleotide(s) substitutions to a G base in key spacer positions (1, 2, 11, 15, 16, 17) can significantly improve the silencing efficiency of crRNAs (FIG. 12). These findings demonstrate the importance of a G-rich motif at the 5′ end of the space. Indeed, when crRNA design choices are restricted, de novo design of crRNAs incorporating a novel G-rich motif at their 5′end can substantially increase their potency despite introducing nucleotide mismatches with the target.

### Example 5

### Comprehensive Mutagenesis of PspCas13b Spacer-Target Interaction Revealed the Interface Between Mismatch Tolerance and Loss of Activity

Understanding PspCas13b specificity, off-targeting potential, and its capability to discriminate between two transcripts that share extensive sequence homology is extremely important to evaluate the potential and define the limitations of Cas13-based RNA silencing. To study PspCas13b specificity and its targeting resolution we conducted a comprehensive spacer mutagenesis study where we altered spacer-target interactions at various positions. We used a potent crRNA (crRNA12; SEQ ID NO: 17) targeting mCherry as a model. First, we introduced 3, 6, 9, 12, 15, 18, 21, 24, 27, and 30-nt successive mismatches between the target and the crRNA through the mutagenesis of the 3′ and 5′ends of the spacer (FIGS. 10A and 10B). This experiment showed that 3-nt mismatches at the 3′end of spacers (position 28-30) did not affect the silencing efficiency, whereas mismatches greater that 3-nt completely abrogated silencing (FIG. 10A). In contrast to the 3′end, all 5′end mismatches resulted in complete loss of silencing including 3-nt mismatches at the 5′ end (FIG. 10B). Silencing loss consequent to the introduction of a 3-nt mutation at the 5′end is likely attributable to the substitution of a GGG motif by a CCC sequence rather than spacer-target mismatch itself, thus reaffirming the importance of a G-rich motif at the 5′end of potent crRNAs as described elsewhere herein (FIGS. 6 and 7).

To gain a better understanding of mismatch tolerance across various regions of the spacer, we created crRNA constructs harboring 6-nt, 5-nt, 4-nt, and 3-nt mismatches at different spacer positions and probed their silencing efficiency in live cells (FIGS. 10C-10F). Overall, 6-nt mismatches largely compromised the efficiency of PspCas13b regardless of mismatch position (FIG. 10C). 5-nt mismatches at positions 6-10, 11-15, and 26-30 exhibited a partial loss of silencing ranging from 25 to 50%, while mismatches at positions 1-5, 16-20, and 21-25 led to a near complete or complete loss of silencing (FIG. 10D). 4-nt mismatches at positions 9-12, 13-16, and 17-20 retained partial silencing activity, whereas mismatches at positions 1-4, 5-8, 21-24, and 25-28 yielded a complete loss of silencing (FIG. 10E). Notably, crRNA constructs harboring 3-nt mismatches at various spacer positions were well tolerated and yielded no or minor loss of silencing, except for mutations at position 1-3 that led to a total loss of silencing (FIG. 10F). The systematic loss of silencing efficiency when mutations are incorporated to the 5′end of the spacer is likely due to GGG substitution with CCC, which is concordant with our previous findings (FIGS. 6 and 7). Successive 6 nucleotide mismatches or higher are not tolerated regardless of their position within spacer-target duplex (FIGS. 10A-10C). Taken together, this comprehensive mutagenesis analysis revealed spatial asymmetry of mismatch tolerance. Thus, PspCas13b nuclease activation appears to demand at least ˜24-nt base-pairing with the target, indicating that this tool is extremely specific considering the exceptionally low probability that another endogenous transcript will share perfect homology for 24 nucleotides with the target transcriptome wide.

Whilst the preceding experiments established the tolerance for consecutive spacer-target mismatches, we questioned whether the silencing profile of non-consecutive mismatches may differ. We destabilized the spacer-target interaction by introducing 2, 3, 4, 5, 6, 7, 10, and 15 non-consecutive mismatches spread throughout the spacer (FIG. 10H). 2, 3, and 4 non-consecutive mismatches were tolerated and led to negligible loss of silencing. However, 5-nt non-consecutive mismatches led to a substantial loss of silencing, while 6 or more non-consecutive mismatches completely abolished crRNA silencing activity. Likewise, multiple successive 2 or 3 nucleotide mismatches spread throughout the spacer sequence also completely abolished its silencing activity (FIG. 10H). These data revealed the targeting resolution of PspCas13b and suggest that 5-nt or higher non-consecutive mismatches critically destabilize spacer-target interaction and compromise PspCas13b activity. In addition, the data also suggest that endogenous targets with partial sequence homology are unlikely to be impacted by off-target silencing due to the required minimum ˜24 nucleotide complementarity. These mutagenesis data provide further evidence that highly effective crRNAs can be readily designed with minimal or no off-target effects.

### Example 6

### PspCas13b crRNAs can Silence Tumor Drivers with Fluctuating Efficiencies

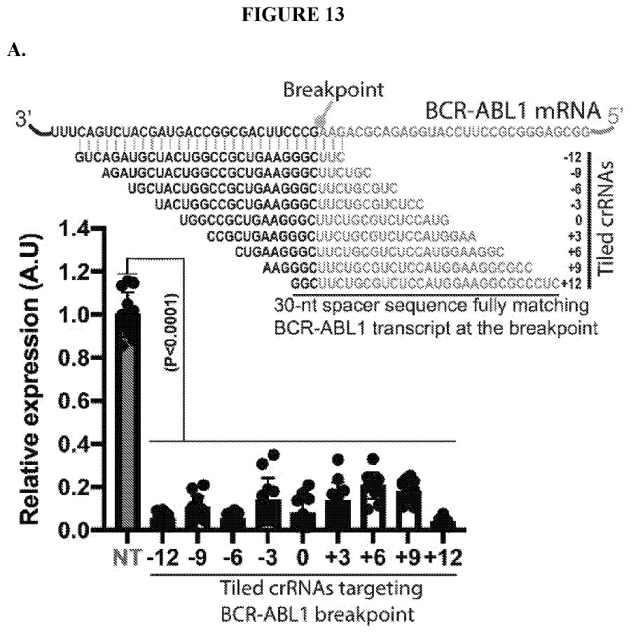

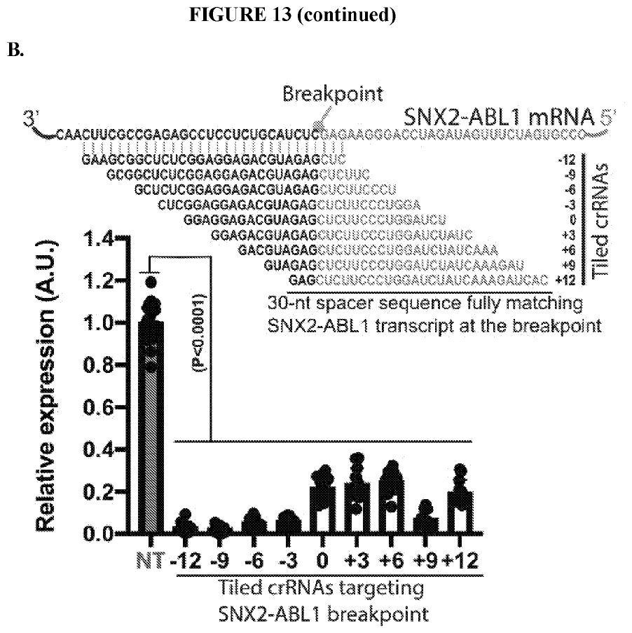

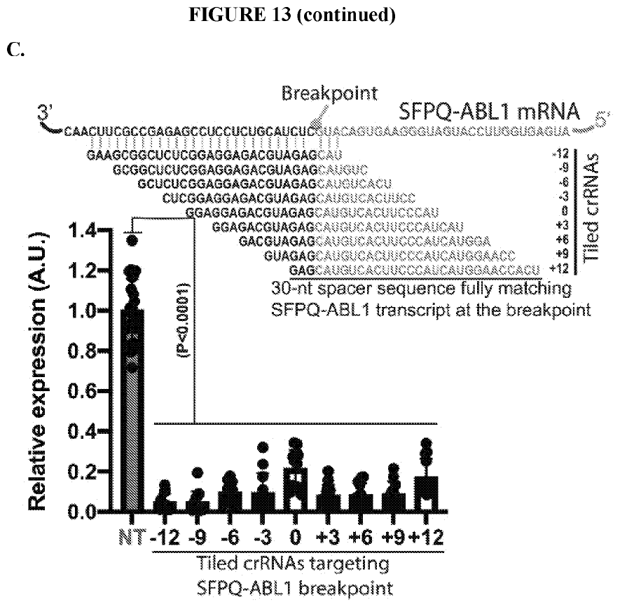

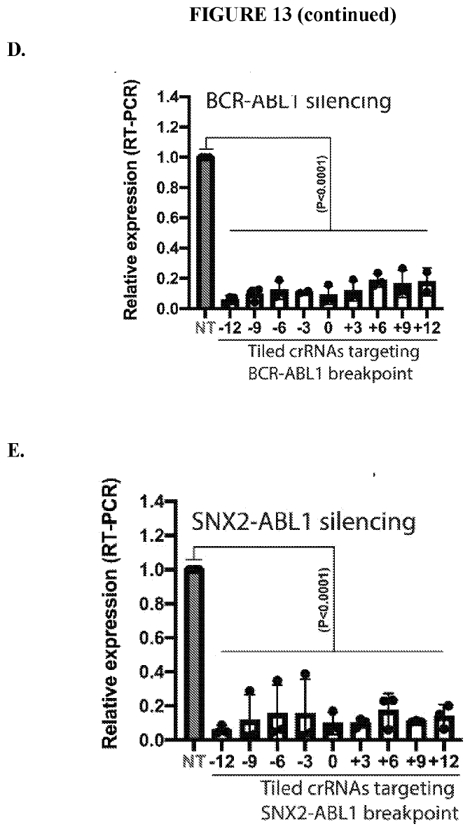

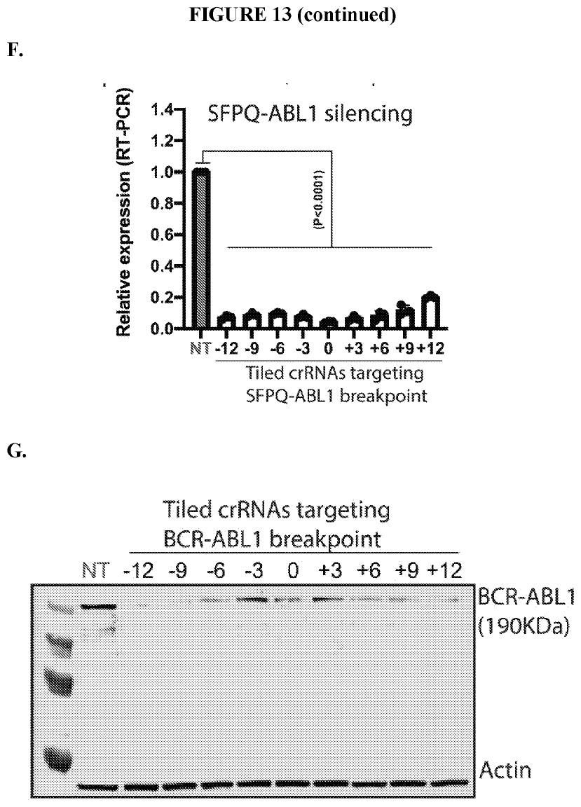

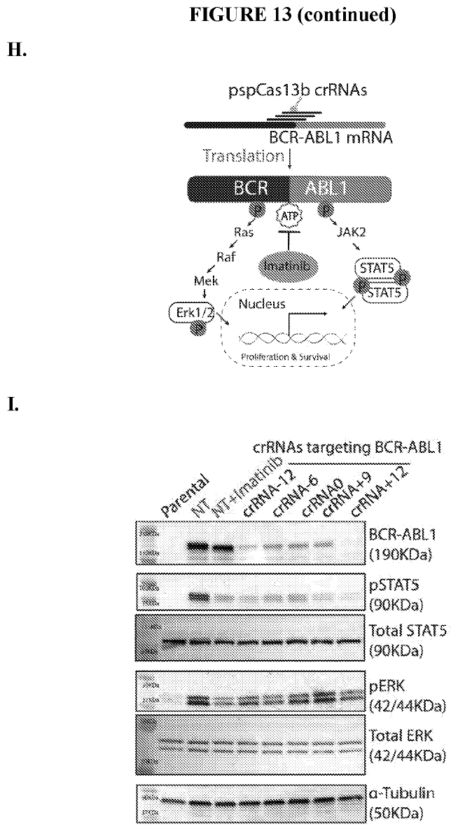

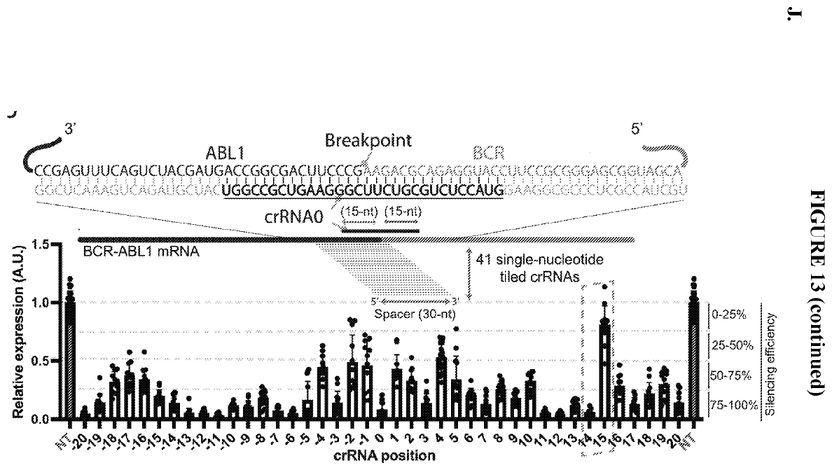

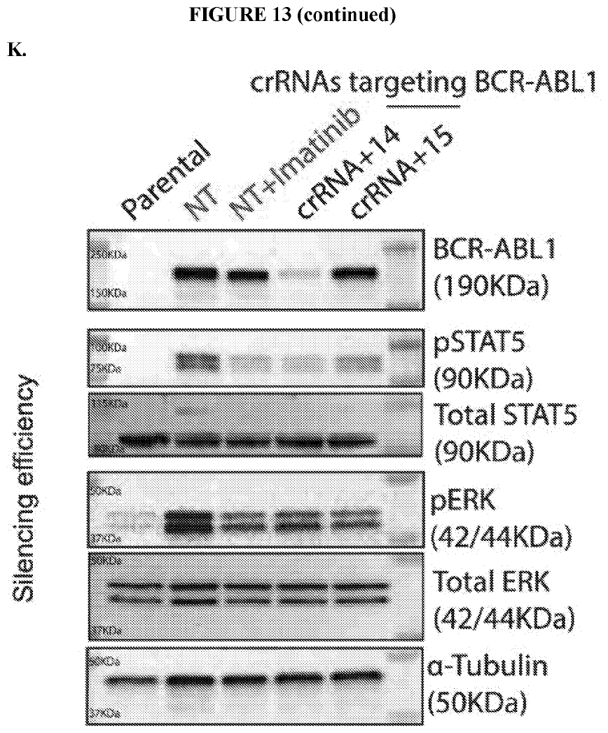

Gene fusions are genomic aberrations that result from chromosomal translocations and often generate oncogenic chimeras. The breakpoint at the interface between the two genes offers a unique targetable sequence at the RNA level. Considering the data described above, we anticipated that various crRNAs targeting the gene fusion breakpoint transcript may yield contrasting silencing profiles. Therefore, we designed 9 tiled crRNAs (3-nucleotide resolution) targeting the breakpoint of 3 oncogenic gene fusions BCR-ABL1, SFPQ-ABL1, and SXN2-ABL1 that are established drivers of various human malignancies. The gene fusions were each cloned into a reporter construct followed by an internal ribosomal entry site (IRES) and a GFP sequence, enabling co-transcription of the gene fusion and GFP, which are subsequently translated into separate proteins due to the presence of the IRES sequence. In this reporter assay, efficient recognition of the gene fusion transcript by PspCas13b is anticipated to lead to loss of GFP fluorescence due to sequence-specific recognition, cleavage, and degradation of the fusion-GFP transcript. We transfected HEK 293T cells with plasmids encoding the gene fusion of interest, PspCas13b-BFP, and various tiled crRNAs targeting the breakpoints. A non-targeting (NT) crRNA served as a control. Overall, microscopy data from 3-nucleotide resolution tiled crRNAs showed high silencing efficiency of all 3 gene fusions, although, once more the silencing efficiency varied depending on the position of the crRNA (FIGS. 13A-13C). For instance, crRNAs targeting BCR-ABL1 matching the positions −12, −6, 0, and +12 achieved higher silencing efficiency compared to the other crRNAs (FIG. 13A). Analysis of mRNA levels of gene transcripts by RT-qPCR confirmed high silencing efficiency with numerous crRNAs, although the magnitude of variance between crRNAs was less pronounced than suggested by the microscopy assay (FIGS. 13D-13F), possibly due to an additional Cas13-mediated protein translation regulation. Western blot analysis of the BCR-ABL1 protein expression also confirmed high silencing of BCR-ABL1 at the protein level, which, consistent with the microscopy data, was dependent on the position of crRNAs tested. −12, −9 and +12 crRNAs exhibited the highest silencing efficiencies (FIG. 13G). Analysis of Stat5 and ERK phosphorylation, a hallmark of BCR-ABL1 dependent oncogenic signaling (FIG. 13H), confirmed that potent crRNAs can efficiently suppress BCR-ABL1 and its downstream oncogenic networks (FIG. 13I). Imatinib, a small inhibitory molecule that blocks the tyrosine kinase domain of ABL1 (FIG. 13H), inhibited BCR-ABL1 mediated phosphorylation of Stat5 and ERK without altering the expression levels of BCR-ABL1 protein, whereas PspCas13b crRNAs efficiently silenced BCR-ABL1 protein expression and the downstream phosphorylation of Stat5 and ERK (FIG. 13I). Interestingly, the most potent crRNA+12 showed greater suppression of Stat5 phosphorylation than Imatinib, consistent with its high efficacy in depleting the BCR-ABL1 protein through mRNA silencing (FIG. 13I).

We also cloned and deployed 41 tiled crRNAs across the breakpoint (FIG. 13J; SEQ ID NOs: 103-143). Again, we observed that the silencing efficiency highly varied even between neighboring crRNAs. For instance, despite 96.6% sequence homology and only a single nucleotide position shift, crRNA+14 (SEQ ID NO: 137) achieved >90% silencing while crRNA+15 (SEQ ID NO: 138) exhibited no silencing, with consistent results evident in both quantitative microscopy and Western blot analyses (FIGS. 13J and 13K). The potent crRNA+14 (SEQ ID NO: 137) also exhibited higher silencing of downstream Stat5 phosphorylation (FIG. 13K). The contrasted silencing activity obtained with single-base resolved crRNAs within the same targeted region suggests the presence of key RNA sequences or features that profoundly influence PspCas13b activity.

Taken together, these data demonstrated the utility of PspCas13b as a versatile tool to efficiently silence tumor drivers such as fusion transcripts and alter their oncogenic signaling networks while remaining potent against treatment-resistance mutant. The data also indicate the presence of RNA microfeatures or sequences that determine PspCas13b silencing.

### Example 7

### PspCas13b can Efficiently Discriminate Between Translocated Tumor RNAs and Wild-Type RNAs Despite Extensive Sequence Homology

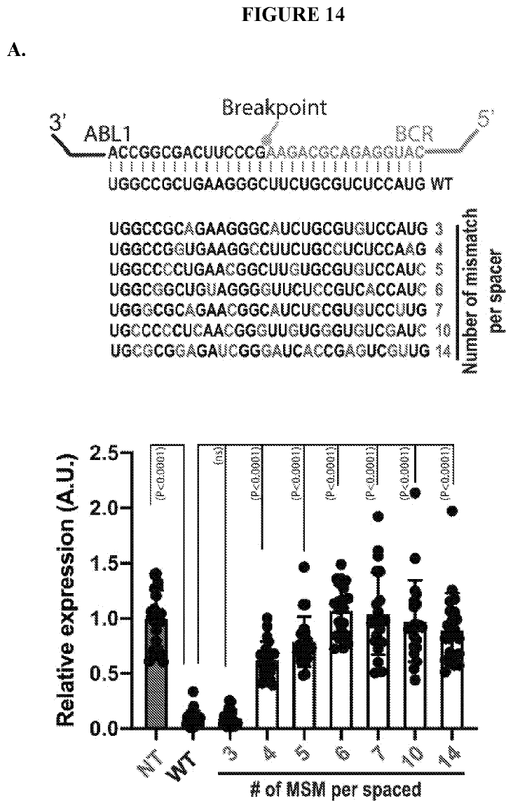

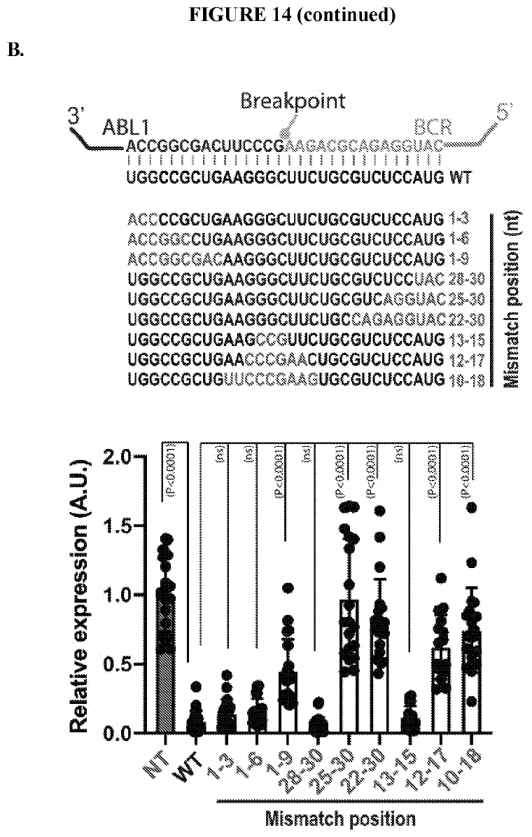

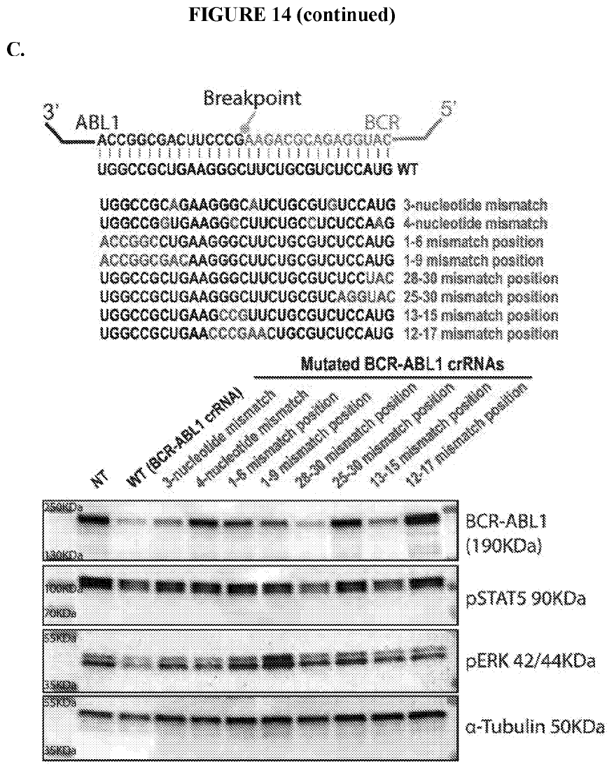

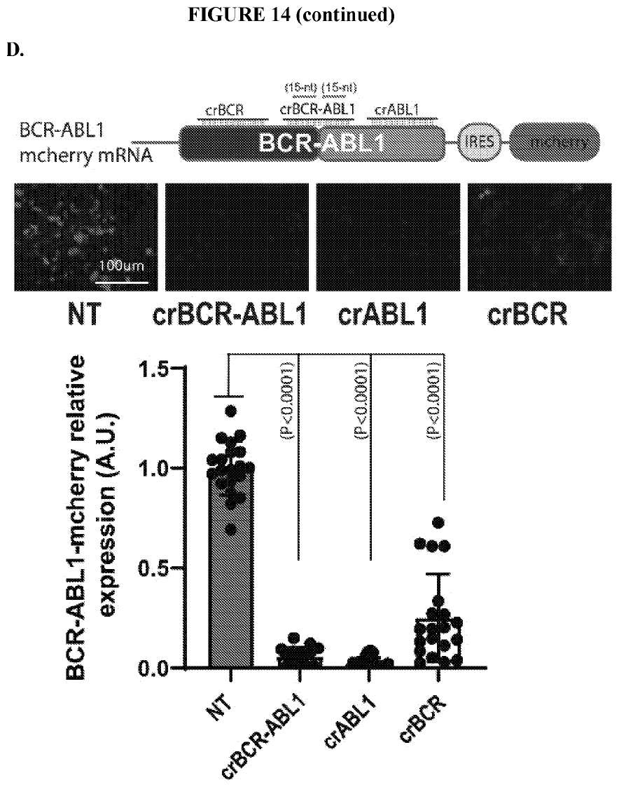

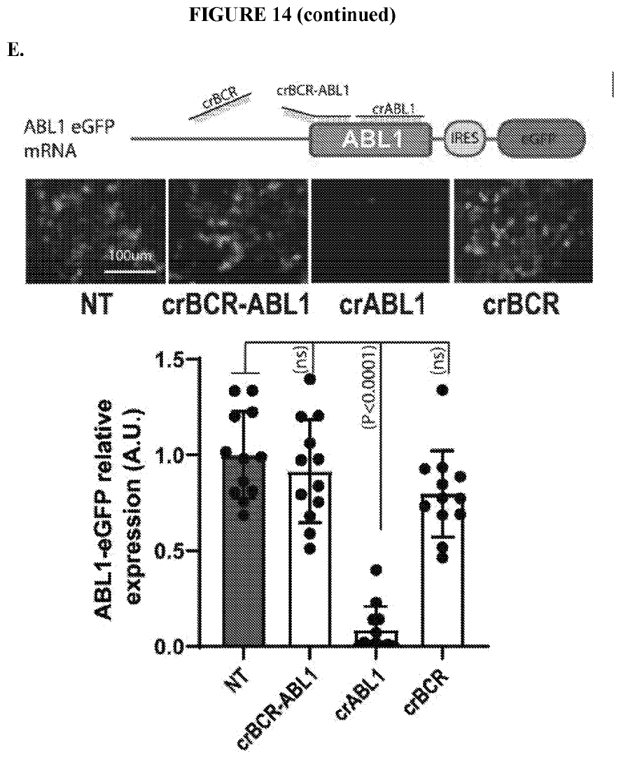

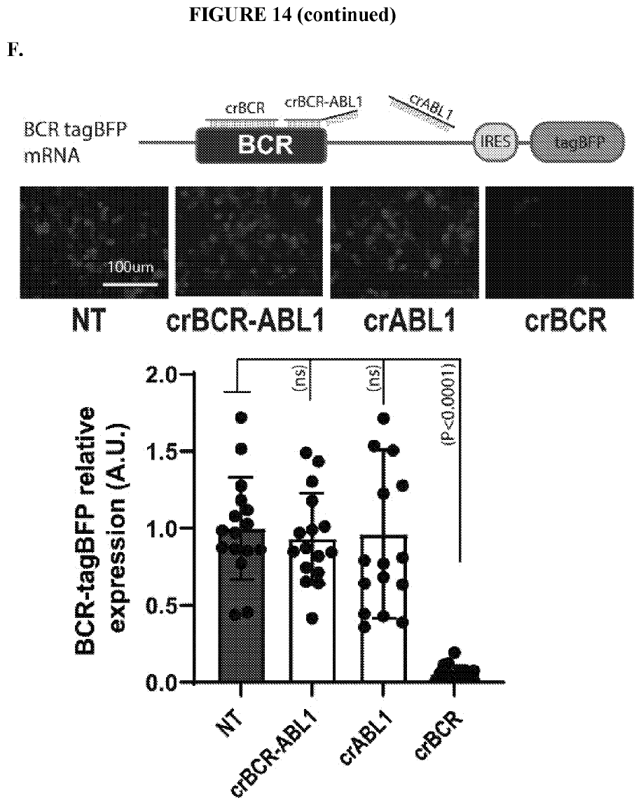

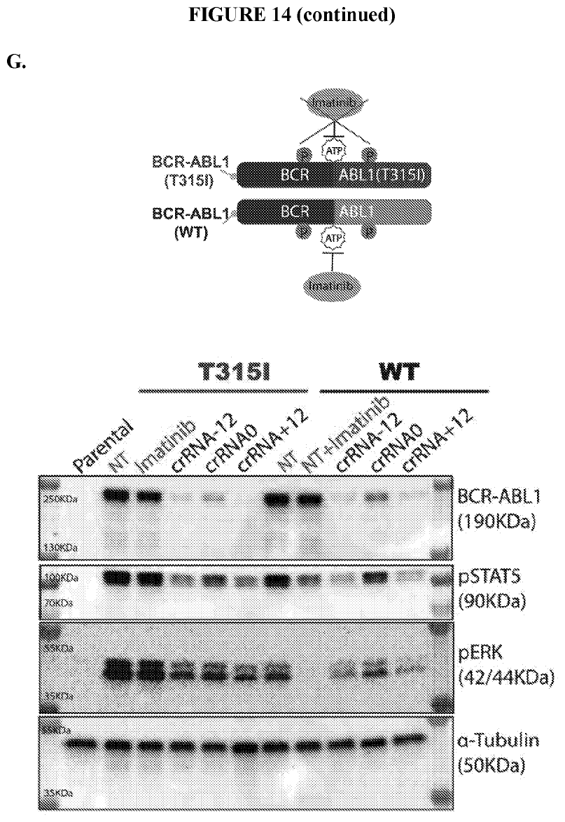

We investigated whether non-consecutive and consecutive mismatches impact BCR-ABL1 silencing to a similar degree observed in the mCherry model. To test this, we introduced 3, 4, 5, 6, 7, 10, and 14 non-consecutive mismatches between the spacer of BCR-ABL1 crRNA (crBCR-ABL1; SEQ ID NO: 123) and the targeted breakpoint sequence (FIG. 14A; SEQ ID NOs: 344-350). The data revealed that 3 nucleotide mismatches were well tolerated and didn't result in any significant loss of silencing. However, 4 or higher number of non-consecutive nucleotide mismatches drastically impaired crRNA silencing efficiency (FIG. 14A). Next, we introduced 3, 6, and 9 consecutive nucleotide mismatches to the 5′end, 3′end, or central regions of this spacer (SEQ ID NOs: 335-342) and measured their impact on the silencing efficiency. 3 consecutive nucleotide mismatches at various positions did not affect the silencing of BCR-ABL1. 6 consecutive nucleotide mismatches were also well tolerated when positioned at the 5′ end of the spacer (1-6), however, when positioned at the 3′end (25-30) or at the central region (13-18) they led to notable loss of silencing. 9 consecutive nucleotide mismatches dramatically curtailed silencing irrespective of position (FIG. 14B). This mutagenesis analysis of crRNAs targeting the breakpoint of BCR-ABL1 confirmed the asymmetry of mismatch tolerance and again demonstrated higher sensitivity to non-consecutive nucleotide mismatches relative to consecutive mismatch. Western blot analysis of BCR-ABL1 protein expression confirmed these data and showed that 3-nucleotide mismatches are well tolerated, while 4-nucleotide mismatches or higher led to substantial or complete loss of silencing (FIG. 14C). Overall, the data highlights the specificity of PspCas13b and its potential to discriminate between transcripts despite extensive sequence homology.

To confirm this specificity, we tested BCR-ABL1 fusion targeting crRNAs against wild type untranslocated BCR and ABL1 transcripts expressed in normal tissues. We cloned constructs encoding partial mRNA sequences of the BCR-ABL1 fusion, BCR alone, and ABL1 alone in frame with mCherry, eGFP, or TagBFP fluorescent reporters, respectively (FIGS. 14D and 14E). We designed 3 crRNAs targeting the BCR-ABL1 breakpoint sequence (crBCR-ABL1, SEQ ID NO: 123), BCR sequence (crBCR), or ABL1 sequence (crABL1) that we tested against the aforementioned constructs. The fluorescence signals from mCherry, eGFP, and TagBFP enable accurate quantification of on-target and off-target silencing with these crRNAs. As anticipated, all 3 crRNAs silenced the bona fide BCR-ABL1 transcript as this mRNA possesses completely complementary spacer binding sites for all three crRNAs (FIG. 14D). However, ABL1 and BCR transcripts were silenced only by their cognate crABL1 and crBCR crRNAs (FIGS. 14E and 14F). Notably, crBCR-ABL1 targeting the breakpoint sequence had no effect on either BCR or ABL1 wild type transcripts despite 15-nucleotide sequence base pairing (FIGS. 14D-14F). These data demonstrate the high-resolution capability of PspCas13b and its utility to specifically silence oncogenic gene fusion drivers at the RNA level while sparing non-translocated wild type transcripts expressed in normal cells.

Acquired drug resistance to all approved ABL1 kinase inhibitors through secondary mutations remains a major challenge in the treatment of BCR-ABL1 driven cancers. For instance, the BCR-ABL1 kinase domain mutation Thr315Ile (T315I) confers resistance to imatinib and drives tumor relapse. We hypothesized that unlike imatinib, targeting the breakpoint of BCR-ABL1 transcript with potent crRNAs will remain effective against both BCR-ABL1 variants as the mutation is located outside the targeted sequences at the breakpoint. We tested the potency of imatinib or three PspCas13b crRNAs targeting the ancestral or T315I BCR-ABL1 variants. As anticipated, imatinib efficiently inhibited the oncogenic signaling of ancestral BCR-ABL1 but failed to effectively suppress T315I activation and the downstream signaling (FIG. 14G). Notably, all three PspCas13b crRNAs we tested largely inhibited the expression of ancestral and T315I BCR-ABL1 proteins and their downstream oncogenic signaling as exemplified by phospho-STAT5 and phospho-ERK inhibition. Consistent with previous data, crRNA-12 (SEQ ID NO: 111) and crRNA+12 (SEQ ID NO: 135) achieved the highest inhibitory effect due to higher silencing potency (FIG. 14G). These data demonstrate that targeting the breakpoint of BCR-ABL1 transcript can overcome drug resistance commonly observed in recurrent leukemia.

### Example 8

### crRNAs Achieve Equipotent Silencing of Wild Type and Single Nucleotide Variant Tumor Transcripts

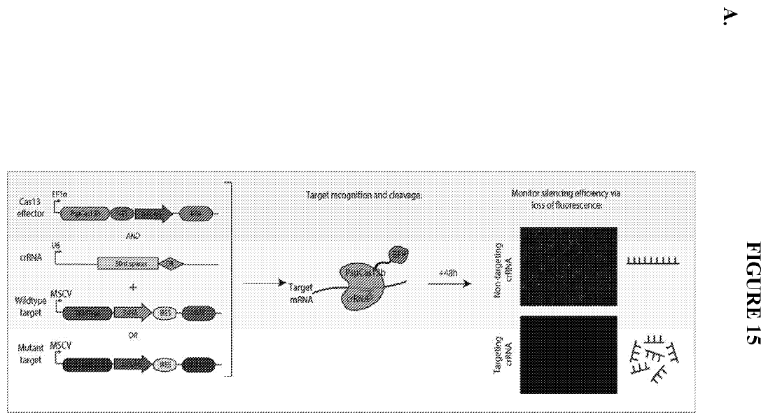

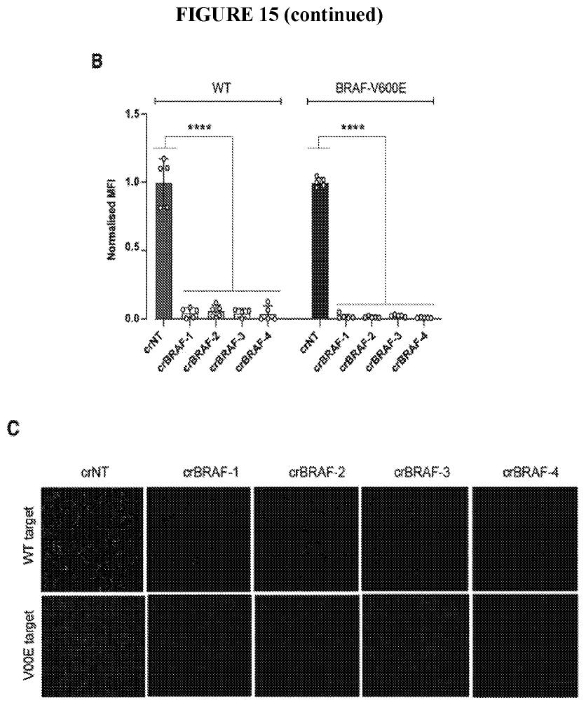

To model the silencing specificity of crRNAs in point-mutated versus wild-type transcripts, we designed a simple reporter assay that allows us to monitor silencing efficiency via loss-of-fluorescence signal (FIG. 15A). Here, DNA regions approximately 250 bp up- and down-stream of the single nucleotide variant are first cloned into a MSCV plasmid backbone that also encodes a fluorescent reporter. As the DNA sequence of interest and the fluorescent protein are linked via an internal ribosome entry site (IRES), the two are co-transcribed; in this case, truncated wild type BRAF is co-transcribed with GFP and truncated BRAF-V600E is co-transcribed with mCherry. Each of these constructs were then transfected into HEK293T cells alongside two other plasmids encoding (i) a PspCas13b effector and (ii) a crRNA comprising a spacer sequence that was capable of hybridizing to the target RNA sequence. As the target RNA sequence and the fluorescent reporter are transcribed as a single mRNA molecule, efficient cleavage of the target RNA sequence results in a proportional loss of fluorescence signal, which can be evaluated via fluorescence microscopy at 48 h post-transfection.

When this assay was employed using four crRNAs which tile the V600E mutation (crBRAF1-4; SEQ ID NOs: 419-422), incredibly potent mRNA cleavage relative to the non-targeting control (crNT) was observed for all crRNAs tested, with negligible difference in silencing efficiency between the wild-type BRAF and V600E-mutated transcripts (FIGS. 15B and 15C).

### Example 9

### Single Nucleotide Mutagenesis of Parental crRNAs Allows for Single Nucleotide Variant Transcriptional Repression

As BRAF^{V600E} results from a T>A substitution, the spacer sequences used in any BRAF^{V600E} crRNA will inherently have a one nucleotide mismatch when targeting the wild-type BRAF sequence. As any additional perturbations to the crRNA sequence would be made in additional to the original T>A substitution, the number of mismatches in the wild-type sequence will always be n+1, where n is the number of mismatches in the spacer sequence when the V600E pathogenic mutation is comprised in the target RNA sequence.

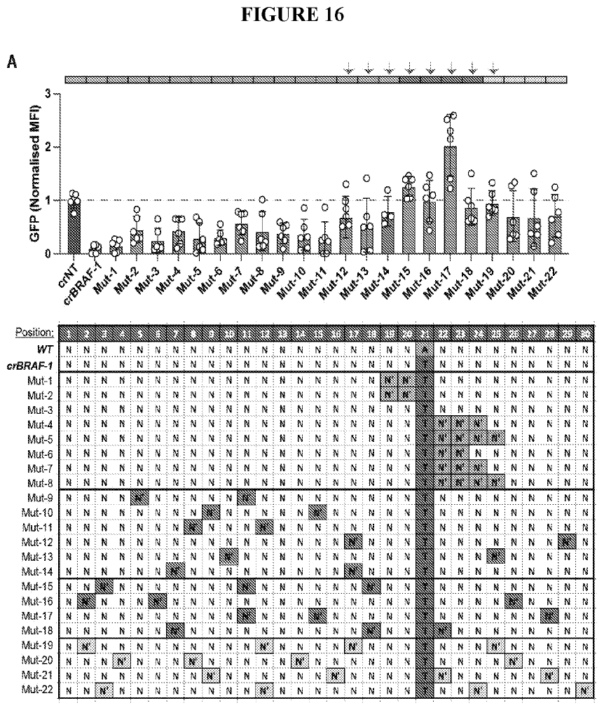

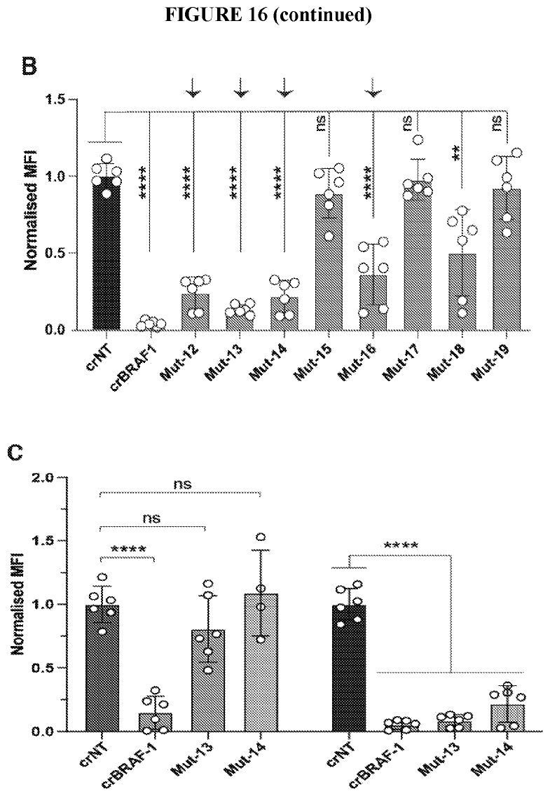

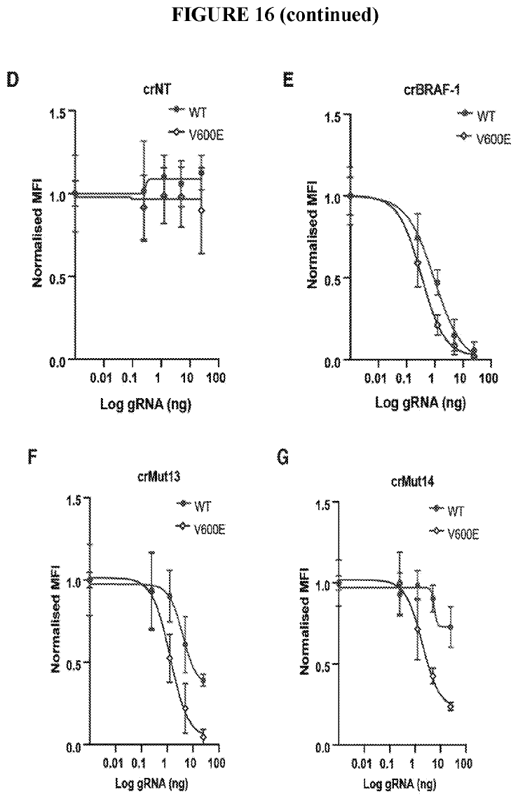

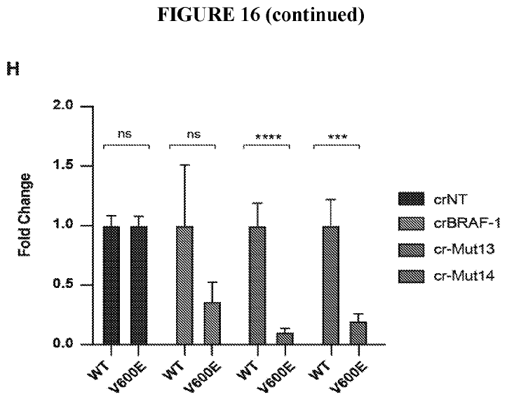

The lack of selective silencing when using the V600E crRNAs described herein (FIGS. 15B and 15C) indicated that a single-nucleotide mismatch with the wild-type sequence was not sufficient to confer single nucleotide variant-specific silencing. Thus, we sought to determine the mismatch tolerance threshold that would confer preferential silencing of the V600E transcript. To do so, we serially mutagenized the perfect match crBRAF1 by introducing either contiguous blocks of 2-4 mismatched nucleotides flanking the SNV site (FIG. 16A, lower panel, orange), or a series of two (FIG. 16A, lower panel, orange), three (FIG. 16A, lower panel, blue), or four (FIG. 16A, lower panel, orange) single-nucleotide mismatched that were distributed along the spacer sequence.

Screening this panel of crRNAs (crMut-1-crMut-22; SEQ ID NOs: 424-445) against the BRAF-WT-GFP construct revealed a wide variety of silencing efficiencies (FIG. 16A, upper panel). Whilst some crRNAs retained their ability to silence the wild-type transcript, often with efficiencies comparable with the parental crBRAF-1 (SEQ ID NO: 419), our comprehensive mutagenesis revealed that certain crRNAs, with further mutation, completely lost their silencing capacity (FIG. 16A, upper panel, green arrows). The crRNAs that were least efficient at silencing BRAF-WT-GFP transcripts were over represented in the mutagenesis groups where spacers had two- or three mismatches in the V600E spacer (corresponding to three and four mismatches with the wild type, respectively).

However, when these candidate crRNAs were re-screened against the BRAF^{V600E}-mCherry construct, only a subset retained the ability to silence the mutated transcript (FIG. 16B, red arrows). Consequently, we escalated the two most promising candidates, crMut-13 and crMut-14 (SEQ ID NOs: 436 and 437), for parallel screening in the WT-GFP and V600E-mCherry constructs and confirmed that these two guides could differentially target the V600E transcript (FIGS. 16C and 16H). Titration of these crRNAs established that this differential silencing was dose-dependent (FIGS. 16D and 16G). This highlighted the flexibility of this targeting strategy, as the concentration of crRNA required to maximize silencing of the single nucleotide variant transcript could be optimized to minimize off-target silencing of the wild-type transcript.

### Example 10

### V600E-Specific Silencing of Full Length BRAF is Achievable with Cas13b but not Cas9

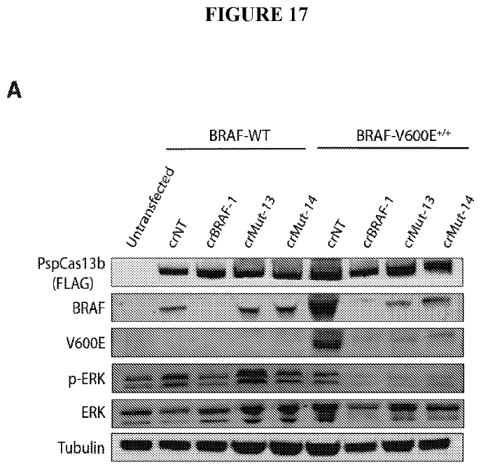

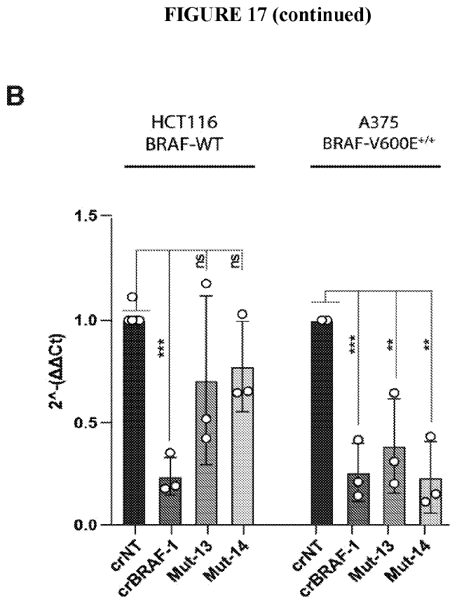

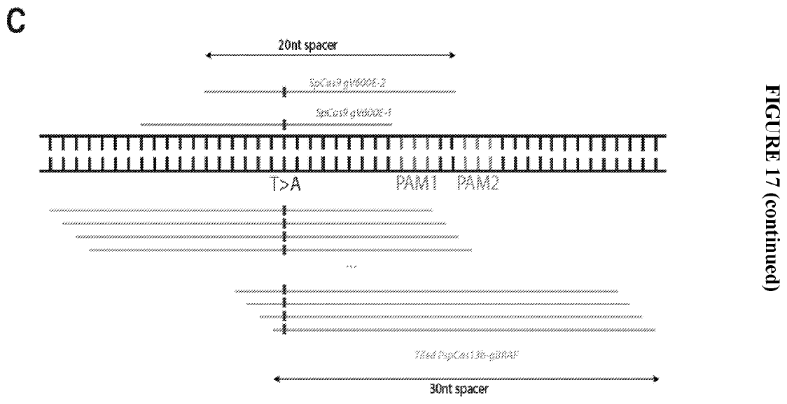

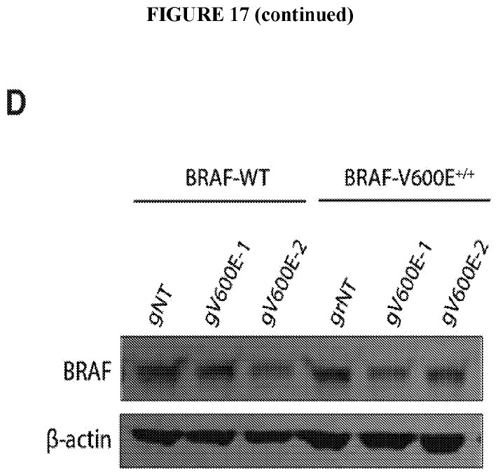

As the constructs utilized thus far encoded a truncated, single nucleotide variant-spanning region of the target gene, we next sought to confirm that the V600E-specificity of crMut-13 and crMut-14 (SEQ ID NOs: 436 and 437) would be retained when targeting the full-length BRAF transcript. HEK293T cells transfected with constructs encoding full-length BRAF-WT or BRAF^{V600E} retained the expected pattern of silencing, with crMut-13 and crMut-14 (SEQ ID NOs: 436 and 437) preferentially knocking down BRAF^{V600E} at the protein level, indicating that the silencing efficiency of these pre-validated crRNAs was not disrupted by any potential secondary structures present in the full-length transcripts (FIG. 17A). V600E knockdown in the V600E-transfected HEK cells resulted in potent shutdown of the MAPK pathway, as indicated by reduced expression of phosphorylated ERK (FIG. 17A). Conversely, pERK downregulation was only observed in cells transfected with wild-type BRAF in the crBRAF-P1 condition, indicating that crMut-13 and crMut-14 (SEQ ID NOs: 436 and 437) have limited efficacy in silencing wild-type BRAF (FIG. 17A).

As our validation experiments were performed in HEK293T cells, selected due to their high transfectability, we next questioned whether our crRNAs would effectively silence oncogenic BRAF in a cancer cell context. To test this, we compared the silencing efficiency of crMut-13 and crMut-14 (SEQ ID NOs: 436 and 437) in colorectal adenocarcinoma HCT116 cells, which express wild-type BRAF, and A375 melanoma cells, which harbor a homozygous BRAF^{V600E} mutation. qPCR analysis demonstrated that significant BRAF silencing was observed for all crRNAs in the V600E-mutated A375 cells, but only in the parental crBRAF-1 (SEQ ID NO: 419) condition for HCT116 cells, confirming that the efficiency and specificity of our crRNAs was retained in the endogenous context (FIG. 17B).

To compare the silencing efficiency of PspCas13b-compatible crRNAs against other CRISPR modalities, we designed V600E-spanning guides that would enable cleavage by the archetypical DNA-cleaving SpCas9.

SpCas9 cleavage mandates the presence of a protospacer-adjacent motif (PAM) 2-6 nucleotides upstream of the target DNA sequence, thereby restricting the regions targetable with this CRISPR effector. Given these restrictions, there are only two possible V600E-spanning gRNAs that fulfil the PAM requirements for Cas9 (FIG. 17C) and each of these shows only moderate silencing of BRAF^{v600E} at the protein level (FIG. 17D). This poor efficacy is possibly due to the single nucleotide variant location in one of the most 3′ exons of the BRAF gene, therefore reducing the likelihood of a Cas9-induced indel having a deleterious effect on protein translation. Conversely, there is no evidence that PspCas13b requires a PAM-like sequence, so the 30-nucleotide spacer sequence could begin at any position relative to the T>A SNV (FIG. 17C). Thus, 30 possible crRNAs can be generated for any given single nucleotide variant, thereby increasing the likelihood that one of these will show high silencing efficiency.

### Example 11

### V600E-Specific Silencing of Full Length BRAF is Achievable with Cas13d Ortholog, RfxCas12d

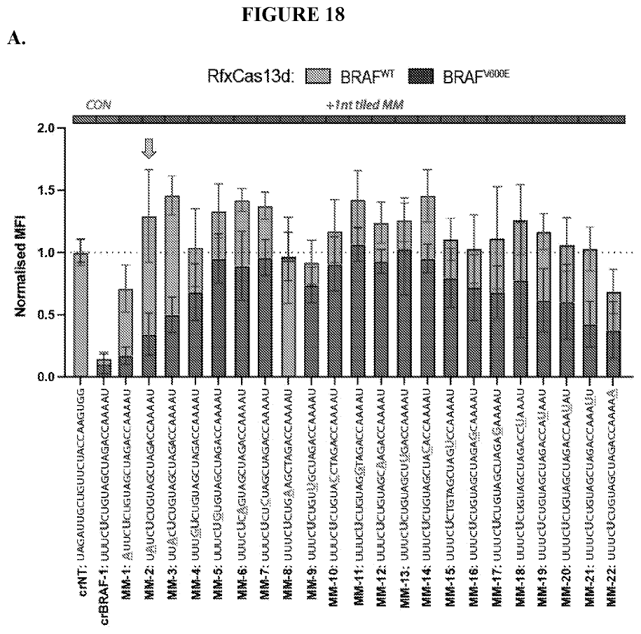

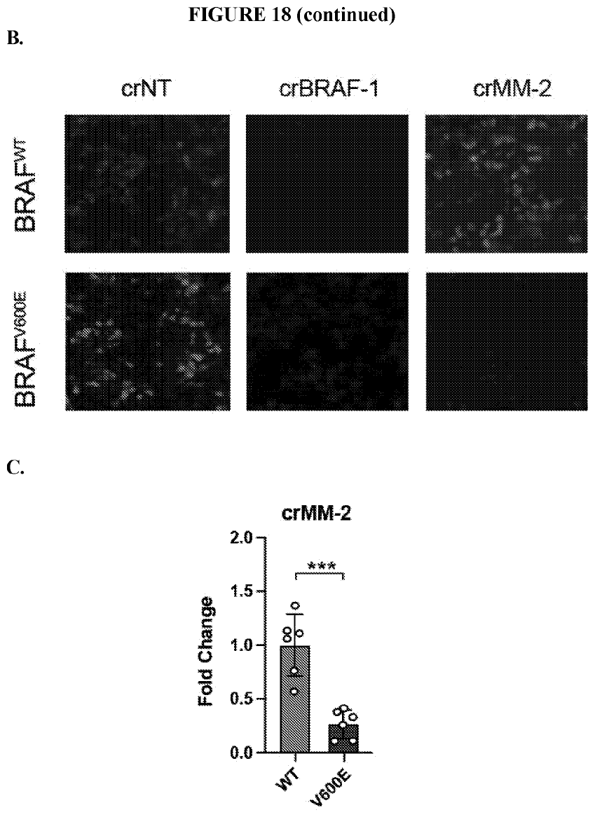

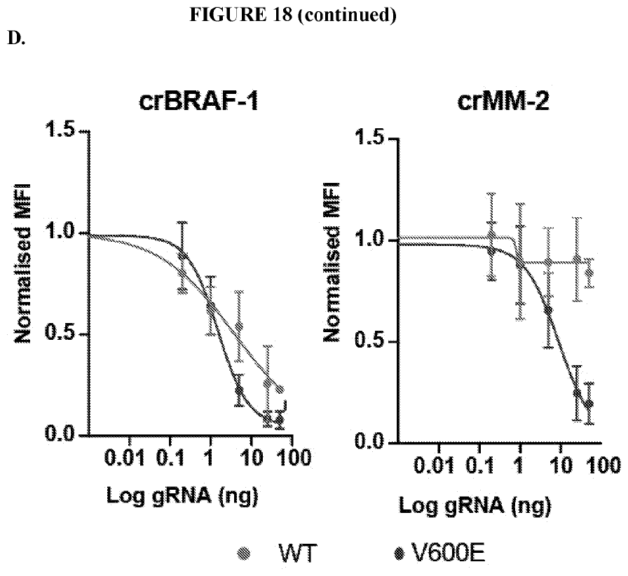

A ‘perfect-match’ (i.e., 100% sequence homology) crRNA targeting the V600E transcript (i.e., crBRAF-1; SEQ ID NO: 466) that showed equipotent silencing of both BRAF WT and V600E-mutated BRAF (FIG. 18A) was used as a template to systematically introduced one synthetic mismatch at each nucleotide position along the 23 nucleotide-long spacer sequence, thus generating a pool of 22 single-mismatch crRNAs (FIG. 18A; SEQ ID NOs: 467-488).

Using the methods described elsewhere herein (see, e.g., Example 8), HEK293T cells were co-transfected with three plasmids encoding (i) RfxCas13d (ii) a crRNA and (iii) fluorescently tagged BRAF WT or BRAF-V600E, then screening for silencing efficiency at 48 h post-transfection. Of the crRNA screened, crMM2 (SEQ ID NO: 468) demonstrated preferential silencing of BRAF-V600E relative to BRAF WT (FIG. 18A-C). crMM2 (SEQ ID NO: 468) exhibited SNV-selective silencing with minimal off-target silencing of the WT transcript, which was not observed for using crBRAF-1 (FIG. 18D). Collectively, these data demonstrate that V600E-mutated BRAF transcripts can be selectively silenced with RfxCas13d.

### Example 12

### G12-Specific Silencing of KRAS is Achievable with RfxCas12b

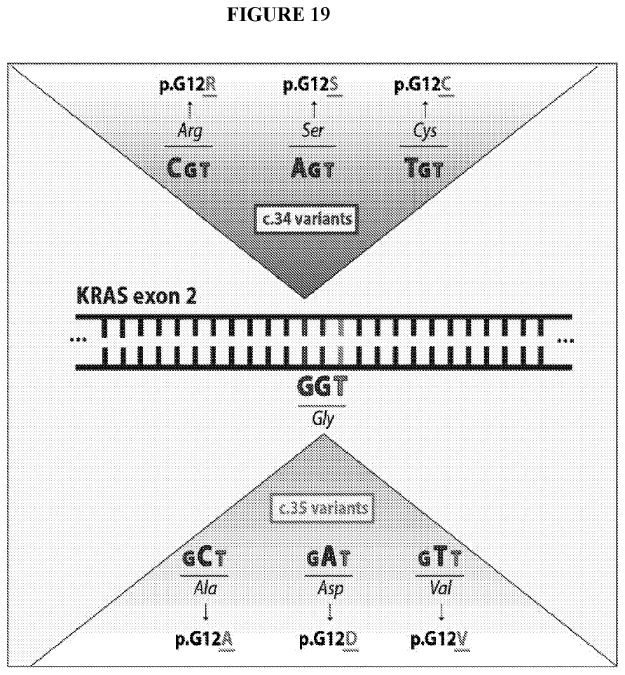

There are 6 KRAS G12X mutations, which all result from SNVs in exon 2 of the KRAS gene, at nucleotide positions 34 (G12C, G12R, G12S) or 35 (G12A, G12D, G12V) (FIG. 19). Targeting the full suite of G12X mutants additionally offers a unique opportunity to validate the relative importance of (i) the type of nucleotide substitution (i.e., G>A, or G>T) and (ii) the position of the SNV in the spacer sequence (i.e., c.34, or c.35) for efficient Cas13-mediated silencing.

For example, both KRAS G12C and KRAS G12R occur at nucleotide position 34 of the KRAS sequence, resulting from G>T and G>C substitutions, respectively. If the identity of the nucleotide that generates the missense mutation is not important for Cas13-mediated silencing, any SNV-specific crRNAs would be cross-reactive with the other SNVs that occur at the same position (i.e., the silencing efficiency for both G12C and G12R would be similar when using the same crRNA). Similarly, if the position of the nucleotide in the spacer sequence is important, it is possible that all c.34 variants would have similar silencing efficiency, and that this efficiency would differ from the c.35 variants.

With these questions in mind, we generated a library of KRAS G12X plasmids. Constructs expressing each of the six different KRAS mutations (SEQ ID NOs: 563-568), as well as a KRAS WT control (SEQ ID NO: 562), were cloned via site-directed mutagenesis of a KRAS-G12S-IRES-mCherry plasmid.

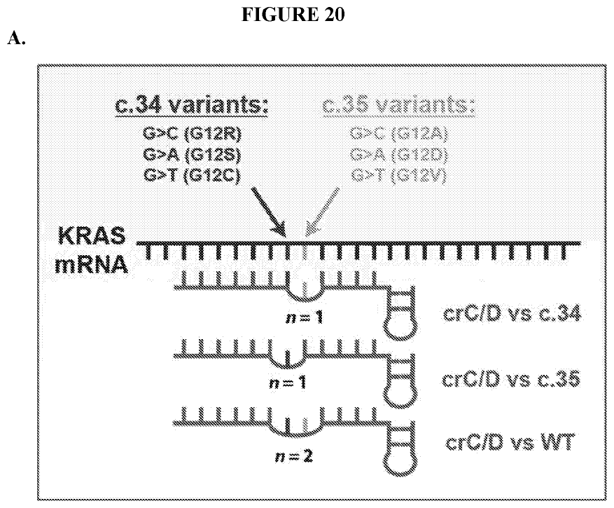

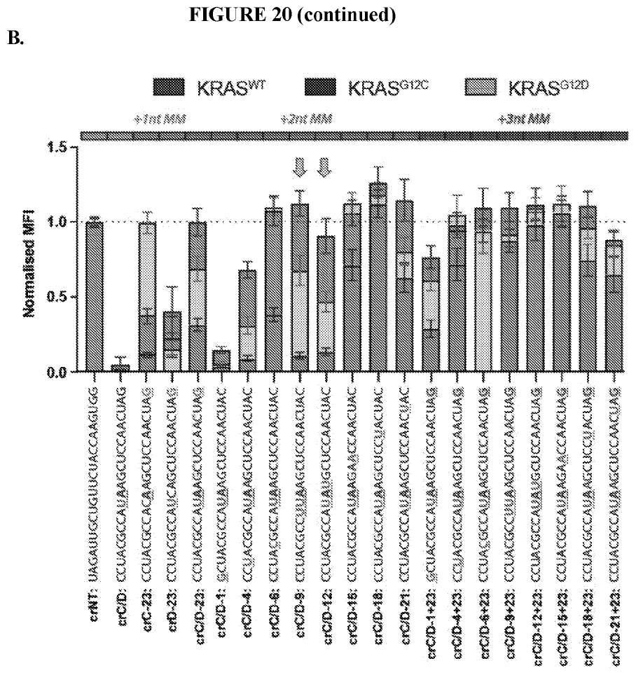

To discriminate between WT and G12 mutant KRAS transcripts, bi-specific crRNAs targeting the G12 hotspot were engineered (FIG. 20A). For example, rather than targeting the G12C and G12D mutations with separate crRNAs, we designed a parental crRNA that targets both KRAS G12C and G12D mutations (i.e., crC/D; SEQ ID NO: 492) by incorporating the complementary nucleotides for both the G12C and G12D SNVs in a single spacer sequence. This design strategy can be extrapolated to any combination of c.34 and c.35 variants and ensures that, even in the absence of additional synthetic mismatches, these bi-specific crRNAs will have at least a one-nucleotide mismatch with any other G12 variant, but at least two mismatches with KRAS wild type.

crC/D (SEQ ID NO: 492) was shown to efficiently silence both G12C and G12D KRAS mutant transcripts, but also was shown to non-discriminately silence the KRAS WT transcript (FIG. 20B). Thus, crC/D was mutagenized using the methods described elsewhere herein, systematically adding 1-3 synthetic mismatches into various positions along the spacer sequence. The screen identified two crRNAs, crC/D-9 (SEQ ID NO: 496) and crC/D-12 (SEQ ID NO: 494), with efficient silencing of G12C KRAS mutant transcripts, moderate silencing of G12D KRAS mutant transcripts, and limited silencing of the KRAS WT transcript (FIG. 20B). These crRNAs adopt the sequence of the parental crC/D (SEQ ID NO: 492) but contain an additional synthetic mismatch at position 9 or 12 of the spacer sequence, respectively. Titration of these engineered crRNAs confirmed preferential, dose-dependent silencing of G12C and G12D KRAS mutant transcripts with limited activity against the KRAS WT transcript (FIG. 21).

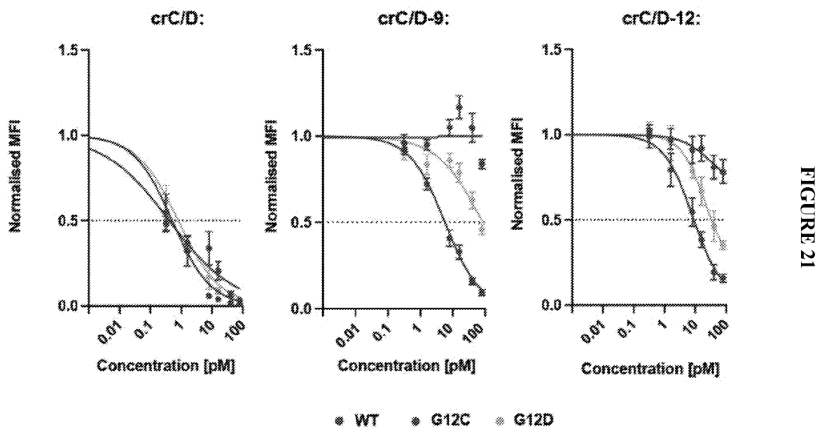

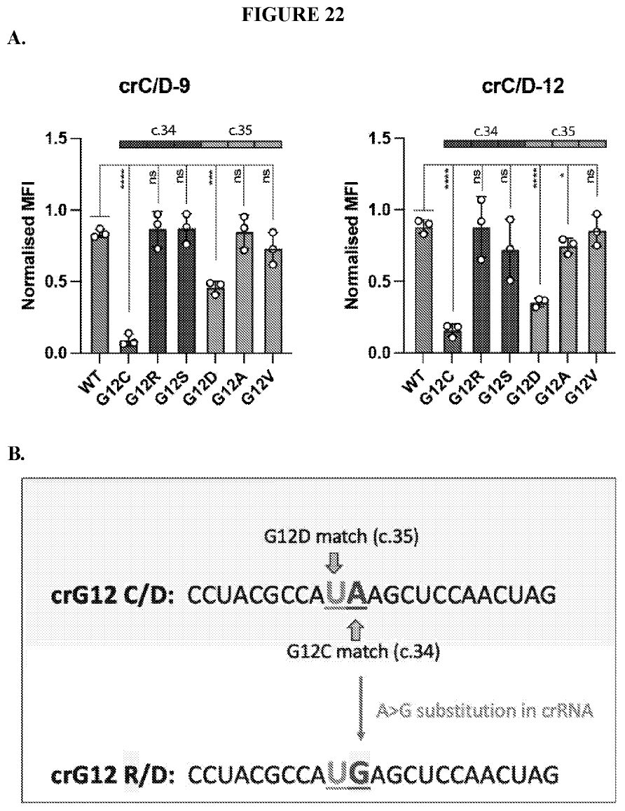

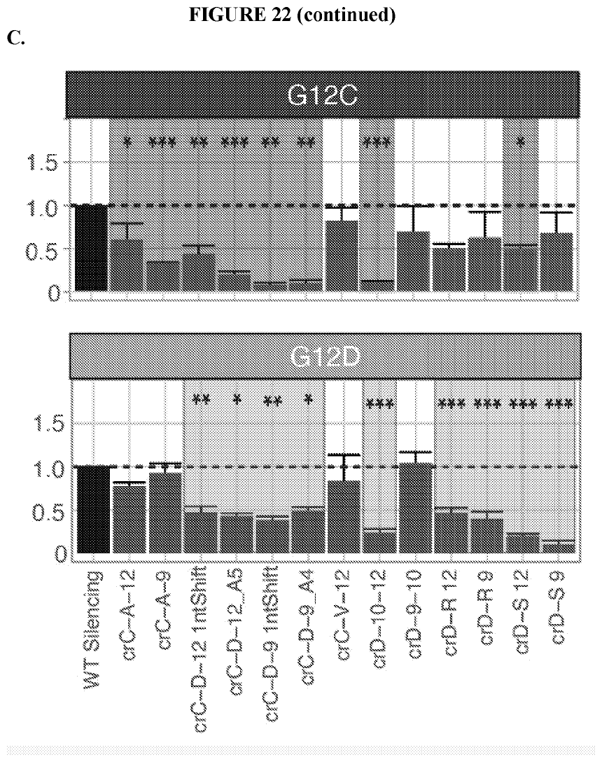

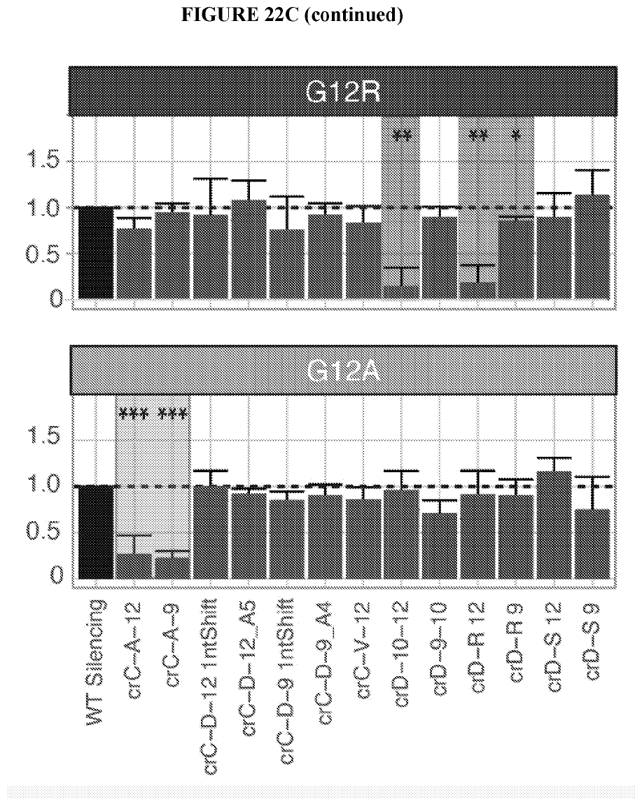

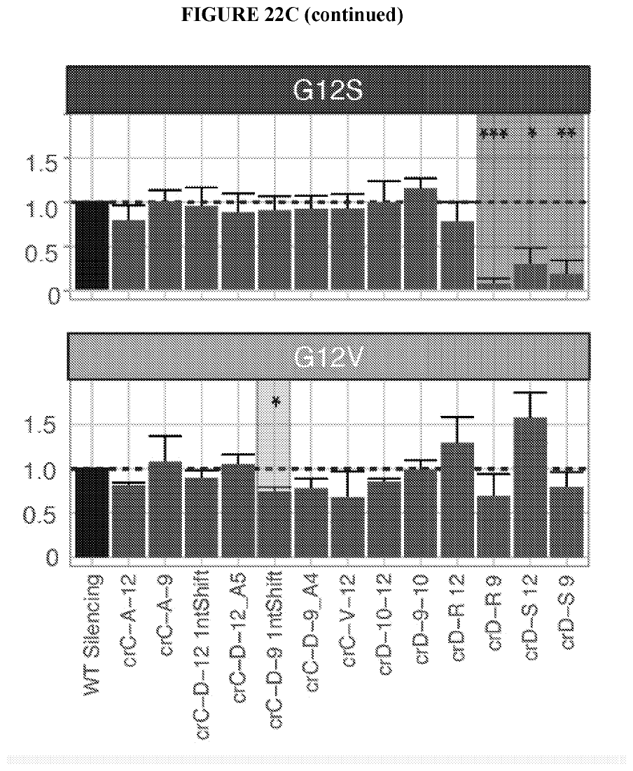

Despite high on-target activity against G12C and G12D mutant transcripts, the crC/D-9 (SEQ ID NO: 496) and crC/D-12 (SEQ ID NO: 494) crRNAs did not show efficient silencing of the G12X variants, G12R, G12S, G12A and G12V (FIG. 22A). Specificity of the crC/D-9 (SEQ ID NO: 496) and crC/D-12 (SEQ ID NO: 494) crRNAs were “switched” from one G12X variant to another by substituting the appropriate nucleotide at the c.34 or c.35 positions in the crRNA spacer (FIG. 22B). For example, the crC/D guide contains an “A” nucleotide in the spacer position complementary to KRAS c.34, such that it can hybridize with the “T” nucleotide substitution found in G12C-mutated KRAS (c.34 G>T); exchanging the spacer “A” for “G”, promotes base-pairing with the “C” substitution present in G12R-mutated KRAS (c.34 G>C), thereby “switching” the silencing activity from G12C to G12K Using this mutagenesis strategy, a series of crRNAs with the structure of the crC/D-9 and crC/D-12 crRNAs, but with nucleotide substitutions at the spacer positions involved in c.34 and c.35 base pairing.

This mutagenesis strategy generated at least one crRNA capable of selectively silencing each of the six possible G12 SNV mutants (FIG. 22C). Whilst certain crRNAs proved extremely specific for their encoded targets (e.g., crC/A-12 (SEQ ID NO: 490) shows significant silencing of only its intended G12A and G12C targets), other guides displayed high cross-reactivity against multiple G12 variants (e.g., crD/S-12 (SEQ ID NO: 525) can silence the G12S and G12D targets, but also G12C).

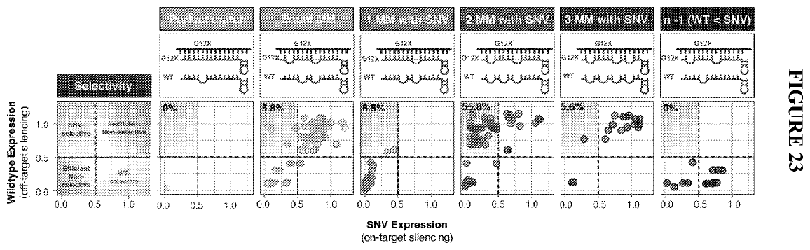

The broad variance in silencing efficiency of our crRNAs observed across a single G12 target (e.g. some G12C-targeting crRNAs are extremely efficient, and others less so) prompted us to investigate whether there are any generalizable features that could differentiate efficient SNV-selective vs inefficient or non-selective crRNAs. To identify any generalizable features that can differentiate efficiency SNV-selective crRNAs from inefficient, or non-selective crRNAs, the silencing data from every crRNA used across all KRAS-targeting screens was pooled and plotted based on silencing efficiency relative to the number of mismatches (with a single G12 target) in their spacer sequences (FIG. 23). The “perfect-match” crRNA for KRAS G12C (i.e., no mismatches with G12C KRAS mutant transcripts, and one mismatch with the KRAS WT transcript) efficiently silenced both WT and SNV transcripts with equipotency (FIG. 23). Similarly, it was shown that when there is an equal number of mismatches in the spacer sequence for both the WT and the SNV transcript (e.g., 2 mismatches with WT but also 2 mismatches with the SNV variant), crRNAs exhibit no selectivity and typically silence both WT or SNV with equivalent efficiency or inefficiency (FIG. 23). crRNAs that contain one mismatch with the SNV transcript (and two with the wild type) are comparably efficient at silencing both WT and G12 variant transcripts (FIG. 23), just as those with three mismatches with the SNV (and four with the wild type) are comparably inefficient (FIG. 23). Over half of all crRNAs containing two mismatches with the SNV (and three with the wild type) exhibit SNV-selective silencing (FIG. 23).

The most potent selective-silencers of G12A (crC/A-9, i.e., crG12A; SEQ ID NO: 491), G12C (crC/D-9-Int-shift, i.e., crG12C; SEQ ID NO: 496), G12R (crD-10-12, i.e., crG12R; SEQ ID NO: 521), G12D (crD/S-9, i.e., crG12D; SEQ ID NO: 526), and D12S (i.e., crD/S 9; SEQ ID NO: 526). Although one crRNA, crC/D-9-1 nt-shift (SEQ ID NO: 495), demonstrated selective silencing of KRAS G12V relative to KRAS WT transcripts (FIG. 22C).

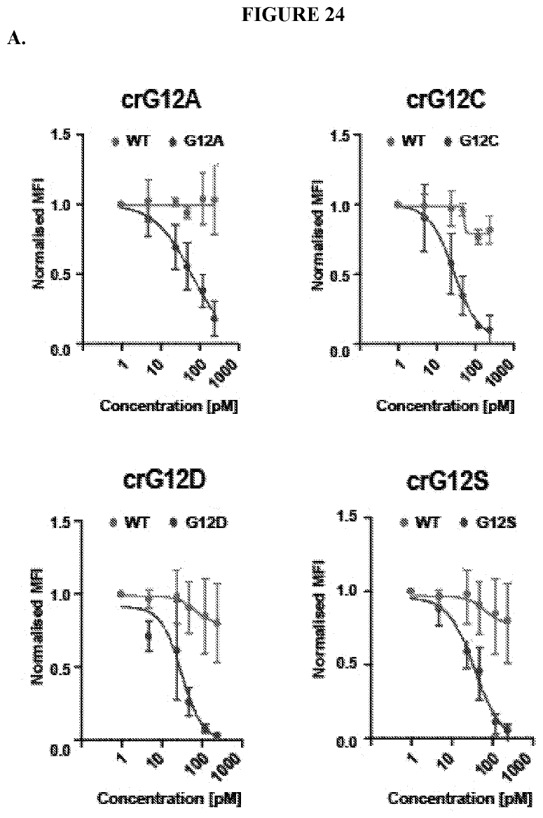

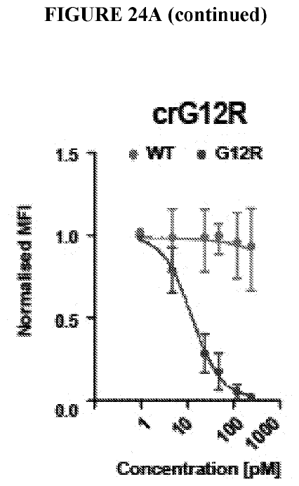

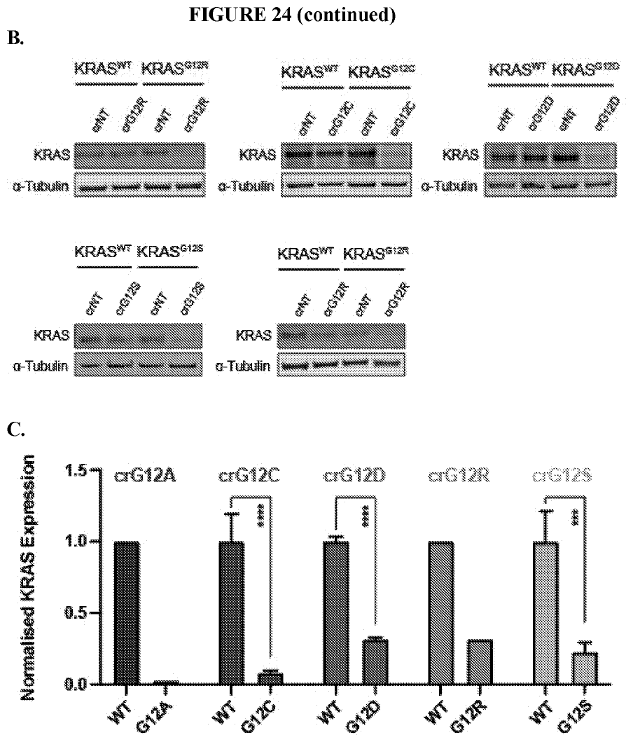

Titration of our top-performing crRNAs confirmed the unexpected, SNV-selective, dose-dependent silencing activity of these crRNAs against all G12 variants tested (FIG. 24A). Moreover, G12-selectivity was maintained at the protein level for all variants (FIG. 24B-C). To the best of our knowledge, this represents the first time that KRAS G12X variants have been selectively silenced using RfxCas13d. This is particularly surprising, given that all KRAS G12X variants (with the exception of G12C) are considered to be undruggable, and thus clinically unactionable. Taken together, these data represent a key advance in the targeting of cancers comprising a KRAS-mutation.

### CONCLUSION

CRISPR tools are anticipated to revolutionize the management of human genetic diseases, including cancers, by enabling sequence-specific editing of aberrant genes. Programmable RNA-targeting Cas13 enzymes can offer effective and specific silencing of the targeted transcripts without the risk of permanent alteration of genomic DNA, making these CRISPR technologies attractive for personalized oncology and beyond. However, the molecular bases that govern RNA target recognition and silencing by recently discovered Cas13 enzymes remain poorly understood. The molecular parameters that determine Cas13 silencing efficiency and specificity have been identified herein, which have been reduced to practice in the generation of RNA editing systems comprising de novo designed crRNAs that consistently outperformed conventional designs. In particular, crRNA comprising spacer sequences enriched for G nucleotides enhanced the potency of RNA editing systems comprising the crRNAs significantly more than would have been expected, for example, the selection of crRNAs with a G-rich motif at the 5′end of the spacer that drastically enhanced the potency of PspCas13b.

Furthermore, ineffective crRNAs can be selected and modified to improve the potency of the crRNAs, even if such modifications result in the incorporation of mismatched nucleotides relative to the target RNA sequence. For example, de novo designed crRNAs harboring target matched or target-mismatched ‘GG’ sequence at the 1^{st} and 2^{nd} nucleotide positions of the spacer can greatly enhance the silencing potency of otherwise poorly effective crRNAs. The ability of this target mismatched ‘GG’ motif to rescue the potency of certain ineffective crRNAs unexpectedly expands the range of effective crRNAs for a given target, which may be particularly important for narrowly defined target sequences, especially when targeting breakpoint region of fusion transcripts, RNA isoforms, or single-nucleotide variants.

The crRNA and RNA editing systems of the present disclosure have been enabled in methods for the alteration of target RNA sequences with single-base resolution, which further expands the targeting spectrum of the Cas13 effector proteins contemplated herein. Namely, PspCas13b can be employed with crRNAs with the optimized features defined herein to efficiently and selectively (i.e., potently) silence oncogenic fusion gene transcripts that drive multiple human malignancies, e.g., leukemia. Fusion gene transcripts are aberrant RNA structures frequently detected in various cancer types resulting from chromosomal translocations. Despite their established role as drivers of oncogenesis, the vast majority of gene fusions remain undruggable. The design-flexibility of Cas13 provides an attractive option to personalize targeting of these fusion genes at the transcript level. Fusion transcripts are ideal targets for Cas13 as they possess a unique chimeric sequence exclusively expressed by tumor cells but absent in normal tissues. As shown herein, the RNA editing systems can efficiently recognize and silence three different fusion transcripts including BCR-ABL1, a well-established driver of chronic myeloid leukemia (CML) and other malignancies. BCR-ABL1 transcript silencing led to subsequent depletion of the fusion protein and thereby inhibited the phosphorylation and activation of downstream STAT5 and ERK signaling pathways that are a hallmark of BCR-ABL1 driven cancers. These data therefore demonstrate the ability of the RNA editing systems described herein to silence major tumor drivers and remodel their oncogenic networks. Importantly, the inhibitory effect of potent crRNAs targeting BCR-ABL1 can outperform the efficiency of imatinib, a tyrosine kinase inhibitor used to treat CML and other BCL-ABL1 dependent malignancies. It has also been demonstrated that optimal design of crRNA can silence the mRNA of oncogenic fusion drivers without suppressing the fusion partners' wild-type RNA variants that are expressed in normal cells. Accordingly, these data enable the use of the crRNA, RNA editing system and methods disclosed herein for targeting of RNA sequences with homology to non-target RNA sequences, with high specificity or a reduced risk of off-target RNA silencing.

Moreover, by specifically targeting the breakpoint of gene fusion transcripts, it has been shown that the RNA editing systems described herein remain highly effective against gene fusion transcripts that have acquired secondary mutations that have been associated with the development of therapeutic resistance to pharmacological treatments, such as imatinib. Accordingly, these data enable methods for the treatment of cancer patients with mutation-driven drug resistance in other tumor streams.

In addition, these data also enable the use of the RNA editing systems described herein to specifically target single nucleotide variant transcripts, such as single nucleotide variant oncogenic transcripts, whilst sparring the corresponding wild-type homolog.

Taken together, these data enable the design, selection and use of potent crRNA and RNA editing systems to alter RNA target sequences in a specific and effective manner. The surprising lack of collateral activity demonstrated using the RNA editing systems described herein is particularly useful in the development of personalized medicine through targeting aberrant RNA sequences that drive genetic disorders, e.g., cancer.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 1
crRNA spacer sequences

| Name | Spacer Sequence (forward strand) | SEQ ID NO: |
|---|---|---|
| NT crRNA | TAGATTGCTGTTCTACCAAGTAA | 5 |
| | TCCATCA | |
| PspmCherry1 (matching | GGCCATGTTATCCTCCTCGCCCT | 6 |
| position 3) | TGCTCAC | |
| PspmCherry2 (matching | GGAGCCCTCCATGTGCACCTTGA | 7 |
| position 49) | AGCGCAT | |
| PspmCherry3 (matching | GGCGGTCTGGGTGCCCTCGTAGG | 8 |
| position 118) | GGCGGCC | |
| PspmCherry4 (matching | GGAGCCGTACATGAACTGAGGG | 9 |
| position 193) | GACAGGAT | |
| PspmCherry5 (matching | GGGGAAGGACAGCTTCAAGTAG | 10 |
| position 250) | TCGGGGAT | |
| PspmCherry6 (matching | GGAGTCCTGGGTCACGGTCACCA | 11 |
| position 319) | CGCCGCC | |
| PspmCherry7 (matching | GGTCTTCTTCTGCATTACGGGGC | 12 |
| position 406) | CGTCGGA | |
| PspmCherry8 (matching | GGGCGCCGTCCTCGGGGTACATC | 13 |
| position 455) | CGCTCGG | |
| PspmCherry9 (matching | GGTGGTCTTGACCTCAGCGTCGT | 14 |
| position 526) | AGTGGCC | |
| PspmCherry 10 (matching | GGTGATGTCCAACTTGATGTTGA | 15 |
| position 593) | CGTTGTA | |
| PspmCherry 11 (matching | GGTGGAGTGGCGGCCCTCGGCG | 16 |
| position 655) | CGTTCGTA | |
| Shift PspmCherry1 | CCATGTTATCCTCCTCGCCCTTGC | 17 |
| (matching position 2) | TCACCA | |
| Shift PspmCherry3 | CACGGAGCCCTCCATGTGCACCT | 18 |
| (matching position 52) | TGAAGCG | |
| Shift PspmCherry5 | TCGGGGAAGGACAGCTTCAAGT | 19 |
| (matching position 252) | AGTCGGGG | |
| Shift PspmCherry6 | TCCTGGGTCACGGTCACCACGCC | 20 |
| (matching position 315) | GCCGTCC | |
| Shift PspmCherry10 | TCCAACTTGATGTTGACGTTGTA | 21 |
| (matching position 585) | GGCGCCG | |
| Pspmcherry8(−15 nt) | TGATCTCGCCCTTCAGGGCGCCG | 22 |
| | TCCTCGG | |
| Pspmcherry8(−12 nt) | TCTCGCCCTTCAGGGCGCCGTCC | 23 |
| | TCGGGGT | |
| Pspmcherry8(−9 nt) | CGCCCTTCAGGGCGCCGTCCTCG | 24 |
| | GGGTACA | |
| Pspmcherry8(−6 nt) | CCTTCAGGGCGCCGTCCTCGGGG | 25 |
| | TACATCC | |
| Pspmcherry8(−3 nt) | TCAGGGCGCCGTCCTCGGGGTAC | 26 |
| | ATCCGCT | |
| Pspmcherry8(+3 nt) | CGCCGTCCTCGGGGTACATCCGC | 27 |
| | TCGGAGG | |
| Pspmcherry8(+6 nt) | CGTCCTCGGGGTACATCCGCTCG | 28 |
| | GAGGAGG | |
| Pspmcherry8(+9 nt) | CCTCGGGGTACATCCGCTCGGAG | 29 |
| | GAGGCCT | |
| Pspmcherry8(+12 nt) | CGGGGTACATCCGCTCGGAGGA | 30 |
| | GGCCTCCC | |
| Pspmcherry8(+15 nt) | GGTACATCCGCTCGGAGGAGGC | 31 |
| | CTCCCAGC | |
| Pspmcherry 11(−15 nt) | CTCGTCCATGCCGCCGGTGGAGT | 32 |
| | GGCGGCC | |
| Pspmcherry 11(−12 nt) | GTCCATGCCGCCGGTGGAGTGGC | 33 |
| | GGCCCTC | |
| Pspmcherry 11(−9 nt) | CATGCCGCCGGTGGAGTGGCGG | 34 |
| | CCCTCGGC | |
| Pspmcherry 11(−6nt)) | GCCGCCGGTGGAGTGGCGGCCCT | 35 |
| | CGGCGCG | |
| Pspmcherry 11(−3 nt) | GCCGGTGGAGTGGCGGCCCTCG | 36 |
| | GCGCGTTC | |
| Pspmcherry 11(+3 nt) | GGAGTGGCGGCCCTCGGCGCGTT | 37 |
| | CGTACTG | |
| Pspmcherry 11(+6 nt) | GTGGCGGCCCTCGGCGCGTTCGT | 38 |
| | ACTGTTC | |
| Pspmcherry 11(+9 nt)_ | GCGGCCCTCGGCGCGTTCGTACT | 39 |
| | GTTCCAC | |
| Pspmcherry 11(+12 nt) | GCCCTCGGCGCGTTCGTACTGTT | 40 |
| | CCACGAT | |
| Pspmcherry 11(+15 nt) | CTCGGCGCGTTCGTACTGTTCCA | 41 |
| | CGATGGT | |
| PspmCherry8 tiled1 | GCTTCAGCCTCTGCTTGATCTCG | 42 |
| (matching position 485) | CCCTTCA | |
| PspmCherry8 tiled2 | CTTCAGCCTCTGCTTGATCTCGC | 43 |
| (matching position 484) | CCTTCAG | |
| PspmCherry8 tiled3 | TTCAGCCTCTGCTTGATCTCGCC | 44 |
| (matching position 483) | CTTCAGG | |
| PspmCherry8 tiled4 | TCAGCCTCTGCTTGATCTCGCCC | 45 |
| (matching position 482) | TTCAGGG | |
| PspmCherry8 tiled5 | CAGCCTCTGCTTGATCTCGCCCT | 46 |
| (matching position 481) | TCAGGGC | |
| PspmCherry8 tiled6 | AGCCTCTGCTTGATCTCGCCCTT | 47 |
| (matching position 480) | CAGGGCG | |
| PspmCherry8 tiled7 | GCCTCTGCTTGATCTCGCCCTTC | 48 |
| (matching position 479) | AGGGCGC | |
| PspmCherry8 tiled8 | CCTCTGCTTGATCTCGCCCTTCA | 49 |
| (matching position 478) | GGGCGCC | |
| PspmCherry8 tiled9 | CTCTGCTTGATCTCGCCCTTCAG | 50 |
| (matching position 477) | GGCGCCG | |
| PspmCherry8 tiled10 | TCTGCTTGATCTCGCCCTTCAGG | 51 |
| (matching position 476) | GCGCCGT | |
| PspmCherry8 tiled11 | CTGCTTGATCTCGCCCTTCAGGG | 52 |
| (matching position 475) | CGCCGTC | |
| PspmCherry8 tiled12 | TGCTTGATCTCGCCCTTCAGGGC | 53 |
| (matching position 474) | GCCGTCC | |
| PspmCherry8 tiled13 | GCTTGATCTCGCCCTTCAGGGCG | 54 |
| (matching position 473) | CCGTCCT | |
| PspmCherry8 tiled14 | CTTGATCTCGCCCTTCAGGGCGC | 55 |
| (matching position 472) | CGTCCTC | |
| PspmCherry8 tiled15 | TTGATCTCGCCCTTCAGGGCGCC | 56 |
| (matching position 471) | GTCCTCG | |
| PspmCherry8 tiled16 | TGATCTCGCCCTTCAGGGCGCCG | 57 |
| (matching position 470) | TCCTCGG | |
| PspmCherry8 tiled17 | GATCTCGCCCTTCAGGGCGCCGT | 58 |
| (matching position 469) | CCTCGGG | |
| PspmCherry8 tiled18 | ATCTCGCCCTTCAGGGCGCCGTC | 59 |
| (matching position 468) | CTCGGGG | |
| PspmCherry8 tiled19 | TCTCGCCCTTCAGGGCGCCGTCC | 60 |
| (matching position 467) | TCGGGGT | |
| PspmCherry8 tiled20 | CTCGCCCTTCAGGGCGCCGTCCT | 61 |
| (matching position 466) | CGGGGTA | |
| PspmCherry8 tiled21 | TCGCCCTTCAGGGCGCCGTCCTC | 62 |
| (matching position 465) | GGGGTAC | |
| PspmCherry8 tiled22 | CGCCCTTCAGGGCGCCGTCCTCG | 63 |
| (matching position 464) | GGGTACA | |
| PspmCherry8 tiled23 | GCCCTTCAGGGCGCCGTCCTCGG | 64 |
| (matching position 463) | GGTACAT | |
| PspmCherry8 tiled24 | CCCTTCAGGGCGCCGTCCTCGGG | 65 |
| (matching position 462) | GTACATC | |
| PspmCherry8 tiled25 | CCTTCAGGGCGCCGTCCTCGGGG | 66 |
| (matching position 461) | TACATCC | |
| PspmCherry8 tiled26 | CTTCAGGGCGCCGTCCTCGGGGT | 67 |
| (matching position 460) | ACATCCG | |
| PspmCherry8 tiled27 | TTCAGGGCGCCGTCCTCGGGGTA | 68 |
| (matching position 459) | CATCCGC | |
| PspmCherry8 tiled28 | TCAGGGCGCCGTCCTCGGGGTAC | 69 |
| (matching position 458) | ATCCGCT | |
| PspmCherry8 tiled29 | CAGGGCGCCGTCCTCGGGGTACA | 70 |
| (matching position 457) | TCCGCTC | |
| PspmCherry8 tiled30 | AGGGCGCCGTCCTCGGGGTACAT | 71 |
| (matching position 456) | CCGCTCG | |
| PspmCherry8 tiled31 | GGGCGCCGTCCTCGGGGTACATC | 72 |
| (matching position 455) (as | CGCTCGG | |
| referred to as | | |
| PspmCherry8) | | |
| PspmCherry8 tiled32 | GGCGCCGTCCTCGGGGTACATCC | 73 |
| (matching position 454) | GCTCGGA | |
| PspmCherry8 tiled33 | GCGCCGTCCTCGGGGTACATCCG | 74 |
| (matching position 453) | CTCGGAG | |
| PspmCherry8 tiled34 | CGCCGTCCTCGGGGTACATCCGC | 75 |
| (matching position 452) | TCGGAGG | |
| PspmCherry8 tiled35 | GCCGTCCTCGGGGTACATCCGCT | 76 |
| (matching position 451) | CGGAGGA | |
| PspmCherry8 tiled36 | CCGTCCTCGGGGTACATCCGCTC | 77 |
| (matching position 450) | GGAGGAG | |
| PspmCherry8 tiled37 | CGTCCTCGGGGTACATCCGCTCG | 78 |
| (matching position 449) | GAGGAGG | |
| PspmCherry8 tiled38 | GTCCTCGGGGTACATCCGCTCGG | 79 |
| (matching position 448) | AGGAGGC | |
| PspmCherry8 tiled39 | TCCTCGGGGTACATCCGCTCGGA | 80 |
| (matching position 447) | GGAGGCC | |
| PspmCherry8 tiled40 | CCTCGGGGTACATCCGCTCGGAG | 81 |
| (matching position 446) | GAGGCCT | |
| PspmCherry8 tiled41 | CTCGGGGTACATCCGCTCGGAGG | 82 |
| (matching position 445) | AGGCCTC | |
| PspmCherry8 tiled42 | TCGGGGTACATCCGCTCGGAGGA | 83 |
| (matching position 444) | GGCCTCC | |
| PspmCherry8 tiled43 | CGGGGTACATCCGCTCGGAGGA | 84 |
| (matching position 443) | GGCCTCCC | |
| PspmCherry8 tiled44 | GGGGTACATCCGCTCGGAGGAG | 85 |
| (matching position 442) | GCCTCCCA | |
| PspmCherry8 titling45 | GGGTACATCCGCTCGGAGGAGG | 86 |
| (matching position 441) | CCTCCCAG | |
| PspmCherry8 tiled46 | GGTACATCCGCTCGGAGGAGGC | 87 |
| (matching position 440) | CTCCCAGC | |
| PspmCherry8 tiled47 | GTACATCCGCTCGGAGGAGGCCT | 88 |
| (matching position 439) | CCCAGCC | |
| PspmCherry8 tiled48 | TACATCCGCTCGGAGGAGGCCTC | 89 |
| (matching position 438) | CCAGCCC | |
| PspmCherry8 tiled49 | ACATCCGCTCGGAGGAGGCCTCC | 90 |
| (matching position 437) | CAGCCCA | |
| PspmCherry8 tiled50 | CATCCGCTCGGAGGAGGCCTCCC | 91 |
| (matching position 436) | AGCCCAT | |
| PspmCherry8 tiled51 | ATCCGCTCGGAGGAGGCCTCCCA | 92 |
| (matching position 435) | GCCCATC | |
| PspmCherry8 tiled52 | TCCGCTCGGAGGAGGCCTCCCAG | 93 |
| (matching position 434) | CCCATCG | |
| PspmCherry8 tiled53 | CCGCTCGGAGGAGGCCTCCCAGC | 94 |
| (matching position 432) | CCATCGT | |
| PspmCherry8 tiled54 | CGCTCGGAGGAGGCCTCCCAGCC | 95 |
| (matching position 431) | CATCGTC | |
| PspmCherry8 tiled55 | GCTCGGAGGAGGCCTCCCAGCCC | 96 |
| (matching position 430) | ATCGTCT | |
| PspmCherry8 tiled56 | CTCGGAGGAGGCCTCCCAGCCCA | 97 |
| (matching position 429) | TCGTCTT | |
| PspmCherry8 tiled57 | TCGGAGGAGGCCTCCCAGCCCAT | 98 |
| (matching position 428) | CGTCTTC | |
| PspmCherry8 tiled58 | CGGAGGAGGCCTCCCAGCCCATC | 99 |
| (matching position 427) | GTCTTCT | |
| PspmCherry8 tiled59 | GGAGGAGGCCTCCCAGCCCATC | 100 |
| (matching position 426) | GTCTTCTT | |
| PspmCherry8 tiled60 | GAGGAGGCCTCCCAGCCCATCGT | 101 |
| (matching position 425) | CTTCTTC | |
| PspmCherry8 tiled61 | AGGAGGCCTCCCAGCCCATCGTC | 102 |
| (matching position 424) | TTCTTCT | |
| BCR-ABL1crRNA−20 | GGCUCAAAGUCAGAUGCUACUG | 103 |
| | GCCGCUGA | |
| BCR-ABL1crRNA−19 | GCUCAAAGUCAGAUGCUACUGG | 104 |
| | CCGCUGAA | |
| BCR-ABL1crRNA−18 | CUCAAAGUCAGAUGCUACUGGC | 105 |
| | CGCUGAAG | |
| BCR-ABL1crRNA−17 | UCAAAGUCAGAUGCUACUGGCC | 106 |
| | GCUGAAGG | |
| BCR-ABL1crRNA−16 | CAAAGUCAGAUGCUACUGGCCG | 107 |
| | CUGAAGGG | |
| BCR-ABL1crRNA−15 | AAAGUCAGAUGCUACUGGCCGC | 108 |
| | UGAAGGGC | |
| BCR-ABL1crRNA−14 | AAGUCAGAUGCUACUGGCCGCU | 109 |
| | GAAGGGCU | |
| BCR-ABL1crRNA−13 | AGUCAGAUGCUACUGGCCGCUG | 110 |
| | AAGGGCUU | |
| BCR-ABL1crRNA−12 | GUCAGAUGCUACUGGCCGCUGA | 111 |
| | AGGGCUUC | |
| BCR-ABL1crRNA−11 | UCAGAUGCUACUGGCCGCUGAA | 112 |
| | GGGCUUCU | |
| BCR-ABL1crRNA−10 | CAGAUGCUACUGGCCGCUGAAG | 113 |
| | GGCUUCUG | |
| BCR-ABL1crRNA−9 | AGAUGCUACUGGCCGCUGAAGG | 114 |
| | GCUUCUGC | |
| BCR-ABL1crRNA−8 | GAUGCUACUGGCCGCUGAAGGG | 115 |
| | CUUCUGCG | |
| BCR-ABL1crRNA−7 | AUGCUACUGGCCGCUGAAGGGC | 116 |
| | UUCUGCGU | |
| BCR-ABL1crRNA−6 | UGCUACUGGCCGCUGAAGGGCU | 117 |
| | UCUGCGUC | |
| BCR-ABL1crRNA−5 | GCUACUGGCCGCUGAAGGGCUU | 118 |
| | CUGCGUCU | |
| BCR-ABL1crRNA−4 | CUACUGGCCGCUGAAGGGCUUC | 119 |
| | UGCGUCUC | |
| BCR-ABL1crRNA−3 | UACUGGCCGCUGAAGGGCUUCU | 120 |
| | GCGUCUCC | |
| BCR-ABL1crRNA−2 | ACUGGCCGCUGAAGGGCUUCUG | 121 |
| | CGUCUCCA | |
| BCR-ABL1crRNA−1 | CUGGCCGCUGAAGGGCUUCUGC | 122 |
| | GUCUCCAU | |
| BCR-ABL1crRNA | UGGCCGCUGAAGGGCUUCUGCG | 123 |
| | UCUCCAUG | |
| BCR-ABL1crRNA+1 | GGCCGCUGAAGGGCUUCUGCGU | 124 |
| | CUCCAUGG | |
| BCR-ABL1crRNA+2 | GCCGCUGAAGGGCUUCUGCGUC | 125 |
| | UCCAUGGA | |
| BCR-ABL1crRNA+3 | CCGCUGAAGGGCUUCUGCGUCU | 126 |
| | CCAUGGAA | |
| BCR-ABL1crRNA+4 | CGCUGAAGGGCUUCUGCGUCUC | 127 |
| | CAUGGAAG | |
| BCR-ABL1crRNA+5 | GCUGAAGGGCUUCUGCGUCUCC | 128 |
| | AUGGAAGG | |
| BCR-ABL1crRNA+6 | CUGAAGGGCUUCUGCGUCUCCA | 129 |
| | UGGAAGGC | |
| BCR-ABL1crRNA+7 | UGAAGGGCUUCUGCGUCUCCAU | 130 |
| | GGAAGGCG | |
| BCR-ABL1crRNA+8 | GAAGGGCUUCUGCGUCUCCAUG | 131 |
| | GAAGGCGC | |
| BCR-ABL1crRNA+9 | AAGGGCUUCUGCGUCUCCAUGG | 132 |
| | AAGGCGCC | |
| BCR-ABL1crRNA+10 | AGGGCUUCUGCGUCUCCAUGGA | 133 |
| | AGGCGCCC | |
| BCR-ABL1crRNA+11 | GGGCUUCUGCGUCUCCAUGGAA | 134 |
| | GGCGCCCU | |
| BCR-ABL1crRNA+12 | GGCUUCUGCGUCUCCAUGGAAG | 135 |
| | GCGCCCUC | |
| BCR-ABL1crRNA+13 | GCUUCUGCGUCUCCAUGGAAGG | 136 |
| | CGCCCUCG | |
| BCR-ABL1crRNA+14 | CUUCUGCGUCUCCAUGGAAGGC | 137 |
| | GCCCUCGC | |
| BCR-ABL1crRNA+15 | UUCUGCGUCUCCAUGGAAGGCG | 138 |
| | CCCUCGCC | |
| BCR-ABL1crRNA+16 | UCUGCGUCUCCAUGGAAGGCGC | 139 |
| | CCUCGCCA | |
| BCR-ABL1crRNA+17 | CUGCGUCUCCAUGGAAGGCGCC | 140 |
| | CUCGCCAU | |
| BCR-ABL1crRNA+18 | UGCGUCUCCAUGGAAGGCGCCC | 141 |
| | UCGCCAUC | |
| BCR-ABL1crRNA+19 | GCGUCUCCAUGGAAGGCGCCCU | 142 |
| | CGCCAUCG | |
| BCR-ABL1crRNA+20 | CGUCUCCAUGGAAGGCGCCCUC | 143 |
| | GCCAUCGU | |
| SNX2-ABL1−12 | GAAGCGGCUCUCGGAGGAGACG | 144 |
| | UAGAGCUC | |
| SNX2-ABL1−9 | GCGGCUCUCGGAGGAGACGUAG | 145 |
| | AGCUCUUC | |
| SNX2-ABL1−6 | GCUCUCGGAGGAGACGUAGAGC | 146 |
| | UCUUCCCU | |
| SNX2-ABL1−3 | CUCGGAGGAGACGUAGAGCUCU | 147 |
| | UCCCUGGA | |
| SNX2-ABL1+0 | GGAGGAGACGUAGAGCUCUUCC | 148 |
| | CUGGAUCU | |
| SNX2-ABL1+3 | GGAGACGUAGAGCUCUUCCCUG | 149 |
| | GAUCUAUC | |
| SNX2-ABL1+6 | GACGUAGAGCUCUUCCCUGGAU | 150 |
| | CUAUCAAA | |
| SNX2-ABL1+9 | GUAGAGCUCUUCCCUGGAUCUA | 151 |
| | UCAAAGAU | |
| SNX2-ABL1+12 | GAGCUCUUCCCUGGAUCUAUCA | 152 |
| | AAGAUCAC | |
| SFPQ-ABL1−12 | GAAGCGGCUCUCGGAGGAGACG | 153 |
| | UAGAGCAU | |
| SFPQ-ABL1−9 | GCGGCUCUCGGAGGAGACGUAG | 154 |
| | AGCAUGUC | |
| SFPQ-ABL1−6 | GCUCUCGGAGGAGACGUAGAGC | 155 |
| | AUGUCACU | |
| SFPQ-ABL1−3 | CUCGGAGGAGACGUAGAGCAUG | 156 |
| | UCACUUCC | |
| SFPQ-ABL1+0 | GGAGGAGACGUAGAGCAUGUCA | 157 |
| | CUUCCCAU | |
| SFPQ-ABL1+3 | GGAGACGUAGAGCAUGUCACUU | 158 |
| | CCCAUCAU | |
| SFPQ-ABL1+6 | GACGUAGAGCAUGUCACUUCCC | 159 |
| | AUCAUGGA | |
| SFPQ-ABL1+9 | GUAGAGCAUGUCACUUCCCAUC | 160 |
| | AUGGAACC | |
| SFPQ-ABL1+12 | GAGCAUGUCACUUCCCAUCAUG | 161 |
| | GAACCACU | |
| Spike crRNA-TIL1 | GTCGCGCACCAGGTTGATTGGGG | 162 |
| | TGTGCTT | |
| Spike crRNA-TIL2 | TCGCGCACCAGGTTGATTGGGGT | 163 |
| | GTGCTTG | |
| Spike crRNA-TIL3 | CGCGCACCAGGTTGATTGGGGTG | 164 |
| | TGCTTGG | |
| Spike crRNA-TIL4 | GCGCACCAGGTTGATTGGGGTGT | 165 |
| | GCTTGGA | |
| Spike crRNA-TIL5 | CGCACCAGGTTGATTGGGGTGTG | 166 |
| | CTTGGAG | |
| Spike crRNA-TIL6 | GCACCAGGTTGATTGGGGTGTGC | 167 |
| | TTGGAGT | |
| Spike crRNA-TIL7 | CACCAGGTTGATTGGGGTGTGCT | 168 |
| | TGGAGTA | |
| Spike crRNA-TIL8 | ACCAGGTTGATTGGGGTGTGCTT | 169 |
| | GGAGTAG | |
| Spike crRNA-TIL9 | CCAGGTTGATTGGGGTGTGCTTG | 170 |
| | GAGTAGA | |
| Spike crRNA-TIL10 | CAGGTTGATTGGGGTGTGCTTGG | 171 |
| | AGTAGAT | |
| Spike crRNA-TIL11 | AGGTTGATTGGGGTGTGCTTGGA | 172 |
| | GTAGATC | |
| Spike crRNA-TIL12 | GGTTGATTGGGGTGTGCTTGGAG | 173 |
| | TAGATCT | |
| Spike crRNA-TIL13 | GTTGATTGGGGTGTGCTTGGAGT | 174 |
| | AGATCTT | |
| Spike crRNA-TIL14 | TTGATTGGGGTGTGCTTGGAGTA | 175 |
| | GATCTTA | |
| Spike crRNA-TIL15 | TGATTGGGGTGTGCTTGGAGTAG | 176 |
| | ATCTTAA | |
| Spike crRNA-TIL16 | GATTGGGGTGTGCTTGGAGTAGA | 177 |
| | TCTTAAA | |
| Spike crRNA-TIL17 | ATTGGGGTGTGCTTGGAGTAGAT | 178 |
| | CTTAAAG | |
| Spike crRNA-TIL18 | TTGGGGTGTGCTTGGAGTAGATC | 179 |
| | TTAAAGT | |
| Spike crRNA-TIL19 | TGGGGTGTGCTTGGAGTAGATCT | 180 |
| | TAAAGTA | |
| Spike crRNA-TIL20 | GGGGTGTGCTTGGAGTAGATCTT | 181 |
| | AAAGTAG | |
| Spike crRNA-TIL21 | GGGTGTGCTTGGAGTAGATCTTA | 182 |
| | AAGTAGC | |
| Spike crRNA-TIL22 | GGTGTGCTTGGAGTAGATCTTAA | 183 |
| | AGTAGCC | |
| Spike crRNA-TIL23 | GTGTGCTTGGAGTAGATCTTAAA | 184 |
| | GTAGCCA | |
| Spike crRNA-TIL24 | TGTGCTTGGAGTAGATCTTAAAG | 185 |
| | TAGCCAT | |
| Spike crRNA-TIL25 | GTGCTTGGAGTAGATCTTAAAGT | 186 |
| | AGCCATC | |
| Spike crRNA-TIL26 | TGCTTGGAGTAGATCTTAAAGTA | 187 |
| | GCCATCG | |
| Spike crRNA-TIL27 | GCTTGGAGTAGATCTTAAAGTAG | 188 |
| | CCATCGA | |
| Spike crRNA-TIL28 | CTTGGAGTAGATCTTAAAGTAGC | 189 |
| | CATCGAT | |
| Spike crRNA-TIL29 | TTGGAGTAGATCTTAAAGTAGCC | 190 |
| | ATCGATG | |
| Spike crRNA-TIL30 | TGGAGTAGATCTTAAAGTAGCCA | 191 |
| | TCGATGT | |
| Spike crRNA-TIL31 | GGAGTAGATCTTAAAGTAGCCAT | 192 |
| | CGATGTT | |
| Spike crRNA-TIL32 | GAGTAGATCTTAAAGTAGCCATC | 193 |
| | GATGTTC | |
| Spike crRNA-TIL33 | AGTAGATCTTAAAGTAGCCATCG | 194 |
| | ATGTTCT | |
| Spike crRNA-TIL34 | GTAGATCTTAAAGTAGCCATCGA | 195 |
| | TGTTCTT | |
| Spike crRNA-TIL35 | TAGATCTTAAAGTAGCCATCGAT | 196 |
| | GTTCTTA | |
| Spike crRNA-TIL36 | AGATCTTAAAGTAGCCATCGATG | 197 |
| | TTCTTAA | |
| Spike crRNA-TIL37 | GATCTTAAAGTAGCCATCGATGT | 198 |
| | TCTTAAA | |
| Spike crRNA-TIL38 | ATCTTAAAGTAGCCATCGATGTT | 199 |
| | CTTAAAC | |
| Spike crRNA-TIL39 | TCTTAAAGTAGCCATCGATGTTC | 200 |
| | TTAAACA | |
| Spike crRNA-TIL40 | CTTAAAGTAGCCATCGATGTTCT | 201 |
| | TAAACAC | |
| Spike crRNA-TIL41 | TTAAAGTAGCCATCGATGTTCTT | 202 |
| | AAACACG | |
| Spike crRNA-TIL42 | TAAAGTAGCCATCGATGTTCTTA | 203 |
| | AACACGA | |
| Spike crRNA-TIL43 | AAAGTAGCCATCGATGTTCTTAA | 204 |
| | ACACGAA | |
| Spike crRNA-TIL44 | AAGTAGCCATCGATGTTCTTAAA | 205 |
| | CACGAAC | |
| Spike crRNA-TIL45 | AGTAGCCATCGATGTTCTTAAAC | 206 |
| | ACGAACT | |
| Spike crRNA-TIL46 | GTAGCCATCGATGTTCTTAAACA | 207 |
| | CGAACTC | |
| Spike crRNA-TIL47 | TAGCCATCGATGTTCTTAAACAC | 208 |
| | GAACTCC | |
| Spike crRNA-TIL48 | AGCCATCGATGTTCTTAAACACG | 209 |
| | AACTCCC | |
| Spike crRNA-TIL49 | GCCATCGATGTTCTTAAACACGA | 210 |
| | ACTCCCG | |
| Spike crRNA-TIL50 | CCATCGATGTTCTTAAACACGAA | 211 |
| | CTCCCGC | |
| Spike crRNA-TIL51 | CATCGATGTTCTTAAACACGAAC | 212 |
| | TCCCGCA | |
| Spike crRNA-TIL52 | ATCGATGTTCTTAAACACGAACT | 213 |
| | CCCGCAG | |
| Spike crRNA-TIL53 | TCGATGTTCTTAAACACGAACTC | 214 |
| | CCGCAGG | |
| Spike crRNA-TIL54 | CGATGTTCTTAAACACGAACTCC | 215 |
| | CGCAGGT | |
| Spike crRNA-TIL55 | GATGTTCTTAAACACGAACTCCC | 216 |
| | GCAGGTT | |
| Spike crRNA-TIL56 | ATGTTCTTAAACACGAACTCCCG | 217 |
| | CAGGTTC | |
| Spike crRNA-TIL57 | TGTTCTTAAACACGAACTCCCGC | 218 |
| | AGGTTCT | |
| Spike crRNA-TIL58 | GTTCTTAAACACGAACTCCCGCA | 219 |
| | GGTTCTT | |
| Spike crRNA-TIL59 | TTCTTAAACACGAACTCCCGCAG | 220 |
| | GTTCTTG | |
| Spike crRNA-TIL60 | TCTTAAACACGAACTCCCGCAGG | 221 |
| | TTCTTGA | |
| Spike crRNA-TIL61 | CTTAAACACGAACTCCCGCAGGT | 222 |
| | TCTTGAA | |
| PspmCherry8 3 nt | CCCCGCCGUCCUCGGGGUACAU | 223 |
| MSM 1-3 nt-mismatches | CCGCUCGG | |
| PspmCherry8 3 nt | GGGGCGCGUCCUCGGGGUACAU | 224 |
| MSM 4-6 nt-mismatches | CCGCUCGG | |
| PspmCherry8 3 nt | GGGCGCGCACCUCGGGGUACAU | 225 |
| MSM 7-9 nt-mismatches | CCGCUCGG | |
| PspmCherry8 3 nt | GGGCGCCGUGGACGGGGUACAU | 226 |
| MSM 10-12 nt-mismatches | CCGCUCGG | |
| PspmCherry8 3 nt | GGGCGCCGUCCUGCCGGUACAU | 227 |
| MSM 13-15 nt-mismatches | CCGCUCGG | |
| PspmCherry8 3 nt | GGGCGCCGUCCUCGGCCAACAU | 228 |
| MSM 16-18 nt-mismatches | CCGCUCGG | |
| PspmCherry8 3 nt | GGGCGCCGUCCUCGGGGUUGUU | 229 |
| MSM 19-21 nt-mismatches | CCGCUCGG | |
| PspmCherry8 3 nt | GGGCGCCGUCCUCGGGGUACAA | 230 |
| MSM 22-24 nt-mismatches | GGGCUCGG | |
| PspmCherry8 3 nt | GGGCGCCGUCCUCGGGGUACAU | 231 |
| MSM 25-27 nt-mismatches | CCCGACGG | |
| PspmCherry8 3 nt | GGGCGCCGUCCUCGGGGUACAU | 232 |
| MSM 28-30 nt-mismatches | CCGCUGCC | |
| PspmCherry8 4 nt | CCCGGCCGTCCTCGGGGTACATC | 233 |
| MSM_1-4 nt-mismatches | CGCTCGG | |
| PspmCherry8 4 nt | GGGCCGGCTCCTCGGGGTACAT | 234 |
| MSM_5-8 nt-mismatches | CCGCTCGG | |
| PspmCherry8 4 nt | GGGCGCCGAGGACGGGGTACAT | 235 |
| MSM_9-12 nt-mismatches | CCGCTCGG | |
| PspmCherry8 4 nt | GGGCGCCGTCCTGCCCGTACATC | 236 |
| MSM_13-16 nt-mismatches | CGCTCGG | |
| PspmCherry8 4 nt | GGGCGCCGTCCTCGGGCATGAT | 237 |
| MSM_17-20 nt-mismatches | CCGCTCGG | |
| PspmCherry8 4 nt | GGGCGCCGTCCTCGGGGTACTA | 238 |
| MSM_21-24 nt-mismatches | GGGCTCGG | |
| PspmCherry8 4 nt | GGGCGCCGTCCTCGGGGTACATC | 239 |
| MSM_25-28 nt-mismatches | CCGAGGG | |
| PspmCherry8 5 nt | CCCGCCCGTCCTCGGGGTACATC | 240 |
| MSM_1-5 nt-mismatches | CGCTCGG | |
| PspmCherry8 5 nt | GGGCGGGCAGCTCGGGGTACAT | 241 |
| MSM_6-10 nt-mismatches | CCGCTCGG | |
| PspmCherry8 5 nt | GGGCGCCGTCGAGCCGGTACAT | 242 |
| MSM_11-15 nt-mismatches | CCGCTCGG | |
| PspmCherry8 5 nt | GGGCGCCGTCCTCGGCCATGAT | 243 |
| MSM_16-20 nt-mismatches | CCGCTCGG | |
| PspmCherry8 5 nt | GGGCGCCGTCCTCGGGGTACTA | 244 |
| MSM_21-25 nt-mismatches | GGCCTCGG | |
| PspmCherry8 5 nt | GGGCGCCGTCCTCGGGGTACATC | 245 |
| MSM_26-30 nt-mismatches | CGGAGCC | |
| PspmCherry8 6 nt | CCCGCGCGTCCTCGGGGTACAT | 246 |
| MSM_1-6 nt-mismatches | CCGCTCGG | |
| PspmCherry8 6 nt | GGGCGCGCAGGACGGGGTACAT | 247 |
| MSM_7-12 nt-mismatches | CCGCTCGG | |
| PspmCherry8 6 nt | GGGCGCCGTCCTGCCCCAACAT | 248 |
| MSM_13-18 nt-mismatches | CCGCTCGG | |
| PspmCherry8 6 nt | GGGCGCCGTCCTCGGGGTTGTA | 249 |
| MSM_19-24 nt-mismatches | GGGCTCGG | |
| PspmCherry8 6 nt | GGGCGCCGTCCTCGGGGTACATC | 250 |
| MSM_25-30 nt-mismatches | CCGAGCC | |
| PspmCherry8 28-30 nt- | GGGCGCCGUCCUCGGGGUACAU | 251 |
| mismatches | CCGCUGCC | |
| PspmCherry8 25-30 nt- | GGGCGCCGUCCUCGGGGUACAU | 252 |
| mismatches | CCCGAGCC | |
| PspmCherry8 22-30 nt- | GGGCGCCGUCCUCGGGGUACAA | 253 |
| mismatches | GGCGAGCC | |
| PspmCherry8 19-30 nt- | GGGCGCCGUCCUCGGGGUUGUA | 254 |
| mismatches | GGCGAGCC | |
| PspmCherry8 16-30 nt- | GGGCGCCGUCCUCGGCCAUGUA | 255 |
| mismatches | GGCGAGCC | |
| PspmCherry8 13-30 nt- | GGGCGCCGUCCUGCCCCAUGUA | 256 |
| mismatches | GGCGAGCC | |
| PspmCherry8 10-30 nt- | GGGCGCCGUGGAGCCCCAUGU | 257 |
| mismatches | AGGCGAGCC | |
| PspmCherry8 7-30 nt- | GGGCGCGCAGGAGCCCCAUGU | 258 |
| mismatches | AGGCGAGCC | |
| PspmCherry8 4-30 nt- | GGGGCGGCAGGAGCCCCAUGU | 259 |
| mismatches | AGGCGAGCC | |
| PspmCherry8 1-30 nt- | CCCGCGGCAGGAGCCCCAUGU | 260 |
| mismatches | AGGCGAGCC | |
| PspmCherry8 1-3 nt- | CCCCGCCGUCCUCGGGGUACAU | 261 |
| mismatches | CCGCUCGG | |
| PspmCherry8 1-6 nt- | CCCGCGCGUCCUCGGGGUACAU | 262 |
| mismatches | CCGCUCGG | |
| PspmCherry8 1-9 nt- | CCCGCGGCACCUCGGGGUACAU | 263 |
| mismatches | CCGCUCGG | |
| PspmCherry8 1-12 nt- | CCCGCGGCAGGACGGGGUACA | 264 |
| mismatches | UCCGCUCGG | |
| PspmCherry8 1-15 nt- | CCCGCGGCAGGAGCCGGUACA | 265 |
| mismatches | UCCGCUCGG | |
| PspmCherry8 1-18 nt- | CCCGCGGCAGGAGCCCCAACA | 266 |
| mismatches | UCCGCUCGG | |
| PspmCherry8 1-21 nt- | CCCGCGGCAGGAGCCCCAUGU | 267 |
| mismatches | UCCGCUCGG | |
| PspmCherry8 1-24 nt- | CCCGCGGCAGGAGCCCCAUGU | 268 |
| mismatches | AGGGCUCGG | |
| PspmCherry8 1-27 nt- | CCCGCGGCAGGAGCCCCAUGU | 269 |
| mismatches | AGGCGACGG | |
| PspmCherry8 1-30 nt- | CCCGCGGCAGGAGCCCCAUGU | 270 |
| mismatches | AGGCGAGCC | |
| PspmCherry 1_1-3 nt- | CCGCATGTTATCCTCCTCGCCCT | 271 |
| mismatches | TGCTCAC | |
| PspmCherry2_1-3 nt- | CCTGCCCTCCATGTGCACCTTGA | 272 |
| mismatches | AGCGCAT | |
| PspmCherry3_1-3 nt- | CCGGGTCTGGGTGCCCTCGTAGG | 273 |
| mismatches | GGCGGCC | |
| PspmCherry4_1-3 nt- | CCTGCCGTACATGAACTGAGGG | 274 |
| mismatches | GACAGGAT | |
| PspmCherry5_1-3 nt- | CCCGAAGGACAGCTTCAAGTAG | 275 |
| mismatches | TCGGGGAT | |
| PspmCherry6_1-3 nt- | CCTGTCCTGGGTCACGGTCACCA | 276 |
| mismatches | CGCCGCC | |
| PspmCherry7_1-3 nt- | CCACTTCTTCTGCATTACGGGGC | 277 |
| mismatches | CGTCGGA | |
| PspmCherry8_1-3 nt- | CCCCGCCGTCCTCGGGGTACATC | 278 |
| mismatches | CGCTCGG | |
| PspmCherry9_1-3 nt- | CCAGGTCTTGACCTCAGCGTCGT | 279 |
| mismatches | AGTGGCC | |
| PspmCherry 10_1-3 nt- | CCAGATGTCCAACTTGATGTTGA | 280 |
| mismatches | CGTTGTA | |
| PspmCherry 11_1-3 nt- | CCAGGAGTGGCGGCCCTCGGCG | 281 |
| mismatches | CGTTCGTA | |
| PspmCherry8_1-2 nt- | CCGCGCCGTCCTCGGGGTACATC | 282 |
| mismatches | CGCTCGG | |
| PspmCherry8_2-3 nt- | GCCCGCCGTCCTCGGGGTACATC | 283 |
| mismatches | CGCTCGG | |
| PspmCherry8_1st&3rdnt- | CGCCGCCGTCCTCGGGGTACATC | 284 |
| mismatches | CGCTCGG | |
| PspmCherry8_1st nt- | CGGCGCCGTCCTCGGGGTACATC | 285 |
| mismatch | CGCTCGG | |
| PspmCherry8_2nd nt- | GGGCGCCGTCCTCGGGGTACATC | 286 |
| mismatch | CGCTCGG | |
| PspmCherry8_3rd nt- | GGCCGCCGTCCTCGGGGTACATC | 287 |
| mismatch | CGCTCGG | |
| mcherry crRNA8 spread 5 | GGGCGGCGTCCACGGGGAACAT | 288 |
| SNP | CGGCTCGC | |
| mcherry crRNA8 spread 4 | GGGCGCGGTCCTCCGGGTACTTC | 289 |
| SNP | CGCTGGG | |
| mcherry crRNA8 spread 3 | GGGCGCCCTCCTCGGCGTACATC | 290 |
| SNP | GGCTCGG | |
| mcherry crRNA8 spread 2 | GGGCGCCGTGCTCGGGGTAGATC | 291 |
| SNP | CGCTCGG | |
| mcherry gRNA8 spread 15 | GCGGGGCCTGCACCGCGAAGAA | 292 |
| SNP | CGGGTGGC | |
| mcherry gRNA8 spread 10 | GGCCGGCGACCACGCGGAACTT | 293 |
| SNP | CGGCACGC | |
| mcherry gRNA8 spread 7 | GGGGGCCCTCCACGGCGTAGATC | 294 |
| SNP | GGCTGGG | |
| mcherry gRNA8 spread 6 | GGGCCCCGTGCTCGCGGTAGATC | 295 |
| SNP | CCCTCGC | |
| mcherry gRNA8 spread 2 | GGCGGCGCTCGACGCCGTTGATG | 296 |
| MSM | GGCAGGG | |
| mcherry gRNA8 spread 3 | GGGGCGCGTGGACGGCCAACAA | 297 |
| MSM | GGGCTGCC | |
| gmcherry8(−7 nt) 1G | GCCTTCAGGGCGCCGTCCTCGGG | 298 |
| | GTACATC | |
| gmcherry8(−6 nt) 1G | GCTTCAGGGCGCCGTCCTCGGGG | 299 |
| | TACATCC | |
| gmcherry8(−5 nt) 1G | GTTCAGGGCGCCGTCCTCGGGGT | 300 |
| | ACATCCG | |
| gmcherry8(−4 nt) 1G | GTCAGGGCGCCGTCCTCGGGGTA | 301 |
| | CATCCGC | |
| gmcherry8(−3 nt) 1G | GCAGGGCGCCGTCCTCGGGGTAC | 302 |
| | ATCCGCT | |
| gmcherry8(−7 nt) 2G | GGCTTCAGGGCGCCGTCCTCGGG | 303 |
| | GTACATC | |
| gmcherry8(−6 nt) 1G | GGTTCAGGGCGCCGTCCTCGGGG | 304 |
| | TACATCC | |
| gmcherry8(−5 nt) 2G | GGTCAGGGCGCCGTCCTCGGGGT | 305 |
| | ACATCCG | |
| gmcherry8(−4 nt) 2G | GGCAGGGCGCCGTCCTCGGGGT | 306 |
| | ACATCCGC | |
| gmcherry8(−3 nt) 2G | GGAGGGCGCCGTCCTCGGGGTA | 307 |
| | CATCCGCT | |
| gmcherry8(+8 nt) extra g | GTCCTCGGGGTACATCCGCTCGG | 308 |
| | AGGAGGCC | |
| gmcherry8(+9 nt) extra g | GCCTCGGGGTACATCCGCTCGGA | 309 |
| | GGAGGCCT | |
| gmcherry8(+8 nt) 1g | GCCTCGGGGTACATCCGCTCGGA | 310 |
| | GGAGGCC | |
| gmcherry8(+9 nt) 1g | GCTCGGGGTACATCCGCTCGGAG | 311 |
| | GAGGCCT | |
| gmcherry8(+8 nt) 2g | GGCTCGGGGTACATCCGCTCGGA | 312 |
| | GGAGGCC | |
| gmcherry8(+9 nt) 2g | GGTCGGGGTACATCCGCTCGGAG | 313 |
| | GAGGCCT | |
| gmcherry8(−7 nt) extra g | GCCCTTCAGGGCGCCGTCCTCGG | 314 |
| | GGTACATC | |
| gmcherry8(−6 nt) extra g | GCCTTCAGGGCGCCGTCCTCGGG | 315 |
| | GTACATCC | |
| gmcherry8(−5 nt) extra g | GCTTCAGGGCGCCGTCCTCGGGG | 316 |
| | TACATCCG | |
| gmcherry8(−4 nt) extra g | GTTCAGGGCGCCGTCCTCGGGGT | 317 |
| | ACATCCGC | |
| gmcherry8(−3 nt) extra g | GTCAGGGCGCCGTCCTCGGGGTA | 318 |
| | CATCCGCT | |
| BCR crRNA -3 nt G1-2 | GGCTGGCCGCTGAAGGGCTTCTG | 319 |
| | CGTCTCC | |
| BCR crRNA +6 nt G1-2 | GGGAAGGGCTTCTGCGTCTCCAT | 320 |
| | GGAAGGC | |
| BCR crRNA +9 nt G1-2 | GGGGGCTTCTGCGTCTCCATGGA | 321 |
| | AGGCGCC | |
| BCR crRNA +9 nt G15- | AGGCGCC | 322 |
| 17-18 | AAGGGCTTCTGCGTGTGGATGGA | |
| BCR crRNA +9 nt G17- | AAGGGCTTCTGCGTCTGGATGGA | 323 |
| 18 | AGGCGCC | |
| BCR crRNA +9 nt G1-2- | GGGGGCTTCTGCGTGTGGATGGA | 324 |
| 15-17-18 | AGGCGCC | |
| SNX2 crRNA +6 nt G2 | GGCGTAGAGCTCTTCCCTGGATC | 325 |
| | TATCAAA | |
| SNX2 crRNA +6 nt G15- | GACGTAGAGCTCTTGGCTGGATC | 326 |
| 16 | TATCAAA | |
| SNX2 crRNA +6 nt G15- | GACGTAGAGCTCTTGGGTGGATC | 327 |
| 16-17 | TATCAAA | |
| SNX2 crRNA +6 nt G2- | GGCGTAGAGCTCTTGGGTGGATC | 328 |
| 15-16-17 | TATCAAA | |
| SNX2 crRNA +12 nt G2 | GGGCTCTTCCCTGGATCTATCAA | 329 |
| | AGATCAC | |
| SNX2 crRNA +12 nt G11 | GAGCTCTTCCGTGGATCTATCAA | 330 |
| | AGATCAC | |
| SNX2 crRNA +12 nt G17 | GAGCTCTTCCCTGGATGTATCAA | 331 |
| | AGATCAC | |
| SNX2 crRNA +12 nt G2- | GGGCTCTTCCGTGGATGTATCAA | 332 |
| 11-17 | AGATCAC | |
| SNX2 crRNA +3 nt G15- | GGAGACGTAGAGCTGTTGCCTGG | 333 |
| 18 | ATCTATC | |
| SNX2 crRNA +3 nt G15 | GGAGACGTAGAGCTGTTCCCTGG | 334 |
| | ATCTATC | |
| 5′ 3 nt MSM BCR-ABL1 | ACCCCGCTGAAGGGCTTCTGCGT | 335 |
| | CTCCATG | |
| 5′ 6 nt MSM BCR-ABL1 | ACCGGCCTGAAGGGCTTCTGCGT | 336 |
| | CTCCATG | |
| 5′ 9 nt MSM BCR-ABL1 | ACCGGCGACAAGGGCTTCTGCGT | 337 |
| | CTCCATG | |
| 3′ 3 nt MSM BCR-ABL1 | TGGCCGCTGAAGGGCTTCTGCGT | 338 |
| | CTCCTAC | |
| 3′ 6 nt MSM BCR-ABL1 | TGGCCGCTGAAGGGCTTCTGCGT | 339 |
| | CAGGTAC | |
| 3′ 9 nt MSM BCR-ABL1 | TGGCCGCTGAAGGGCTTCTGCCA | 340 |
| | GAGGTAC | |
| Central 3 nt MSM BCR- | TGGCCGCTGAAGCCGTTCTGCGT | 341 |
| ABL1 | CTCCATG | |
| central 6 nt MSM BCR- | TGGCCGCTGAACCCGAACTGCGT | 342 |
| ABL1 | CTCCATG | |
| central 9 nt MSM BCR- | TGGCCGCTGTTCCCGAAGTGCGT | 343 |
| ABL1 | CTCCATG | |
| spread 3 SNP BCR-ABL1 | TGGCCGCAGAAGGGCATCTGCGT | 344 |
| | GTCCATG | |
| spread 4 SNP BCR-ABL1 | TGGCCGGTGAAGGCCTTCTGCCT | 345 |
| | CTCCAAG | |
| spread 5 SNP BCR-ABL1 | TGGCCCCTGAACGGCTTGTGCGT | 346 |
| | GTCCATC | |
| spread 6 SNP BCR-ABLI | TGGCGGCTGTAGGGGTTCTCCGT | 347 |
| | CACCATC | |
| spread 7 SNP BCR-ABL1 | TGGGCGCAGAACGGCATCTCCGT | 348 |
| | GTCCTTG | |
| spread 10 SNP BCR- | TGCCCCCTCAACGGGTTGTGGGT | 349 |
| ABL1 | GTCGATC | |
| 2 nt MSM spread BCR- | TGCGCGGAGATCGGGATCACCG | 350 |
| ABL1 | AGTCGTTG | |
| Ineffective EGFP crRNA1 | CCGGTGAACAGCTCCTCGCCCTT | 351 |
| | GCTCACC | |
| Ineffective EGFP crRNA2 | CAGGATGGGCACCACCCCGGTG | 352 |
| | AACAGCTC | |
| Ineffective EGFP crRNA3 | CTTGTAGTTGCCGTCGTCCTTGA | 353 |
| | AGAAGAT | |
| Ineffective EGFP crRNA4 | CACCAGGGTGTCGCCCTCGAAC | 354 |
| | TTCACCTC | |
| Ineffective EGFP crRNA5 | CAGGATGTTGCCGTCCTCCTTGA | 355 |
| | AGTCGAT | |
| Ineffective EGFP crRNA6 | CTTCTGCTTGTCGGCCATGATAT | 356 |
| | AGACGTT | |
| Ineffective EGFP crRNA7 | CTGCACGCTGCCGTCCTCGATGT | 357 |
| | TGTGGCG | |
| Ineffective EGFP crRNA8 | CTTGTACAGCTCGTCCATGCCGA | 358 |
| | GAGTGAT | |
| Ineffective EGFP crRNA9 | TACTCCAGCTTGTGCCCCAGGAT | 359 |
| | GTTGCCG | |
| Potent EGFP crRNA1 | GGTCAGCTTGCCGTAGGTGGCAT | 360 |
| | CGCCCTC | |
| Potent EGFP crRNA2 | GGTCACGAGGGTGGGCCAGGGC | 361 |
| | ACGGGCAG | |
| Potent EGFP crRNA3 | GGTAGCGGCTGAAGCACTGCAC | 362 |
| | GCCGTAGG | |
| Potent EGFP crRNA4 | GGGCATGGCGGACTTGAAGAAG | 363 |
| | TCGTGCTG | |
| Potent EGFP crRNA5 | GGTGCGCTCCTGGACGTAGCCTT | 364 |
| | CGGGCAT | |
| Potent EGFP crRNA6 | GGCGCGGGTCTTGTAGTTGCCGT | 365 |
| | CGTCCTT | |
| Potent EGFP crRNA7 | GGCGAGCTGCACGCTGCCGTCCT | 366 |
| | CGATGTT | |
| Potent EGFP crRNA8 | GGTAGTGGTCGGCGAGCTGCAC | 367 |
| | GCTGCCGT | |
| Potent EGFP crRNA9 | GGGCCGTCGCCGATGGGGGTGT | 368 |
| | TCTGCTGG | |
| Potent EGFP crRNA10 | GGGCAGCAGCACGGGGCCGTCG | 369 |
| | CCGATGGG | |
| Potent EGFP crRNA11 | GGGGGTGTTCTGCTGGTAGTGGT | 370 |
| | CGGCGAG | |
| Ineffective Tagbfp crRNA1 | CTCGGTGAAGGCCTCCCAGCCGA | 371 |
| | GTGTTTT | |
| Ineffective Tagbfp crRNA2 | CGGTCAGCACGCCCCCGTCCTCG | 372 |
| | TATGTGG | |
| Ineffective Tagbfp crRNA3 | CAGATGGCTCCCGCCCACGAGCT | 373 |
| | TCAGGGC | |
| Ineffective Tagbfp crRNA4 | CCATGTCGTTTCTGCCTTCCAGG | 374 |
| | CCGCCGT | |
| Ineffective Tagbfp crRNA5 | CGATCAGATGGCTCCCGCCCACG | 375 |
| | AGCTTCA | |
| Ineffective Tagbfp crRNA6 | CTCGTTGTTGGCCTCCTTGATTCT | 376 |
| | TTCCAG | |
| Ineffective Tagbfp crRNA7 | CAATTAAGCTTGTGCCCCAGTTT | 377 |
| | GCTAGGG | |
| Ineffective Tagbfp crRNA8 | CCCATGTGAAGCCCTCAGGGAA | 378 |
| | GGACTGCT | |
| Ineffective Tagbfp crRNA9 | ATGTGAAGTTCACCCCTCTGATC | 379 |
| | TTGACGT | |
| Potent Tagbfp crRNA1 | GGCGAAGGGGAGAGGGCCGCCC | 380 |
| | TCGACCAC | |
| Potent Tagbfp crRNA2 | GGTCACGAGGGTGGGCCAGGGC | 381 |
| | ACGGGCAG | |
| Potent Tagbfp crRNA3 | GGTGACTCTCTCCCATGTGAAGC | 382 |
| | CCTCAGG | |
| Potent Tagbfp crRNA4 | GGGGTACAGGGTCTCGGTGAAG | 383 |
| | GCCTCCCA | |
| Potent Tagbfp crRNA5 | GGTTTCTTGGATCTATATGTGGT | 384 |
| | CTTGATG | |
| Potent Tagbfp crRNA6 | GGTCTGGGTGCCCTCGTAGGGCT | 385 |
| | TGCCTTC | |
| Potent Tagbfp crRNA7 | GGATGTCGAAGGCGAAGGGGAG | 386 |
| | AGGGCCGC | |
| Potent Tagbfp crRNA8 | GGTCTTGCTGCCGTAGAGGAAGC | 387 |
| | TAGTAGC | |
| Potent Tagbfp crRNA9 | GGGATGCCCTGGGTGTGGTTGAT | 388 |
| | GAAGGTC | |
| Potent Tagbfp crRNA10 | GGGAGGTCGCAGTATCTGGCCAC | 389 |
| | TGCCACC | |
| RfxCas13d mcherry | TCCTCGAAGTTCATCACGCGCTC | 390 |
| efficient 1 | | |
| RfxCas13d mcherry | CGAAGTTCATCACGCGCTCCCAC | 391 |
| efficient 2 | | |
| RfxCas13d mcherry | TCGAAGTTCATCACGCGCTCCCA | 392 |
| efficient 3 | | |
| RfxCas13d mcherry | GAAGTTCATCACGCGCTCCCACT | 393 |
| efficient 4 | | |
| RfxCas13d mcherry | CGTCCTCGAAGTTCATCACGCGC | 394 |
| efficient 5 | | |
| RfxCas13d mcherry | CTCGAAGTTCATCACGCGCTCCC | 395 |
| efficient 6 | | |
| RfxCas13d mcherry | CCTCGAAGTTCATCACGCGCTCC | 396 |
| efficient 7 | | |
| RfxCas13d mcherry | GTCCTCGAAGTTCATCACGCGCT | 397 |
| efficient 8 | | |
| RfxCas13d mcherry | CCGTCCTCGAAGTTCATCACGCG | 398 |
| efficient 9 | | |
| RfxCas13d mcherry | GCCGTCCTCGAAGTTCATCACGC | 399 |
| efficient 10 | | |
| PspBRAF-1 | GGTCTCTGTAGCTAGACCAAAAT | 419 |
| | CACCTAT | |
| PspBRAF-2 | GGGAGATTTCTCTGTAGCTAGAC | 420 |
| | CAAAATCA | |
| PspBRAF-3 | GGTCCATCGAGATTTCTCTGTAG | 421 |
| | CTAGACC | |
| PspBRAF-4 | GGACCCACTCCATCGAGATTTCT | 422 |
| | CTGTAGC | |
| PspBRAF-5 | GGATGGGACCCACTCCATCGAG | 423 |
| | ATTTCTCT | |
| PspBRAF-Mut1 | GGAGTCTGTAGCTAGACCAAAAT | 424 |
| | CACCTAT | |
| PspBRAF-Mut2 | GGCGTCTGTAGCTAGACCAAAAT | 425 |
| | CACCTAT | |
| PspBRAF-Mut3 | GGTCTGAGTAGCTAGACCAAAAT | 426 |
| | CACCTAT | |
| PspBRAF-Mut4 | GGTCTGACTAGCTAGACCAAAAT | 427 |
| | CACCTAT | |
| PspBRAF-Mut5 | GGTCTGACAAGCTAGACCAAAA | 428 |
| | TCACCTAT | |
| PspBRAF-Mut6 | GGTCTGCGTAGCTAGACCAAAAT | 429 |
| | CACCTAT | |
| PspBRAF-Mut7 | GGTCTGCCTAGCTAGACCAAAAT | 430 |
| | CACCTAT | |
| PspBRAF-Mut8 | GGTCTGCCCAGCTAGACCAAAAT | 431 |
| | CACCTAT | |
| PspBRAF-Mut9 | GGTGTCTCTAGCTAGACCAAAAT | 432 |
| | CACCTAT | |
| PspBRAF-Mut10 | GGTGTCTGTACCTAGACCAAAAT | 433 |
| | CACCTAT | |
| PspBRAF-Mut11 | GGTGTCTGTAGCTACACCAAAAT | 434 |
| | CACCTAT | |
| PspBRAF-Mut12 | GGTCTCTCTACCTAGACCAAAAT | 435 |
| | CACCTAT | |
| PspBRAF-Mut13 | GGTCTCTCTAGCTACACCAAAAT | 436 |
| | CACCTAT | |
| PspBRAF-Mut14 | GGTCTCTGTACCTACACCAAAAT | 437 |
| | CACCTAT | |
| PspBRAF-Mut15 | GGTCTCTCTACCTACACCAAAAT | 438 |
| | CACCTAT | |
| PspBRAF-Mut16 | GGTGTCTGTAGCTACACCAAAAT | 439 |
| | CAGCTAT | |
| PspBRAF-Mut17 | GGTCTGTGTAGCTAGAGCAAAAT | 440 |
| | CACCAAT | |
| PspBRAF-Mut18 | GGTCTCTCTAGCTACACCAAATT | 441 |
| | CACCTAT | |
| PspBRAF-Mut19 | GGTGTCTGTACCTAGACGAAAAT | 442 |
| | CTCCTAT | |
| PspBRAF-Mut20 | GGTCTGTGTAGCAAGACCATAAT | 443 |
| | CAGCTAT | |
| PspBRAF-Mut21 | GGTCTCTGTAGCTACACCAAATT | 444 |
| | CACCAAT | |
| PspBRAF-Mut22 | GGTCTCTGTAGCTAGAGCAAAAT | 445 |
| | GACCTAA | |
| SpCas9 gNT | GTAGATTGCTGTTCTACCAAG | 446 |
| SpCas9-gBRAF1 | TAGCTACAGAGAAATCTCGA | 447 |
| SpCas9-gBRAF2 | ACAGAGAAATCTCGATGGAG | 448 |
| crBCR-ABL1−3 nt | UGGCCGCAGAAGGGCAUCUGCG | 457 |
| mismatch-1 | UGUCCAUG | |
| crBCR-ABL1−3 nt | ACCCCGCUGAAGGGCUUCUGCG | 458 |
| mismatch-2 | UCUCCAUG | |
| crBCR-ABL1−6 nt | ACCGGCCUGAAGGGCUUCUGCG | 459 |
| mismatch | UCUCCAUG | |
| crBCR-ABL1−3 nt | UGGCCGCUGAAGGGCUUCUGCG | 460 |
| mismatch-3 | UCUCCUAC | |
| crBCR-ABL1−3 nt | UGGCCGCUGAAGCCGUUCUGCG | 461 |
| mismatch-4 | UCUCCAUG | |
| crBCR-ABL1−3 nt | UGGCCGAUGAAGGGCAUCUGCG | 462 |
| mismatch-5 | UGUCCAUG | |
| crNT | UAGAUUGCUGUUCUACCAAGUG | 465 |
| | G | |
| crBRAF-1 | UUUCUCUGUAGCUAGACCAAAA | 466 |
| | U | |
| crMM-1 | AUUCUCUGUAGCUAGACCAAAA | 467 |
| | U | |
| crMM-2 | UAUCUCUGUAGCUAGACCAAAA | 468 |
| | U | |
| crMM-3 | UUACUCUGUAGCUAGACCAAAA | 469 |
| | U | |
| crMM-4 | UUUGUCUGUAGCUAGACCAAAA | 470 |
| | U | |
| crMM-5 | UUUCUGUGUAGCUAGACCAAAA | 471 |
| | U | |
| crMM-6 | UUUCUCAGUAGCUAGACCAAAA | 472 |
| | U | |
| crMM-7 | UUUCUCUCUAGCUAGACCAAAA | 473 |
| | U | |
| crMM-8 | UUUCUCUGAAGCUAGACCAAAA | 474 |
| | U | |
| crMM-9 | UUUCUCUGUUGCUAGACCAAAA | 475 |
| | U | |
| crMM-10 | UUUCUCUGUACCUAGACCAAAA | 476 |
| | U | |
| crMM-11 | UUUCUCUGUAGGUAGACCAAAA | 477 |
| | U | |
| crMM-12 | UUUCUCUGUAGCAAGACCAAAA | 478 |
| | U | |
| crMM-13 | UUUCUCUGUAGCUUGACCAAAA | 479 |
| | U | |
| crMM-14 | UUUCUCUGUAGCUACACCAAAA | 480 |
| | U | |
| crMM-15 | UUUCUCUGUAGCUAGUCCAAAA | 481 |
| | U | |
| crMM-16 | UUUCUCUGUAGCUAGAGCAAAA | 482 |
| | U | |
| crMM-17 | UUUCUCUGUAGCUAGACGAAAA | 483 |
| | U | |
| crMM-18 | UUUCUCUGUAGCUAGACCUAAA | 484 |
| | U | |
| crMM-19 | UUUCUCUGUAGCUAGACCAUAA | 485 |
| | U | |
| crMM-20 | UUUCUCUGUAGCUAGACCAAUA | 486 |
| | U | |
| crMM-21 | UUUCUCUGUAGCUAGACCAAAU | 487 |
| | U | |
| crMM-22 | UUUCUCUGUAGCUAGACCAAAA | 488 |
| | A | |
| crC | CCTACGCCACAAGCTCCAACTAC | 489 |
| crC/A-12 | CCTACGCCAGATGCTCCAACTAC | 490 |
| crC/A-9 (i.e., crG12A) | CCTACGCCTGAAGCTCCAACTAC | 491 |
| crC/D | CCTACGCCATAAGCTCCAACTAC | 492 |
| crC/D-12 1ntShift | GCCTACGCCATATGCTCCAACTA | 493 |
| crC/D-12 | CCTACGCCATATGCTCCAACTAC | 494 |
| crC/D-9 1ntShift (i.e., | GCCTACGCCTTAAGCTCCAACTA | 495 |
| crG12C) | | |
| crC/D-9 | CCTACGCCTTAAGCTCCAACTAC | 496 |
| crC/V-12 | CCTACGCCAAATGCTCCAACTAC | 497 |
| crC/V-9 | CCTACGCCTAAAGCTCCAACTAC | 498 |
| crC-WT-12 | CCTACGCCACATGCTCCAACTAC | 499 |
| crC-WT-9 | CCTACGCCTCAAGCTCCAACTAC | 500 |
| crC23 | CCTACGCCACAAGCTCCAACTAG | 501 |
| crCD | CCTACGCCATAAGCTCCAACTAC | 502 |
| crCD1 | GCTACGCCATAAGCTCCAACTAC | 503 |
| crCD12 | CCTACGCCATATGCTCCAACTAC | 504 |
| crCD15 | CCTACGCCATAAGCACCAACTAC | 505 |
| crCD18 | CCTACGCCATAAGCTCCTACTAC | 506 |
| crCD2_1 | GCTACGCCATAAGCTCCAACTAG | 507 |
| crCD2_12 | CCTACGCCATATGCTCCAACTAG | 508 |
| crCD2_15 | CCTACGCCATAAGCACCAACTAG | 509 |
| crCD2_18 | CCTACGCCATAAGCTCCTACTAG | 510 |
| crCD2_21 | CCTACGCCATAAGCTCCAACAAG | 511 |
| crCD2_4 | CCTTCGCCATAAGCTCCAACTAG | 512 |
| crCD2_6 | CCTACCCCATAAGCTCCAACTAG | 513 |
| crCD2_9 | CCTACGCCTTAAGCTCCAACTAG | 514 |
| crCD21 | CCTACGCCATAAGCTCCAACAAC | 515 |
| crCD23 | CCTACGCCATAAGCTCCAACTAG | 516 |
| crCD4 | CCTTCGCCATAAGCTCCAACTAC | 517 |
| crCD6 | CCTACCCCATAAGCTCCAACTAC | 518 |
| crCD9 | CCTACGCCTTAAGCTCCAACTAC | 519 |
| crD | CCTACGCCATCAGCTCCAACTAC | 520 |
| crD-10-12 (i.e., crG12R) | GCCTACGCCTTGAGCTCCAACTA | 521 |
| crD-9-10 | GCCTACGCGTTCAGCTCCAACTA | 522 |
| crD-R 12 | CCTACGCCATGTGCTCCAACTAC | 523 |
| crD-R 9 | CCTACGCCTTGAGCTCCAACTAC | 524 |
| crD/S 12 | CCTACGCCATTTGCTCCAACTAC | 525 |
| crD/S 9 (i.e., crG12D; | CCTACGCCTTTAGCTCCAACTAC | 526 |
| crD12S) | | |
| crD-WT 12 | CCTACGCCATCTGCTCCAACTAC | 527 |
| crD-WT 9 | CCTACGCCTTCAGCTCCAACTAC | 528 |
| crD1 | GCTACGCCATCAGCTCCAACTAC | 529 |
| crD11 | CCTACGCCATGAGCTCCAACTAC | 530 |
| crD12 | CCTACGCCATCTGCTCCAACTAC | 531 |
| crD13 | CCTACGCCATCACCTCCAACTAC | 532 |
| crD14 | CCTACGCCATCAGGTCCAACTAC | 533 |
| crD15 | CCTACGCCATCAGCACCAACTAC | 534 |
| crD16 | CCTACGCCATCAGCTGCAACTAC | 535 |
| crD17 | CCTACGCCATCAGCTCGAACTAC | 536 |
| crD18 | CCTACGCCATCAGCTCCTACTAC | 537 |
| crD19 | CCTACGCCATCAGCTCCATCTAC | 538 |
| crD2 | CGTACGCCATCAGCTCCAACTAC | 539 |
| crD20 | CCTACGCCATCAGCTCCAAGTAC | 540 |
| crD21 | CCTACGCCATCAGCTCCAACAAC | 541 |
| crD22 | CCTACGCCATCAGCTCCAACTTC | 542 |
| crD23 (G12D) | CCTACGCCATCAGCTCCAACTAG | 543 |
| crD23 (G12C) | CCTACGCCATCAGCTCCAACTAG | 544 |
| crD3 | CCAACGCCATCAGCTCCAACTAC | 545 |
| crD4 | CCTTCGCCATCAGCTCCAACTAC | 546 |
| crD5 | CCTAGGCCATCAGCTCCAACTAC | 547 |
| crD6 | CCTACCCCATCAGCTCCAACTAC | 548 |
| crD7 | CCTACGGCATCAGCTCCAACTAC | 549 |
| crD8 | CCTACGCGATCAGCTCCAACTAC | 550 |
| crD9 | CCTACGCCTTCAGCTCCAACTAC | 551 |
| SV9 | CCTACGCCTATAGCTCCAACTAC | 552 |
| RV9 | CCTACGCCTAGAGCTCCAACTAC | 553 |
| SV12 | CCTACGCCAATTGCTCCAACTAC | 554 |
| RV12 | CCTACGCCAAGTGCTCCAACTAC | 555 |
| SV9_1ntShift | GCCTACGCCTATAGCTCCAACTA | 556 |
| RV9_1ntShift | GCCTACGCCTAGAGCTCCAACTA | 557 |
| SV12_1ntShift | GCCTACGCCAATTGCTCCAACTA | 558 |
| CV9_23 | CCTACGCCTAAAGCTCCAACTAG | 559 |
| CV12_23 | CCTACGCCAAATGCTCCAACTAG | 560 |

TABLE 2
Primers for Sanger sequencing and RT-PCR

| | | SEQ |
|---|---|---|
| | | ID |
| Primer name | Sequence | NO: |
| IRES forward | TGGCTCTCCTCAAGCGTATT | 407 |
| M13 reverse | CAGGAAACAGCTATGAC | 408 |
| BCR-ABL1 p194640 forward | CAGATCTGGCCCAACGAT | 409 |
| BCR-ABL1 p190 reverse | CCTGAGGCTCAAAGTCAGAT | 410 |
| SFPQ-ABL1 forward | TGGTTCCATGATGGGAAGTG | 411 |
| SFPQ-ABL1 reverse | ATAATGGAGCGTGGTGATGAG | 412 |
| SNX2-ABL1 forward | CTGCTCCCGTGATCTTTGATAG | 413 |
| SNX2-ABL1 R reverse | GATAATGGAGCGTGGTGATGAG | 414 |
| HSP90A1B forward | AGAAATTGCCCAACTCATGTCC | 415 |
| HSP90A1B reverse | ATCAACTCCCGAAGGAAAATCTC | 416 |
| GAPDH forward | GGAGCGAGATCCCTCCAAAAT | 417 |
| GAPDH reverse | GGCTGTTGTCATACTTCTCATGG | 418 |
| BRAF forward | CCATATCATTGAGACCAAATTTGAGATG | 449 |
| BRAF reverse | GGCACTCTGCCATTAATCTCTTCATGG | 450 |

TABLE 3
Transfection conditions used in 96, 24 and 12 well plates

| Component (per well) | 96-well | 24-well | 12-well |
|---|---|---|---|
| # of seeded HEK | 30,000 | 150,000 | 300,000 |
| 293T cells | | | |
| Plasmids DNA | 100 ng | 500 ng | 1000 ng |
| amount | | | |
| P3000 reagent | 0.2 μL | 1 μL | 2 μl |
| Lipofectamine | 0.3 μL | 1.5 μL | 3 μl |
| 3000 reagent | | | |
| Opti-MEM | Up to | Up to | Up to |
| | 10 μL | 50 μL | 100 μL |

TABLE 4
Western blot antibodies

| Antibody | Manufacturer | Identifier |
|---|---|---|
| β actin monoclonal antibody | Sigma-Aldrich | A2228 |
| (source: mouse application: 1:2000) | | |
| α-Tubulin Antibody (source: rabbit, | Sigma-Aldrich | SAB4500087 |
| application: 1:2000) | | |
| Phospho-Stat5 (Tyr694) (D47E7) | Cell Signalling | 4322 |
| (source: rabbit, application 1:2000) | Technology | |
| Stat5 (D2O6Y) (source: rabbit, | Cell Signalling | 94205 |
| application 1:2000) | Technology | |
| P44/42 MAPK (Erk½) (source: | Cell Signalling | 9102 |
| rabbit, application 1:1000) | Technology | |
| p44/42 MAPK (Erk½) P | Cell Signalling | 9101 |
| Thr202/Tyr204 (source: rabbit, | Technology | |
| application 1:1000) | | |
| BRAF total (source: rabbit, | Sigma | HPA001328 |
| application 1:800) | | |
| BRAF V600E (source: rabbit, | Sigma | SAB4200772 |
| application 1:400) | | |
| (Horseradish peroxidase) HRP | Abcam | ab97023 |
| conjugated goat anti-mouse IgG | | |
| secondary Antibody (application: | | |
| 1:10,000) | | |
| HRP conjugated goat anti-rabbit | Abcam | ab205718 |
| IgG secondary Antibody | | |
| (application: 1:2000) | | |
| IRDye ® 680RD Goat anti-Rabbit | Li-cor | 92668071 |
| IgG Secondary Antibody (0.1 mg) | | |
| (application: 1:10,000) | | |
| IRDye ® 800CW Donkey anti- | Li-cor | 92532212 |
| Mouse IgG Secondary Antibody | | |
| (0.1mg) (application: 1:10,000) | | |

TABLE 5
Description of the sequences

| SEQ | |
|---|---|
| ID NO: | Description |
| 1 | Potent crRNA spacer sequence |
| 2 | Potent crRNA spacer sequence (G in 1^{st} position) |
| 3 | Potent crRNA spacer sequence (G in 1^{st} and 2^{nd} position) |
| 4 | Ineffective crRNA spacer sequence |
| 5 | NT crRNA |
| 6 | PspmCherry1 (matching position 3) spacer sequence |
| 7 | PspmCherry2 (matching position 49) spacer sequence |
| 8 | PspmCherry3 (matching position 118) spacer sequence |
| 9 | PspmCherry4 (matching position 193) spacer sequence |
| 10 | PspmCherry5 (matching position 250) spacer sequence |
| 11 | PspmCherry6 (matching position 319) spacer sequence |
| 12 | PspmCherry7 (matching position 406) spacer sequence |
| 13 | PspmCherry8 (matching position 455) spacer sequence |
| 14 | PspmCherry9 (matching position 526) spacer sequence |
| 15 | PspmCherry10 (matching position 593) spacer sequence |
| 16 | PspmCherry11 (matching position 655) spacer sequence |
| 17 | Shift PspmCherry1 (matching position 2) spacer sequence |
| 18 | Shift PspmCherry3 (matching position 52) spacer sequence |
| 19 | Shift PspmCherry5 (matching position 252) spacer sequence |
| 20 | Shift PspmCherry6 (matching position 315) spacer sequence |
| 21 | Shift PspmCherry10 (matching position 585) spacer sequence |
| 22 | Pspmcherry8(−15nt) spacer sequence |
| 23 | Pspmcherry8(−12nt) spacer sequence |
| 24 | Pspmcherry8(−9nt) spacer sequence |
| 25 | Pspmcherry8(−6nt) spacer sequence |
| 26 | Pspmcherry8(−3nt) spacer sequence |
| 27 | Pspmcherry8(+3nt) spacer sequence |
| 28 | Pspmcherry8(+6nt) spacer sequence |
| 29 | Pspmcherry8(+9nt) spacer sequence |
| 30 | Pspmcherry8(+12nt) spacer sequence |
| 31 | Pspmcherry8(+15nt) spacer sequence |
| 32 | Pspmcherry11(−15nt) spacer sequence |
| 33 | Pspmcherry11(−12nt) spacer sequence |
| 34 | Pspmcherry11(−9nt) spacer sequence |
| 35 | Pspmcherry11(−6nt) spacer sequence |
| 36 | Pspmcherry11(−3nt) spacer sequence |
| 37 | Pspmcherry11(+3nt) spacer sequence |
| 38 | Pspmcherry11(+6nt) spacer sequence |
| 39 | Pspmcherry11(+9nt) spacer sequence |
| 40 | Pspmcherry11(+12nt) spacer sequence |
| 41 | Pspmcherry11(+15nt) spacer sequence |
| 42 | PspmCherry8 tiled1 (matching position 485) spacer sequence |
| 43 | PspmCherry8 tiled2 (matching position 484) spacer sequence |
| 44 | PspmCherry8 tiled3 (matching position 483) spacer sequence |
| 45 | PspmCherry8 tiled4 (matching position 482) spacer sequence |
| 46 | PspmCherry8 tiled5 (matching position 481) spacer sequence |
| 47 | PspmCherry8 tiled6 (matching position 480) spacer sequence |
| 48 | PspmCherry8 tiled7 (matching position 479) spacer sequence |
| 49 | PspmCherry8 tiled8 (matching position 478) spacer sequence |
| 50 | PspmCherry8 tiled9 (matching position 477) spacer sequence |
| 51 | PspmCherry8 tiled10 (matching position 476) spacer sequence |
| 52 | PspmCherry8 tiled11 (matching position 475) spacer sequence |
| 53 | PspmCherry8 tiled12 (matching position 474) spacer sequence |
| 54 | PspmCherry8 tiled13 (matching position 473) spacer sequence |
| 55 | PspmCherry8 tiled14 (matching position 472) spacer sequence |
| 56 | PspmCherry8 titling15 (matching position 471) spacer sequence |
| 57 | PspmCherry8 tiled16 (matching position 470) spacer sequence |
| 58 | PspmCherry8 tiled17 (matching position 469) spacer sequence |
| 59 | PspmCherry8 tiled18 (matching position 468) spacer sequence |
| 60 | PspmCherry8 tiled19 (matching position 467) spacer sequence |
| 61 | PspmCherry8 tiled20 (matching position 466) spacer sequence |
| 62 | PspmCherry8 tiled21 (matching position 465) spacer sequence |
| 63 | PspmCherry8 tiled22 (matching position 464) spacer sequence |
| 64 | PspmCherry8 tiled23 (matching position 463) spacer sequence |
| 65 | PspmCherry8 tiled24 (matching position 462) spacer sequence |
| 66 | PspmCherry8 tiled25 (matching position 461) spacer sequence |
| 67 | PspmCherry8 tiled26 (matching position 460) spacer sequence |
| 68 | PspmCherry8 tiled27 (matching position 459) spacer sequence |
| 69 | PspmCherry8 tiled28 (matching position 458) spacer sequence |
| 70 | PspmCherry8 tiled29 (matching position 457) spacer sequence |
| 71 | PspmCherry8 tiled30 (matching position 456) spacer sequence |
| 72 | PspmCherry8 tiled31 (matching position 455) spacer sequence |
| 73 | PspmCherry8 tiled32 (matching position 454) spacer sequence |
| 74 | PspmCherry8 tiled33 (matching position 453) spacer sequence |
| 75 | PspmCherry8 tiled34 (matching position 452) spacer sequence |
| 76 | PspmCherry8 tiled35 (matching position 451) spacer sequence |
| 77 | PspmCherry8 tiled36 (matching position 450) spacer sequence |
| 78 | PspmCherry8 tiled37 (matching position 449) spacer sequence |
| 79 | PspmCherry8 tiled38 (matching position 448) spacer sequence |
| 80 | PspmCherry8 tiled39 (matching position 447) spacer sequence |
| 81 | PspmCherry8 tiled40 (matching position 446) spacer sequence |
| 82 | PspmCherry8 tiled41 (matching position 445) spacer sequence |
| 83 | PspmCherry8 tiled42 (matching position 444) spacer sequence |
| 84 | PspmCherry8 tiled43 (matching position 443) spacer sequence |
| 85 | PspmCherry8 tiled44 (matching position 442) spacer sequence |
| 86 | PspmCherry8 titling45 (matching position 441) spacer sequence |
| 87 | PspmCherry8 tiled46 (matching position 440) spacer sequence |
| 88 | PspmCherry8 tiled47 (matching position 439) spacer sequence |
| 89 | PspmCherry8 tiled48 (matching position 438) spacer sequence |
| 90 | PspmCherry8 tiled49 (matching position 437) spacer sequence |
| 91 | PspmCherry8 tiled50 (matching position 436) spacer sequence |
| 92 | PspmCherry8 tiled51 (matching position 435) spacer sequence |
| 93 | PspmCherry8 tiled52 (matching position 434) spacer sequence |
| 94 | PspmCherry8 tiled53 (matching position 432) spacer sequence |
| 95 | PspmCherry8 tiled54 (matching position 431) spacer sequence |
| 96 | PspmCherry8 tiled55 (matching position 430) spacer sequence |
| 97 | PspmCherry8 tiled56 (matching position 429) spacer sequence |
| 98 | PspmCherry8 tiled57 (matching position 428) spacer sequence |
| 99 | PspmCherry8 tiled58 (matching position 427) spacer sequence |
| 100 | PspmCherry8 tiled59 (matching position 426) spacer sequence |
| 101 | PspmCherry8 tiled60 (matching position 425) spacer sequence |
| 102 | PspmCherry8 tiled61 (matching position 424) spacer sequence |
| 103 | BCR-ABL1crRNA−20 spacer sequence |
| 104 | BCR-ABL1crRNA−19 spacer sequence |
| 105 | BCR-ABL1crRNA−18 spacer sequence |
| 106 | BCR-ABL1crRNA−17 spacer sequence |
| 107 | BCR-ABL1crRNA−16 spacer sequence |
| 108 | BCR-ABL1crRNA−15 spacer sequence |
| 109 | BCR-ABL1crRNA−14 spacer sequence |
| 110 | BCR-ABL1crRNA−13 spacer sequence |
| 111 | BCR-ABL1crRNA−12 spacer sequence |
| 112 | BCR-ABL1crRNA−11 spacer sequence |
| 113 | BCR-ABL1crRNA−10 spacer sequence |
| 114 | BCR-ABL1crRNA−9 spacer sequence |
| 115 | BCR-ABL1crRNA−8 spacer sequence |
| 116 | BCR-ABL1crRNA−7 spacer sequence |
| 117 | BCR-ABL1crRNA−6 spacer sequence |
| 118 | BCR-ABL1crRNA−5 spacer sequence |
| 119 | BCR-ABL1crRNA−4 spacer sequence |
| 120 | BCR-ABL1crRNA−3 spacer sequence |
| 121 | BCR-ABL1crRNA−2 spacer sequence |
| 122 | BCR-ABL1crRNA−1 spacer sequence |
| 123 | BCR-ABL1crRNA spacer sequence |
| 124 | BCR-ABL1crRNA1 spacer sequence |
| 125 | BCR-ABL1crRNA2 spacer sequence |
| 126 | BCR-ABL1crRNA3 spacer sequence |
| 127 | BCR-ABL1crRNA4 spacer sequence |
| 128 | BCR-ABL1crRNA5 spacer sequence |
| 129 | BCR-ABL1crRNA6 spacer sequence |
| 130 | BCR-ABL1crRNA7 spacer sequence |
| 131 | BCR-ABL1crRNA8 spacer sequence |
| 132 | BCR-ABL1crRNA9 spacer sequence |
| 133 | BCR-ABL1crRNA10 spacer sequence |
| 134 | BCR-ABL1crRNA11 spacer sequence |
| 135 | BCR-ABL1crRNA12 spacer sequence |
| 136 | BCR-ABL1crRNA13 spacer sequence |
| 137 | BCR-ABL1crRNA14 spacer sequence |
| 138 | BCR-ABL1crRNA15 spacer sequence |
| 139 | BCR-ABL1crRNA16 spacer sequence |
| 140 | BCR-ABL1crRNA17 spacer sequence |
| 141 | BCR-ABL1crRNA18 spacer sequence |
| 142 | BCR-ABL1crRNA19 spacer sequence |
| 143 | BCR-ABL1crRNA20 spacer sequence |
| 144 | SNX2-ABL1−12 spacer sequence |
| 145 | SNX2-ABL1−9 spacer sequence |
| 146 | SNX2-ABL1−6 spacer sequence |
| 147 | SNX2-ABL1−3 spacer sequence |
| 148 | SNX2-ABL1+0 spacer sequence |
| 149 | SNX2-ABL1+3 spacer sequence |
| 150 | SNX2-ABL1+6 spacer sequence |
| 151 | SNX2-ABL1+9 spacer sequence |
| 152 | SNX2-ABL1+12 spacer sequence |
| 153 | SFPQ-ABL1−12 spacer sequence |
| 154 | SFPQ-ABL1−9 spacer sequence |
| 155 | SFPQ-ABL1−6 spacer sequence |
| 156 | SFPQ-ABL1−3 spacer sequence |
| 157 | SFPQ-ABL1+0 spacer sequence |
| 158 | SFPQ-ABL1+3 spacer sequence |
| 159 | SFPQ-ABL1+6 spacer sequence |
| 160 | SFPQ-ABL1+9 spacer sequence |
| 161 | SFPQ-ABL1+12 spacer sequence |
| 162 | Spike crRNA −TIL1 spacer sequence |
| 163 | Spike crRNA−TIL2 spacer sequence |
| 164 | Spike crRNA−TIL3 spacer sequence |
| 165 | Spike crRNA−TIL4 spacer sequence |
| 166 | Spike crRNA−TILF5 spacer sequence |
| 167 | Spike crRNA−TIL6 spacer sequence |
| 168 | Spike crRNA−TIL7 spacer sequence |
| 169 | Spike crRNA−TIL8 spacer sequence |
| 170 | Spike crRNA−TILF9 spacer sequence |
| 171 | Spike crRNA−TIL10 spacer sequence |
| 172 | Spike crRNA−TIL11 spacer sequence |
| 173 | Spike crRNA−TIL12 spacer sequence |
| 174 | Spike crRNA−TIL13 spacer sequence |
| 175 | Spike crRNA−TIL14 spacer sequence |
| 176 | Spike crRNA−TIL15 spacer sequence |
| 177 | Spike crRNA−TIL16 spacer sequence |
| 178 | Spike crRNA−TIL17 spacer sequence |
| 179 | Spike crRNA−TIL18 spacer sequence |
| 180 | Spike crRNA−TIL19 spacer sequence |
| 181 | Spike crRNA−TIL20 spacer sequence |
| 182 | Spike crRNA−TIL21 spacer sequence |
| 183 | Spike crRNA−TIL22 spacer sequence |
| 184 | Spike crRNA−TIL23 spacer sequence |
| 185 | Spike crRNA−TIL24 spacer sequence |
| 186 | Spike crRNA−TIL25 spacer sequence |
| 187 | Spike crRNA−TIL26 spacer sequence |
| 188 | Spike crRNA−TIL27 spacer sequence |
| 189 | Spike crRNA−TIL28 spacer sequence |
| 190 | Spike crRNA−TIL29 spacer sequence |
| 191 | Spike crRNA−TIL30 spacer sequence |
| 192 | Spike crRNA−TIL31 spacer sequence |
| 193 | Spike crRNA−TIL32 spacer sequence |
| 194 | Spike crRNA−TIL33 spacer sequence |
| 195 | Spike crRNA−TIL34 spacer sequence |
| 196 | Spike crRNA−TIL35 spacer sequence |
| 197 | Spike crRNA−TIL36 spacer sequence |
| 198 | Spike crRNA−TIL37 spacer sequence |
| 199 | Spike crRNA−TIL38 spacer sequence |
| 200 | Spike crRNA−TIL39 spacer sequence |
| 201 | Spike crRNA−TIL40 spacer sequence |
| 202 | Spike crRNA−TIL41 spacer sequence |
| 203 | Spike crRNA−TIL42 spacer sequence |
| 204 | Spike crRNA−TIL43 spacer sequence |
| 205 | Spike crRNA−TIL44 spacer sequence |
| 206 | Spike crRNA−TIL45 spacer sequence |
| 207 | Spike crRNA−TIL46 spacer sequence |
| 208 | Spike crRNA−TIL47 spacer sequence |
| 209 | Spike crRNA−TIL48 spacer sequence |
| 210 | Spike crRNA−TIL49 spacer sequence |
| 211 | Spike crRNA−TIL50 spacer sequence |
| 212 | Spike crRNA−TIL51 spacer sequence |
| 213 | Spike crRNA−TIL52 spacer sequence |
| 214 | Spike crRNA−TIL53 spacer sequence |
| 215 | Spike crRNA−TIL54 spacer sequence |
| 216 | Spike crRNA−TIL55 spacer sequence |
| 217 | Spike crRNA−TIL56 spacer sequence |
| 218 | Spike crRNA−TIL57 spacer sequence |
| 219 | Spike crRNA−TIL58 spacer sequence |
| 220 | Spike crRNA−TIL59 spacer sequence |
| 221 | Spike crRNA−TIL60 spacer sequence |
| 222 | Spike crRNA−TIL61 spacer sequence |
| 223 | PspmCherry8 3ntMSM 1-3nt-mismatches spacer sequence |
| 224 | PspmCherry8 3ntMSM 4-6nt-mismatches spacer sequence |
| 225 | PspmCherry8 3ntMSM 7-9nt-mismatches spacer sequence |
| 226 | PspmCherry8 3ntMSM 10-12nt-mismatches spacer sequence |
| 227 | PspmCherry8 3ntMSM 13-15nt-mismatches spacer sequence |
| 228 | PspmCherry8 3ntMSM 16-18nt-mismatches spacer sequence |
| 229 | PspmCherry8 3ntMSM 19-21nt-mismatches spacer sequence |
| 230 | PspmCherry8 3ntMSM 22-24nt-mismatches spacer sequence |
| 231 | PspmCherry8 3ntMSM 25-27nt-mismatches spacer sequence |
| 232 | PspmCherry8 3ntMSM 28-30nt-mismatches spacer sequence |
| 233 | PspmCherry8 4ntMSM_1-4nt-mismatches spacer sequence |
| 234 | PspmCherry8 4ntMSM_5-8nt-mismatches spacer sequence |
| 235 | PspmCherry8 4ntMSM_9-12nt-mismatches spacer sequence |
| 236 | PspmCherry8 4ntMSM_13-16nt-mismatches spacer sequence |
| 237 | PspmCherry8 4ntMSM_17-20nt-mismatches spacer sequence |
| 238 | PspmCherry8 4ntMSM_21-24nt-mismatches spacer sequence |
| 239 | PspmCherry8 4ntMSM_25-28nt-mismatches spacer sequence |
| 240 | PspmCherry8 5ntMSM_1-5nt-mismatches spacer sequence |
| 241 | PspmCherry8 5ntMSM_6-10nt-mismatches spacer sequence |
| 242 | PspmCherry8 5ntMSM_11-15nt-mismatches spacer sequence |
| 243 | PspmCherry8 5ntMSM_16-20nt-mismatches spacer sequence |
| 244 | PspmCherry8 5ntMSM_21-25nt-mismatches spacer sequence |
| 245 | PspmCherry8 5ntMSM_26-30nt-mismatches spacer sequence |
| 246 | PspmCherry8 6ntMSM_1-6nt-mismatches spacer sequence |
| 247 | PspmCherry8 6ntMSM_7-12nt-mismatches spacer sequence |
| 248 | PspmCherry8 6ntMSM_13-18nt-mismatches spacer sequence |
| 249 | PspmCherry8 6ntMSM_19-24nt-mismatches spacer sequence |
| 250 | PspmCherry8 6ntMSM_25-30nt-mismatches spacer sequence |
| 251 | PspmCherry8 28-30nt-mismatches spacer sequence |
| 252 | PspmCherry8 25-30nt-mismatches spacer sequence |
| 253 | PspmCherry8 22-30nt-mismatches spacer sequence |
| 254 | PspmCherry8 19-30nt-mismatches spacer sequence |
| 255 | PspmCherry8 16-30nt-mismatches spacer sequence |
| 256 | PspmCherry8 13-30nt-mismatches spacer sequence |
| 257 | PspmCherry8 10-30nt-mismatches spacer sequence |
| 258 | PspmCherry8 7-30nt-mismatches spacer sequence |
| 259 | PspmCherry8 4-30nt-mismatches spacer sequence |
| 260 | PspmCherry8 1-30nt-mismatches spacer sequence |
| 261 | PspmCherry8 1-3nt-mismatches spacer sequence |
| 262 | PspmCherry8 1-6nt-mismatches spacer sequence |
| 263 | PspmCherry8 1-9nt-mismatches spacer sequence |
| 264 | PspmCherry8 1-12nt-mismatches spacer sequence |
| 265 | PspmCherry8 1-15nt-mismatches spacer sequence |
| 266 | PspmCherry8 1-18nt-mismatches spacer sequence |
| 267 | PspmCherry8 1-21nt-mismatches spacer sequence |
| 268 | PspmCherry8 1-24nt-mismatches spacer sequence |
| 269 | PspmCherry8 1-27nt-mismatches spacer sequence |
| 270 | PspmCherry8 1-30nt-mismatches spacer sequence |
| 271 | PspmCherry1_1-3nt-mismatches spacer sequence |
| 272 | PspmCherry2_1-3nt-mismatches spacer sequence |
| 273 | PspmCherry3_1-3nt-mismatches spacer sequence |
| 274 | PspmCherry4_1-3nt-mismatches spacer sequence |
| 275 | PspmCherry5_1-3nt-mismatches spacer sequence |
| 276 | PspmCherry6_1-3nt-mismatches spacer sequence |
| 277 | PspmCherry7_1-3nt-mismatches spacer sequence |
| 278 | PspmCherry8_1-3nt-mismatches spacer sequence |
| 279 | PspmCherry9_1-3nt-mismatches spacer sequence |
| 280 | PspmCherry10_1-3nt-mismatches spacer sequence |
| 281 | PspmCherry11_1-3nt-mismatches spacer sequence |
| 282 | PspmCherry8_1-2nt-mismatches spacer sequence |
| 283 | PspmCherry8_2-3nt-mismatches spacer sequence |
| 284 | PspmCherry8_1st&3rdnt-mismatches spacer sequence |
| 285 | PspmCherry8_1^{st}nt-mismatch spacer sequence |
| 286 | PspmCherry8_2^{nd}nt-mismatch spacer sequence |
| 287 | PspmCherry8_3^{rd}nt-mismatch spacer sequence |
| 288 | mcherry crRNA8 spread 5 SNP spacer sequence |
| 289 | mcherry crRNA8 spread 4 SNP spacer sequence |
| 290 | mcherry crRNA8 spread 3 SNP spacer sequence |
| 291 | mcherry crRNA8 spread 2 SNP spacer sequence |
| 292 | mcherry gRNA8 spread 15 SNP spacer sequence |
| 293 | mcherry gRNA8 spread 10 SNP spacer sequence |
| 294 | mcherry gRNA8 spread 7 SNP spacer sequence |
| 295 | mcherry gRNA8 spread 6 SNP spacer sequence |
| 296 | mcherry gRNA8 spread 2 MSM spacer sequence |
| 297 | mcherry gRNA8 spread 3 MSM spacer sequence |
| 298 | gmcherry8(−7nt) 1G spacer sequence |
| 299 | gmcherry8(−6nt) 1G spacer sequence |
| 300 | gmcherry8(−5nt) 1G spacer sequence |
| 301 | gmcherry8(−4nt) 1G spacer sequence |
| 302 | gmcherry8(−3nt) 1G spacer sequence |
| 303 | gmcherry8(−7nt) 2G spacer sequence |
| 304 | gmcherry8(−6nt) 1G spacer sequence |
| 305 | gmcherry8(−5nt) 2G spacer sequence |
| 306 | gmcherry8(−4nt) 2G spacer sequence |
| 307 | gmcherry8(−3nt) 2G spacer sequence |
| 308 | gmcherry8(+8nt) extra g spacer sequence |
| 309 | gmcherry8(+9nt) extra g spacer sequence |
| 310 | gmcherry8(+8nt) 1g spacer sequence |
| 311 | gmcherry8(+9nt) 1g spacer sequence |
| 312 | gmcherry8(+8nt) 2g spacer sequence |
| 313 | gmcherry8(+9nt) 2g spacer sequence |
| 314 | gmcherry8(−7nt) extra g spacer sequence |
| 315 | gmcherry8(−6nt) extra g spacer sequence |
| 316 | gmcherry8(−5nt) extra g spacer sequence |
| 317 | gmcherry8(−4nt) extra g spacer sequence |
| 318 | gmcherry8(−3nt) extra g spacer sequence |
| 319 | BCR crRNA −3nt G1-2 spacer sequence |
| 320 | BCR crRNA +6nt G1-2 spacer sequence |
| 321 | BCR crRNA +9nt G1-2 spacer sequence |
| 322 | BCR crRNA +9nt G15-17-18 spacer sequence |
| 323 | BCR crRNA +9nt G17-18 spacer sequence |
| 324 | BCR crRNA +9nt G1-2-15-17-18 spacer sequence |
| 325 | SNX2 crRNA +6nt G2 spacer sequence |
| 326 | SNX2 crRNA +6nt G15-16 spacer sequence |
| 327 | SNX2 crRNA +6nt G15-16-17 spacer sequence |
| 328 | SNX2 crRNA +6nt G2-15-16-17 spacer sequence |
| 329 | SNX2 crRNA +12nt G2 spacer sequence |
| 330 | SNX2 crRNA +12nt G11 spacer sequence |
| 331 | SNX2 crRNA +12nt G17 spacer sequence |
| 332 | SNX2 crRNA +12nt G2-11-17 spacer sequence |
| 333 | SNX2 crRNA +3nt G15-18 spacer sequence |
| 334 | SNX2 crRNA +3nt G15 spacer sequence |
| 335 | 5′ 3nt MSM BCR-ABL1 spacer sequence |
| 336 | 5′ 6nt MSM BCR-ABL1 spacer sequence |
| 337 | 5′ 9nt MSM BCR-ABL1 spacer sequence |
| 338 | 3′ 3nt MSM BCR-ABL1 spacer sequence |
| 339 | 3′ 6nt MSM BCR-ABL1 spacer sequence |
| 340 | 3′ 9nt MSM BCR-ABL1 spacer sequence |
| 341 | Central 3nt MSM BCR-ABL1 spacer sequence |
| 342 | central 6nt MSM BCR-ABL1 spacer sequence |
| 343 | central 9nt MSM BCR-ABL1 spacer sequence |
| 344 | spread 3 SNP BCR-ABL1 spacer sequence |
| 345 | spread 4 SNP BCR-ABL1 spacer sequence |
| 346 | spread 5 SNP BCR-ABL1 spacer sequence |
| 347 | spread 6 SNP BCR-ABL1 spacer sequence |
| 348 | spread 7 SNP BCR-ABL1 spacer sequence |
| 349 | spread 10 SNP BCR-ABL1 spacer sequence |
| 350 | 2nt MSM spread BCR-ABL1 spacer sequence |
| 351 | Ineffective EGFP crRNAl spacer sequence |
| 352 | Ineffective EGFP crRNA2 spacer sequence |
| 353 | Ineffective EGFP crRNA3 spacer sequence |
| 354 | Ineffective EGFP crRNA4 spacer sequence |
| 355 | Ineffective EGFP crRNA5 spacer sequence |
| 356 | Ineffective EGFP crRNA6 spacer sequence |
| 357 | Ineffective EGFP crRNA7 spacer sequence |
| 358 | Ineffective EGFP crRNA8 spacer sequence |
| 359 | Ineffective EGFP crRNA9 spacer sequence |
| 360 | Potent EGFP crRNAl spacer sequence |
| 361 | Potent EGFP crRNA2 spacer sequence |
| 362 | Potent EGFP crRNA3 spacer sequence |
| 363 | Potent EGFP crRNA4 spacer sequence |
| 364 | Potent EGFP crRNA5 spacer sequence |
| 365 | Potent EGFP crRNA6 spacer sequence |
| 366 | Potent EGFP crRNA7 spacer sequence |
| 367 | Potent EGFP crRNA8 spacer sequence |
| 368 | Potent EGFP crRNA9 spacer sequence |
| 369 | Potent EGFP crRNA10 spacer sequence |
| 370 | Potent EGFP crRNA11 spacer sequence |
| 371 | Ineffective Tagbfp crRNAl spacer sequence |
| 372 | Ineffective Tagbfp crRNA2 spacer sequence |
| 373 | Ineffective Tagbfp crRNA3 spacer sequence |
| 374 | Ineffective Tagbfp crRNA4 spacer sequence |
| 375 | Ineffective Tagbfp crRNA5 spacer sequence |
| 376 | Ineffective Tagbfp crRNA6 spacer sequence |
| 377 | Ineffective Tagbfp crRNA7 spacer sequence |
| 378 | Ineffective Tagbfp crRNA8 spacer sequence |
| 379 | Ineffective Tagbfp crRNA9 spacer sequence |
| 380 | Potent Tagbfp crRNAl spacer sequence |
| 381 | Potent Tagbfp crRNA2 spacer sequence |
| 382 | Potent Tagbfp crRNA3 spacer sequence |
| 383 | Potent Tagbfp crRNA4 spacer sequence |
| 384 | Potent Tagbfp crRNA5 spacer sequence |
| 385 | Potent Tagbfp crRNA6 spacer sequence |
| 386 | Potent Tagbfp crRNA7 spacer sequence |
| 387 | Potent Tagbfp crRNA8 spacer sequence |
| 388 | Potent Tagbfp crRNA9 spacer sequence |
| 389 | Potent Tagbfp crRNA10 spacer sequence |
| 390 | RfxCas13d mcherry efficient 1 spacer sequence |
| 391 | RfxCas13d mcherry efficient 2 spacer sequence |
| 392 | RfxCas13d mcherry efficient 3 spacer sequence |
| 393 | RfxCas13d mcherry efficient 4 spacer sequence |
| 394 | RfxCas13d mcherry efficient 5 spacer sequence |
| 395 | RfxCas13d mcherry efficient 6 spacer sequence |
| 396 | RfxCas13d mcherry efficient 7 spacer sequence |
| 397 | RfxCas13d mcherry efficient 8 spacer sequence |
| 398 | RfxCas13d mcherry efficient 9 spacer sequence |
| 399 | RfxCas13d mcherry efficient 10 spacer sequence |
| 400 | M-Cherry |
| 401 | SNX2-ABL1 sequence (full length cloned into MSCV-IRES-EGFP) |
| 402 | P190 BCR-ABL1 sequence (full length cloned into MSCV-IRES-EGFP) |
| 403 | SFPQ-ABL1 (full length cloned into MSCV-IRES-EGFP) |
| 404 | ABL1 (partial)-3XHA tag (cloned into MSCV-IRES-EGFP) |
| 405 | BCR (partial)-3XHA tag (cloned into MSCV-IRES-tagBFP) |
| 406 | BCR-ABL1 (partial)-3XHA tag (cloned into MSCV-IRES-m-cherry) |
| 407 | IRES forward primer |
| 408 | M13 reverse primer |
| 409 | BCR-ABL1 p194640 forward primer |
| 410 | BCR-ABL1 p190 reverse primer |
| 411 | SFPQ-ABL1 forward primer |
| 412 | SFPQ-ABL1 reverse primer |
| 413 | SNX2-ABL1 forward primer |
| 414 | SNX2-ABL1 R reverse primer |
| 415 | HSP90A1B forward primer |
| 416 | HSP90A1B reverse primer |
| 417 | GAPDH forward primer |
| 418 | GAPDH reverse primer |
| 419 | crBRAF-1 (Psp) spacer sequence |
| 420 | crBRAF-2 (Psp) spacer sequence |
| 421 | crBRAF-3 (Psp) spacer sequence |
| 422 | crBRAF-4 (Psp) spacer sequence |
| 423 | crBRAF-5 (Psp) spacer sequence |
| 424 | PspBRAF-Mut1 spacer sequence |
| 425 | PspBRAF-Mut2 spacer sequence |
| 426 | PspBRAF-Mut3 spacer sequence |
| 427 | PspBRAF-Mut4 spacer sequence |
| 428 | PspBRAF-Mut5 spacer sequence |
| 429 | PspBRAF-Mut6 spacer sequence |
| 430 | PspBRAF-Mut7 spacer sequence |
| 431 | PspBRAF-Mut8 spacer sequence |
| 432 | PspBRAF-Mut9 spacer sequence |
| 433 | PspBRAF-Mut10 spacer sequence |
| 434 | PspBRAF-Mut11 spacer sequence |
| 435 | PspBRAF-Mut12 spacer sequence |
| 436 | PspBRAF-Mut13 spacer sequence |
| 437 | PspBRAF-Mut14 spacer sequence |
| 438 | PspBRAF-Mut15 spacer sequence |
| 439 | PspBRAF-Mut16 spacer sequence |
| 440 | PspBRAF-Mut17 spacer sequence |
| 441 | PspBRAF-Mut18 spacer sequence |
| 442 | PspBRAF-Mut19 spacer sequence |
| 443 | PspBRAF-Mut20 spacer sequence |
| 444 | PspBRAF-Mut21 spacer sequence |
| 445 | PspBRAF-Mut22 spacer sequence |
| 446 | SpCas9 gNT spacer sequence |
| 447 | SpCas9-gBRAF1spacer sequence |
| 448 | SpCas9-gBRAF2 spacer sequence |
| 449 | BRAF forward primer |
| 450 | BRAF reverse primer |
| 451 | PspCas 13b amino acid sequence |
| 452 | PspCas13b nucleic acid sequence |
| 453 | pC0043-PspCas13b crRNA backbone (Addgene#103854) |
| 454 | Ef1a-PspCas13b-NES-3xFLAG-T2A-BFP (Addgene #173029) |
| 455 | pC0046-EF1a-PspCas13b-NES-HIV (Addgene #103862) |
| 456 | FUCas9-mCherry (Addgene #70182) |
| 457 | crBCR-ABL1−3nt mismatch-1 |
| 458 | crBCR-ABL1−3nt mismatch-2 |
| 459 | crBCR-ABL1−6nt mismatch |
| 460 | crBCR-ABL1−3nt mismatch-3 |
| 461 | crBCR-ABL1−3nt mismatch-4 |
| 462 | crBCR-ABL1−3nt mismatch-5 |
| 463 | EcoRI-BRAFwt-3xFLAG-STOP-XhoI |
| 464 | EcoRI-BRAF^{V600E}-3xHA-STOP-XhoI |
| 465 | crNT crRNA (Rfx) |
| 466 | crBRAF-1 (Rfx) |
| 467 | crMM-1 (RfxBRAF V600E crRNA spacer sequence) |
| 468 | crMM-2 (RfxBRAF V600E crRNA spacer sequence) |
| 469 | crMM-3 (RfxBRAF V600E crRNA spacer sequence) |
| 470 | crMM-4 (RfxBRAF V600E crRNA spacer sequence) |
| 471 | crMM-5 (RfxBRAF V600E crRNA spacer sequence) |
| 472 | crMM-6 (RfxBRAF V600E crRNA spacer sequence) |
| 473 | crMM-7 (RfxBRAF V600E crRNA spacer sequence) |
| 474 | crMM-8 (RfxBRAF V600E crRNA spacer sequence) |
| 475 | crMM-9 (RfxBRAF V600E crRNA spacer sequence) |
| 476 | crMM-10 (RfxBRAF V600E crRNA spacer sequence) |
| 477 | crMM-11 (RfxBRAF V600E crRNA spacer sequence) |
| 478 | crMM-12 (RfxBRAF V600E crRNA spacer sequence) |
| 479 | crMM-13 (RfxBRAF V600E crRNA spacer sequence) |
| 480 | crMM-14 (RfxBRAF V600E crRNA spacer sequence) |
| 481 | crMM-15 (RfxBRAF V600E crRNA spacer sequence) |
| 482 | crMM-16 (RfxBRAF V600E crRNA spacer sequence) |
| 483 | crMM-17 (RfxBRAF V600E crRNA spacer sequence) |
| 484 | crMM-18 (RfxBRAF V600E crRNA spacer sequence) |
| 485 | crMM-19 (RfxBRAF V600E crRNA spacer sequence) |
| 486 | crMM-20 (RfxBRAF V600E crRNA spacer sequence) |
| 487 | crMM-21 (RfxBRAF V600E crRNA spacer sequence) |
| 488 | crMM-22 (RfxBRAF V600E crRNA spacer sequence) |
| 489 | crC (RfxKRAS G12C crRNA spacer sequence) |
| 490 | crC/A-12 (RfxKRAS G12C, G12A crRNA spacer sequence) |
| 491 | crC/A-9 (RfxKRAS G12C, G12A) spacer sequence |
| 492 | crC/D (RfxKRAS G12C, G12D) spacer sequence |
| 493 | crC/D-12 1ntShift (RfxKRAS G12C, G12D crRNA spacer sequence) |
| 494 | crC/D-12 (RfxKRAS G12C, G12D crRNA spacer sequence) |
| 495 | crC/D-9 1ntShift (RfxKRAS G12C, G12D crRNA spacer sequence) |
| 496 | crC/D-9 (RfxKRAS G12C, G12D crRNA spacer sequence) |
| 497 | crC/V-12 (RfxKRAS G12C, G12V crRNA spacer sequence) |
| 498 | crC/V-9 (RfxKRAS G12C, G12V crRNA spacer sequence) |
| 499 | crC-WT-12 (RfxKRAS WT, G12C crRNA spacer sequence) |
| 500 | crC-WT-9 (RfxKRAS WT, G12C crRNA spacer sequence) |
| 501 | crC23 (RfxKRAS G12C) crRNA spacer sequence) |
| 502 | crCD (RfxKRAS G12C, G12D crRNA spacer sequence) |
| 503 | crCD1 (RfxKRAS G12C, G12D crRNA spacer sequence) |
| 504 | crCD12 (RfxKRAS G12C, G12D crRNA spacer sequence) |
| 505 | crCD15 (RfxKRAS G12C, G12D crRNA spacer sequence) |
| 506 | crCD18 (RfxKRAS G12C, G12D crRNA spacer sequence) |
| 507 | crCD2_1 (RfxKRAS G12C, G12D crRNA spacer sequence) |
| 508 | crCD2_12 (RfxKRAS G12C, G12D crRNA spacer sequence) |
| 509 | crCD2_15 (RfxKRAS G12C, G12D crRNA spacer sequence) |
| 510 | crCD2_18 (RfxKRAS G12C, G12D crRNA spacer sequence) |
| 511 | crCD2_21 (RfxKRAS G12C, G12D crRNA spacer sequence) |
| 512 | crCD2_4 (RfxKRAS G12C, G12D crRNA spacer sequence) |
| 513 | crCD2_6 (RfxKRAS G12C, G12D crRNA spacer sequence) |
| 514 | crCD2_9 (RfxKRAS G12C, G12D crRNA spacer sequence) |
| 515 | crCD21 (RfxKRAS G12C, G12D crRNA spacer sequence) |
| 516 | crCD23 (RfxKRAS G12C, G12D crRNA spacer sequence) |
| 517 | crCD4 (RfxKRAS G12C, G12D crRNA spacer sequence) |
| 518 | crCD6 (RfxKRAS G12C, G12D crRNA spacer sequence) |
| 519 | crCD9 (RfxKRAS G12C, G12D crRNA spacer sequence) |
| 520 | crD (RfxKRAS G12D crRNA spacer sequence) |
| 521 | crD-10-12 (RfxKRAS G12D, G12R crRNA spacer sequence) |
| 522 | crD-9-10 (RfxKRAS G12D crRNA spacer sequence) |
| 523 | crD-R 12 (RfxKRAS G12D, G12R crRNA spacer sequence) |
| 524 | crD-R 9 (RfxKRAS G12D, G12R crRNA spacer sequence) |
| 525 | crD-S 12 (RfxKRAS G12D, G12S crRNA spacer sequence) |
| 526 | crD-S 9 (RfxKRAS G12D, G12S crRNA spacer sequence) |
| 527 | crD-WT 12 (RfxKRAS WT, G12D crRNA spacer sequence) |
| 528 | crD-WT 9 (RfxKRAS WT, G12D crRNA spacer sequence) |
| 529 | crD1 (RfxKRAS G12D crRNA spacer sequence) |
| 530 | crD11 (RfxKRAS G12D crRNA spacer sequence) |
| 531 | crD12 (RfxKRAS G12D crRNA spacer sequence) |
| 532 | crD13 (RfxKRAS G12D crRNA spacer sequence) |
| 533 | crD14 (RfxKRAS G12D crRNA spacer sequence) |
| 534 | crD15 (RfxKRAS G12D crRNA spacer sequence) |
| 535 | crD16 (RfxKRAS G12D crRNA spacer sequence) |
| 536 | crD17 (RfxKRAS G12D crRNA spacer sequence) |
| 537 | crD18 (RfxKRAS G12D crRNA spacer sequence) |
| 538 | crD19 (RfxKRAS G12D crRNA spacer sequence) |
| 539 | crD2 (RfxKRAS G12D crRNA spacer sequence) |
| 540 | rD20 (RfxKRAS G12D crRNA spacer sequence) |
| 541 | crD21(RfxKRAS G12D crRNA spacer sequence) |
| 542 | crD22 (RfxKRAS G12D crRNA spacer sequence) |
| 543 | crD23 (RfxKRAS G12D crRNA spacer sequence) |
| 544 | crD23 (RfxKRAS G12C crRNA spacer sequence) |
| 545 | crD3 (RfxKRAS G12D crRNA spacer sequence) |
| 546 | crD4 (RfxKRAS G12D crRNA spacer sequence) |
| 547 | crD5 (RfxKRAS G12D crRNA spacer sequence) |
| 548 | crD6 (RfxKRAS G12D crRNA spacer sequence) |
| 549 | crD7 (RfxKRAS G12D crRNA spacer sequence) |
| 550 | crD8 (RfxKRAS G12D crRNA spacer sequence) |
| 551 | crD9 (RfxKRAS G12D crRNA spacer sequence) |
| 552 | SV9 (RfxKRAS G12S, G12V crRNA spacer sequence) |
| 553 | RV9 (RfxKRAS G12R, G12V crRNA spacer sequence) |
| 554 | SV12 (RfxKRAS G12S, G12V crRNA spacer sequence) |
| 555 | RV12 (RfxKRAS G12R, G12V crRNA spacer sequence) |
| 556 | SV9_IntShift (RfxKRAS G12S, G12V crRNA spacer sequence) |
| 557 | RV9_IntShift (RfxKRAS G12R, G12V crRNA spacer sequence) |
| 558 | SV12_IntShift (RfxKRAS G12S, G12V crRNA spacer sequence) |
| 559 | CV9_23 (RfxKRAS G12C, G12V crRNA spacer sequence) |
| 560 | CV12_23 (RfxKRAS G12C, G12V crRNA spacer sequence) |
| 561 | RfxCas13d |
| 562 | BamH1-KRAS-WT |
| 563 | BamH1-KRAS-G12A |
| 564 | BamH1-KRAS-G12C |
| 565 | BamH1-KRAS-G12D |
| 566 | BamH1-KRAS-G12R |
| 567 | BamH1-KRAS-G12S |
| 568 | BamH1-KRAS-G12V |

1. A crRNA comprising from 5′ to 3′:
a. a spacer sequence that is capable of hybridizing to a target RNA sequence; and
b. a direct repeat sequence,
wherein the nucleotide content of the spacer sequence has been enriched for G nucleotides.

2. (canceled)

3. The crRNA of claim 1, wherein one or more or all of the following apply:
a. the spacer sequence comprises from about 20 nucleotides to about 40 nucleotides:
b. wherein the nucleotide content of the 5′ end of the spacer has been enriched for G nucleotides;
c. the spacer sequence comprises a G nucleotide at positions 1 and 2;
d. the spacer sequence comprises from about 20 to about 30 nucleotides that are capable of hybridizing to the target RNA sequence;
e. the spacer sequence comprises at least one mismatched nucleotide, wherein each nucleotide of the target RNA sequence.

4.-7. (canceled)

8. The crRNA of claim 1, wherein the spacer sequence comprises:
a. the nucleotide sequence of DDNNNNNNNNDDNNDDDNNNNNNNNNNNN (SEQ ID NO:1), wherein N is a G, U, A or C nucleotide and D is a G, U or A nucleotide; or
b. the nucleotide sequence of GDNNNNNNNNDDNNDDDNNNNNNNNNNNNN (SEQ ID NO:2), wherein N is a G, U, A or C nucleotide and D is a G, U or A nucleotide; or
c. the nucleotide sequence of GGNNNNNNNNDDNNDDDNNNNNNNNNNNNN (SEQ ID NO:3), wherein N is a G, U, A or C nucleotide and D is a G, U or A nucleotide.

9.-10. (canceled)

11. The crRNA of claim 8, wherein D is a G nucleotide.

12.-17. (canceled)

18. A crRNA comprising a spacer sequence that is capable of hybridizing to a target RNA sequence, wherein the target RNA sequence is within a variant transcript, wherein the spacer sequence comprises at least one nucleotide mismatch relative to a corresponding nucleotide of the target RNA sequence, and wherein the crRNA selectively targets the variant transcript relative to a corresponding wild-type transcript from the same gene locus.

19. (canceled)

20. The crRNA of claim 18, wherein the variant transcript comprises at least one single nucleotide variant (SNV) relative to a corresponding wild-type transcript from the same gene locus.

21. The crRNA of claim 20, wherein the spacer sequence comprises one or both of:
a. one or two mismatched nucleotides relative to a corresponding nucleotide of the target RNA sequence; and
b. from about one to about 3 mismatched nucleotides relative to the corresponding wild-type transcript from the same gene locus.

22. The crRNA of claim 1, wherein the spacer sequence comprises, consists, or consists essentially of the nucleotide sequence of any one of SEQ ID NOs: 419-423, 435-437, 439, 441 and 465-560, or a nucleotide sequence which is at least 90% identical to the nucleotide sequence of any one of SEQ ID NOs: 419-423, 435-437, 439, 441 and 465-560.

23. An RNA editing system comprising:
a. a Cas13b effector protein, or a polynucleotide encoding the Cas13b effector protein; and
b. the crRNA of claim 1, or a polynucleotide encoding the crRNA.

24.-25. (canceled)

26. The RNA editing system of claim 23, wherein the Cas13b is an ortholog selected from the group consisting of Prevotella buccae Cas13b (PbuCas13b), Prevotella sp. P5-125 Cas13b (PspCas13b), Bergeyella zoohelcum Cas13b (BzCas13b), and Porphyromonas gulae (PguCas13b).

27.-32. (canceled)

33. A cell or cell extract comprising the RNA editing system of claim 23.

34. (canceled)

35. A method of altering a target RNA sequence in a cell, the method comprising providing to the cell the RNA editing system of claim 23, wherein the Cas13b effector protein when in conjunction with the crRNA, hybridizes to the target RNA sequence, and wherein the Cas13b effector alters the hybridized target RNA sequence.

36.-37. (canceled)

38. The method of claim 35, wherein the target RNA sequence shares homology with one or more non-target RNA sequences.

39. The method of claim 38, wherein the target RNA sequence is within a transcript selected from the group consisting of an RNA isoform, a variant transcript comprising at least one SNV, a gene fusion transcript, and a wild-type transcript.

40. The method of claim 39, wherein one or more or all of the following apply:
a. the target RNA sequence is within a gene fusion transcript, wherein the gene fusion transcript is selected from the group consisting of BCR-ABL1, SFPQ-ABL1 and SXN2-ABL1;
b. the target RNA sequence comprises the fusion breakpoint of the gene fusion transcript; and
c. the target RNA sequence is within a variant transcript comprising at least one SNV.

41.-44. (canceled)

45. The method of claim 40, wherein the SNV is a pathogenic mutation selected from the group consisting of BRAF V600E, KRAS G12C, KRAS G12R, KRAS G12S, KRAS G12A, KRAS G12D, KRAS G12V, and combinations of the foregoing.

46. (canceled)

47. A method for selecting a potent crRNA, the method comprising:
a. generating a plurality of crRNA in silico, wherein each of the plurality of crRNA comprises from 5′ to 3′: (i) a spacer sequence that is capable of hybridizing to the target RNA sequence, and (ii) a direct repeat sequence;
b. determining the spacer nucleotide content for each of the plurality of crRNA; and
c. selecting potent crRNA from the plurality of crRNA, wherein potent crRNA comprise a spacer sequence that is enriched for G nucleotides.

48. (canceled)

49. The method of claim 47, further comprising selecting ineffective crRNA for modification to improve potency, wherein ineffective crRNA comprises one or more or all of the following:
a. a spacer sequence that is enriched for C nucleotides;
b. a spacer sequence comprising a C nucleotide at a position selected from 1, 2, 3, 4, 11, 12, 15, 16, 17, and combinations of the foregoing, and
c. a spacer sequence comprising the nucleotide sequence of CCCCNNNNNNCCNNCCCHNNNNNNNNNNNN (SEQ ID NO:4), wherein N is a C, U, A, or G nucleotide and H is a C, U, or A nucleotide.

50.-53. (canceled)

54. The method of claim 49, wherein the modification is one or both of:
a. the addition of at least one G nucleotide; and
b. the substitution of at least one A, U or C nucleotide to a G nucleotide.

55. A method for selecting a crRNA having a spacer sequence that hybridizes to a target RNA sequence within a variant transcript comprising at least one SNV relative to a corresponding wild-type transcript from the same gene locus, the method comprising:
a. generating a plurality of crRNA in silico, wherein each of the plurality of crRNA comprises a spacer sequence that is capable of hybridizing to the target RNA sequence within the variant transcript;
b. determining the spacer nucleotide content for each of the plurality of crRNA; and
c. selecting a crRNA from the plurality of crRNA, wherein the selected crRNA comprises a spacer sequence comprising at least one nucleotide mismatch relative to a corresponding nucleotide of the target RNA sequence, and wherein the selected crRNA selectively targets the variant transcript relative to a corresponding wild-type transcript from the same gene locus.

56. (canceled)


SEQUENCE LISTING (568 sequences)

SEQ ID NO: 1-3 - skipped (3 entries)

SEQ ID NO: 4 - RNA, 30 nt, synthetic construct
  source 1..30: mol_type = other RNA
ccccnnnnnnccnnccchnnnnnnnnnnnn

SEQ ID NO: 5 - RNA, 30 nt, synthetic construct
  source 1..30: mol_type = other RNA
tagattgctgttctaccaagtaatccatca

SEQ ID NO: 6 - RNA, 30 nt, synthetic construct
  source 1..30: mol_type = other RNA
ggccatgttatcctcctcgcccttgctcac

SEQ ID NO: 7 - RNA, 30 nt, synthetic construct
  source 1..30: mol_type = other RNA
ggagccctccatgtgcaccttgaagcgcat

SEQ ID NO: 8 - RNA, 30 nt, synthetic construct
  source 1..30: mol_type = other RNA
ggcggtctgggtgccctcgtaggggcggcc

SEQ ID NO: 9 - RNA, 30 nt, synthetic construct
  source 1..30: mol_type = other RNA
ggagccgtacatgaactgaggggacaggat

SEQ ID NO: 10 - RNA, 30 nt, synthetic construct
  source 1..30: mol_type = other RNA
ggggaaggacagcttcaagtagtcggggat

SEQ ID NO: 11 - RNA, 30 nt, synthetic construct
  source 1..30: mol_type = other RNA
ggagtcctgggtcacggtcaccacgccgcc

SEQ ID NO: 12 - RNA, 30 nt, synthetic construct
  source 1..30: mol_type = other RNA
ggtcttcttctgcattacggggccgtcgga

SEQ ID NO: 13 - RNA, 30 nt, synthetic construct
  source 1..30: mol_type = other RNA
gggcgccgtcctcggggtacatccgctcgg

SEQ ID NO: 14 - RNA, 30 nt, synthetic construct
  source 1..30: mol_type = other RNA
ggtggtcttgacctcagcgtcgtagtggcc

SEQ ID NO: 15 - RNA, 30 nt, synthetic construct
  source 1..30: mol_type = other RNA
ggtgatgtccaacttgatgttgacgttgta

SEQ ID NO: 16 - RNA, 30 nt, synthetic construct
  source 1..30: mol_type = other RNA
ggtggagtggcggccctcggcgcgttcgta

SEQ ID NO: 17 - RNA, 30 nt, synthetic construct
  source 1..30: mol_type = other RNA
ccatgttatcctcctcgcccttgctcacca

SEQ ID NO: 18 - RNA, 30 nt, synthetic construct
  source 1..30: mol_type = other RNA
cacggagccctccatgtgcaccttgaagcg

SEQ ID NO: 19 - RNA, 30 nt, synthetic construct
  source 1..30: mol_type = other RNA
tcggggaaggacagcttcaagtagtcgggg

SEQ ID NO: 20 - RNA, 30 nt, synthetic construct
  source 1..30: mol_type = other RNA
tcctgggtcacggtcaccacgccgccgtcc

SEQ ID NO: 21 - RNA, 30 nt, synthetic construct
  source 1..30: mol_type = other RNA
tccaacttgatgttgacgttgtaggcgccg

SEQ ID NO: 22 - RNA, 30 nt, synthetic construct
  source 1..30: mol_type = other RNA
tgatctcgcccttcagggcgccgtcctcgg

SEQ ID NO: 23 - RNA, 30 nt, synthetic construct
  source 1..30: mol_type = other RNA
tctcgcccttcagggcgccgtcctcggggt

SEQ ID NO: 24 - RNA, 30 nt, synthetic construct
  source 1..30: mol_type = other RNA
cgcccttcagggcgccgtcctcggggtaca

SEQ ID NO: 25 - RNA, 30 nt, synthetic construct
  source 1..30: mol_type = other RNA
ccttcagggcgccgtcctcggggtacatcc

SEQ ID NO: 26 - RNA, 30 nt, synthetic construct
  source 1..30: mol_type = other RNA
tcagggcgccgtcctcggggtacatccgct

SEQ ID NO: 27 - RNA, 30 nt, synthetic construct
  source 1..30: mol_type = other RNA
cgccgtcctcggggtacatccgctcggagg

SEQ ID NO: 28 - RNA, 30 nt, synthetic construct
  source 1..30: mol_type = other RNA
cgtcctcggggtacatccgctcggaggagg

SEQ ID NO: 29 - RNA, 30 nt, synthetic construct
  source 1..30: mol_type = other RNA
cctcggggtacatccgctcggaggaggcct

SEQ ID NO: 30 - RNA, 30 nt, synthetic construct
  source 1..30: mol_type = other RNA
cggggtacatccgctcggaggaggcctccc

SEQ ID NO: 31 - RNA, 30 nt, synthetic construct
  source 1..30: mol_type = other RNA
ggtacatccgctcggaggaggcctcccagc

SEQ ID NO: 32 - RNA, 30 nt, synthetic construct
  source 1..30: mol_type = other RNA
ctcgtccatgccgccggtggagtggcggcc

SEQ ID NO: 33 - RNA, 30 nt, synthetic construct
  source 1..30: mol_type = other RNA
gtccatgccgccggtggagtggcggccctc

SEQ ID NO: 34 - RNA, 30 nt, synthetic construct
  source 1..30: mol_type = other RNA
catgccgccggtggagtggcggccctcggc

SEQ ID NO: 35 - RNA, 30 nt, synthetic construct
  source 1..30: mol_type = other RNA
gccgccggtggagtggcggccctcggcgcg

SEQ ID NO: 36 - RNA, 30 nt, synthetic construct
  source 1..30: mol_type = other RNA
gccggtggagtggcggccctcggcgcgttc

SEQ ID NO: 37 - RNA, 30 nt, synthetic construct
  source 1..30: mol_type = other RNA
ggagtggcggccctcggcgcgttcgtactg

SEQ ID NO: 38 - RNA, 30 nt, synthetic construct
  source 1..30: mol_type = other RNA
gtggcggccctcggcgcgttcgtactgttc

SEQ ID NO: 39 - RNA, 30 nt, synthetic construct
  source 1..30: mol_type = other RNA
gcggccctcggcgcgttcgtactgttccac

SEQ ID NO: 40 - RNA, 30 nt, synthetic construct
  source 1..30: mol_type = other RNA
gccctcggcgcgttcgtactgttccacgat

SEQ ID NO: 41 - RNA, 30 nt, synthetic construct
  source 1..30: mol_type = other RNA
ctcggcgcgttcgtactgttccacgatggt

SEQ ID NO: 42 - RNA, 30 nt, synthetic construct
  source 1..30: mol_type = other RNA
gcttcagcctctgcttgatctcgcccttca

SEQ ID NO: 43 - RNA, 30 nt, synthetic construct
  source 1..30: mol_type = other RNA
cttcagcctctgcttgatctcgcccttcag

SEQ ID NO: 44 - RNA, 30 nt, synthetic construct
  source 1..30: mol_type = other RNA
ttcagcctctgcttgatctcgcccttcagg

SEQ ID NO: 45 - RNA, 30 nt, synthetic construct
  source 1..30: mol_type = other RNA
tcagcctctgcttgatctcgcccttcaggg

SEQ ID NO: 46 - RNA, 30 nt, synthetic construct
  source 1..30: mol_type = other RNA
cagcctctgcttgatctcgcccttcagggc

SEQ ID NO: 47 - RNA, 30 nt, synthetic construct
  source 1..30: mol_type = other RNA
agcctctgcttgatctcgcccttcagggcg

SEQ ID NO: 48 - RNA, 30 nt, synthetic construct
  source 1..30: mol_type = other RNA
gcctctgcttgatctcgcccttcagggcgc

SEQ ID NO: 49 - RNA, 30 nt, synthetic construct
  source 1..30: mol_type = other RNA
cctctgcttgatctcgcccttcagggcgcc

SEQ ID NO: 50 - RNA, 30 nt, synthetic construct
  source 1..30: mol_type = other RNA
ctctgcttgatctcgcccttcagggcgccg

SEQ ID NO: 51 - RNA, 30 nt, synthetic construct
  source 1..30: mol_type = other RNA
tctgcttgatctcgcccttcagggcgccgt

SEQ ID NO: 52 - RNA, 30 nt, synthetic construct
  source 1..30: mol_type = other RNA
ctgcttgatctcgcccttcagggcgccgtc

SEQ ID NO: 53 - RNA, 30 nt, synthetic construct
  source 1..30: mol_type = other RNA
tgcttgatctcgcccttcagggcgccgtcc

SEQ ID NO: 54 - RNA, 30 nt, synthetic construct
  source 1..30: mol_type = other RNA
gcttgatctcgcccttcagggcgccgtcct

SEQ ID NO: 55 - RNA, 30 nt, synthetic construct
  source 1..30: mol_type = other RNA
cttgatctcgcccttcagggcgccgtcctc

SEQ ID NO: 56 - RNA, 30 nt, synthetic construct
  source 1..30: mol_type = other RNA
ttgatctcgcccttcagggcgccgtcctcg

SEQ ID NO: 57 - RNA, 30 nt, synthetic construct; identical to SEQ ID NO: 22
  source 1..30: mol_type = other RNA

SEQ ID NO: 58 - RNA, 30 nt, synthetic construct
  source 1..30: mol_type = other RNA
gatctcgcccttcagggcgccgtcctcggg

SEQ ID NO: 59 - RNA, 30 nt, synthetic construct
  source 1..30: mol_type = other RNA
atctcgcccttcagggcgccgtcctcgggg

SEQ ID NO: 60 - RNA, 30 nt, synthetic construct; identical to SEQ ID NO: 23
  source 1..30: mol_type = other RNA

SEQ ID NO: 61 - RNA, 30 nt, synthetic construct
  source 1..30: mol_type = other RNA
ctcgcccttcagggcgccgtcctcggggta

SEQ ID NO: 62 - RNA, 30 nt, synthetic construct
  source 1..30: mol_type = other RNA
tcgcccttcagggcgccgtcctcggggtac

SEQ ID NO: 63 - RNA, 30 nt, synthetic construct; identical to SEQ ID NO: 24
  source 1..30: mol_type = other RNA

SEQ ID NO: 64 - RNA, 30 nt, synthetic construct
  source 1..30: mol_type = other RNA
gcccttcagggcgccgtcctcggggtacat

SEQ ID NO: 65 - RNA, 30 nt, synthetic construct
  source 1..30: mol_type = other RNA
cccttcagggcgccgtcctcggggtacatc

SEQ ID NO: 66 - RNA, 30 nt, synthetic construct; identical to SEQ ID NO: 25
  source 1..30: mol_type = other RNA

SEQ ID NO: 67 - RNA, 30 nt, synthetic construct
  source 1..30: mol_type = other RNA
cttcagggcgccgtcctcggggtacatccg

SEQ ID NO: 68 - RNA, 30 nt, synthetic construct
  source 1..30: mol_type = other RNA
ttcagggcgccgtcctcggggtacatccgc

SEQ ID NO: 69 - RNA, 30 nt, synthetic construct; identical to SEQ ID NO: 26
  source 1..30: mol_type = other RNA

SEQ ID NO: 70 - RNA, 30 nt, synthetic construct
  source 1..30: mol_type = other RNA
cagggcgccgtcctcggggtacatccgctc

SEQ ID NO: 71 - RNA, 30 nt, synthetic construct
  source 1..30: mol_type = other RNA
agggcgccgtcctcggggtacatccgctcg

SEQ ID NO: 72 - RNA, 30 nt, synthetic construct; identical to SEQ ID NO: 13
  source 1..30: mol_type = other RNA

SEQ ID NO: 73 - RNA, 30 nt, synthetic construct
  source 1..30: mol_type = other RNA
ggcgccgtcctcggggtacatccgctcgga

SEQ ID NO: 74 - RNA, 30 nt, synthetic construct
  source 1..30: mol_type = other RNA
gcgccgtcctcggggtacatccgctcggag

SEQ ID NO: 75 - RNA, 30 nt, synthetic construct; identical to SEQ ID NO: 27
  source 1..30: mol_type = other RNA

SEQ ID NO: 76 - RNA, 30 nt, synthetic construct
  source 1..30: mol_type = other RNA
gccgtcctcggggtacatccgctcggagga

SEQ ID NO: 77 - RNA, 30 nt, synthetic construct
  source 1..30: mol_type = other RNA
ccgtcctcggggtacatccgctcggaggag

SEQ ID NO: 78 - RNA, 30 nt, synthetic construct; identical to SEQ ID NO: 28
  source 1..30: mol_type = other RNA

SEQ ID NO: 79 - RNA, 30 nt, synthetic construct
  source 1..30: mol_type = other RNA
gtcctcggggtacatccgctcggaggaggc

SEQ ID NO: 80 - RNA, 30 nt, synthetic construct
  source 1..30: mol_type = other RNA
tcctcggggtacatccgctcggaggaggcc

SEQ ID NO: 81 - RNA, 30 nt, synthetic construct; identical to SEQ ID NO: 29
  source 1..30: mol_type = other RNA

SEQ ID NO: 82 - RNA, 30 nt, synthetic construct
  source 1..30: mol_type = other RNA
ctcggggtacatccgctcggaggaggcctc

SEQ ID NO: 83 - RNA, 30 nt, synthetic construct
  source 1..30: mol_type = other RNA
tcggggtacatccgctcggaggaggcctcc

SEQ ID NO: 84 - RNA, 30 nt, synthetic construct; identical to SEQ ID NO: 30
  source 1..30: mol_type = other RNA

SEQ ID NO: 85 - RNA, 30 nt, synthetic construct
  source 1..30: mol_type = other RNA
ggggtacatccgctcggaggaggcctccca

SEQ ID NO: 86 - RNA, 30 nt, synthetic construct
  source 1..30: mol_type = other RNA
gggtacatccgctcggaggaggcctcccag

SEQ ID NO: 87 - RNA, 30 nt, synthetic construct; identical to SEQ ID NO: 31
  source 1..30: mol_type = other RNA

SEQ ID NO: 88 - RNA, 30 nt, synthetic construct
  source 1..30: mol_type = other RNA
gtacatccgctcggaggaggcctcccagcc

SEQ ID NO: 89 - RNA, 30 nt, synthetic construct
  source 1..30: mol_type = other RNA
tacatccgctcggaggaggcctcccagccc

SEQ ID NO: 90 - RNA, 30 nt, synthetic construct
  source 1..30: mol_type = other RNA
acatccgctcggaggaggcctcccagccca

SEQ ID NO: 91 - RNA, 30 nt, synthetic construct
  source 1..30: mol_type = other RNA
catccgctcggaggaggcctcccagcccat

SEQ ID NO: 92 - RNA, 30 nt, synthetic construct
  source 1..30: mol_type = other RNA
atccgctcggaggaggcctcccagcccatc

SEQ ID NO: 93 - RNA, 30 nt, synthetic construct
  source 1..30: mol_type = other RNA
tccgctcggaggaggcctcccagcccatcg

SEQ ID NO: 94 - RNA, 30 nt, synthetic construct
  source 1..30: mol_type = other RNA
ccgctcggaggaggcctcccagcccatcgt

SEQ ID NO: 95 - RNA, 30 nt, synthetic construct
  source 1..30: mol_type = other RNA
cgctcggaggaggcctcccagcccatcgtc

SEQ ID NO: 96 - RNA, 30 nt, synthetic construct
  source 1..30: mol_type = other RNA
gctcggaggaggcctcccagcccatcgtct

SEQ ID NO: 97 - RNA, 30 nt, synthetic construct
  source 1..30: mol_type = other RNA
ctcggaggaggcctcccagcccatcgtctt

SEQ ID NO: 98 - RNA, 30 nt, synthetic construct
  source 1..30: mol_type = other RNA
tcggaggaggcctcccagcccatcgtcttc

SEQ ID NO: 99 - RNA, 30 nt, synthetic construct
  source 1..30: mol_type = other RNA
cggaggaggcctcccagcccatcgtcttct

SEQ ID NO: 100 - RNA, 30 nt, synthetic construct
  source 1..30: mol_type = other RNA
ggaggaggcctcccagcccatcgtcttctt

SEQ ID NO: 101 - RNA, 30 nt, synthetic construct
  source 1..30: mol_type = other RNA
gaggaggcctcccagcccatcgtcttcttc

SEQ ID NO: 102 - RNA, 30 nt, synthetic construct
  source 1..30: mol_type = other RNA
aggaggcctcccagcccatcgtcttcttct

SEQ ID NO: 103 - RNA, 30 nt, synthetic construct
  source 1..30: mol_type = other RNA
ggctcaaagtcagatgctactggccgctga

SEQ ID NO: 104 - RNA, 30 nt, synthetic construct
  source 1..30: mol_type = other RNA
gctcaaagtcagatgctactggccgctgaa

SEQ ID NO: 105 - RNA, 30 nt, synthetic construct
  source 1..30: mol_type = other RNA
ctcaaagtcagatgctactggccgctgaag

SEQ ID NO: 106 - RNA, 30 nt, synthetic construct
  source 1..30: mol_type = other RNA
tcaaagtcagatgctactggccgctgaagg

SEQ ID NO: 107 - RNA, 30 nt, synthetic construct
  source 1..30: mol_type = other RNA
caaagtcagatgctactggccgctgaaggg

SEQ ID NO: 108 - RNA, 30 nt, synthetic construct
  source 1..30: mol_type = other RNA
aaagtcagatgctactggccgctgaagggc

SEQ ID NO: 109 - RNA, 30 nt, synthetic construct
  source 1..30: mol_type = other RNA
aagtcagatgctactggccgctgaagggct

SEQ ID NO: 110 - RNA, 30 nt, synthetic construct
  source 1..30: mol_type = other RNA
agtcagatgctactggccgctgaagggctt

SEQ ID NO: 111 - RNA, 30 nt, synthetic construct
  source 1..30: mol_type = other RNA
gtcagatgctactggccgctgaagggcttc

SEQ ID NO: 112 - RNA, 30 nt, synthetic construct
  source 1..30: mol_type = other RNA
tcagatgctactggccgctgaagggcttct

SEQ ID NO: 113 - RNA, 30 nt, synthetic construct
  source 1..30: mol_type = other RNA
cagatgctactggccgctgaagggcttctg

SEQ ID NO: 114 - RNA, 30 nt, synthetic construct
  source 1..30: mol_type = other RNA
agatgctactggccgctgaagggcttctgc

SEQ ID NO: 115 - RNA, 30 nt, synthetic construct
  source 1..30: mol_type = other RNA
gatgctactggccgctgaagggcttctgcg

SEQ ID NO: 116 - RNA, 30 nt, synthetic construct
  source 1..30: mol_type = other RNA
atgctactggccgctgaagggcttctgcgt

SEQ ID NO: 117 - RNA, 30 nt, synthetic construct
  source 1..30: mol_type = other RNA
tgctactggccgctgaagggcttctgcgtc

SEQ ID NO: 118 - RNA, 30 nt, synthetic construct
  source 1..30: mol_type = other RNA
gctactggccgctgaagggcttctgcgtct

SEQ ID NO: 119 - RNA, 30 nt, synthetic construct
  source 1..30: mol_type = other RNA
ctactggccgctgaagggcttctgcgtctc

SEQ ID NO: 120 - RNA, 30 nt, synthetic construct
  source 1..30: mol_type = other RNA
tactggccgctgaagggcttctgcgtctcc

SEQ ID NO: 121 - RNA, 30 nt, synthetic construct
  source 1..30: mol_type = other RNA
actggccgctgaagggcttctgcgtctcca

SEQ ID NO: 122 - RNA, 30 nt, synthetic construct
  source 1..30: mol_type = other RNA
ctggccgctgaagggcttctgcgtctccat

SEQ ID NO: 123 - RNA, 30 nt, synthetic construct
  source 1..30: mol_type = other RNA
tggccgctgaagggcttctgcgtctccatg

SEQ ID NO: 124 - RNA, 30 nt, synthetic construct
  source 1..30: mol_type = other RNA
ggccgctgaagggcttctgcgtctccatgg

SEQ ID NO: 125 - RNA, 30 nt, synthetic construct
  source 1..30: mol_type = other RNA
gccgctgaagggcttctgcgtctccatgga

SEQ ID NO: 126 - RNA, 30 nt, synthetic construct
  source 1..30: mol_type = other RNA
ccgctgaagggcttctgcgtctccatggaa

SEQ ID NO: 127 - RNA, 30 nt, synthetic construct
  source 1..30: mol_type = other RNA
cgctgaagggcttctgcgtctccatggaag

SEQ ID NO: 128 - RNA, 30 nt, synthetic construct
  source 1..30: mol_type = other RNA
gctgaagggcttctgcgtctccatggaagg

SEQ ID NO: 129 - RNA, 30 nt, synthetic construct
  source 1..30: mol_type = other RNA
ctgaagggcttctgcgtctccatggaaggc

SEQ ID NO: 130 - RNA, 30 nt, synthetic construct
  source 1..30: mol_type = other RNA
tgaagggcttctgcgtctccatggaaggcg

SEQ ID NO: 131 - RNA, 30 nt, synthetic construct
  source 1..30: mol_type = other RNA
gaagggcttctgcgtctccatggaaggcgc

SEQ ID NO: 132 - RNA, 30 nt, synthetic construct
  source 1..30: mol_type = other RNA
aagggcttctgcgtctccatggaaggcgcc

SEQ ID NO: 133 - RNA, 30 nt, synthetic construct
  source 1..30: mol_type = other RNA
agggcttctgcgtctccatggaaggcgccc

SEQ ID NO: 134 - RNA, 30 nt, synthetic construct
  source 1..30: mol_type = other RNA
gggcttctgcgtctccatggaaggcgccct

SEQ ID NO: 135 - RNA, 30 nt, synthetic construct
  source 1..30: mol_type = other RNA
ggcttctgcgtctccatggaaggcgccctc

SEQ ID NO: 136 - RNA, 30 nt, synthetic construct
  source 1..30: mol_type = other RNA
gcttctgcgtctccatggaaggcgccctcg

SEQ ID NO: 137 - RNA, 30 nt, synthetic construct
  source 1..30: mol_type = other RNA
cttctgcgtctccatggaaggcgccctcgc

SEQ ID NO: 138 - RNA, 30 nt, synthetic construct
  source 1..30: mol_type = other RNA
ttctgcgtctccatggaaggcgccctcgcc

SEQ ID NO: 139 - RNA, 30 nt, synthetic construct
  source 1..30: mol_type = other RNA
tctgcgtctccatggaaggcgccctcgcca

SEQ ID NO: 140 - RNA, 30 nt, synthetic construct
  source 1..30: mol_type = other RNA
ctgcgtctccatggaaggcgccctcgccat

SEQ ID NO: 141 - RNA, 30 nt, synthetic construct
  source 1..30: mol_type = other RNA
tgcgtctccatggaaggcgccctcgccatc

SEQ ID NO: 142 - RNA, 30 nt, synthetic construct
  source 1..30: mol_type = other RNA
gcgtctccatggaaggcgccctcgccatcg

SEQ ID NO: 143 - RNA, 30 nt, synthetic construct
  source 1..30: mol_type = other RNA
cgtctccatggaaggcgccctcgccatcgt

SEQ ID NO: 144 - RNA, 30 nt, synthetic construct
  source 1..30: mol_type = other RNA
gaagcggctctcggaggagacgtagagctc

SEQ ID NO: 145 - RNA, 30 nt, synthetic construct
  source 1..30: mol_type = other RNA
gcggctctcggaggagacgtagagctcttc

SEQ ID NO: 146 - RNA, 30 nt, synthetic construct
  source 1..30: mol_type = other RNA
gctctcggaggagacgtagagctcttccct

SEQ ID NO: 147 - RNA, 30 nt, synthetic construct
  source 1..30: mol_type = other RNA
ctcggaggagacgtagagctcttccctgga

SEQ ID NO: 148 - RNA, 30 nt, synthetic construct
  source 1..30: mol_type = other RNA
ggaggagacgtagagctcttccctggatct

SEQ ID NO: 149 - RNA, 30 nt, synthetic construct
  source 1..30: mol_type = other RNA
ggagacgtagagctcttccctggatctatc

SEQ ID NO: 150 - RNA, 30 nt, synthetic construct
  source 1..30: mol_type = other RNA
gacgtagagctcttccctggatctatcaaa

SEQ ID NO: 151 - RNA, 30 nt, synthetic construct
  source 1..30: mol_type = other RNA
gtagagctcttccctggatctatcaaagat

SEQ ID NO: 152 - RNA, 30 nt, synthetic construct
  source 1..30: mol_type = other RNA
gagctcttccctggatctatcaaagatcac

SEQ ID NO: 153 - RNA, 30 nt, synthetic construct
  source 1..30: mol_type = other RNA
gaagcggctctcggaggagacgtagagcat

SEQ ID NO: 154 - RNA, 30 nt, synthetic construct
  source 1..30: mol_type = other RNA
gcggctctcggaggagacgtagagcatgtc

SEQ ID NO: 155 - RNA, 30 nt, synthetic construct
  source 1..30: mol_type = other RNA
gctctcggaggagacgtagagcatgtcact

SEQ ID NO: 156 - RNA, 30 nt, synthetic construct
  source 1..30: mol_type = other RNA
ctcggaggagacgtagagcatgtcacttcc

SEQ ID NO: 157 - RNA, 30 nt, synthetic construct
  source 1..30: mol_type = other RNA
ggaggagacgtagagcatgtcacttcccat

SEQ ID NO: 158 - RNA, 30 nt, synthetic construct
  source 1..30: mol_type = other RNA
ggagacgtagagcatgtcacttcccatcat

SEQ ID NO: 159 - RNA, 30 nt, synthetic construct
  source 1..30: mol_type = other RNA
gacgtagagcatgtcacttcccatcatgga

SEQ ID NO: 160 - RNA, 30 nt, synthetic construct
  source 1..30: mol_type = other RNA
gtagagcatgtcacttcccatcatggaacc

SEQ ID NO: 161 - RNA, 30 nt, synthetic construct
  source 1..30: mol_type = other RNA
gagcatgtcacttcccatcatggaaccact

SEQ ID NO: 162 - RNA, 30 nt, synthetic construct
  source 1..30: mol_type = other RNA
gtcgcgcaccaggttgattggggtgtgctt

SEQ ID NO: 163 - RNA, 30 nt, synthetic construct
  source 1..30: mol_type = other RNA
tcgcgcaccaggttgattggggtgtgcttg

SEQ ID NO: 164 - RNA, 30 nt, synthetic construct
  source 1..30: mol_type = other RNA
cgcgcaccaggttgattggggtgtgcttgg

SEQ ID NO: 165 - RNA, 30 nt, synthetic construct
  source 1..30: mol_type = other RNA
gcgcaccaggttgattggggtgtgcttgga

SEQ ID NO: 166 - RNA, 30 nt, synthetic construct
  source 1..30: mol_type = other RNA
cgcaccaggttgattggggtgtgcttggag

SEQ ID NO: 167 - RNA, 30 nt, synthetic construct
  source 1..30: mol_type = other RNA
gcaccaggttgattggggtgtgcttggagt

SEQ ID NO: 168 - RNA, 30 nt, synthetic construct
  source 1..30: mol_type = other RNA
caccaggttgattggggtgtgcttggagta

SEQ ID NO: 169 - RNA, 30 nt, synthetic construct
  source 1..30: mol_type = other RNA
accaggttgattggggtgtgcttggagtag

SEQ ID NO: 170 - RNA, 30 nt, synthetic construct
  source 1..30: mol_type = other RNA
ccaggttgattggggtgtgcttggagtaga

SEQ ID NO: 171 - RNA, 30 nt, synthetic construct
  source 1..30: mol_type = other RNA
caggttgattggggtgtgcttggagtagat

SEQ ID NO: 172 - RNA, 30 nt, synthetic construct
  source 1..30: mol_type = other RNA
aggttgattggggtgtgcttggagtagatc

SEQ ID NO: 173 - RNA, 30 nt, synthetic construct
  source 1..30: mol_type = other RNA
ggttgattggggtgtgcttggagtagatct

SEQ ID NO: 174 - RNA, 30 nt, synthetic construct
  source 1..30: mol_type = other RNA
gttgattggggtgtgcttggagtagatctt

SEQ ID NO: 175 - RNA, 30 nt, synthetic construct
  source 1..30: mol_type = other RNA
ttgattggggtgtgcttggagtagatctta

SEQ ID NO: 176 - RNA, 30 nt, synthetic construct
  source 1..30: mol_type = other RNA
tgattggggtgtgcttggagtagatcttaa

SEQ ID NO: 177 - RNA, 30 nt, synthetic construct
  source 1..30: mol_type = other RNA
gattggggtgtgcttggagtagatcttaaa

SEQ ID NO: 178 - RNA, 30 nt, synthetic construct
  source 1..30: mol_type = other RNA
attggggtgtgcttggagtagatcttaaag

SEQ ID NO: 179 - RNA, 30 nt, synthetic construct
  source 1..30: mol_type = other RNA
ttggggtgtgcttggagtagatcttaaagt

SEQ ID NO: 180 - RNA, 30 nt, synthetic construct
  source 1..30: mol_type = other RNA
tggggtgtgcttggagtagatcttaaagta

SEQ ID NO: 181 - RNA, 30 nt, synthetic construct
  source 1..30: mol_type = other RNA
ggggtgtgcttggagtagatcttaaagtag

SEQ ID NO: 182 - RNA, 30 nt, synthetic construct
  source 1..30: mol_type = other RNA
gggtgtgcttggagtagatcttaaagtagc

SEQ ID NO: 183 - RNA, 30 nt, synthetic construct
  source 1..30: mol_type = other RNA
ggtgtgcttggagtagatcttaaagtagcc

SEQ ID NO: 184 - RNA, 30 nt, synthetic construct
  source 1..30: mol_type = other RNA
gtgtgcttggagtagatcttaaagtagcca

SEQ ID NO: 185 - RNA, 30 nt, synthetic construct
  source 1..30: mol_type = other RNA
tgtgcttggagtagatcttaaagtagccat

SEQ ID NO: 186 - RNA, 30 nt, synthetic construct
  source 1..30: mol_type = other RNA
gtgcttggagtagatcttaaagtagccatc

SEQ ID NO: 187 - RNA, 30 nt, synthetic construct
  source 1..30: mol_type = other RNA
tgcttggagtagatcttaaagtagccatcg

SEQ ID NO: 188 - RNA, 30 nt, synthetic construct
  source 1..30: mol_type = other RNA
gcttggagtagatcttaaagtagccatcga

SEQ ID NO: 189 - RNA, 30 nt, synthetic construct
  source 1..30: mol_type = other RNA
cttggagtagatcttaaagtagccatcgat

SEQ ID NO: 190 - RNA, 30 nt, synthetic construct
  source 1..30: mol_type = other RNA
ttggagtagatcttaaagtagccatcgatg

SEQ ID NO: 191 - RNA, 30 nt, synthetic construct
  source 1..30: mol_type = other RNA
tggagtagatcttaaagtagccatcgatgt

SEQ ID NO: 192 - RNA, 30 nt, synthetic construct
  source 1..30: mol_type = other RNA
ggagtagatcttaaagtagccatcgatgtt

SEQ ID NO: 193 - RNA, 30 nt, synthetic construct
  source 1..30: mol_type = other RNA
gagtagatcttaaagtagccatcgatgttc

SEQ ID NO: 194 - RNA, 30 nt, synthetic construct
  source 1..30: mol_type = other RNA
agtagatcttaaagtagccatcgatgttct

SEQ ID NO: 195 - RNA, 30 nt, synthetic construct
  source 1..30: mol_type = other RNA
gtagatcttaaagtagccatcgatgttctt

SEQ ID NO: 196 - RNA, 30 nt, synthetic construct
  source 1..30: mol_type = other RNA
tagatcttaaagtagccatcgatgttctta

SEQ ID NO: 197 - RNA, 30 nt, synthetic construct
  source 1..30: mol_type = other RNA
agatcttaaagtagccatcgatgttcttaa

SEQ ID NO: 198 - RNA, 30 nt, synthetic construct
  source 1..30: mol_type = other RNA
gatcttaaagtagccatcgatgttcttaaa

SEQ ID NO: 199 - RNA, 30 nt, synthetic construct
  source 1..30: mol_type = other RNA
atcttaaagtagccatcgatgttcttaaac

SEQ ID NO: 200 - RNA, 30 nt, synthetic construct
  source 1..30: mol_type = other RNA
tcttaaagtagccatcgatgttcttaaaca

SEQ ID NO: 201 - RNA, 30 nt, synthetic construct
  source 1..30: mol_type = other RNA
cttaaagtagccatcgatgttcttaaacac

SEQ ID NO: 202 - RNA, 30 nt, synthetic construct
  source 1..30: mol_type = other RNA
ttaaagtagccatcgatgttcttaaacacg

SEQ ID NO: 203 - RNA, 30 nt, synthetic construct
  source 1..30: mol_type = other RNA
taaagtagccatcgatgttcttaaacacga

SEQ ID NO: 204 - RNA, 30 nt, synthetic construct
  source 1..30: mol_type = other RNA
aaagtagccatcgatgttcttaaacacgaa

SEQ ID NO: 205 - RNA, 30 nt, synthetic construct
  source 1..30: mol_type = other RNA
aagtagccatcgatgttcttaaacacgaac

SEQ ID NO: 206 - RNA, 30 nt, synthetic construct
  source 1..30: mol_type = other RNA
agtagccatcgatgttcttaaacacgaact

SEQ ID NO: 207 - RNA, 30 nt, synthetic construct
  source 1..30: mol_type = other RNA
gtagccatcgatgttcttaaacacgaactc

SEQ ID NO: 208 - RNA, 30 nt, synthetic construct
  source 1..30: mol_type = other RNA
tagccatcgatgttcttaaacacgaactcc

SEQ ID NO: 209 - RNA, 30 nt, synthetic construct
  source 1..30: mol_type = other RNA
agccatcgatgttcttaaacacgaactccc

SEQ ID NO: 210 - RNA, 30 nt, synthetic construct
  source 1..30: mol_type = other RNA
gccatcgatgttcttaaacacgaactcccg

SEQ ID NO: 211 - RNA, 30 nt, synthetic construct
  source 1..30: mol_type = other RNA
ccatcgatgttcttaaacacgaactcccgc

SEQ ID NO: 212 - RNA, 30 nt, synthetic construct
  source 1..30: mol_type = other RNA
catcgatgttcttaaacacgaactcccgca

SEQ ID NO: 213 - RNA, 30 nt, synthetic construct
  source 1..30: mol_type = other RNA
atcgatgttcttaaacacgaactcccgcag

SEQ ID NO: 214 - RNA, 30 nt, synthetic construct
  source 1..30: mol_type = other RNA
tcgatgttcttaaacacgaactcccgcagg

SEQ ID NO: 215 - RNA, 30 nt, synthetic construct
  source 1..30: mol_type = other RNA
cgatgttcttaaacacgaactcccgcaggt

SEQ ID NO: 216 - RNA, 30 nt, synthetic construct
  source 1..30: mol_type = other RNA
gatgttcttaaacacgaactcccgcaggtt

SEQ ID NO: 217 - RNA, 30 nt, synthetic construct
  source 1..30: mol_type = other RNA
atgttcttaaacacgaactcccgcaggttc

SEQ ID NO: 218 - RNA, 30 nt, synthetic construct
  source 1..30: mol_type = other RNA
tgttcttaaacacgaactcccgcaggttct

SEQ ID NO: 219 - RNA, 30 nt, synthetic construct
  source 1..30: mol_type = other RNA
gttcttaaacacgaactcccgcaggttctt

SEQ ID NO: 220 - RNA, 30 nt, synthetic construct
  source 1..30: mol_type = other RNA
ttcttaaacacgaactcccgcaggttcttg

SEQ ID NO: 221 - RNA, 30 nt, synthetic construct
  source 1..30: mol_type = other RNA
tcttaaacacgaactcccgcaggttcttga

SEQ ID NO: 222 - RNA, 30 nt, synthetic construct
  source 1..30: mol_type = other RNA
cttaaacacgaactcccgcaggttcttgaa

SEQ ID NO: 223 - RNA, 30 nt, synthetic construct
  source 1..30: mol_type = other RNA
ccccgccgtcctcggggtacatccgctcgg

SEQ ID NO: 224 - RNA, 30 nt, synthetic construct
  source 1..30: mol_type = other RNA
ggggcgcgtcctcggggtacatccgctcgg

SEQ ID NO: 225 - RNA, 30 nt, synthetic construct
  source 1..30: mol_type = other RNA
gggcgcgcacctcggggtacatccgctcgg

SEQ ID NO: 226 - RNA, 30 nt, synthetic construct
  source 1..30: mol_type = other RNA
gggcgccgtggacggggtacatccgctcgg

SEQ ID NO: 227 - RNA, 30 nt, synthetic construct
  source 1..30: mol_type = other RNA
gggcgccgtcctgccggtacatccgctcgg

SEQ ID NO: 228 - RNA, 30 nt, synthetic construct
  source 1..30: mol_type = other RNA
gggcgccgtcctcggccaacatccgctcgg

SEQ ID NO: 229 - RNA, 30 nt, synthetic construct
  source 1..30: mol_type = other RNA
gggcgccgtcctcggggttgttccgctcgg

SEQ ID NO: 230 - RNA, 30 nt, synthetic construct
  source 1..30: mol_type = other RNA
gggcgccgtcctcggggtacaagggctcgg

SEQ ID NO: 231 - RNA, 30 nt, synthetic construct
  source 1..30: mol_type = other RNA
gggcgccgtcctcggggtacatcccgacgg

SEQ ID NO: 232 - RNA, 30 nt, synthetic construct
  source 1..30: mol_type = other RNA
gggcgccgtcctcggggtacatccgctgcc

SEQ ID NO: 233 - RNA, 30 nt, synthetic construct
  source 1..30: mol_type = other RNA
cccggccgtcctcggggtacatccgctcgg

SEQ ID NO: 234 - RNA, 30 nt, synthetic construct
  source 1..30: mol_type = other RNA
gggccggctcctcggggtacatccgctcgg

SEQ ID NO: 235 - RNA, 30 nt, synthetic construct
  source 1..30: mol_type = other RNA
gggcgccgaggacggggtacatccgctcgg

SEQ ID NO: 236 - RNA, 30 nt, synthetic construct
  source 1..30: mol_type = other RNA
gggcgccgtcctgcccgtacatccgctcgg

SEQ ID NO: 237 - RNA, 30 nt, synthetic construct
  source 1..30: mol_type = other RNA
gggcgccgtcctcgggcatgatccgctcgg

SEQ ID NO: 238 - RNA, 30 nt, synthetic construct
  source 1..30: mol_type = other RNA
gggcgccgtcctcggggtactagggctcgg

SEQ ID NO: 239 - RNA, 30 nt, synthetic construct
  source 1..30: mol_type = other RNA
gggcgccgtcctcggggtacatcccgaggg

SEQ ID NO: 240 - RNA, 30 nt, synthetic construct
  source 1..30: mol_type = other RNA
cccgcccgtcctcggggtacatccgctcgg

SEQ ID NO: 241 - RNA, 30 nt, synthetic construct
  source 1..30: mol_type = other RNA
gggcgggcagctcggggtacatccgctcgg

SEQ ID NO: 242 - RNA, 30 nt, synthetic construct
  source 1..30: mol_type = other RNA
gggcgccgtcgagccggtacatccgctcgg

SEQ ID NO: 243 - RNA, 30 nt, synthetic construct
  source 1..30: mol_type = other RNA
gggcgccgtcctcggccatgatccgctcgg

SEQ ID NO: 244 - RNA, 30 nt, synthetic construct
  source 1..30: mol_type = other RNA
gggcgccgtcctcggggtactaggcctcgg

SEQ ID NO: 245 - RNA, 30 nt, synthetic construct
  source 1..30: mol_type = other RNA
gggcgccgtcctcggggtacatccggagcc

SEQ ID NO: 246 - RNA, 30 nt, synthetic construct
  source 1..30: mol_type = other RNA
cccgcgcgtcctcggggtacatccgctcgg

SEQ ID NO: 247 - RNA, 30 nt, synthetic construct
  source 1..30: mol_type = other RNA
gggcgcgcaggacggggtacatccgctcgg

SEQ ID NO: 248 - RNA, 30 nt, synthetic construct
  source 1..30: mol_type = other RNA
gggcgccgtcctgccccaacatccgctcgg

SEQ ID NO: 249 - RNA, 30 nt, synthetic construct
  source 1..30: mol_type = other RNA
gggcgccgtcctcggggttgtagggctcgg

SEQ ID NO: 250 - RNA, 30 nt, synthetic construct
  source 1..30: mol_type = other RNA
gggcgccgtcctcggggtacatcccgagcc

SEQ ID NO: 251 - RNA, 30 nt, synthetic construct; identical to SEQ ID NO: 232
  source 1..30: mol_type = other RNA

SEQ ID NO: 252 - RNA, 30 nt, synthetic construct; identical to SEQ ID NO: 250
  source 1..30: mol_type = other RNA

SEQ ID NO: 253 - RNA, 30 nt, synthetic construct
  source 1..30: mol_type = other RNA
gggcgccgtcctcggggtacaaggcgagcc

SEQ ID NO: 254 - RNA, 30 nt, synthetic construct
  source 1..30: mol_type = other RNA
gggcgccgtcctcggggttgtaggcgagcc

SEQ ID NO: 255 - RNA, 30 nt, synthetic construct
  source 1..30: mol_type = other RNA
gggcgccgtcctcggccatgtaggcgagcc

SEQ ID NO: 256 - RNA, 30 nt, synthetic construct
  source 1..30: mol_type = other RNA
gggcgccgtcctgccccatgtaggcgagcc

SEQ ID NO: 257 - RNA, 30 nt, synthetic construct
  source 1..30: mol_type = other RNA
gggcgccgtggagccccatgtaggcgagcc

SEQ ID NO: 258 - RNA, 30 nt, synthetic construct
  source 1..30: mol_type = other RNA
gggcgcgcaggagccccatgtaggcgagcc

SEQ ID NO: 259 - RNA, 30 nt, synthetic construct
  source 1..30: mol_type = other RNA
ggggcggcaggagccccatgtaggcgagcc

SEQ ID NO: 260 - RNA, 30 nt, synthetic construct
  source 1..30: mol_type = other RNA
cccgcggcaggagccccatgtaggcgagcc

SEQ ID NO: 261 - RNA, 30 nt, synthetic construct; identical to SEQ ID NO: 223
  source 1..30: mol_type = other RNA

SEQ ID NO: 262 - RNA, 30 nt, synthetic construct; identical to SEQ ID NO: 246
  source 1..30: mol_type = other RNA

SEQ ID NO: 263 - RNA, 30 nt, synthetic construct
  source 1..30: mol_type = other RNA
cccgcggcacctcggggtacatccgctcgg

SEQ ID NO: 264 - RNA, 30 nt, synthetic construct
  source 1..30: mol_type = other RNA
cccgcggcaggacggggtacatccgctcgg

SEQ ID NO: 265 - RNA, 30 nt, synthetic construct
  source 1..30: mol_type = other RNA
cccgcggcaggagccggtacatccgctcgg

SEQ ID NO: 266 - RNA, 30 nt, synthetic construct
  source 1..30: mol_type = other RNA
cccgcggcaggagccccaacatccgctcgg

SEQ ID NO: 267 - RNA, 30 nt, synthetic construct
  source 1..30: mol_type = other RNA
cccgcggcaggagccccatgttccgctcgg

SEQ ID NO: 268 - RNA, 30 nt, synthetic construct
  source 1..30: mol_type = other RNA
cccgcggcaggagccccatgtagggctcgg

SEQ ID NO: 269 - RNA, 30 nt, synthetic construct
  source 1..30: mol_type = other RNA
cccgcggcaggagccccatgtaggcgacgg

SEQ ID NO: 270 - RNA, 30 nt, synthetic construct; identical to SEQ ID NO: 260
  source 1..30: mol_type = other RNA

SEQ ID NO: 271 - RNA, 30 nt, synthetic construct
  source 1..30: mol_type = other RNA
ccgcatgttatcctcctcgcccttgctcac

SEQ ID NO: 272 - RNA, 30 nt, synthetic construct
  source 1..30: mol_type = other RNA
cctgccctccatgtgcaccttgaagcgcat

SEQ ID NO: 273 - RNA, 30 nt, synthetic construct
  source 1..30: mol_type = other RNA
ccgggtctgggtgccctcgtaggggcggcc

SEQ ID NO: 274 - RNA, 30 nt, synthetic construct
  source 1..30: mol_type = other RNA
cctgccgtacatgaactgaggggacaggat

SEQ ID NO: 275 - RNA, 30 nt, synthetic construct
  source 1..30: mol_type = other RNA
cccgaaggacagcttcaagtagtcggggat

SEQ ID NO: 276 - RNA, 30 nt, synthetic construct
  source 1..30: mol_type = other RNA
cctgtcctgggtcacggtcaccacgccgcc

SEQ ID NO: 277 - RNA, 30 nt, synthetic construct
  source 1..30: mol_type = other RNA
ccacttcttctgcattacggggccgtcgga

SEQ ID NO: 278 - RNA, 30 nt, synthetic construct; identical to SEQ ID NO: 223
  source 1..30: mol_type = other RNA

SEQ ID NO: 279 - RNA, 30 nt, synthetic construct
  source 1..30: mol_type = other RNA
ccaggtcttgacctcagcgtcgtagtggcc

SEQ ID NO: 280 - RNA, 30 nt, synthetic construct
  source 1..30: mol_type = other RNA
ccagatgtccaacttgatgttgacgttgta

SEQ ID NO: 281 - RNA, 30 nt, synthetic construct
  source 1..30: mol_type = other RNA
ccaggagtggcggccctcggcgcgttcgta

SEQ ID NO: 282 - RNA, 30 nt, synthetic construct
  source 1..30: mol_type = other RNA
ccgcgccgtcctcggggtacatccgctcgg

SEQ ID NO: 283 - RNA, 30 nt, synthetic construct
  source 1..30: mol_type = other RNA
gcccgccgtcctcggggtacatccgctcgg

SEQ ID NO: 284 - RNA, 30 nt, synthetic construct
  source 1..30: mol_type = other RNA
cgccgccgtcctcggggtacatccgctcgg

SEQ ID NO: 285 - RNA, 30 nt, synthetic construct
  source 1..30: mol_type = other RNA
cggcgccgtcctcggggtacatccgctcgg

SEQ ID NO: 286 - RNA, 30 nt, synthetic construct; identical to SEQ ID NO: 13
  source 1..30: mol_type = other RNA

SEQ ID NO: 287 - RNA, 30 nt, synthetic construct
  source 1..30: mol_type = other RNA
ggccgccgtcctcggggtacatccgctcgg

SEQ ID NO: 288 - RNA, 30 nt, synthetic construct
  source 1..30: mol_type = other RNA
gggcggcgtccacggggaacatcggctcgc

SEQ ID NO: 289 - RNA, 30 nt, synthetic construct
  source 1..30: mol_type = other RNA
gggcgcggtcctccgggtacttccgctggg

SEQ ID NO: 290 - RNA, 30 nt, synthetic construct
  source 1..30: mol_type = other RNA
gggcgccctcctcggcgtacatcggctcgg

SEQ ID NO: 291 - RNA, 30 nt, synthetic construct
  source 1..30: mol_type = other RNA
gggcgccgtgctcggggtagatccgctcgg

SEQ ID NO: 292 - RNA, 30 nt, synthetic construct
  source 1..30: mol_type = other RNA
gcggggcctgcaccgcgaagaacgggtggc

SEQ ID NO: 293 - RNA, 30 nt, synthetic construct
  source 1..30: mol_type = other RNA
ggccggcgaccacgcggaacttcggcacgc

SEQ ID NO: 294 - RNA, 30 nt, synthetic construct
  source 1..30: mol_type = other RNA
gggggccctccacggcgtagatcggctggg

SEQ ID NO: 295 - RNA, 30 nt, synthetic construct
  source 1..30: mol_type = other RNA
gggccccgtgctcgcggtagatcccctcgc

SEQ ID NO: 296 - RNA, 30 nt, synthetic construct
  source 1..30: mol_type = other RNA
ggcggcgctcgacgccgttgatgggcaggg

SEQ ID NO: 297 - RNA, 30 nt, synthetic construct
  source 1..30: mol_type = other RNA
ggggcgcgtggacggccaacaagggctgcc

SEQ ID NO: 298 - RNA, 30 nt, synthetic construct
  source 1..30: mol_type = other RNA
gccttcagggcgccgtcctcggggtacatc

SEQ ID NO: 299 - RNA, 30 nt, synthetic construct
  source 1..30: mol_type = other RNA
gcttcagggcgccgtcctcggggtacatcc

SEQ ID NO: 300 - RNA, 30 nt, synthetic construct
  source 1..30: mol_type = other RNA
gttcagggcgccgtcctcggggtacatccg

SEQ ID NO: 301 - RNA, 30 nt, synthetic construct
  source 1..30: mol_type = other RNA
gtcagggcgccgtcctcggggtacatccgc

SEQ ID NO: 302 - RNA, 30 nt, synthetic construct
  source 1..30: mol_type = other RNA
gcagggcgccgtcctcggggtacatccgct

SEQ ID NO: 303 - RNA, 30 nt, synthetic construct
  source 1..30: mol_type = other RNA
ggcttcagggcgccgtcctcggggtacatc

SEQ ID NO: 304 - RNA, 30 nt, synthetic construct
  source 1..30: mol_type = other RNA
ggttcagggcgccgtcctcggggtacatcc

SEQ ID NO: 305 - RNA, 30 nt, synthetic construct
  source 1..30: mol_type = other RNA
ggtcagggcgccgtcctcggggtacatccg

SEQ ID NO: 306 - RNA, 30 nt, synthetic construct
  source 1..30: mol_type = other RNA
ggcagggcgccgtcctcggggtacatccgc

SEQ ID NO: 307 - RNA, 30 nt, synthetic construct
  source 1..30: mol_type = other RNA
ggagggcgccgtcctcggggtacatccgct

SEQ ID NO: 308 - RNA, 31 nt, synthetic construct
  source 1..31: mol_type = other RNA
gtcctcggggtacatccgctcggaggaggcc

SEQ ID NO: 309 - RNA, 31 nt, synthetic construct
  source 1..31: mol_type = other RNA
gcctcggggtacatccgctcggaggaggcct

SEQ ID NO: 310 - RNA, 30 nt, synthetic construct
  source 1..30: mol_type = other RNA
gcctcggggtacatccgctcggaggaggcc

SEQ ID NO: 311 - RNA, 30 nt, synthetic construct
  source 1..30: mol_type = other RNA
gctcggggtacatccgctcggaggaggcct

SEQ ID NO: 312 - RNA, 30 nt, synthetic construct
  source 1..30: mol_type = other RNA
ggctcggggtacatccgctcggaggaggcc

SEQ ID NO: 313 - RNA, 30 nt, synthetic construct
  source 1..30: mol_type = other RNA
ggtcggggtacatccgctcggaggaggcct

SEQ ID NO: 314 - RNA, 31 nt, synthetic construct
  source 1..31: mol_type = other RNA
gcccttcagggcgccgtcctcggggtacatc

SEQ ID NO: 315 - RNA, 31 nt, synthetic construct
  source 1..31: mol_type = other RNA
gccttcagggcgccgtcctcggggtacatcc

SEQ ID NO: 316 - RNA, 31 nt, synthetic construct
  source 1..31: mol_type = other RNA
gcttcagggcgccgtcctcggggtacatccg

SEQ ID NO: 317 - RNA, 31 nt, synthetic construct
  source 1..31: mol_type = other RNA
gttcagggcgccgtcctcggggtacatccgc

SEQ ID NO: 318 - RNA, 31 nt, synthetic construct
  source 1..31: mol_type = other RNA
gtcagggcgccgtcctcggggtacatccgct

SEQ ID NO: 319 - RNA, 30 nt, synthetic construct
  source 1..30: mol_type = other RNA
ggctggccgctgaagggcttctgcgtctcc

SEQ ID NO: 320 - RNA, 30 nt, synthetic construct
  source 1..30: mol_type = other RNA
gggaagggcttctgcgtctccatggaaggc

SEQ ID NO: 321 - RNA, 30 nt, synthetic construct
  source 1..30: mol_type = other RNA
gggggcttctgcgtctccatggaaggcgcc

SEQ ID NO: 322 - RNA, 30 nt, synthetic construct
  source 1..30: mol_type = other RNA
aagggcttctgcgtgtggatggaaggcgcc

SEQ ID NO: 323 - RNA, 30 nt, synthetic construct
  source 1..30: mol_type = other RNA
aagggcttctgcgtctggatggaaggcgcc

SEQ ID NO: 324 - RNA, 30 nt, synthetic construct
  source 1..30: mol_type = other RNA
gggggcttctgcgtgtggatggaaggcgcc

SEQ ID NO: 325 - RNA, 30 nt, synthetic construct
  source 1..30: mol_type = other RNA
ggcgtagagctcttccctggatctatcaaa

SEQ ID NO: 326 - RNA, 30 nt, synthetic construct
  source 1..30: mol_type = other RNA
gacgtagagctcttggctggatctatcaaa

SEQ ID NO: 327 - RNA, 30 nt, synthetic construct
  source 1..30: mol_type = other RNA
gacgtagagctcttgggtggatctatcaaa

SEQ ID NO: 328 - RNA, 30 nt, synthetic construct
  source 1..30: mol_type = other RNA
ggcgtagagctcttgggtggatctatcaaa

SEQ ID NO: 329 - RNA, 30 nt, synthetic construct
  source 1..30: mol_type = other RNA
gggctcttccctggatctatcaaagatcac

SEQ ID NO: 330 - RNA, 30 nt, synthetic construct
  source 1..30: mol_type = other RNA
gagctcttccgtggatctatcaaagatcac

SEQ ID NO: 331 - RNA, 30 nt, synthetic construct
  source 1..30: mol_type = other RNA
gagctcttccctggatgtatcaaagatcac

SEQ ID NO: 332 - RNA, 30 nt, synthetic construct
  source 1..30: mol_type = other RNA
gggctcttccgtggatgtatcaaagatcac

SEQ ID NO: 333 - RNA, 30 nt, synthetic construct
  source 1..30: mol_type = other RNA
ggagacgtagagctgttgcctggatctatc

SEQ ID NO: 334 - RNA, 30 nt, synthetic construct
  source 1..30: mol_type = other RNA
ggagacgtagagctgttccctggatctatc

SEQ ID NO: 335 - RNA, 30 nt, synthetic construct
  source 1..30: mol_type = other RNA
accccgctgaagggcttctgcgtctccatg

SEQ ID NO: 336 - RNA, 30 nt, synthetic construct
  source 1..30: mol_type = other RNA
accggcctgaagggcttctgcgtctccatg

SEQ ID NO: 337 - RNA, 30 nt, synthetic construct
  source 1..30: mol_type = other RNA
accggcgacaagggcttctgcgtctccatg

SEQ ID NO: 338 - RNA, 30 nt, synthetic construct
  source 1..30: mol_type = other RNA
tggccgctgaagggcttctgcgtctcctac

SEQ ID NO: 339 - RNA, 30 nt, synthetic construct
  source 1..30: mol_type = other RNA
tggccgctgaagggcttctgcgtcaggtac

SEQ ID NO: 340 - RNA, 30 nt, synthetic construct
  source 1..30: mol_type = other RNA
tggccgctgaagggcttctgccagaggtac

SEQ ID NO: 341 - RNA, 30 nt, synthetic construct
  source 1..30: mol_type = other RNA
tggccgctgaagccgttctgcgtctccatg

SEQ ID NO: 342 - RNA, 30 nt, synthetic construct
  source 1..30: mol_type = other RNA
tggccgctgaacccgaactgcgtctccatg

SEQ ID NO: 343 - RNA, 30 nt, synthetic construct
  source 1..30: mol_type = other RNA
tggccgctgttcccgaagtgcgtctccatg

SEQ ID NO: 344 - RNA, 30 nt, synthetic construct
  source 1..30: mol_type = other RNA
tggccgcagaagggcatctgcgtgtccatg

SEQ ID NO: 345 - RNA, 30 nt, synthetic construct
  source 1..30: mol_type = other RNA
tggccggtgaaggccttctgcctctccaag

SEQ ID NO: 346 - RNA, 30 nt, synthetic construct
  source 1..30: mol_type = other RNA
tggcccctgaacggcttgtgcgtgtccatc

SEQ ID NO: 347 - RNA, 30 nt, synthetic construct
  source 1..30: mol_type = other RNA
tggcggctgtaggggttctccgtcaccatc

SEQ ID NO: 348 - RNA, 30 nt, synthetic construct
  source 1..30: mol_type = other RNA
tgggcgcagaacggcatctccgtgtccttg

SEQ ID NO: 349 - RNA, 30 nt, synthetic construct
  source 1..30: mol_type = other RNA
tgccccctcaacgggttgtgggtgtcgatc

SEQ ID NO: 350 - RNA, 30 nt, synthetic construct
  source 1..30: mol_type = other RNA
tgcgcggagatcgggatcaccgagtcgttg

SEQ ID NO: 351 - RNA, 30 nt, synthetic construct
  source 1..30: mol_type = other RNA
ccggtgaacagctcctcgcccttgctcacc

SEQ ID NO: 352 - RNA, 30 nt, synthetic construct
  source 1..30: mol_type = other RNA
caggatgggcaccaccccggtgaacagctc

SEQ ID NO: 353 - RNA, 30 nt, synthetic construct
  source 1..30: mol_type = other RNA
cttgtagttgccgtcgtccttgaagaagat

SEQ ID NO: 354 - RNA, 30 nt, synthetic construct
  source 1..30: mol_type = other RNA
caccagggtgtcgccctcgaacttcacctc

SEQ ID NO: 355 - RNA, 30 nt, synthetic construct
  source 1..30: mol_type = other RNA
caggatgttgccgtcctccttgaagtcgat

SEQ ID NO: 356 - RNA, 30 nt, synthetic construct
  source 1..30: mol_type = other RNA
cttctgcttgtcggccatgatatagacgtt

SEQ ID NO: 357 - RNA, 30 nt, synthetic construct
  source 1..30: mol_type = other RNA
ctgcacgctgccgtcctcgatgttgtggcg

SEQ ID NO: 358 - RNA, 30 nt, synthetic construct
  source 1..30: mol_type = other RNA
cttgtacagctcgtccatgccgagagtgat

SEQ ID NO: 359 - RNA, 30 nt, synthetic construct
  source 1..30: mol_type = other RNA
tactccagcttgtgccccaggatgttgccg

SEQ ID NO: 360 - RNA, 30 nt, synthetic construct
  source 1..30: mol_type = other RNA
ggtcagcttgccgtaggtggcatcgccctc

SEQ ID NO: 361 - RNA, 30 nt, synthetic construct
  source 1..30: mol_type = other RNA
ggtcacgagggtgggccagggcacgggcag

SEQ ID NO: 362 - RNA, 30 nt, synthetic construct
  source 1..30: mol_type = other RNA
ggtagcggctgaagcactgcacgccgtagg

SEQ ID NO: 363 - RNA, 30 nt, synthetic construct
  source 1..30: mol_type = other RNA
gggcatggcggacttgaagaagtcgtgctg

SEQ ID NO: 364 - RNA, 30 nt, synthetic construct
  source 1..30: mol_type = other RNA
ggtgcgctcctggacgtagccttcgggcat

SEQ ID NO: 365 - RNA, 30 nt, synthetic construct
  source 1..30: mol_type = other RNA
ggcgcgggtcttgtagttgccgtcgtcctt

SEQ ID NO: 366 - RNA, 30 nt, synthetic construct
  source 1..30: mol_type = other RNA
ggcgagctgcacgctgccgtcctcgatgtt

SEQ ID NO: 367 - RNA, 30 nt, synthetic construct
  source 1..30: mol_type = other RNA
ggtagtggtcggcgagctgcacgctgccgt

SEQ ID NO: 368 - RNA, 30 nt, synthetic construct
  source 1..30: mol_type = other RNA
gggccgtcgccgatgggggtgttctgctgg

SEQ ID NO: 369 - RNA, 30 nt, synthetic construct
  source 1..30: mol_type = other RNA
gggcagcagcacggggccgtcgccgatggg

SEQ ID NO: 370 - RNA, 30 nt, synthetic construct
  source 1..30: mol_type = other RNA
gggggtgttctgctggtagtggtcggcgag

SEQ ID NO: 371 - RNA, 30 nt, synthetic construct
  source 1..30: mol_type = other RNA
ctcggtgaaggcctcccagccgagtgtttt

SEQ ID NO: 372 - RNA, 30 nt, synthetic construct
  source 1..30: mol_type = other RNA
cggtcagcacgcccccgtcctcgtatgtgg

SEQ ID NO: 373 - RNA, 30 nt, synthetic construct
  source 1..30: mol_type = other RNA
cagatggctcccgcccacgagcttcagggc

SEQ ID NO: 374 - RNA, 30 nt, synthetic construct
  source 1..30: mol_type = other RNA
ccatgtcgtttctgccttccaggccgccgt

SEQ ID NO: 375 - RNA, 30 nt, synthetic construct
  source 1..30: mol_type = other RNA
cgatcagatggctcccgcccacgagcttca

SEQ ID NO: 376 - RNA, 30 nt, synthetic construct
  source 1..30: mol_type = other RNA
ctcgttgttggcctccttgattctttccag

SEQ ID NO: 377 - RNA, 30 nt, synthetic construct
  source 1..30: mol_type = other RNA
caattaagcttgtgccccagtttgctaggg

SEQ ID NO: 378 - RNA, 30 nt, synthetic construct
  source 1..30: mol_type = other RNA
cccatgtgaagccctcagggaaggactgct

SEQ ID NO: 379 - RNA, 30 nt, synthetic construct
  source 1..30: mol_type = other RNA
atgtgaagttcacccctctgatcttgacgt

SEQ ID NO: 380 - RNA, 30 nt, synthetic construct
  source 1..30: mol_type = other RNA
ggcgaaggggagagggccgccctcgaccac

SEQ ID NO: 381 - RNA, 30 nt, synthetic construct; identical to SEQ ID NO: 361
  source 1..30: mol_type = other RNA

SEQ ID NO: 382 - RNA, 30 nt, synthetic construct
  source 1..30: mol_type = other RNA
ggtgactctctcccatgtgaagccctcagg

SEQ ID NO: 383 - RNA, 30 nt, synthetic construct
  source 1..30: mol_type = other RNA
ggggtacagggtctcggtgaaggcctccca

SEQ ID NO: 384 - RNA, 30 nt, synthetic construct
  source 1..30: mol_type = other RNA
ggtttcttggatctatatgtggtcttgatg

SEQ ID NO: 385 - RNA, 30 nt, synthetic construct
  source 1..30: mol_type = other RNA
ggtctgggtgccctcgtagggcttgccttc

SEQ ID NO: 386 - RNA, 30 nt, synthetic construct
  source 1..30: mol_type = other RNA
ggatgtcgaaggcgaaggggagagggccgc

SEQ ID NO: 387 - RNA, 30 nt, synthetic construct
  source 1..30: mol_type = other RNA
ggtcttgctgccgtagaggaagctagtagc

SEQ ID NO: 388 - RNA, 30 nt, synthetic construct
  source 1..30: mol_type = other RNA
gggatgccctgggtgtggttgatgaaggtc

SEQ ID NO: 389 - RNA, 30 nt, synthetic construct
  source 1..30: mol_type = other RNA
gggaggtcgcagtatctggccactgccacc

SEQ ID NO: 390 - RNA, 23 nt, synthetic construct
  source 1..23: mol_type = other RNA
tcctcgaagttcatcacgcgctc

SEQ ID NO: 391 - RNA, 23 nt, synthetic construct
  source 1..23: mol_type = other RNA
cgaagttcatcacgcgctcccac

SEQ ID NO: 392 - RNA, 23 nt, synthetic construct
  source 1..23: mol_type = other RNA
tcgaagttcatcacgcgctccca

SEQ ID NO: 393 - RNA, 23 nt, synthetic construct
  source 1..23: mol_type = other RNA
gaagttcatcacgcgctcccact

SEQ ID NO: 394 - RNA, 23 nt, synthetic construct
  source 1..23: mol_type = other RNA
cgtcctcgaagttcatcacgcgc

SEQ ID NO: 395 - RNA, 23 nt, synthetic construct
  source 1..23: mol_type = other RNA
ctcgaagttcatcacgcgctccc

SEQ ID NO: 396 - RNA, 23 nt, synthetic construct
  source 1..23: mol_type = other RNA
cctcgaagttcatcacgcgctcc

SEQ ID NO: 397 - RNA, 23 nt, synthetic construct
  source 1..23: mol_type = other RNA
gtcctcgaagttcatcacgcgct

SEQ ID NO: 398 - RNA, 23 nt, synthetic construct
  source 1..23: mol_type = other RNA
ccgtcctcgaagttcatcacgcg

SEQ ID NO: 399 - RNA, 23 nt, synthetic construct
  source 1..23: mol_type = other RNA
gccgtcctcgaagttcatcacgc

SEQ ID NO: 400 - DNA, 711 bp, synthetic construct
  source 1..711: mol_type = other DNA
atggtgagcatggacgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 401 - DNA, 3309 bp, Homo sapiens
  source 1..3309: mol_type = other DNA
cgtacgcgtgctcacgtgacgggtccgcgaggcccagctcgcgcagtcgttcgggtgagcgaagatggcggccgagagggaacctcctccgctgggggacgggaagcccaccgactttgaggatctggaggacggagaggacctgttcaccagcactgtctccaccctagagtcaagtccatcatctccagaaccagctagtcttcctgcagaagatattagtgcaaactccaatggcccaaaacccacagaagttgtattagatgatgacagagaagatctttttgcagaagccacagaagaagtttctttggacagccctgaaagggaacctatcctatcctcggaaccttctcctgcagtcacacctgtcactcctactacactcattgctcctagaattgaatcaaagagtatgtctgctcccgtgatctttgatagatccagggaagagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtagctcgagctact

SEQ ID NO: 402 - DNA, 4593 bp, Homo sapiens
  source 1..4593: mol_type = other DNA
atggtggacccggtgggcttcgcggaggcgtggaaggcgcagttcccggactcagagcccccgcgcatggagctgcgctcagtgggcgacatcgagcaggagctggagcgctgcaaggcctccattcggcgcctggagcaggaggtgaaccaggagcgcttccgcatgatctacctgcagacgttgctggccaaggaaaagaagagctatgaccggcagcgatggggcttccggcgcgcggcgcaggcccccgacggcgcctccgagccccgagcgtccgcgtcgcgcccgcagccagcgcccgccgacggagccgacccgccgcccgccgaggagcccgaggcccggcccgacggcgagggttctccgggtaaggccaggcccgggaccgcccgcaggcccggggcagccgcgtcgggggaacgggacgaccggggaccccccgccagcgtggcggcgctcaggtccaacttcgagcggatccgcaagggccatggccagcccggggcggacgccgagaagcccttctacgtgaacgtcgagtttcaccacgagcgcggcctggtgaaggtcaacgacaaagaggtgtcggaccgcatcagctccctgggcagccaggccatgcagatggagcgcaaaaagtcccagcacggcgcgggctcgagcgtgggggatgcatccaggcccccttaccggggacgctcctcggagagcagctgcggcgtcgacggcgactacgaggacgccgagttgaacccccgcttcctgaaggacaacctgatcgacgccaatggcggtagcaggcccccttggccgcccctggagtaccagccctaccagagcatctacgtcgggggcatgatggaaggggagggcaagggcccgctcctgcgcagccagagcacctctgagcaggagaagcgccttacctggccccgcaggtcctactccccccggagttttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctactcgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtag

SEQ ID NO: 403 - DNA, 4886 bp, synthetic construct
  source 1..4886: mol_type = other DNA
acgcgtagcgtcttcttcgcttttgcctcgaccgccccttgaccacagacatgtctcgggatcggttccggagtcgtggcggtggcggtggtggcttccacaggcgtggaggaggcggcggccgcggcggcctccacgacttccgttctccgccgcccggcatgggcctcaatcagaatcgcggccccatgggtcctggcccgggccagagcggccctaagcctccgatcccgccaccgcctccacaccaacagcagcaacagccaccaccgcagcagccaccgccgcagcagccgccaccgcatcagccgccgccgcatccacagccgcatcagcagcagcagccgccgccaccgccgcaggactcttccaagcccgtcgttgctcagggacccggccccgctcccggagtaggcagcgcaccaccagcctccagctcggccccgcccgccactccaccaacctcgggggccccgccagggtccgggccaggcccgactccgaccccgccgcctgcagtcacctcggcccctcccggggcgccgccacccaccccgccaagcagcggggtccctaccacacctcctcaggccggaggcccgccgcctccgcccgcggcagtcccgggcccgggtccagggcctaagcagggcccaggtccgggtggtcccaaaggcggcaaaatgcctggcgggccgaagccaggtggcggcccgggcctaagtacgcctggcggccaccccaagccgccgcatcgaggcggcggggagccccgcgggggccgccagcaccacccgccctaccaccagcagcatcaccaggggcccccgcccggcgggcccggcggccgcagcgaggagaagatctcggactcggaggggtttaaagccaatttgtctctcttgaggaggcctggagagaaaacttacacacagcgatgtcggttgtttgttgggaatctacctgctgatatcacggaggatgaattcaaaagactatttgctaaatatggagaaccaggagaagtttttatcaacaaaggcaaaggattcggatttattaagcttgaatctagagctttggctgaaattgccaaagccgaactggatgatacacccatgagaggtagacagcttcgagttcgctttgccacacatgctgctgccctttctgttcgtaatctttcaccttatgtttccaatgaactgttggaagaagcctttagccaatttggtcctattgaaagggctgttgtaatagtggatgatcgtggaagatctacagggaaaggcattgttgaatttgcttctaagccagcagcaagaaaggcatttgaacgatgcagtgaaggtgttttcttactgacgacaactcctcgtccagtcattgtggaaccacttgaacaactagatgatgaagatggtcttcctgaaaaacttgcccagaagaatccaatgtatcaaaaggagagagaaacccctcctcgttttgcccagcatggcacgtttgagtacgaatattctcagcgatggaagtctttggatgaaatggaaaaacagcaaagggaacaagttgaaaaaaacatgaaagatgcaaaagacaaattggaaagtgaaatggaagatgcctatcatgaacatcaggcaaatcttttgcgccaagatctgatgagacgacaggaagaattaagacgcatggaagaacttcacaatcaagaaatgcagaaacgtaaagaaatgcaattgaggcaagaggaggaacgacgtagaagagaggaagagatgatgattcgtcaacgtgagatggaagaacaaatgaggcgccaaagagaggaaagttacagccgaatgggctacatggatccacgggaaagagacatgcgaatgggtggcggaggagcaatgaacatgggagatccctatggttcaggaggccagaaatttccacctctaggaggtggtggtggcataggttatgaagctaatcctggcgttccaccagcaaccatgagtggttccatgatgggaagtgacatgctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctagtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctagagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgaccgttcccaggtgtatgagctgctagagaaggactaccgcatgaagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgactaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgtctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagcctggaaaggcagtgccttagggacccctgctgcagctgagccagtgacccccaccagcaaagcaggctcaggtgcaccaaggggcaccagcaagggccccgccgaggagtccagagtgaggaggcacaagcactcctctgagtcgccagggagggacaaggggaaattgtccaagctcaaacctgccccgccgcccccaccagcagcctctgcagggaaggctggaggaaagccctcgcagaggcccggccaggaggctgccggggaggcagtcttgggcgcaaagacaaaagccacgagtctggttgatgctgtgaacagtgacgctgccaagcccagccagccggcagagggcctcaaaaagcccgtgctcccggccactccaaagccacaccccgccaagccgtcggggacccccatcagcccagcccccgttcccctttccacgttgccatcagcatcctcggccttggcaggggaccagccgtcttccactgccttcatccctctcatatcaacccgagtgtctcttcggaaaacccgccagcctccagagcgggccagcggcgccatcaccaagggcgtggtcttggacagcaccgaggcgctgtgcctcgccatctctgggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgtcagcaggcagtggtccggcggccactcaggacttcagcaagctcctcagttcggtgaaggaaatcagtgacatagtgcagaggtagctcgag

SEQ ID NO: 404 - DNA, 474 bp, synthetic construct
  source 1..474: mol_type = other DNA
atggggcagcagcctggaaaagtacttggggaccaaagaaggccaagcttgcctgccctgcattttatcaaaggagcagggaagaaggaatcatcgaggcatgggggtccacactgcaatgtttttgtggaacatgaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggtagttcctacccatacgatgttccagattacgcttatccctacgacgtgcctgattatgcatacccatatgatgtccccgactatgcctaa

SEQ ID NO: 405 - DNA, 474 bp, synthetic construct
  source 1..474: mol_type = other DNA
atgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccacctcagcagcaagggcaggggcagccgggatgcgctggtctcgggagccctggagtccactaaagcgagtggtagttcctacccatacgatgttccagattacgcttatccctacgacgtgcctgattatgcatacccatatgatgtccccgactatgcctaa

SEQ ID NO: 406 - DNA, 474 bp, synthetic construct
  source 1..474: mol_type = other DNA
atgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctcggtagttcctacccatacgatgttccagattacgcttatccctacgacgtgcctgattatgcatacccatatgatgtccccgactatgcctaa

SEQ ID NO: 407 - DNA, 20 bp, synthetic construct
  source 1..20: mol_type = other DNA
tggctctcctcaagcgtatt

SEQ ID NO: 408 - DNA, 17 bp, synthetic construct
  source 1..17: mol_type = other DNA
caggaaacagctatgac

SEQ ID NO: 409 - DNA, 18 bp, synthetic construct
  source 1..18: mol_type = other DNA
cagatctggcccaacgat

SEQ ID NO: 410 - DNA, 20 bp, synthetic construct
  source 1..20: mol_type = other DNA
cctgaggctcaaagtcagat

SEQ ID NO: 411 - DNA, 20 bp, synthetic construct
  source 1..20: mol_type = other DNA
tggttccatgatgggaagtg

SEQ ID NO: 412 - DNA, 21 bp, synthetic construct
  source 1..21: mol_type = other DNA
ataatggagcgtggtgatgag

SEQ ID NO: 413 - DNA, 22 bp, synthetic construct
  source 1..22: mol_type = other DNA
ctgctcccgtgatctttgatag

SEQ ID NO: 414 - DNA, 22 bp, synthetic construct
  source 1..22: mol_type = other DNA
gataatggagcgtggtgatgag

SEQ ID NO: 415 - DNA, 22 bp, synthetic construct
  source 1..22: mol_type = other DNA
agaaattgcccaactcatgtcc

SEQ ID NO: 416 - DNA, 23 bp, synthetic construct
  source 1..23: mol_type = other DNA
atcaactcccgaaggaaaatctc

SEQ ID NO: 417 - DNA, 21 bp, synthetic construct
  source 1..21: mol_type = other DNA
ggagcgagatccctccaaaat

SEQ ID NO: 418 - DNA, 23 bp, synthetic construct
  source 1..23: mol_type = other DNA
ggctgttgtcatacttctcatgg

SEQ ID NO: 419 - RNA, 30 nt, synthetic construct
  source 1..30: mol_type = other RNA
ggtctctgtagctagaccaaaatcacctat

SEQ ID NO: 420 - RNA, 31 nt, synthetic construct
  source 1..31: mol_type = other RNA
gggagatttctctgtagctagaccaaaatca

SEQ ID NO: 421 - RNA, 30 nt, synthetic construct
  source 1..30: mol_type = other RNA
ggtccatcgagatttctctgtagctagacc

SEQ ID NO: 422 - RNA, 30 nt, synthetic construct
  source 1..30: mol_type = other RNA
ggacccactccatcgagatttctctgtagc

SEQ ID NO: 423 - RNA, 30 nt, synthetic construct
  source 1..30: mol_type = other RNA
ggatgggacccactccatcgagatttctct

SEQ ID NO: 424 - RNA, 30 nt, synthetic construct
  source 1..30: mol_type = other RNA
ggagtctgtagctagaccaaaatcacctat

SEQ ID NO: 425 - RNA, 30 nt, synthetic construct
  source 1..30: mol_type = other RNA
ggcgtctgtagctagaccaaaatcacctat

SEQ ID NO: 426 - RNA, 30 nt, synthetic construct
  source 1..30: mol_type = other RNA
ggtctgagtagctagaccaaaatcacctat

SEQ ID NO: 427 - RNA, 30 nt, synthetic construct
  source 1..30: mol_type = other RNA
ggtctgactagctagaccaaaatcacctat

SEQ ID NO: 428 - RNA, 30 nt, synthetic construct
  source 1..30: mol_type = other RNA
ggtctgacaagctagaccaaaatcacctat

SEQ ID NO: 429 - RNA, 30 nt, synthetic construct
  source 1..30: mol_type = other RNA
ggtctgcgtagctagaccaaaatcacctat

SEQ ID NO: 430 - RNA, 30 nt, synthetic construct
  source 1..30: mol_type = other RNA
ggtctgcctagctagaccaaaatcacctat

SEQ ID NO: 431 - RNA, 30 nt, synthetic construct
  source 1..30: mol_type = other RNA
ggtctgcccagctagaccaaaatcacctat

SEQ ID NO: 432 - RNA, 30 nt, synthetic construct
  source 1..30: mol_type = other RNA
ggtgtctctagctagaccaaaatcacctat

SEQ ID NO: 433 - RNA, 30 nt, synthetic construct
  source 1..30: mol_type = other RNA
ggtgtctgtacctagaccaaaatcacctat

SEQ ID NO: 434 - RNA, 30 nt, synthetic construct
  source 1..30: mol_type = other RNA
ggtgtctgtagctacaccaaaatcacctat

SEQ ID NO: 435 - RNA, 30 nt, synthetic construct
  source 1..30: mol_type = other RNA
ggtctctctacctagaccaaaatcacctat

SEQ ID NO: 436 - RNA, 30 nt, synthetic construct
  source 1..30: mol_type = other RNA
ggtctctctagctacaccaaaatcacctat

SEQ ID NO: 437 - RNA, 30 nt, synthetic construct
  source 1..30: mol_type = other RNA
ggtctctgtacctacaccaaaatcacctat

SEQ ID NO: 438 - RNA, 30 nt, synthetic construct
  source 1..30: mol_type = other RNA
ggtctctctacctacaccaaaatcacctat

SEQ ID NO: 439 - RNA, 30 nt, synthetic construct
  source 1..30: mol_type = other RNA
ggtgtctgtagctacaccaaaatcagctat

SEQ ID NO: 440 - RNA, 30 nt, synthetic construct
  source 1..30: mol_type = other RNA
ggtctgtgtagctagagcaaaatcaccaat

SEQ ID NO: 441 - RNA, 30 nt, synthetic construct
  source 1..30: mol_type = other RNA
ggtctctctagctacaccaaattcacctat

SEQ ID NO: 442 - RNA, 30 nt, synthetic construct
  source 1..30: mol_type = other RNA
ggtgtctgtacctagacgaaaatctcctat

SEQ ID NO: 443 - RNA, 30 nt, synthetic construct
  source 1..30: mol_type = other RNA
ggtctgtgtagcaagaccataatcagctat

SEQ ID NO: 444 - RNA, 30 nt, synthetic construct
  source 1..30: mol_type = other RNA
ggtctctgtagctacaccaaattcaccaat

SEQ ID NO: 445 - RNA, 30 nt, synthetic construct
  source 1..30: mol_type = other RNA
ggtctctgtagctagagcaaaatgacctaa

SEQ ID NO: 446 - RNA, 21 nt, synthetic construct
  source 1..21: mol_type = other RNA
gtagattgctgttctaccaag

SEQ ID NO: 447 - RNA, 20 nt, synthetic construct
  source 1..20: mol_type = other RNA
tagctacagagaaatctcga

SEQ ID NO: 448 - RNA, 20 nt, synthetic construct
  source 1..20: mol_type = other RNA
acagagaaatctcgatggag

SEQ ID NO: 449 - DNA, 28 bp, synthetic construct
  source 1..28: mol_type = other DNA
ccatatcattgagaccaaatttgagatg

SEQ ID NO: 450 - DNA, 27 bp, synthetic construct
  source 1..27: mol_type = other DNA
ggcactctgccattaatctcttcatgg

SEQ ID NO: 451 - protein, 1090 aa, Prevotella pectinovora
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGVVEIKALPEIAMSIKKAFGEYAIMK

SEQ ID NO: 452 - DNA, 3273 bp, Prevotella pectinovora
  source 1..3273: mol_type = other DNA
atgaatatacctgcattagtggaaaaccagaagaagtatttcggtacatactctgtcatggcaatgctgaacgcacagaccgtactcgaccacatacagaaagtggcagacatagagggggaacagaacgagaacaacgaaaatctatggttccaccccgtaatgagccatttatacaatgcgaagaatggatatgacaaacagccggaaaagactatgttcatcatcgaacgtctgcagtcatattttcctttcctcaagattatggctgaaaaccaaagagagtattcgaacggaaaatacaaacagaatagggttgaggtgaacagcaacgacatctttgaagttctgaaaagggctttcggcgtgctgaaaatgtatcgcgacctaaccaaccattacaaaacctacgaagaaaagctgaacgacggctgcgagtttctgacgagtactgagcagcctctttccggaatgataaacaactactacactgtagccctgcgaaacatgaatgagcgttatggatacaaaaccgaagacctcgcctttatacaggacaaaagattcaaattcgtcaaagacgcatacggaaaaaagaaatcacaagtaaataccggtttcttcctcagtctgcaagactacaacggcgacacacaaaagaaactccacttgtctggagtcggcatagccttgcttatctgtctgttcctcgacaagcagtatatcaacatcttcctgtcaaggcttcctatcttctcttcctataatgcacaatcggaagagcgacgcatcatcatccgttcttttggcataaacagcatcaaactgcccaaagaccgcatccactctgaaaagagcaacaagtcggtggctatggatatgctgaatgaggtgaagcgttgtccggacgaactgttcactaccctctcggctgaaaagcagtctcgattcagaattatttccgacgaccacaacgaggtgcttatgaaacgtagttccgacagattcgtgccattgctattgcaatatattgactacggcaagctgttcgaccacatccgttttcatgtgaatatggggaaactgcgctatctgctcaaagccgacaagacctgcatcgacggtcagacacgagtgagggtgatagaacagcctctcaacggtttcggaagattggaagaggctgaaacgatgcgaaagcaagaaaacggaactttcggcaatagcggcattcgcattcgcgattttgagaacatgaaaagagacgacgccaatccggcaaactatccatatatcgtggacacatatactcactatatattggagaacaacaaggtggaaatgttcataaacgataaagaagactcagccccattgttgcctgtgattgaggatgacagatatgtggtaaagaccattccatcctgccgtatgagcactcttgaaatacctgccatggcatttcatatgttcctgttcggtagcaaaaagaccgaaaagcttattgtcgatgttcacaacaggtataagagattgttccaagcgatgcaaaaagaagaggtaacagctgaaaacatagcctctttcggcattgctgaaagcgatctgccccagaagattcttgatctcatcagcggaaatgctcatggcaaggatgttgacgccttcatcaggcttaccgtcgacgacatgcttaccgataccgaacggcgcatcaagagatttaaagacgaccgcaaatccatacgttctgccgacaacaagatgggcaagcgcggtttcaaacagatttctacaggtaagctggcggatttcctcgccaaagacatcgttttgttccaaccttctgtaaatgatggcgagaacaagattacgggattgaactaccgcatcatgcagtctgccatcgccgtttacgacagtggcgacgattatgaagccaagcaacagttcaagctgatgtttgagaaagcccggctcatcggcaaaggcacaacagagcctcatcctttcttatataaggtctttgcgagatctatccctgccaatgccgtggagttctatgagaggtatcttatagagcgcaaattctatctcaccggactctccaacgagataaagaagggaaacagggtggatgtgcctttcatcagacgtgaccagaacaaatggaaaacaccagcgatgaagactctcggcagaatctactctgaagacctgccggtggagttgcctcgacagatgtttgacaacgaaatcaagagccacctgaaatcgctgccacagatggagggcattgatttcaacaatgccaacgtcacgtatctcattgcggaatatatgaaacgggtattggatgatgatttccagacattctaccaatggaaccgcaactatcgctatatggacatgctgaagggtgaatacgacaggaaaggttcgctgcagcactgcttcacatccgtagaagaaagagaaggtttatggaaagaacgtgcaagccggactgaacgctacaggaaacaggcttcaaacaaaatcaggtctaacagacagatgcgtaatgcctcttctgaagaaatcgaaacgatattagacaagcgtttgagtaacagccgcaacgaataccagaaaagtgagaaggtgatccgcagatacagagtacaggatgccctgctcttcttattggcaaagaagactctgacggaactggcggatttcgacggcgagagattcaagctgaaagagattatgcctgatgcagaaaagggaatcctgtctgagattatgcctatgtcattcacttttgaaaagggcggcaagaaatataccataacatcggaaggcatgaagctgaagaactatggtgatttcttcgtcttggcaagcgacaagagaatcggcaacttgttagaacttgttggctccgacattgtatccaaggaggacatcatggaagaattcaataagtatgaccaatgccgacctgaaatatcgagcatcgtattcaacttagagaaatgggcattcgacacttatcctgagttgtcagcgagagttgacagagaagaaaaggttgatttcaagtccatcttaaagattctcctaaacaacaagaatatcaataaagaacaaagtgacatattgcgaaaaatcaggaatgctttcgaccacaacaactatcccgacaaaggtgttgttgagataaaggctttgcctgaaattgccatgagcataaagaaagcattcggagaatatgccatcatgaaatag

SEQ ID NO: 453 - DNA, 2962 bp, synthetic construct
  source 1..2962: mol_type = other DNA
agtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacgcaggaaacagctatgaccatgattacgccagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccatgtcttcctgggacgaagacaagttgtggaaggtccagttttgaggggctattacaacttttttggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttacc

SEQ ID NO: 454 - DNA, 13352 bp, synthetic construct
  source 1..13352: mol_type = other DNA
gaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactggactataaggaccacgacggagactacaaggatcatgatattgattacaaagacgatgacgataagggcggcgggtccggaggagagggcagaggaagtctcctaacatgcggtgacgtggaggagaatcctggcccaatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagaccctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactg

SEQ ID NO: 455 - DNA, 12607 bp, synthetic construct
  source 1..12607: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaactgcctccacttgaaagactgacactgggtagttcctacccatacgatgttccagattacgcttatccctacgacgtgcctgattatgcatacccatatgatgtccccgactatgcctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtt

SEQ ID NO: 456 - DNA, 14268 bp, synthetic construct
  source 1..14268: mol_type = other DNA
ttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaacccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgactctagaggatctgaattctggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagggcggtgagggcagaggaagtctgctaacatgcggtgacgtcgaggagaatcctggcccaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagtgacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagag

SEQ ID NO: 457 - RNA, 30 nt, synthetic construct; identical to SEQ ID NO: 344
  source 1..30: mol_type = other RNA

SEQ ID NO: 458 - RNA, 30 nt, synthetic construct; identical to SEQ ID NO: 335
  source 1..30: mol_type = other RNA

SEQ ID NO: 459 - RNA, 30 nt, synthetic construct; identical to SEQ ID NO: 336
  source 1..30: mol_type = other RNA

SEQ ID NO: 460 - RNA, 30 nt, synthetic construct; identical to SEQ ID NO: 338
  source 1..30: mol_type = other RNA

SEQ ID NO: 461 - RNA, 30 nt, synthetic construct; identical to SEQ ID NO: 341
  source 1..30: mol_type = other RNA

SEQ ID NO: 462 - RNA, 30 nt, synthetic construct
  source 1..30: mol_type = other RNA
tggccgatgaagggcatctgcgtgtccatg

SEQ ID NO: 463 - DNA, 474 bp, synthetic construct; identical to SEQ ID NO: 406
  source 1..474: mol_type = other DNA

SEQ ID NO: 464 - DNA, 474 bp, synthetic construct; identical to SEQ ID NO: 405
  source 1..474: mol_type = other DNA

SEQ ID NO: 465 - RNA, 23 nt, synthetic construct
  source 1..23: mol_type = other RNA
tagattgctgttctaccaagtgg

SEQ ID NO: 466 - RNA, 23 nt, synthetic construct
  source 1..23: mol_type = other RNA
tttctctgtagctagaccaaaat

SEQ ID NO: 467 - RNA, 23 nt, synthetic construct
  source 1..23: mol_type = other RNA
attctctgtagctagaccaaaat

SEQ ID NO: 468 - RNA, 23 nt, synthetic construct
  source 1..23: mol_type = other RNA
tatctctgtagctagaccaaaat

SEQ ID NO: 469 - RNA, 23 nt, synthetic construct
  source 1..23: mol_type = other RNA
ttactctgtagctagaccaaaat

SEQ ID NO: 470 - RNA, 23 nt, synthetic construct
  source 1..23: mol_type = other RNA
tttgtctgtagctagaccaaaat

SEQ ID NO: 471 - RNA, 23 nt, synthetic construct
  source 1..23: mol_type = other RNA
tttctgtgtagctagaccaaaat

SEQ ID NO: 472 - RNA, 23 nt, synthetic construct
  source 1..23: mol_type = other RNA
tttctcagtagctagaccaaaat

SEQ ID NO: 473 - RNA, 23 nt, synthetic construct
  source 1..23: mol_type = other RNA
tttctctctagctagaccaaaat

SEQ ID NO: 474 - RNA, 23 nt, synthetic construct
  source 1..23: mol_type = other RNA
tttctctgaagctagaccaaaat

SEQ ID NO: 475 - RNA, 23 nt, synthetic construct
  source 1..23: mol_type = other RNA
tttctctgttgctagaccaaaat

SEQ ID NO: 476 - RNA, 23 nt, synthetic construct
  source 1..23: mol_type = other RNA
tttctctgtacctagaccaaaat

SEQ ID NO: 477 - RNA, 23 nt, synthetic construct
  source 1..23: mol_type = other RNA
tttctctgtaggtagaccaaaat

SEQ ID NO: 478 - RNA, 23 nt, synthetic construct
  source 1..23: mol_type = other RNA
tttctctgtagcaagaccaaaat

SEQ ID NO: 479 - RNA, 23 nt, synthetic construct
  source 1..23: mol_type = other RNA
tttctctgtagcttgaccaaaat

SEQ ID NO: 480 - RNA, 23 nt, synthetic construct
  source 1..23: mol_type = other RNA
tttctctgtagctacaccaaaat

SEQ ID NO: 481 - RNA, 23 nt, synthetic construct
  source 1..23: mol_type = other RNA
tttctctgtagctagtccaaaat

SEQ ID NO: 482 - RNA, 23 nt, synthetic construct
  source 1..23: mol_type = other RNA
tttctctgtagctagagcaaaat

SEQ ID NO: 483 - RNA, 23 nt, synthetic construct
  source 1..23: mol_type = other RNA
tttctctgtagctagacgaaaat

SEQ ID NO: 484 - RNA, 23 nt, synthetic construct
  source 1..23: mol_type = other RNA
tttctctgtagctagacctaaat

SEQ ID NO: 485 - RNA, 23 nt, synthetic construct
  source 1..23: mol_type = other RNA
tttctctgtagctagaccataat

SEQ ID NO: 486 - RNA, 23 nt, synthetic construct
  source 1..23: mol_type = other RNA
tttctctgtagctagaccaatat

SEQ ID NO: 487 - RNA, 23 nt, synthetic construct
  source 1..23: mol_type = other RNA
tttctctgtagctagaccaaatt

SEQ ID NO: 488 - RNA, 23 nt, synthetic construct
  source 1..23: mol_type = other RNA
tttctctgtagctagaccaaaaa

SEQ ID NO: 489 - RNA, 23 nt, synthetic construct
  source 1..23: mol_type = other RNA
cctacgccacaagctccaactac

SEQ ID NO: 490 - RNA, 23 nt, synthetic construct
  source 1..23: mol_type = other RNA
cctacgccagatgctccaactac

SEQ ID NO: 491 - RNA, 23 nt, synthetic construct
  source 1..23: mol_type = other RNA
cctacgcctgaagctccaactac

SEQ ID NO: 492 - RNA, 23 nt, synthetic construct
  source 1..23: mol_type = other RNA
cctacgccataagctccaactac

SEQ ID NO: 493 - RNA, 23 nt, synthetic construct
  source 1..23: mol_type = other RNA
gcctacgccatatgctccaacta

SEQ ID NO: 494 - RNA, 23 nt, synthetic construct
  source 1..23: mol_type = other RNA
cctacgccatatgctccaactac

SEQ ID NO: 495 - RNA, 23 nt, synthetic construct
  source 1..23: mol_type = other RNA
gcctacgccttaagctccaacta

SEQ ID NO: 496 - RNA, 23 nt, synthetic construct
  source 1..23: mol_type = other RNA
cctacgccttaagctccaactac

SEQ ID NO: 497 - RNA, 23 nt, synthetic construct
  source 1..23: mol_type = other RNA
cctacgccaaatgctccaactac

SEQ ID NO: 498 - RNA, 23 nt, synthetic construct
  source 1..23: mol_type = other RNA
cctacgcctaaagctccaactac

SEQ ID NO: 499 - RNA, 23 nt, synthetic construct
  source 1..23: mol_type = other RNA
cctacgccacatgctccaactac

SEQ ID NO: 500 - RNA, 23 nt, synthetic construct
  source 1..23: mol_type = other RNA
cctacgcctcaagctccaactac

SEQ ID NO: 501 - RNA, 23 nt, synthetic construct
  source 1..23: mol_type = other RNA
cctacgccacaagctccaactag

SEQ ID NO: 502 - RNA, 23 nt, synthetic construct
  source 1..23: mol_type = other RNA
cctacgccataagctccaactac

SEQ ID NO: 503 - RNA, 23 nt, synthetic construct
  source 1..23: mol_type = other RNA
gctacgccataagctccaactac

SEQ ID NO: 504 - RNA, 23 nt, synthetic construct
  source 1..23: mol_type = other RNA
cctacgccatatgctccaactac

SEQ ID NO: 505 - RNA, 23 nt, synthetic construct
  source 1..23: mol_type = other RNA
cctacgccataagcaccaactac

SEQ ID NO: 506 - RNA, 23 nt, synthetic construct
  source 1..23: mol_type = other RNA
cctacgccataagctcctactac

SEQ ID NO: 507 - RNA, 23 nt, synthetic construct
  source 1..23: mol_type = other RNA
gctacgccataagctccaactag

SEQ ID NO: 508 - RNA, 23 nt, synthetic construct
  source 1..23: mol_type = other RNA
cctacgccatatgctccaactag

SEQ ID NO: 509 - RNA, 23 nt, synthetic construct
  source 1..23: mol_type = other RNA
cctacgccataagcaccaactag

SEQ ID NO: 510 - RNA, 23 nt, synthetic construct
  source 1..23: mol_type = other RNA
cctacgccataagctcctactag

SEQ ID NO: 511 - RNA, 23 nt, synthetic construct
  source 1..23: mol_type = other RNA
cctacgccataagctccaacaag

SEQ ID NO: 512 - RNA, 23 nt, synthetic construct
  source 1..23: mol_type = other RNA
ccttcgccataagctccaactag

SEQ ID NO: 513 - RNA, 23 nt, synthetic construct
  source 1..23: mol_type = other RNA
cctaccccataagctccaactag

SEQ ID NO: 514 - RNA, 23 nt, synthetic construct
  source 1..23: mol_type = other RNA
cctacgccttaagctccaactag

SEQ ID NO: 515 - RNA, 23 nt, synthetic construct
  source 1..23: mol_type = other RNA
cctacgccataagctccaacaac

SEQ ID NO: 516 - RNA, 23 nt, synthetic construct
  source 1..23: mol_type = other RNA
cctacgccataagctccaactag

SEQ ID NO: 517 - RNA, 23 nt, synthetic construct
  source 1..23: mol_type = other RNA
ccttcgccataagctccaactac

SEQ ID NO: 518 - RNA, 23 nt, synthetic construct
  source 1..23: mol_type = other RNA
cctaccccataagctccaactac

SEQ ID NO: 519 - RNA, 23 nt, synthetic construct
  source 1..23: mol_type = other RNA
cctacgccttaagctccaactac

SEQ ID NO: 520 - RNA, 23 nt, synthetic construct
  source 1..23: mol_type = other RNA
cctacgccatcagctccaactac

SEQ ID NO: 521 - RNA, 23 nt, synthetic construct
  source 1..23: mol_type = other RNA
gcctacgccttgagctccaacta

SEQ ID NO: 522 - RNA, 23 nt, synthetic construct
  source 1..23: mol_type = other RNA
gcctacgcgttcagctccaacta

SEQ ID NO: 523 - RNA, 23 nt, synthetic construct
  source 1..23: mol_type = other RNA
cctacgccatgtgctccaactac

SEQ ID NO: 524 - RNA, 23 nt, synthetic construct
  source 1..23: mol_type = other RNA
cctacgccttgagctccaactac

SEQ ID NO: 525 - RNA, 23 nt, synthetic construct
  source 1..23: mol_type = other RNA
cctacgccatttgctccaactac

SEQ ID NO: 526 - RNA, 23 nt, synthetic construct
  source 1..23: mol_type = other RNA
cctacgcctttagctccaactac

SEQ ID NO: 527 - RNA, 23 nt, synthetic construct
  source 1..23: mol_type = other RNA
cctacgccatctgctccaactac

SEQ ID NO: 528 - RNA, 23 nt, synthetic construct
  source 1..23: mol_type = other RNA
cctacgccttcagctccaactac

SEQ ID NO: 529 - RNA, 23 nt, synthetic construct
  source 1..23: mol_type = other RNA
gctacgccatcagctccaactac

SEQ ID NO: 530 - RNA, 23 nt, synthetic construct
  source 1..23: mol_type = other RNA
cctacgccatgagctccaactac

SEQ ID NO: 531 - RNA, 23 nt, synthetic construct
  source 1..23: mol_type = other RNA
cctacgccatctgctccaactac

SEQ ID NO: 532 - RNA, 23 nt, synthetic construct
  source 1..23: mol_type = other RNA
cctacgccatcacctccaactac

SEQ ID NO: 533 - RNA, 23 nt, synthetic construct
  source 1..23: mol_type = other RNA
cctacgccatcaggtccaactac

SEQ ID NO: 534 - RNA, 23 nt, synthetic construct
  source 1..23: mol_type = other RNA
cctacgccatcagcaccaactac

SEQ ID NO: 535 - RNA, 23 nt, synthetic construct
  source 1..23: mol_type = other RNA
cctacgccatcagctgcaactac

SEQ ID NO: 536 - RNA, 23 nt, synthetic construct
  source 1..23: mol_type = other RNA
cctacgccatcagctcgaactac

SEQ ID NO: 537 - RNA, 23 nt, synthetic construct
  source 1..23: mol_type = other RNA
cctacgccatcagctcctactac

SEQ ID NO: 538 - RNA, 23 nt, synthetic construct
  source 1..23: mol_type = other RNA
cctacgccatcagctccatctac

SEQ ID NO: 539 - RNA, 23 nt, synthetic construct
  source 1..23: mol_type = other RNA
cgtacgccatcagctccaactac

SEQ ID NO: 540 - RNA, 23 nt, synthetic construct
  source 1..23: mol_type = other RNA
cctacgccatcagctccaagtac

SEQ ID NO: 541 - RNA, 23 nt, synthetic construct
  source 1..23: mol_type = other RNA
cctacgccatcagctccaacaac

SEQ ID NO: 542 - RNA, 23 nt, synthetic construct
  source 1..23: mol_type = other RNA
cctacgccatcagctccaacttc

SEQ ID NO: 543 - RNA, 23 nt, synthetic construct
  source 1..23: mol_type = other RNA
cctacgccatcagctccaactag

SEQ ID NO: 544 - RNA, 23 nt, synthetic construct
  source 1..23: mol_type = other RNA
cctacgccatcagctccaactag

SEQ ID NO: 545 - RNA, 23 nt, synthetic construct
  source 1..23: mol_type = other RNA
ccaacgccatcagctccaactac

SEQ ID NO: 546 - RNA, 23 nt, synthetic construct
  source 1..23: mol_type = other RNA
ccttcgccatcagctccaactac

SEQ ID NO: 547 - RNA, 23 nt, synthetic construct
  source 1..23: mol_type = other RNA
cctaggccatcagctccaactac

SEQ ID NO: 548 - RNA, 23 nt, synthetic construct
  source 1..23: mol_type = other RNA
cctaccccatcagctccaactac

SEQ ID NO: 549 - RNA, 23 nt, synthetic construct
  source 1..23: mol_type = other RNA
cctacggcatcagctccaactac

SEQ ID NO: 550 - RNA, 23 nt, synthetic construct
  source 1..23: mol_type = other RNA
cctacgcgatcagctccaactac

SEQ ID NO: 551 - RNA, 23 nt, synthetic construct
  source 1..23: mol_type = other RNA
cctacgccttcagctccaactac

SEQ ID NO: 552 - RNA, 23 nt, synthetic construct
  source 1..23: mol_type = other RNA
cctacgcctatagctccaactac

SEQ ID NO: 553 - RNA, 23 nt, synthetic construct
  source 1..23: mol_type = other RNA
cctacgcctagagctccaactac

SEQ ID NO: 554 - RNA, 23 nt, synthetic construct
  source 1..23: mol_type = other RNA
cctacgccaattgctccaactac

SEQ ID NO: 555 - RNA, 23 nt, synthetic construct
  source 1..23: mol_type = other RNA
cctacgccaagtgctccaactac

SEQ ID NO: 556 - RNA, 23 nt, synthetic construct
  source 1..23: mol_type = other RNA
gcctacgcctatagctccaacta

SEQ ID NO: 557 - RNA, 23 nt, synthetic construct
  source 1..23: mol_type = other RNA
gcctacgcctagagctccaacta

SEQ ID NO: 558 - RNA, 23 nt, synthetic construct
  source 1..23: mol_type = other RNA
gcctacgccaattgctccaacta

SEQ ID NO: 559 - RNA, 23 nt, synthetic construct
  source 1..23: mol_type = other RNA
cctacgcctaaagctccaactag

SEQ ID NO: 560 - RNA, 23 nt, synthetic construct
  source 1..23: mol_type = other RNA
cctacgccaaatgctccaactag

SEQ ID NO: 561 - DNA, 2898 bp, Ruminococcus flavefaciens
  source 1..2898: mol_type = other DNA
atcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattcc

SEQ ID NO: 562 - DNA, 669 bp, synthetic construct
  source 1..669: mol_type = other DNA
ggatccatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgggtagttcctacccatacgatgttccagattacgcttatccctacgacgtgcctgattatgcatacccatatgatgtccccgactatgcctaactcgag

SEQ ID NO: 563 - DNA, 669 bp, synthetic construct
  source 1..669: mol_type = other DNA
ggatccatgactgaatataaacttgtggtagttggagctagtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgggtagttcctacccatacgatgttccagattacgcttatccctacgacgtgcctgattatgcatacccatatgatgtccccgactatgcctaactcgag

SEQ ID NO: 564 - DNA, 669 bp, synthetic construct
  source 1..669: mol_type = other DNA
ggatccatgactgaatataaacttgtggtagttggagcttgtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgggtagttcctacccatacgatgttccagattacgcttatccctacgacgtgcctgattatgcatacccatatgatgtccccgactatgcctaactcgag

SEQ ID NO: 565 - DNA, 669 bp, synthetic construct
  source 1..669: mol_type = other DNA
ggatccatgactgaatataaacttgtggtagttggagctgatggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgggtagttcctacccatacgatgttccagattacgcttatccctacgacgtgcctgattatgcatacccatatgatgtccccgactatgcctaactcgag

SEQ ID NO: 566 - DNA, 669 bp, synthetic construct
  source 1..669: mol_type = other DNA
ggatccatgactgaatataaacttgtggtagttggagctcgtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgggtagttcctacccatacgatgttccagattacgcttatccctacgacgtgcctgattatgcatacccatatgatgtccccgactatgcctaactcgag

SEQ ID NO: 567 - DNA, 669 bp, synthetic construct; identical to SEQ ID NO: 563
  source 1..669: mol_type = other DNA

SEQ ID NO: 568 - DNA, 669 bp, synthetic construct
  source 1..669: mol_type = other DNA
ggatccatgactgaatataaacttgtggtagttggagctgttggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgggtagttcctacccatacgatgttccagattacgcttatccctacgacgtgcctgattatgcatacccatatgatgtccccgactatgcctaactcgag